(19) United States
(12) Patent Application Publication
(10) Pub. No.: US 2026/0272918 A1
(43) Pub. Date: Sep. 17, 2026

(54) PYRAZOLE-FUSED COMPOUNDS AS VRK1 INHIBITORS

(71) Applicant: Incyte Corporation, Wilmington, DE (US)

(72) Inventors: Yong Li, Newark, DE (US); Yanran Ai, West Chester, PA (US); Huan Chen, Glen Mills, PA (US); Kelly Federowicz, Garnet Valley, PA (US); Yehao Qiu, Wilmington, DE (US); Leslie Brooke Epling McQuirter, Wilmington, DE (US); Cooper Ashley Taylor, Wilmington, DE (US); Xiaozhao Wang, Moorestown, NJ (US); Eddy W. Yue, Landenberg, PA (US); Guofeng Zhang, Kennett Square, PA (US); Le Zhao, Wilmington, DE (US); Zhitong Zheng, Glen Mills, PA (US)

(21) Appl. No.: 19/566,792

(22) Filed: Mar. 13, 2026

Related U.S. Application Data
(60) Provisional application No. 63/771,190, filed on Mar. 13, 2025.
(60) Provisional application No. 63/771,166, filed on Mar. 13, 2025.
(60) Provisional application No. 63/771,181, filed on Mar. 13, 2025.
(60) Provisional application No. 63/818,492, filed on Jun. 5, 2025.
(60) Provisional application No. 63/938,661, filed on Dec. 11, 2025.
(60) Provisional application No. 63/938,611, filed on Dec. 11, 2025.
(60) Provisional application No. 63/938,639, filed on Dec. 11, 2025.

(51) Int. Cl. A61K 31/4985; A61K 31/499; A61K 31/5377; A61K 31/538; A61P 35/00; C07B 59/00; C07D 487/04; C07D 487/10; C07D 519/00

(52) U.S. Cl. CPC A61K 31/4985; A61K 31/499; A61K 31/5377; A61K 31/538; A61P 35/00; C07B 59/002; C07D 487/04; C07D 487/10; C07D 519/00

(57) ABSTRACT

Disclosed are compounds of Formula (I):

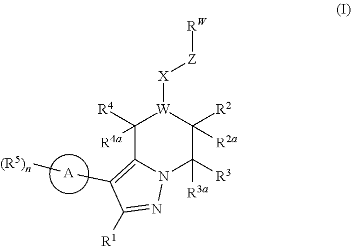

and pharmaceutically acceptable salts thereof, and methods of using the compounds for treatment of cancer and inhibiting VRK1, as well as pharmaceutical compositions comprising such compounds.

### CLAIM OF PRIORITY

This application claims the benefit of priority of U.S. Prov. Appl. Nos. 63/771,166, filed Mar. 13, 2025; 63/771,181, filed Mar. 13, 2025; 63/771,190, filed Mar. 13, 2025; 63/818,492, filed Jun. 5, 2025; 63/938,611, filed Dec. 11, 2025; 63/938,639, filed Dec. 11, 2025; and 63/938,661, filed Dec. 11, 2025, each of which are incorporated herein by reference in their entirety.

### FIELD OF THE DISCLOSURE

The present application is concerned with pharmaceutically active compounds. The disclosure provides compounds as well as their compositions and methods of use. The compounds are inhibitors of VRK1 and are useful in the treatment of various diseases including cancer.

### BACKGROUND

Vaccinia related kinase (VRK) family are characterized by notable sequence homology to the virus-encoded B1 kinase (vvB1). VRK1 is a member of a subgroup of the casein kinase-1 (CK1) family that also contains VRK2 and the pseudokinase VRK3 (J. Biol. Chem. 279:7934-7946 (2004)). VRK1 and VRK2 have been associated with various cellular processes, including cell replication, chromatin remodeling, and DNA damage response (ACS Med. Chem. Lett. 2019, 10, 9, 1266-1271).

VRK1 is a serine/threonine-protein kinase that plays a pivotal role in regulating cell cycle, DNA damage response, nuclear condensation, and transcriptional control. VRK1 has been found to be overexpressed in multiple cancer types, including breast cancer, lung cancer, colorectal cancer, head and neck carcinomas, liver cancer, multiple myeloma, esophageal carcinoma, and all hematopoietic tumors, suggesting its involvement in tumorigenesis. Functionally, VRK1 phosphorylates multiple substrates involved in both cell cycle progression and cell-cycle arrest. VRK1 has been reported to phosphorylate histone H3 and H2AX to regulate chromatin remodeling, transcription factors including p53, c-Jun, ATF2, CREB and Sox2 to activate of transcription, regulate assembly and disassembly of the nuclear envelop by phosphorylating BANF1 in cell cycle progression (Cancer Letters, Volume 503, 10 Apr. 2021, Pages 117-128). Due to its role in cell proliferation and survival of tumor cells, VRK1 has emerged as a potential therapeutic target for cancer treatment.

Genetic knockdown evidence suggests that VRK1 depletion sensitizes cells to cancer treatments based on DNA damage such as ionizing radiation or doxorubicin by impairing the DNA damage response (Cellular and Molecular Life Sciences (2018) 75:2375-2388). Depletion of VRK1 also slows tumor cell proliferation and tumor growth and metastasis in vivo (Oncogenesis volume 2, page 48 (2013)). Genome-scale CRISPR/Cas9 loss of functions screens have identified a paralog-based synthetic lethality role of VRK1 in adult and pediatric gliomas and neuroblastoma, where VRK2 is silenced by promoter methylation (JCI Insight. 2022; 7(19):e158755; Cancer Res. 2022 Nov. 2; 82(21):4044-4057). The VRK1-VRK2 synthetic-lethal interaction was dependent on VKR1 kinase activity and was rescued by ectopic expression of VRK2 (Cancer Res. 2022 Nov. 2; 82(21):4044-4057). These results indicated that inhibiting VRK1 kinase activity with small molecule inhibitors could be a viable therapeutic strategy in VRK2-methylated GBM and neuroblastoma. Other then glioblastoma and neuroblastoma, preclinical data indicated other cancer types where VRK1 plays a critical role in cancer cell proliferation, including but not limited to Ewing Sarcoma, Astrocytoma, Diffuse Glioma, Bone Sarcoma, Solid tumors e.g. esophageal squamous cell carcinoma, colorectal adenocarcinoma (DepMap; Oncotarget. 2017; 8:65642-65658; Cancer Cell 28, 668-681, Nov. 9, 2014; Pathology—Research and Practice 214 (2018) 112-11). These observations suggests that inhibitors of VRK1 can be of potential use in cancer treatments, by themselves or in combinations, by facilitating inhibition of proliferation and at the same time sensitizing cells to treatments based on DNA damage.

Currently, there are no potent and specific small molecule inhibitors of VRK1. Some studies that have made progresses in developing VRK1 inhibitors including: (1) one study discovered an aminopyridine scaffold as a basis for VRK1 inhibitors and the most potent compound in this study had an IC50 value of 150 nM (ACS Med. Chem. Lett. 2019, 10, 9, 1266-1271); (2) Another study developed VRK1 inhibitors based on BI-D1870, a pteridinone inhibitor of RSK kinases. The optimized VRK1 inhibitor #36 was more selective and mimicked the cellular outcomes of VRK1 depletion (J. Med. Chem. 2024, 67, 11, 8609-8629).

Accordingly, there is a need for new compounds that inhibit VRK1 and which are useful in treating cancers. This application address this need and others.

### SUMMARY

The present disclosure provides, inter alia, compounds of Formula (I):

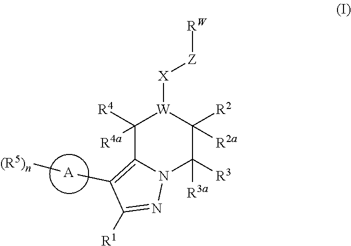

or a pharmaceutically acceptable salt thereof, wherein constituent variables are defined herein.

The present disclosure further provides a VRK1 inhibitor, or a pharmaceutically acceptable salt thereof.

The present disclosure further provides a pharmaceutical composition comprising a compound disclosed herein, or a pharmaceutically acceptable salt thereof, and one or more pharmaceutically acceptable excipient or carrier.

The present disclosure further provides methods of inhibiting VRK1, comprising contacting the VRK1 with a VRK1 inhibitor, or a pharmaceutically acceptable salt thereof.

Alternatively, in the preceding embodiment, R^{W} is a means for covalently binding to cysteine residue in a protein (e.g., VRK1). In some embodiments, R^{W} is a means for covalently binding to cysteine residue in the VRK1 protein.

The present disclosure further provides methods of treating a disease or disorder associated with VRK1 activity, comprising administering to a patient in need thereof a therapeutically effective amount of a compound of disclosed herein, or a pharmaceutically acceptable salt thereof. In some embodiments, the method is a method of treating sarcoma, colorectal adenocarcinoma, acute myeloid leukemia, glioblastoma, or neuroblastoma. In some embodiments, the method is a method of treating bone cancer, cancer of the neck and head, rectal cancer, esophageal cancer, sarcoma of soft tissue, neoplasm of the central nervous system (CNS), brain stem, glioma, colon cancer, Ewing's sarcoma, brain cancer, astrocytoma, neuroblastoma, intestinal cancer, nervous system cancers, osteosarcoma, cancer of the small bowel (adenocarcinoma, lymphoma, carcinoid tumors, Kaposi's sarcoma, leiomyoma, hemangioma, lipoma, neurofibroma, fibroma), or cancer of the large bowel (adenocarcinoma, tubular adenoma, villous adenoma, hamartoma, leiomyoma).

The present disclosure also provides use of a compound of disclosed herein, or a pharmaceutically acceptable salt thereof, for preparation of a medicament for use in any of the methods described herein.

The present disclosure also provides a compound of disclosed herein, or a pharmaceutically acceptable salt thereof, for use in any of the methods described herein.

### DETAILED DESCRIPTION

The present disclosure provides a VRK1 inhibitor, or a pharmaceutically acceptable salt thereof.

In some embodiments, the VRK1 inhibitor, or a pharmaceutically acceptable salt thereof, covalently binds with a cysteine in VRK1. In some embodiments, the VRK1 inhibitor, or a pharmaceutically acceptable salt thereof, binds with a water in the binding pocket of VRK1. In some embodiments, the VRK1 inhibitor, or a pharmaceutically acceptable salt thereof, binds with Phe134 of VRK1. In some embodiments, the VRK1 inhibitor, or a pharmaceutically acceptable salt thereof, binds with Lys71 of VRK1. In some embodiments, the VRK1 inhibitor, or a pharmaceutically acceptable salt thereof, binds with Asp132 of VRK1. In some embodiments, the VRK1 inhibitor, or a pharmaceutically acceptable salt thereof, binds with the phosphate-binding loop of VRK1.

In some embodiments, the VRK1 inhibitor, or a pharmaceutically acceptable salt thereof, covalently binds with a cysteine in VRK1, a water in the binding pocket of VRK1, and Phe134 of VRK1.

In some embodiments, the VRK1 inhibitor, or a pharmaceutically acceptable salt thereof, covalently binds with a cysteine in VRK1, binds with a water in the binding pocket of VRK1, binds with Phe134 of VRK1, binds with Lys71 of VRK1, binds with Asp132 of VRK1, and binds with the phosphate-binding loop of VRK1.

The VRK1 inhibitor can include a compound of the present disclosure, or a pharmaceutically acceptable salt thereof.

The present disclosure provides a compound of Formula (I) is:

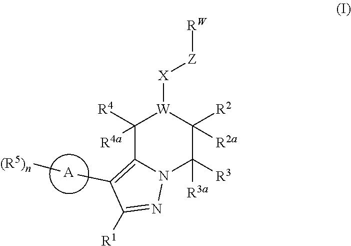

or a pharmaceutically acceptable salt thereof, wherein:
n is 0, 1, 2, 3, 4, 5, or 6;
W is CH or N;
when W is N, then X is CR⁶R^{6a}, C(O), S(O), or S(O)₂;
when W is CH, then X is NR^{6b}, CR⁶R^{6a} C(O), S(O), or S(O)₂;
Z is -Cy¹- or -Cy¹-Cy²-, wherein Cy¹ is bound to X, provided that when Z is -Cy¹-Cy²-, then Cy² is bound to R^{W};
Cy¹ is C₃₋₁₂ cycloalkyl, 6-10 membered aryl, 4-12 membered heterocycloalkyl, and 5-10 membered heteroaryl, each of which is optionally substituted by 1, 2, 3, 4, 5, or 6 independently selected R⁷ substituents;
Cy² is C₃₋₁₂ cycloalkyl, 6-10 membered aryl, 4-12 membered heterocycloalkyl, and 5-10 membered heteroaryl, each of which is optionally substituted by 1, 2, 3, 4, 5, or 6 independently selected R⁷ substituents;
Ring moiety A is C₃₋₁₀ cycloalkyl, 6-10 membered aryl, 4-10 membered heterocycloalkyl, or 5-10 membered heteroaryl;
R¹ is H, D, halo, CN, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, or C₁₋₃ alkoxy-C₁₋₄ alkyl;
R² and R^{2a} are each independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2} NR^{c2}R^{d2}, NR^{c2}C(O)R^{b2}, NR^{c2}C(O)OR^{a2}, NR^{c2}C(O)NR^{c2}R^{d2} NR^{c2}S(O)NR^{c2}R^{d2} NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or R² and R^{2a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R² and R^{2a}, taken together with the carbon atom to which they are attached, form a C(O) group;
each R^{a2}, R^{c2}, and R^{d2} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c2} and R^{d2} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
R³ and R^{3a} are each independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3} NR^{c3}S(O)NR^{c3}R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3} S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c3} and R^{d3} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents;
each R^{b3} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
R⁴ and R^{4a} are each independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c4} and R^{d4} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents;
each R^{b4} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R⁵ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
or, any R^{c5} and R^{d5} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)NR^{c51}R^{d51}, NR^{c51}S(O)R^{b51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, S(O)(═NR^{e51})R^{b51}, SF₅, P(O)R^{f51}R^{g51}, OP(O)(OR^{h51})(ORⁱ⁵¹), P(O)(OR^{h51})(ORⁱ⁵¹), and BR^{j51}R^{k51}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5B} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5B} substituents;
or, any R^{c51} and R^{d51} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{5B} substituents;
each R^{b51} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{5B} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f51} and R^{g51} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h51} and Rⁱ⁵¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a52}, SR^{a52}, NHOR^{a52}, C(O)R^{b52}, C(O)NR^{c52}R^{d52}, C(O)NR^{c52}(OR^{a52}), C(O)OR^{a52}, OC(O)R^{b52}, OC(O)NR^{c52}R^{d52}, NR^{c52}R^{d52}, NR^{c52}NR^{c52}R^{d52}, NR^{c52}C(O)R^{b52}, NR^{c52}C(O)OR^{a52}, NR^{c52}C(O)NR^{c52}R^{d52}, C(═NR^{e52})R^{b52}, C(═NR^{e52})NR^{c52}R^{d52}, NR^{c52}C(═NR^{e52})NR^{c52}R^{d52}, NR^{c52}C(═NR^{e52})R^{b52}, NR^{c52}S(O)NR^{c52}R^{d52}, NR^{c52}S(O)R^{b52}, NR^{c52}S(O)₂R^{b52}, NR^{c52}S(O)(═NR^{e52})R^{b52}, NR^{c52}S(O)₂NR^{c52}R^{d52}, S(O)R^{b52}, S(O)NR^{c52}R^{d52}, S(O)₂R^{b52}, S(O)₂NR^{c52}R^{d52}, OS(O)(═NR^{e52})R^{b52}, OS(O)₂R^{b52}, S(O)(═NR^{e52})R^{b52}, SF₅, P(O)R^{f52}R^{g52}, OP(O)(OR^{h52})(ORⁱ⁵²), P(O)(OR^{h52})(ORⁱ⁵²), and BR^{j52}R^{k52}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a52}, R^{c52}, and R^{d52} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c52} and R^{d52} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b52} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e52} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f52} and R^{g52} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h52} and Rⁱ⁵² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j52} and R^{k52} is independently selected from OH, C₁₋₆ alkoxy, and C₁₋₆ haloalkoxy;
or any R^{j52} and R^{k52} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁶ and R^{6a} are each independently selected from H, D, halo, CN, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
R^{6b} is selected from H, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R⁷ is independently selected from D, halo, CN, NO₂, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)NR^{c7}R^{d7}, NR^{c7}S(O)R^{b7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, OS(O)(═NR^{e7})R^{b7}, OS(O)₂R^{b7}, S(O)(═NR^{e7})R^{b7}, SF₅, P(O)R^{f7}R^{g7}, OP(O)(OR^{h7})(ORⁱ⁷), P(O)(OR^{h7})(ORⁱ⁷), and BR^{j7}R^{k7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
or, any R^{c7} and R^{d7} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h7} and Rⁱ⁷ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j7} and R^{k7} is independently selected from OH, C₁₋₆ alkoxy, and C₁₋₆ haloalkoxy;
or any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, NHOR^{a71}, C(O)R^{b71}, C(O)NR^{c71}R^{d71}, C(O)NR^{c71}(OR^{a71}), C(O)OR^{a71}, OC(O)R^{b71}, OC(O)NR^{c71}R^{d71}, NR^{c71}R^{d71}, NR^{c71}NR^{c71}R^{d71}, NR^{c71}C(O)R^{b71}, NR^{c71}C(O)OR^{a71}, NR^{c71}C(O)NR^{c71}R^{d71}, C(═NR^{e71})R^{b71}, C(═NR^{e71})NR^{c71}R^{d71}, NR^{c71}C(═NR^{e71})NR^{c71}R^{d71}, NR^{c71}C(═NR^{e71})R^{b71}, NR^{c71}S(O)NR^{c71}R^{d71}, NR^{c71}S(O)R^{b71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, OS(O)₂R^{b71}, S(O)(═NR^{e71})R^{b71}, SF₅, P(O)R^{f1}R^{g71}, OP(O)(OR^{h71})(ORⁱ⁷¹), P(O)(OR^{h71})(ORⁱ⁷), and BR^{j71}R^{k71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f71} and R^{g71} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h71} and Rⁱ⁷¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j71} and R^{k71} is independently selected from OH, C₁₋₆ alkoxy, and C₁₋₆ haloalkoxy;
or any R^{j71} and R^{k71} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a72}, SR^{a72}, NHOR^{a72}, C(O)R^{b72}, C(O)NR^{c72}R^{d72}, C(O)NR^{c72}(OR^{a72}), C(O)OR^{a72}, OC(O)R^{b72}, OC(O)NR^{c72}R^{d72}, NR^{c72}R^{d72}, NR^{c72}NR^{c72}R^{d72}, NR^{c72}C(O)R^{b72}, NR^{c72}C(O)OR^{a72}, NR^{c72}C(O)NR^{c72}R^{d72}, C(═NR^{e72})R^{b72}, C(═NR^{e72})NR^{c72}R^{d72}, NR^{c72}C(═NR^{e72})NR^{c72}R^{d72}, NR^{c72}C(═NR^{e72})R^{b72}, NR^{c72}S(O)NR^{c72}R^{d72}, NR^{c72}S(O)R^{b72}, NR^{c72}S(O)₂R^{b72}, NR^{c72}S(O)(═NR^{e72})R^{b72}, NR^{c72}S(O)₂NR^{c72}R^{d72}, S(O)R^{b72}, S(O)NR^{c72}R^{d72}, S(O)₂R^{b72}, S(O)₂NR^{c72}R^{d72}, OS(O)(═NR^{e72})R^{b72}, OS(O)₂R^{b72}, S(O)(═NR^{e72})R^{b72}, SF₅, P(O)R^{f72}R^{g72}, OP(O)(OR^{h72})(ORⁱ⁷²), P(O)(OR^{h72})(ORⁱ⁷²), and BR^{j72}R^{k72}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a72}, R^{c72}, and R^{d72} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c72} and R^{d72} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b72} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e72} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f72} and R^{g72} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h72} and Rⁱ⁷² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j72} and R^{k72} is independently selected from OH, C₁₋₆ alkoxy, and C₁₋₆ haloalkoxy;
or any R^{j72} and R^{k72} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{W} is a covalent warhead moiety having a reactive functional group capable of covalently binding to one or more cysteine residues in a protein; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Alternatively, in the preceding embodiment, R^{W} is a means for covalently binding to cysteine residue in a protein. It has been unexpectedly discovered that the binding pocket for VRK1 contains a cysteine not found in VRK2, which can be targeted by a warhead moiety capable of covalently binding to such cysteine.

Chemical moieties capable of binding to cysteine are known in the art and can include, but are not limited to those described in Huang, et al., “CovalentWarheads. Targeting Cysteine Residue: The Promising Approach in Drug Development”, Molecules 2022, 27, 7728; and the sections discussing cysteine warheads in the following references: Mehta, et al., “The expanding repertoire of covalent warheads for drug discovery”, Drug Discov Today 2023, 28(12):103799; and Martin, et al., “Characterising covalent warhead reactivity”, Bioorg Med Chem 2019, 15; 27(10):2066-2074, each of which is incorporated herein in its entirety.

In some embodiments, R^{W} is:

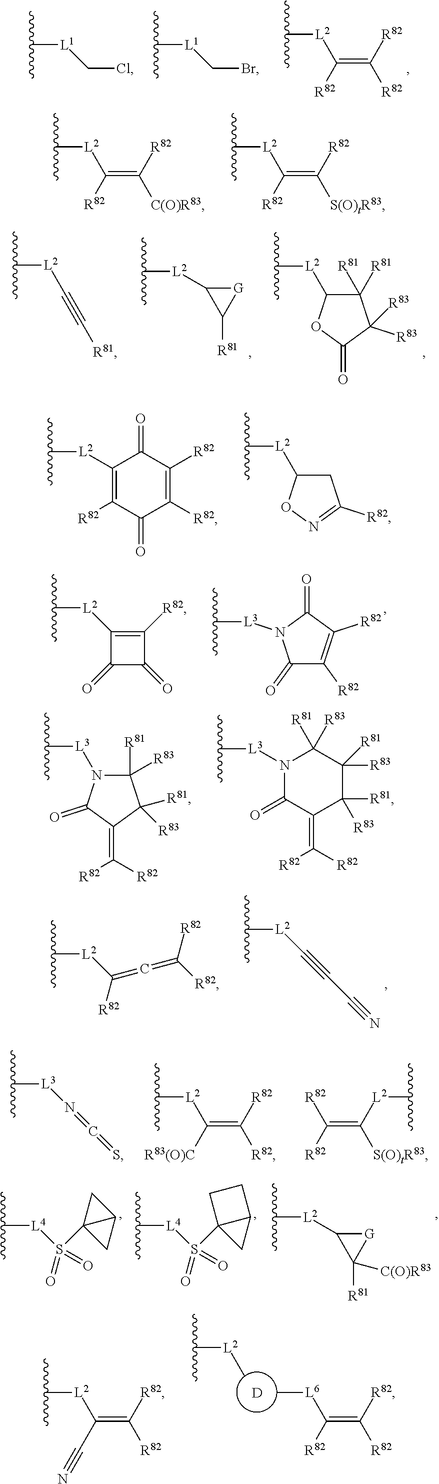

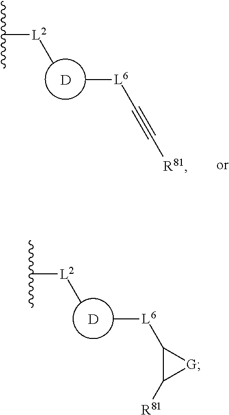

L¹ is -L-C(O)—, -L-NR⁹C(O)—, -L-OC(O)—, -L-S(O)—, -L-S(O)₂—, -L-NR⁹S(O)—, -L-OS(O)—, -L-NR⁹S(O)₂—, or -L-OS(O)₂—, wherein L¹ is attached to Z through the L linking group;
L² is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹S(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L² is attached to Z through the L linking group;
L³ is -L-, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)₂—, or -L-OS(O)₂—, wherein L³ is attached to Z through the L linking group;
L⁴ is -L-, -L-O—, -L-S—, or -L-NR⁹—, wherein L⁴ is attached to Z through the L linking group;
L⁶ is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NRS(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L⁶ is attached to Ring moiety D through the L linking group;
L is a bond or C₁₋₆ alkylene, wherein said C₁₋₆ alkylene is optionally substituted by 1, 2, 3, or 4 independently selected R^{G} substituents; or
L is —O—C₁₋₆ alkyl or —N(R^{N})—C₁₋₆ alkyl, wherein L is attached to Ring moiety A via the oxygen of the —O—C₁₋₆ alkyl or the nitrogen atom of the —N(R^{N})—C₁₋₆ alkyl group;
each R^{N} is independently H or C₁₋₆ alkyl;
Ring D is a 4-12 membered heterocycloalkyl, C₃₋₁₂ cycloalkyl, C₆₋₁₀ aryl, or a 5-10 membered heteroaryl, each of which is optionally substituted with 1, 2, 3, or 4 independently selected C₁₋₆ alkyl groups;
G is O or NR⁹;
each t is independently 0, 1, 2, or 3;
each R⁸¹ and R⁸³ are independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{c8}R^{d8}, NR^{c8}R^{d8}, NR^{c8}NR^{c8}R^{d8}, NR^{c8}C(O)R^{b8}, NR^{c8}(O)OR^{a8}, NR^{c8}C(O)NR^{c8}R^{d8}, C(═NR^{e8})R^{b8}, C(═NR^{e8})NR^{c8}R^{d8}, NR^{c8}C(═NR^{e8})R^{c8}R^{d8}, NR^{c8}C(═NR^{e8})R^{b8}, NR^{c8}S(O)NR^{c8}R^{d8}, NR^{c8}S(O)R^{b8}, NR^{c8}S(O)₂R^{b8}, NR^{c8}S(O)(═NR^{e8})R^{b8}, NR^{c8}S(O)₂NR^{c8}R^{d8}, S(O)R^{b8}, S(O)NR^{c8}R^{d8}, S(O)₂R^{b8}, S(O)₂NR^{c8}R^{d8}, OS(O)(═NR^{e8})R^{b8}, OS(O)₂R^{b8}, S(O)(═NR^{e8})R^{b8}, SF₅, P(O)R^{f8}R^{g8}, OP(O)(OR^{h8})(ORⁱ⁸), P(O)(OR^{h8})(ORⁱ⁸), and BR^{j8}R^{k8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R⁸² is independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, C(O)R^{b8}, C(O)NR^{c8}R^{d8}, C(O)OR^{a8}, C(═NR^{e8})R^{b8}, C(═NR^{e8})NR^{c8}R^{d8}, S(O)R^{b8}, S(O)NR^{c8}R^{d8}, S(O)₂R^{b8}, and S(O)₂NR^{c8}R^{d8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b8} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h8} and Rⁱ⁸ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j8} and R^{k8} is independently selected from OH, C₁₋₆ alkoxy, and C₁₋₆ haloalkoxy;
or any R^{j8} and R^{k8} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
or any two R⁸¹ and R⁸² together with the atoms to which they are attached, form C₃₋₇ cycloalkyl, 4-7 membered heterocycloalkyl, phenyl, or 5-6-membered heteroaryl ring, each of which is optionally substituted with 1, 2, 3, or 4 independently selected R^{G} substitutents;
each R⁹ is independently selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, OR^{a9}, SR^{a9}, NHOR^{a9}, C(O)R^{b9}, C(O)NR^{c9}R^{d9}, C(O)NR^{c9}(OR^{a9}), C(O)OR^{a9}, OC(O)R^{b9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, C(═NR^{e9})R^{b9}, C(═NR^{e9})NR^{c9}R^{d9}, NR^{c9}C(═NR^{e9})NR^{c9}R^{d9}, NR^{c9}C(═NR^{e9})R^{b9}, NR^{c9}S(O)NR^{c9}R^{d9}, NR^{c9}S(O)R^{b9}, NR^{c9}S(O)₂R^{b9}, NR^{c9}S(O)(═NR^{e9})R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, OS(O)(═NR^{e9})R^{b9}, OS(O)₂R^{b9}, and S(O)(═NR^{e9})R^{b9}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a9}, R^{c9}, and R^{d9} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c9} and R^{d9} attached to the same N atom, together with the N atom to which they are attached, form a 4-7 membered heterocycloalkyl group, which is optionally substituted with 1, 2, 3, or 4 independently selected R^{G} substituents;
each R^{b9} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents; and
each R^{e9} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl.

In some embodiments, R^{W} is selected from:

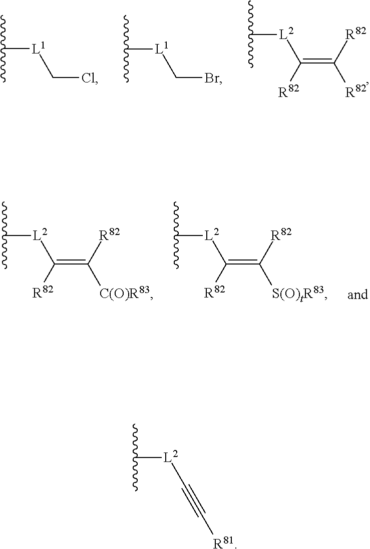

In some embodiments, R^{W} is selected from:

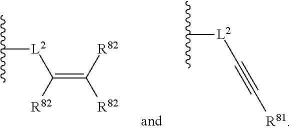

In some embodiments, R^{W} is

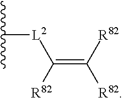

In some embodiments, L¹ is -L-C(O)—, -L-NR⁹C(O)—, -L-S(O)₂—, -L-NR⁹S(O)—, or -L-NR⁹S(O)₂—, or wherein L¹ is attached to Z through the L linking group; L is a bond; and R⁹ is selected from H and C₁₋₆ alkyl.

In some embodiments, L² is -L-C(O)—, -L-NR⁹C(O)—, -L-S(O)₂—, or -L-NR⁹S(O)₂—, wherein L² is attached to Z through the L linking group; L is a bond; and R⁹ is selected from H and C₁₋₆ alkyl.

In some embodiments, L² is —C(O)—, —NR⁹C(O)—, —S(O)₂—, or —NR⁹S(O)₂—; and R⁹ is selected from H and C₁₋₃ alkyl.

In some embodiments, when L² is —C(O)— or —S(O)₂— and Z is Cy¹, then L² is attached to a nitrogen ring member of Cy¹; and when L² is —C(O)— or —S(O)₂— and Z is -Cy¹-Cy²-, then then L² is attached to a nitrogen ring member of Cy².

In some embodiments, R⁸¹, R⁸², and R⁸³ are each independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents.

In some embodiments, R⁸¹, R⁸², and R⁸³ are each independently selected from H, halo, CN, C₁₋₆ alkyl, C₁₋₆ haloalkyl, C₃₋₇ cycloalkyl, 4-7 membered heterocycloalkyl, C₃₋₇ cycloalkyl-C₁₋₄ alkyl, and 4-7 membered heterocycloalkyl-C₁₋₄ alkyl, wherein said C₁₋₆ alkyl, C₁₋₆ haloalkyl, C₃₋₇ cycloalkyl, 4-7 membered heterocycloalkyl, C₃₋₇ cycloalkyl-C₁₋₄ alkyl, and 4-7 membered heterocycloalkyl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, amino, C₁₋₃ alkylamino, and di(C₁₋₃ alkyl)amino.

In some embodiments, R⁸¹, R⁸², and R⁸³ are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

In some embodiments, n is 0, 1, 2, 3, or 4.

In some embodiments, n is 1, 2, or 3.

In some embodiments, n is 1.

In some embodiments, n is 2.

In some embodiments, n is 3.

In some embodiments, W is CH.

In some embodiments, W is N.

In some embodiments, R⁶ and R^{6a} are each independently selected from H, D, halo, CN, C₁₋₃ alkyl, and C₁₋₃ haloalkyl; and R^{6b} is selected from H, C₁₋₃ alkyl, and C₁₋₃ haloalkyl.

In some embodiments, R^{6b} is H.

In some embodiments, X is CR⁶R^{6a}, C(O) or S(O)₂.

In some embodiments, R⁶ and R^{6a} are each H.

In some embodiments, X is C(O).

In some embodiments, X is CR⁶R^{6a}.

In some embodiments, X is CH₂.

In some embodiments, X is S(O)₂.

In some embodiments, Z is Cy¹.

In some embodiments, Cy¹ is phenyl, naphthyl, 5-10 membered heteroaryl, or 9-12 membered heterocycloalkyl.

In some embodiments, Cy¹ is phenyl, naphthyl, 5-6 membered heteroaryl, 9-10 membered heteroaryl, or 9-12 membered heterocycloalkyl.

In some embodiments, Cy¹ is selected from phenyl, naphthyl, pyridinyl, pyrazolyl, thiazolyl, indolinyl, isoindolinyl, 2,3-dihydro-1H-pyrrolo[2,3-b]pyridinyl, 1,2,3,4-tetrahydroquinolinyl, 2,3-dihydrobenzo[b][1,4]dioxinyl, 2,3-dihydro-1H-pyrrolo[3,4-c]pyridinyl, 3,4-dihydro-2H-benzo[b][1,4]oxazinyl, and isoquinolinyl.

In some embodiments, Cy¹ is phenyl.

In some embodiments, Cy¹ is naphthyl.

In some embodiments, Cy¹ is 5-6 membered heteroaryl.

In some embodiments, Cy¹ is pyridinyl.

In some embodiments, Cy¹ is 9-10 membered heteroaryl.

In some embodiments, Cy¹ is 9-12 membered heterocycloalkyl.

In some embodiments, Cy¹ is

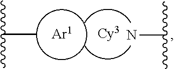

wherein Ar¹ is fused to Cy³, Ar¹ is bonded to X, Ar¹ is phenyl or 5-6 membered heteroaryl, Cy³ is 4-8 membered heterocycloalkyl, and Cy¹ is optionally substituted with 1, 2, 3, or 4 independently selected R⁷ substituents.

In some embodiments, Cy¹ is phenyl, naphthyl, 5-6 membered heteroaryl, 9-10 membered heteroaryl, or

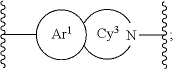

wherein:
Ar¹ is fused to Cy³ and Ar¹ is bonded to X;
Ar¹ is phenyl or 5-6 membered heteroaryl;
Cy³ is 4-8 membered heterocycloalkyl; and
Cy¹ is optionally substituted with 1, 2, 3, or 4 independently selected R⁷ substituents.

In some embodiments, R¹ selected from H, halo, CN, C₁₋₆ alkyl, and C₁₋₆ haloalkyl.

In some embodiments, R¹ selected from H, halo, CN, CH₃ and CF₃.

In some embodiments, R¹ is H.

In some embodiments, Ring moiety A is 6-10 membered aryl or 5-10 membered heteroaryl.

In some embodiments, Ring moiety A is phenyl or 5-10 membered heteroaryl.

In some embodiments, Ring moiety A is 6-10 membered aryl.

In some embodiments, Ring moiety A is phenyl.

In some embodiments, Ring moiety A is 5-10 membered heteroaryl.

In some embodiments, Ring moiety A is 5-6 membered heteroaryl or 9-10 membered heteroaryl.

In some embodiments, Ring moiety A is 5-6 membered heteroaryl.

In some embodiments, Ring moiety A is pyridinyl or pyrimidinyl.

In some embodiments, Ring moiety A is pyridinyl.

In some embodiments, Ring moiety A is pyrimidinyl.

In some embodiments, Ring moiety A is 9-10 membered heteroaryl.

In some embodiments, Ring moiety A is phenyl, pyridinyl, or pyrimidinyl.

In some embodiments, R² and R^{2a} are each independently selected from H, D, halo, CN, C₁₋₆ alkyl, and C₁₋₆ haloalkyl, and OR^{b2}, wherein said C₁₋₆ alkyl and C₁₋₆ haloalkyl are each optionally substituted with 1, 2, 3, or 4 independently selected R^{G} substituents;
or R² and R^{2a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R² and R^{2a}, taken together with the carbon atom to which they are attached, form a C(O) group; and
each R^{b2} is independently selected from C₁₋₆ alkyl and C₁₋₆ haloalkyl, which are each optionally substituted with 1, 2, 3, or 4 independently selected R^{G} substituents.

In some embodiments, R² and R^{2a} are each independently selected from H, D, halo, CN, C₁₋₆ alkyl, and C₁₋₆ haloalkyl, and OR^{b2};
or R² and R^{2a}, taken together with the carbon atom to which they are attached, form a C₃₋₇ cycloalkyl ring or a 3-7 membered heterocycloalkyl ring;
or R² and R^{2a}, taken together with the carbon atom to which they are attached, form a C(O) group; and
each R^{b2} is independently selected from C₁₋₆ alkyl and C₁₋₆ haloalkyl.

In some embodiments, R² and R^{2a} are each independently selected from H, halo, CN, C₁₋₆ alkyl, and C₁₋₆ haloalkyl.

In some embodiments, R² and R^{2a} are each independently selected from H, halo, CN, CH₃ and CF₃.

In some embodiments, R² and R^{2a} are each H.

In some embodiments, R² and R^{2a} are each independently selected from H or C₁₋₃ alkyl;
or R² and R^{2a}, taken together with the carbon atom to which they are attached, form a cyclopropyl ring.

In some embodiments, R³ and R^{3a} are each independently selected from H, D, halo, CN, C₁₋₆ alkyl, and C₁₋₆ haloalkyl, and OR^{b3}, wherein said C₁₋₆ alkyl and C₁₋₆ haloalkyl are each optionally substituted with 1, 2, 3, or 4 independently selected R^{G} substituents;
or R³ and R^{3a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R³ and R^{3a}, taken together with the carbon atom to which they are attached, form a C(O) group; and
each R^{b3} is independently selected from C₁₋₆ alkyl and C₁₋₆ haloalkyl, which are each optionally substituted with 1, 2, 3, or 4 independently selected R^{G} substituents.

In some embodiments, R³ and R^{3a} are each independently selected from H, D, halo, CN, C₁₋₆ alkyl, and C₁₋₆ haloalkyl, and OR^{b3};
or R³ and R^{3a}, taken together with the carbon atom to which they are attached, form a C₃₋₇cycloalkyl ring or a 3-7 membered heterocycloalkyl ring;
or R³ and R^{3a}, taken together with the carbon atom to which they are attached, form a C(O) group; and
each R^{b3} is independently selected from C₁₋₆ alkyl and C₁₋₆ haloalkyl.

In some embodiments, R³ and R^{3a} are each independently selected from H, halo, CN, C₁₋₆ alkyl, and C₁₋₆ haloalkyl.

In some embodiments, R³ and R^{3a} are each independently selected from H, halo, CN, CH₃ and CF₃.

In some embodiments, R³ and R^{3a} are each independently selected from H and CN.

In some embodiments, R⁴ and R^{4a} are each independently selected from H, D, halo, CN, C₁₋₆ alkyl, and C₁₋₆ haloalkyl, and OR^{b4}, wherein said C₁₋₆ alkyl and C₁₋₆ haloalkyl are each optionally substituted with 1, 2, 3, or 4 independently selected R^{G} substituents;
or R⁴ and R^{4a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R⁴ and R^{4a}, taken together with the carbon atom to which they are attached, form a C(O) group; and
each R^{b4} is independently selected from C₁₋₆ alkyl and C₁₋₆ haloalkyl, which are each optionally substituted with 1, 2, 3, or 4 independently selected R^{G} substituents.

In some embodiments, R⁴ and R^{4a} are each independently selected from H, D, halo, CN, C₁₋₆ alkyl, and C₁₋₆ haloalkyl;
or R⁴ and R^{4a}, taken together with the carbon atom to which they are attached, form a C₃₋₇ cycloalkyl ring or a 3-7 membered heterocycloalkyl ring;
or R⁴ and R^{4a}, taken together with the carbon atom to which they are attached, form a C(O) group; and
each R^{b4} is independently selected from C₁₋₆ alkyl and C₁₋₆ haloalkyl.

In some embodiments, R⁴ and R^{4a} are each independently selected from H, halo, CN, C₁₋₆ alkyl, and C₁₋₆ haloalkyl.

In some embodiments, R⁴ and R^{4a} are each independently selected from H, halo, CN, CH₃ and CF₃.

In some embodiments, R⁴ and R^{4a} are each H.

In some embodiments, each R⁵ is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-10 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, NHOR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-10 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents.

In some embodiments, each R⁵ is independently selected from D, halo, CN, C₁₋₆ alkyl, C₂₋₆ alkenyl, C₂₋₆ alkynyl, C₁₋₆ haloalkyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, NHOR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents.

In some embodiments, R⁵ is independently selected from D, halo, CN, C₁₋₆ alkyl, C₂₋₆ alkenyl, C₂₋₆ alkynyl, C₁₋₆ haloalkyl, phenyl, 5-10 membered heteroaryl, OR^{a5} and NR^{c5}R^{d5}, wherein said C₁₋₆ alkyl, C₂₋₆ alkenyl, C₂₋₆ alkynyl, C₁₋₆ haloalkyl, phenyl, and 5-10 membered heteroaryl are each optionally substituted with 1, 2, 3, or 4 independently selected R^{5A} substituents.

In some embodiments, each R⁵ is independently selected from D, halo, CN, C₁₋₆ alkyl, C₂₋₆ alkenyl, C₂₋₆ alkynyl, C₁₋₆ haloalkyl, OR^{a5} and NR^{c5}R^{d5}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{5A} substituents.

In some embodiments, each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl which are each optionally substituted with 1, 2, 3, or 4 independently selected R^{5A} substituents; and
each R^{5A} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, wherein said C₁₋₆ alkyl, C₂₋₆ alkenyl, C₂₋₆ alkynyl, C₁₋₆ haloalkyl, C₃₋₄ cycloalkyl, and 4-5 membered heterocycloalkyl are each optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents; and
each R^{5A} is independently selected from OH, CN, halo, C₁₋₃ alkyl, C₁₋₃ haloalkyl, amino, C₁₋₃ alkoxy, C₁₋₃ alkylamino, and di(C₁₋₃ alkyl)amino.

In some embodiments, each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, wherein said C₁₋₆ alkyl, C₂₋₆ alkenyl, C₂₋₆ alkynyl, C₁₋₆ haloalkyl, C₃₋₄ cycloalkyl, and 4-5 membered heterocycloalkyl are each optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents; and
each R^{5A} is independently selected from OH, CN, halo, C₁₋₃ alkyl, C₁₋₃ haloalkyl, amino, C₁₋₃ alkylamino, and di(C₁₋₃ alkyl)amino.

In some embodiments, each R⁵ is independently selected from halo, CN, C₁₋₃ alkyl, C₁₋₃ haloalkyl, OH, C₁₋₃ alkoxy, and C₁₋₃ haloalkoxy.

In some embodiments, each R⁵ is independently selected from halo, CN, C₁₋₃ alkyl, C₁₋₃ haloalkyl, OH, C₁₋₃ alkoxy, C₁₋₃ haloalkoxy, phenyl, pyridinyl, pyrimidinyl, anisolyl, and 2,3-dihydrobenzofuranyl.

In some embodiments, each R⁵ is independently selected from Cl, F, CN, CH₃, OH, and OCH₃.

In some embodiments,

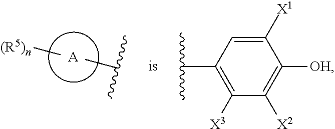

wherein X¹ and X² are each independently Cl or F, and X³ is H, Cl or F.

In some embodiments,

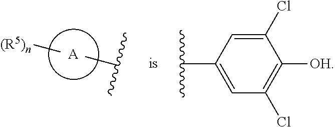

In some embodiments,

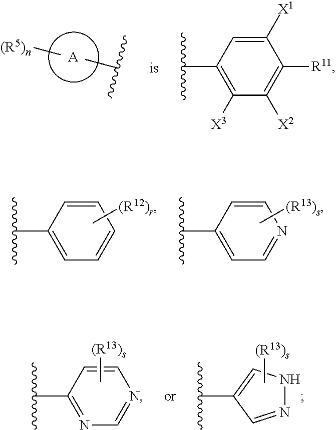

X¹ is H, Cl, F, or OCH₃;
X² is H, Cl, or F;
X³ is H, Cl, F, or OCH₃;
R¹¹ is H, OH, or CN;
r is 2;
s is 0, 1, or 2;
two R¹² substituents, together with the carbon atoms to which they are attached, form a tetrahydrofuranyl, pyrazole, pyrrole, or pyrimidine ring, which is optionally substituted by 1 or 2 independently selected R⁵ substituents selected from halo, CN, and C₁₋₃ alkyl; and
each R¹³ is independently selected from halo, CN, C₁₋₃ alkyl, and OCH₃.

In some embodiments,

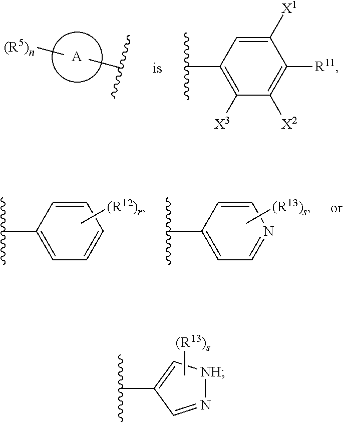

X¹ is Cl, F, or OCH₃;
X² is H, Cl, or F;
X³ is H, Cl or F;
R¹¹ is OH or CN;
r is 2;
s is 0, 1, or 2;
two R¹² substituents, together with the carbon atoms to which they are attached, form a pyrazole, pyrrole, or pyrimidine ring, which is optionally substituted by 1 or 2 independently selected R⁵ substituents selected from halo, CN, and C₁₋₃ alkyl; and
each R¹³ is independently selected from halo, CN, and C₁₋₃ alkyl.

In some embodiments,

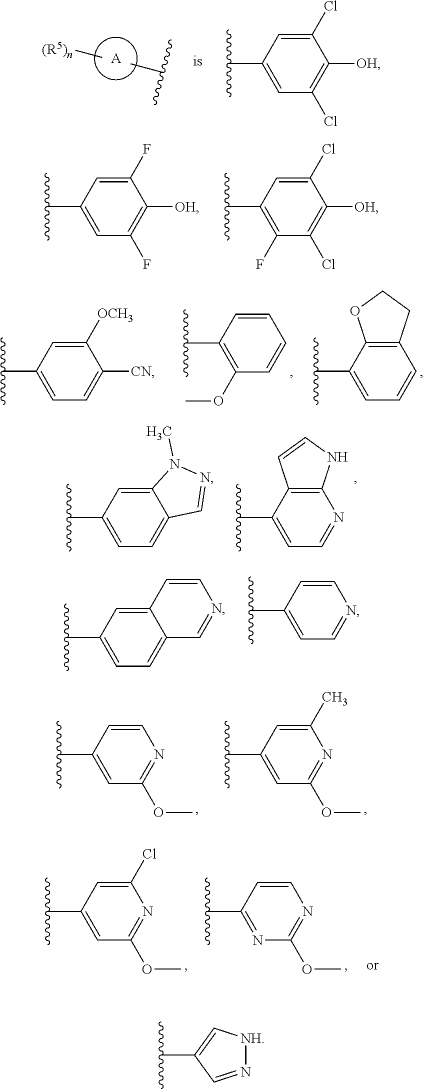

In some embodiments,

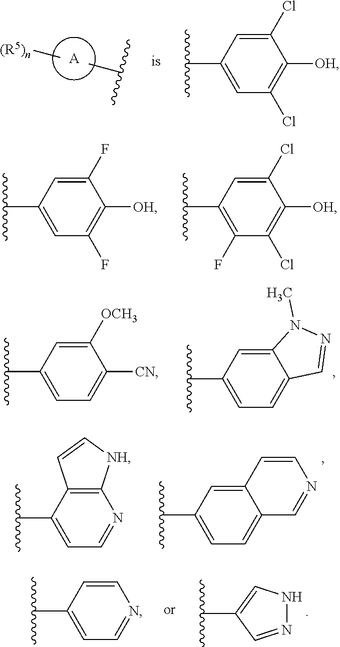

In some embodiments, each R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents.

In some embodiments, each R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents.

In some embodiments, each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents.

In some embodiments, each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c71}C(O)OR^{a71}, NR^{c71}C(O)NR^{c71}R^{d71}, NR^{c71}S(O)₂R^{b71}, NR^{c71}S(O)₂NR^{c71}R^{d71}, S(O)₂R^{b71}, and S(O)₂NR^{c71}R^{d71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl; and
each R^{b71} is independently selected from C₁₋₆ alkyl and C₁₋₆ haloalkyl.

In some embodiments, each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents; and
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino.

In some embodiments, n is 0, 1, 2, 3, or 4;
W is N;
X is CR⁶R^{6a}, C(O), or S(O)₂;
Z is Cy¹;
Cy¹ is phenyl, naphthyl, 5-6 membered heteroaryl, 9-10 membered heteroaryl, or

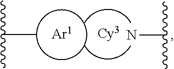

each of which is optionally substituted by 1, 2, 3, 4, 5, or 6 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl or 5-10 membered heteroaryl;
R² and R^{2a} are each independently selected from H, D, halo, CN, C₁₋₆ alkyl, and C₁₋₆ haloalkyl, and OR^{b2}, wherein said C₁₋₆ alkyl and C₁₋₆ haloalkyl are each optionally substituted with 1, 2, 3, or 4 independently selected R^{G} substituents;
or R² and R^{2a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R² and R^{2a}, taken together with the carbon atom to which they are attached, form a C(O) group;
each R^{b2} is independently selected from C₁₋₆ alkyl and C₁₋₆ haloalkyl, which are each optionally substituted with 1, 2, 3, or 4 independently selected R^{G} substituents;
R³ and R^{3a} are each independently selected from H, D, halo, CN, C₁₋₆ alkyl, and C₁₋₆ haloalkyl, and OR^{b3}, wherein said C₁₋₆ alkyl and C₁₋₆ haloalkyl are each optionally substituted with 1, 2, 3, or 4 independently selected R^{G} substituents;
or R³ and R^{3a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R³ and R^{3a}, taken together with the carbon atom to which they are attached, form a C(O) group;
each R^{b3} is independently selected from C₁₋₆ alkyl and C₁₋₆ haloalkyl, which are each optionally substituted with 1, 2, 3, or 4 independently selected R^{G} substituents;
R⁴ and R^{4a} are each independently selected from H, D, halo, CN, C₁₋₆ alkyl, and C₁₋₆ haloalkyl, and OR^{b4}, wherein said C₁₋₆ alkyl and C₁₋₆ haloalkyl are each optionally substituted with 1, 2, 3, or 4 independently selected R^{G} substituents;
or R⁴ and R^{4a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R⁴ and R^{4a}, taken together with the carbon atom to which they are attached, form a C(O) group;
each R^{b4} is independently selected from C₁₋₆ alkyl and C₁₋₆ haloalkyl, which are each optionally substituted with 1, 2, 3, or 4 independently selected R^{G} substituents;
each R⁵ is independently selected from D, halo, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, OR^{a5} and NR^{c5}R^{d5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, and 5-10 membered heteroaryl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, wherein said C₁₋₆ alkyl, C₂₋₆ alkenyl, C₂₋₆ alkynyl, C₁₋₆ haloalkyl, C₃₋₄ cycloalkyl, and 4-5 membered heterocycloalkyl are each optionally substituted with 1, 2, 3, or 4 independently selected R^{5A} substituents;
each R^{5A} is independently selected from OH, OCH₃, CN, halo, C₁₋₃ alkyl, C₁₋₃ haloalkyl, amino, C₁₋₃ alkylamino, and di(C₁₋₃ alkyl)amino;
R⁶ and R^{6a} are each independently selected from H, D, halo, CN, C₁₋₃ alkyl, and C₁₋₃ haloalkyl;
R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c71}C(O)OR^{a71}, NR^{c71}C(O)NR^{c71}R^{d71}, NR^{c71}S(O)₂R^{b71}, NR^{c71}S(O)₂NR^{c71}R^{d71}, S(O)₂R^{b71}, and S(O)₂NR^{c71}R^{d71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{7B} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R^{W} is selected from:

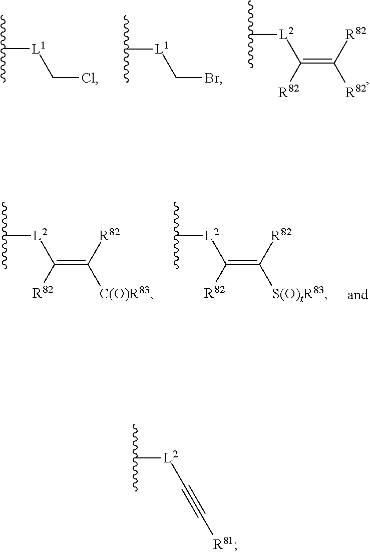

L¹ is -L-C(O)—, -L-NR⁹C(O)—, -L-S(O)₂—, -L-NR⁹S(O)—, or -L-NR⁹S(O)₂—, or wherein L¹ is attached to Z through the L linking group;
L² is -L-C(O)—, -L-NR⁹C(O)—, -L-S(O)₂—, or -L-NR⁹S(O)₂—, wherein L² is attached to Z through the L linking group;
L is a bond;
R⁹ is selected from H and C₁₋₆ alkyl;
R⁸¹, R⁸², and R⁸³ are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each t is independently 0, 1, 2, or 3; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, n is 0, 1, 2, 3, or 4;
W is N;
X is CR⁶R^{6a}, C(O), or S(O)₂;
Z is Cy¹;
Cy¹ is phenyl, naphthyl, 5-6 membered heteroaryl, 9-10 membered heteroaryl, or

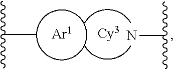

each of which is optionally substituted by 1, 2, 3, 4, 5, or 6 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl or 5-10 membered heteroaryl;
R² and R^{2a} are each independently selected from H, D, halo, CN, C₁₋₆ alkyl, and C₁₋₆ haloalkyl, and OR^{b2}, wherein said C₁₋₆ alkyl and C₁₋₆ haloalkyl are each optionally substituted with 1, 2, 3, or 4 independently selected R^{G} substituents;
or R² and R^{2a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R² and R^{2a}, taken together with the carbon atom to which they are attached, form a C(O) group;
each R^{b2} is independently selected from C₁₋₆ alkyl and C₁₋₆ haloalkyl, which are each optionally substituted with 1, 2, 3, or 4 independently selected R^{G} substituents;
R³ and R^{3a} are each independently selected from H, D, halo, CN, C₁₋₆ alkyl, and C₁₋₆ haloalkyl, and OR^{b3}, wherein said C₁₋₆ alkyl and C₁₋₆ haloalkyl are each optionally substituted with 1, 2, 3, or 4 independently selected R^{G} substituents;
or R³ and R^{3a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R³ and R^{3a}, taken together with the carbon atom to which they are attached, form a C(O) group;
each R^{b3} is independently selected from C₁₋₆ alkyl and C₁₋₆ haloalkyl, which are each optionally substituted with 1, 2, 3, or 4 independently selected R^{G} substituents;
R⁴ and R^{4a} are each independently selected from H, D, halo, CN, C₁₋₆ alkyl, and C₁₋₆ haloalkyl, and OR^{b4}, wherein said C₁₋₆ alkyl and C₁₋₆ haloalkyl are each optionally substituted with 1, 2, 3, or 4 independently selected R^{G} substituents;
or R⁴ and R^{4a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R⁴ and R^{4a}, taken together with the carbon atom to which they are attached, form a C(O) group;
each R^{b4} is independently selected from C₁₋₆ alkyl and C₁₋₆ haloalkyl, which are each optionally substituted with 1, 2, 3, or 4 independently selected R^{G} substituents;
each R⁵ is independently selected from D, halo, CN, C₁₋₆ alkyl, C₂₋₆ alkenyl, C₂₋₆ alkynyl, C₁₋₆ haloalkyl, OR^{a5} and NR^{c5}R^{d5}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, wherein said C₁₋₆ alkyl, C₂₋₆ alkenyl, C₂₋₆ alkynyl, C₁₋₆ haloalkyl, C₃₋₄ cycloalkyl, and 4-5 membered heterocycloalkyl are each optionally substituted with 1, 2, 3, or 4 independently selected R^{5A} substituents;
each R^{5A} is independently selected from OH, CN, halo, C₁₋₃ alkyl, C₁₋₃ haloalkyl, amino, C₁₋₃ alkylamino, and di(C₁₋₃ alkyl)amino;
R⁶ and R^{6a} are each independently selected from H, D, halo, CN, C₁₋₃ alkyl, and C₁₋₃ haloalkyl;
R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁-4 alkyl, 5-6 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c71}C(O)OR^{a71}, NR^{c71}C(O)NR^{c71}R^{d71}, NR^{c71}S(O)₂R^{b71}, NR^{c71}S(O)₂NR^{c71}R^{d71}, S(O)₂R^{b71}, and S(O)₂NR^{c71}R^{d71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{7B} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R^{W} is selected from:

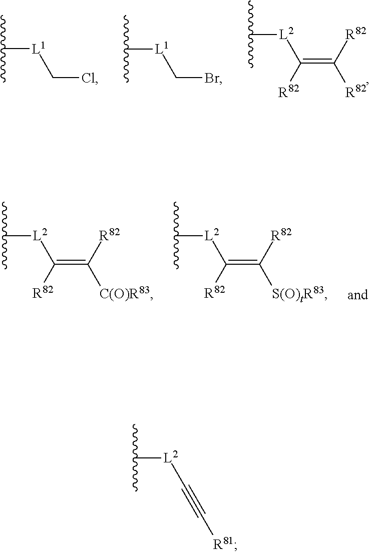

L¹ is -L-C(O)—, -L-NR⁹C(O)—, -L-S(O)₂—, -L-NR⁹S(O)—, or -L-NR⁹S(O)₂—, or wherein L¹ is attached to Z through the L linking group;
L² is -L-C(O)—, -L-NR⁹C(O)—, -L-S(O)₂—, or -L-NR⁹S(O)₂—, wherein L² is attached to Z through the L linking group;
L is a bond;
R⁹ is selected from H and C₁₋₆ alkyl;
R⁸¹, R⁸², and R⁸³ are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each t is independently 0, 1, 2, or 3; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, n is 0, 1, 2, 3, or 4;
W is N;
X is CR⁶R^{6a}, C(O), or S(O)₂;
Z is Cy¹;
Cy¹ is phenyl, naphthyl, 5-6 membered heteroaryl, 9-10 membered heteroaryl, or

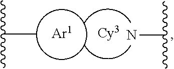

each of which is optionally substituted by 1, 2, 3, 4, 5, or 6 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl;
R¹ is H;
Ring moiety A is phenyl, 5-6 membered heteroaryl, or 9-10 membered heteroaryl;
R² and R^{2a} are each independently selected from H, D, halo, CN, C₁₋₆ alkyl, and C₁₋₆ haloalkyl, and OR^{b2};
or R² and R^{2a}, taken together with the carbon atom to which they are attached, form a C₃₋₇ cycloalkyl ring or a 3-7 membered heterocycloalkyl ring;
each R^{a2} is independently selected from C₁₋₆ alkyl and C₁₋₆ haloalkyl, which are each optionally substituted with 1, 2, 3, or 4 independently selected R^{G} substituents;
R³ and R^{3a} are each independently selected from H, D, halo, CN, C₁₋₆ alkyl, and C₁₋₆ haloalkyl, and OR^{b3};
or R³ and R^{3a}, taken together with the carbon atom to which they are attached, form a C₃₋₇ cycloalkyl ring or a 3-7 membered heterocycloalkyl ring;
R⁴ and R^{4a} are each independently selected from H, D, halo, CN, C₁₋₆ alkyl, and C₁₋₆ haloalkyl, and OR^{b4}, wherein said C₁₋₆ alkyl and C₁₋₆ haloalkyl are each optionally substituted with 1, 2, 3, or 4 independently selected R^{G} substituents;
or R⁴ and R^{4a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
each R^{b4} is independently selected from C₁₋₆ alkyl and C₁₋₆ haloalkyl, which are each optionally substituted with 1, 2, 3, or 4 independently selected R^{G} substituents;
R⁵ is independently selected from D, halo, CN, C₁₋₆ alkyl, C₂₋₆ alkenyl, C₂₋₆ alkynyl, C₁₋₆ haloalkyl, C₃₋₄ cycloalkyl, C₃₋₇ cycloalkyl, phenyl, 4-7 membered heterocycloalkyl, 5-6 membered heteroaryl, OR^{a5} and NR^{c5}R^{d5}, wherein said C₁₋₆ alkyl, C₂₋₆ alkenyl, C₂₋₆ alkynyl, C₁₋₆ haloalkyl, C₃₋₄ cycloalkyl C₃₋₇ cycloalkyl, phenyl, 4-7 membered heterocycloalkyl, and 5-6 membered heteroaryl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents;
each R^{5A} is independently selected from OH, OCH₃, CN, halo, C₁₋₃ alkyl, C₁₋₃ haloalkyl, amino, C₁₋₃ alkylamino, and di(C₁₋₃ alkyl)amino;
R⁶ and R^{6a} are each independently selected from H and C₁₋₃ alkyl;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl;
each R^{b71} is independently selected from C₁₋₆ alkyl and C₁₋₆ haloalkyl;
R^{W} is selected from:

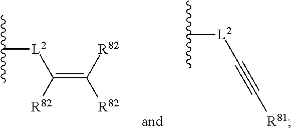

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, n is 0, 1, 2, 3, or 4;
W is N;
X is CR⁶R^{6a}, C(O), or S(O)₂;
Z is Cy¹;
Cy¹ is phenyl, naphthyl, 5-6 membered heteroaryl, 9-10 membered heteroaryl, or

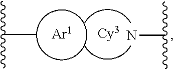

each of which is optionally substituted by 1, 2, 3, 4, 5, or 6 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl, 5-6 membered heteroaryl, or 9-10 membered heteroaryl;
R² and R^{2a} are each independently selected from H, D, halo, CN, C₁₋₆ alkyl, and C₁₋₆ haloalkyl, and OR^{b2};
or R² and R^{2a}, taken together with the carbon atom to which they are attached, form a C₃₋₇ cycloalkyl ring or a 3-7 membered heterocycloalkyl ring;
each R^{a2} is independently selected from C₁₋₆ alkyl and C₁₋₆ haloalkyl, which are each optionally substituted with 1, 2, 3, or 4 independently selected R^{G} substituents;
R³ and R^{3a} are each independently selected from H, D, halo, CN, C₁₋₆ alkyl, and C₁₋₆ haloalkyl, and OR^{b3};
or R³ and R^{3a}, taken together with the carbon atom to which they are attached, form a C₃₋₇ cycloalkyl ring or a 3-7 membered heterocycloalkyl ring;
R⁴ and R^{4a} are each independently selected from H, D, halo, CN, C₁₋₆ alkyl, and C₁₋₆ haloalkyl, and OR^{b4}, wherein said C₁₋₆ alkyl and C₁₋₆ haloalkyl are each optionally substituted with 1, 2, 3, or 4 independently selected R^{G} substituents;
or R⁴ and R^{4a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
each R^{b4} is independently selected from C₁₋₆ alkyl and C₁₋₆ haloalkyl, which are each optionally substituted with 1, 2, 3, or 4 independently selected R^{G} substituents;
R⁵ is independently selected from D, halo, CN, C₁₋₆ alkyl, C₂₋₆ alkenyl, C₂₋₆ alkynyl, C₁₋₆ haloalkyl, C₃₋₄ cycloalkyl, OR^{a5} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents;
each R^{5A} is independently selected from OH, CN, halo, C₁₋₃ alkyl, C₁₋₃ haloalkyl, amino, C₁₋₃ alkylamino, and di(C₁₋₃ alkyl)amino;
R⁶ and R^{6a} are each independently selected from H and C₁₋₃ alkyl;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl;
each R^{b71} is independently selected from C₁₋₆ alkyl and C₁₋₆ haloalkyl;
R^{W} is selected from:

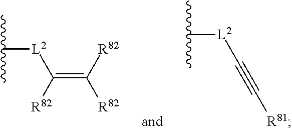

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, n is 0, 1, 2, 3, or 4;
W is N;
X is CH₂, C(O), or S(O)₂;
Z is Cy¹;
Cy¹ is phenyl, naphthyl, 5-6 membered heteroaryl, 9-10 membered heteroaryl, or

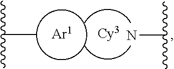

each of which is optionally substituted by 1, 2, or 3 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl, 5-6 membered heteroaryl, or 9-10 membered heteroaryl;
R² and R^{2a} are each independently selected from H, halo, CN, C₁₋₆ alkyl, and C₁₋₆ haloalkyl;
R³ and R^{3a} are each independently selected from H, halo, CN, C₁₋₆ alkyl, and C₁₋₆ haloalkyl;
R⁴ and R^{4a} are each independently selected H, halo, CN, C₁₋₆ alkyl, and C₁₋₆ haloalkyl;
each R⁵ is independently selected from halo, CN, C₁₋₃ alkyl, C₁₋₃ haloalkyl, OH, C₁₋₃ alkoxy, C₁₋₃ haloalkoxy, phenyl, and 5-6 membered heteroaryl, wherein said phenyl and 5-6 membered heteroaryl are each optionally substituted with 1 or 2 independently selected R^{5A} substituents;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from:

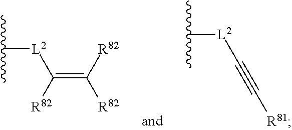

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl; and
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

In some embodiments, n is 0, 1, 2, 3, or 4;
W is N;
X is CH₂, C(O), or S(O)₂;
Z is Cy¹;
Cy¹ is phenyl, naphthyl, 5-6 membered heteroaryl, 9-10 membered heteroaryl, or

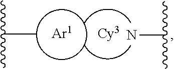

each of which is optionally substituted by 1, 2, or 3 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl, 5-6 membered heteroaryl, or 9-10 membered heteroaryl;
R² and R^{2a} are each independently selected from H, halo, CN, C₁₋₆ alkyl, and C₁₋₆ haloalkyl;
R³ and R^{3a} are each independently selected from H, halo, CN, C₁₋₆ alkyl, and C₁₋₆ haloalkyl;
R⁴ and R^{4a} are each independently selected H, halo, CN, C₁₋₆ alkyl, and C₁₋₆ haloalkyl;
each R⁵ is independently selected from halo, CN, C₁₋₃ alkyl, C₁₋₃ haloalkyl, OH, C₁₋₃ alkoxy, and C₁₋₃ haloalkoxy;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from:

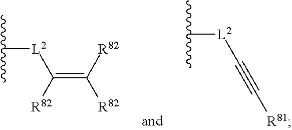

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl; and
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

In some embodiments, n is 0, 1, 2, 3, or 4;
W is N;
X is CH₂, C(O), or S(O)₂;
Z is Cy¹;
Cy¹ is phenyl, naphthyl, 5-6 membered heteroaryl, 9-10 membered heteroaryl, or

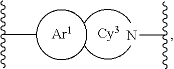

each of which is optionally substituted by 1, 2, or 3 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl, 5-6 membered heteroaryl, or 9-10 membered heteroaryl;
R² and R^{2a} are each independently selected from H, halo, CN, C₁₋₆ alkyl, and C₁₋₆ haloalkyl;
or R² and R^{2a}, taken together with the carbon atom to which they are attached, form a cyclopropyl ring;
R³ and R^{3a} are each independently selected from H, halo, CN, C₁₋₆ alkyl, and C₁₋₆ haloalkyl;
R⁴ and R^{4a} are each independently selected H, halo, CN, C₁₋₆ alkyl, and C₁₋₆ haloalkyl; each R⁵ is independently selected from halo, CN, C₁₋₃ alkyl, C₁₋₃ haloalkyl, OH, C₁₋₃ alkoxy, C₁₋₃ haloalkoxy, phenyl, and 5-6 membered heteroaryl, wherein said phenyl and 5-6 membered heteroaryl are each optionally substituted with 1 or 2 independently selected R^{5A} substituents;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from:

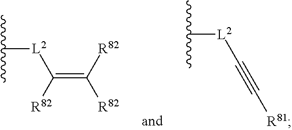

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl; and
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

In some embodiments:
n is 0, 1, 2, 3, or 4;
W is N;
X is CH₂, C(O), or S(O)₂;
Z is Cy¹;
Cy¹ is phenyl, naphthyl, 5-6 membered heteroaryl, 9-10 membered heteroaryl, or

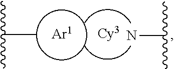

each of which is optionally substituted by 1, 2, or 3 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl, 5-6 membered heteroaryl, or 9-10 membered heteroaryl;
R² and R^{2a} are each independently selected from H, halo, CN, C₁₋₆ alkyl, and C₁₋₆ haloalkyl;
or R² and R^{2a}, taken together with the carbon atom to which they are attached, form a cyclopropyl ring;
R³ and R^{3a} are each independently selected from H, halo, CN, C₁₋₆ alkyl, and C₁₋₆ haloalkyl;
R⁴ and R^{4a} are each independently selected H, halo, CN, C₁₋₆ alkyl, and C₁₋₆ haloalkyl;
each R⁵ is independently selected from halo, CN, C₁₋₃ alkyl, C₁₋₃ haloalkyl, OH, C₁₋₃ alkoxy, and C₁₋₃ haloalkoxy;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from:

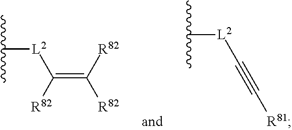

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl; and
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

In some embodiments,

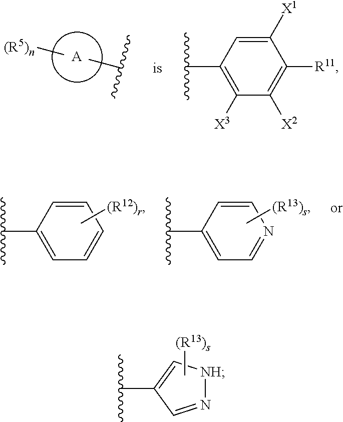

X¹ is Cl, F, or OCH₃;
X² is H, Cl, or F;
X³ is H, Cl or F;
R¹¹ is OH or CN;
r is 2;
s is 0, 1, or 2;
two R¹² substituents, together with the carbon atoms to which they are attached, form a pyrazole, pyrrole, or pyrimidine ring, which is optionally substituted by 1 or 2 independently selected R⁵ substituents selected from halo, CN, and C₁₋₃ alkyl; and
each R¹³ is independently selected from halo, CN, and C₁₋₃ alkyl.

In some embodiments, the compound is selected from:
N-(4-(3-(3,5-dichloro-4-hydroxyphenyl)-4,5,6,7-tetrahydropyrazolo[1,5-a]pyrazine-5-carbonyl)phenyl)acrylamide;
N-(4-(3-(3,5-dichloro-4-hydroxyphenyl)-4,5,6,7-tetrahydropyrazolo[1,5-a]pyrazine-5-carbonyl)-2-methoxyphenyl)acrylamide;
1-(5-(3-(3,5-dichloro-4-hydroxyphenyl)-4,5,6,7-tetrahydropyrazolo[1,5-a]pyrazine-5-carbonyl)isoindolin-2-yl)prop-2-en-1-one;
N-(5-(3-(3,5-dichloro-4-hydroxyphenyl)-4,5,6,7-tetrahydropyrazolo[1,5-a]pyrazine-5-carbonyl)-6-methoxypyridin-2-yl)acrylamide;
N-(6-(3-(3,5-dichloro-4-hydroxyphenyl)-4,5,6,7-tetrahydropyrazolo[1,5-a]pyrazine-5-carbonyl)pyridin-3-yl)acrylamide;
N-(6-(3-(3,5-dichloro-4-hydroxyphenyl)-4,5,6,7-tetrahydropyrazolo[1,5-a]pyrazine-5-carbonyl)-2-methoxypyridin-3-yl)acrylamide;
N-(3-(3-(3,5-dichloro-4-hydroxyphenyl)-4,5,6,7-tetrahydropyrazolo[1,5-a]pyrazine-5-carbonyl)-1-methyl-1H-pyrazol-5-yl)acrylamide;
N-(2-(3-(3,5-dichloro-4-hydroxyphenyl)-4,5,6,7-tetrahydropyrazolo[1,5-a]pyrazine-5-carbonyl)thiazol-5-yl)acrylamide;
N-(5-(3-(3,5-dichloro-4-hydroxyphenyl)-4,5,6,7-tetrahydropyrazolo[1,5-a]pyrazine-5-carbonyl)quinolin-8-yl)acrylamide;
1-(6-(3-(3,5-dichloro-4-hydroxyphenyl)-4,5,6,7-tetrahydropyrazolo[1,5-a]pyrazine-5-carbonyl)-1,3-dihydro-2H-pyrrolo[3,4-c]pyridin-2-yl)prop-2-en-1-one;
1-(5-(3-(3,5-dichloro-4-hydroxyphenyl)-4,5,6,7-tetrahydropyrazolo[1,5-a]pyrazine-5-carbonyl)indolin-1-yl)prop-2-en-1-one;
1-(6-(3-(3,5-dichloro-4-hydroxyphenyl)-4,5,6,7-tetrahydropyrazolo[1,5-a]pyrazine-5-carbonyl)-3,4-dihydroquinolin-1(2H)-yl)prop-2-en-1-one;
N-(4-(3-(3,5-dichloro-4-hydroxyphenyl)-4,5,6,7-tetrahydropyrazolo[1,5-a]pyrazine-5-carbonyl)naphthalen-1-yl)acrylamide;
N-(8-(3-(3,5-dichloro-4-hydroxyphenyl)-4,5,6,7-tetrahydropyrazolo[1,5-a]pyrazine-5-carbonyl)-2,3-dihydrobenzo[b][1,4]dioxin-5-yl)acrylamide;
N-(5-(3-(3,5-dichloro-4-hydroxyphenyl)-4,5,6,7-tetrahydropyrazolo[1,5-a]pyrazine-5-carbonyl)isoquinolin-8-yl)acrylamide;
N-(8-(3-(3,5-dichloro-4-hydroxyphenyl)-4,5,6,7-tetrahydropyrazolo[1,5-a]pyrazine-5-carbonyl)isoquinolin-5-yl)acrylamide;
N-(5-(3-(3,5-dichloro-4-hydroxyphenyl)-4,5,6,7-tetrahydropyrazolo[1,5-a]pyrazine-5-carbonyl)-4-methoxypyridin-2-yl)acrylamide;
N-(6-(3-(3,5-dichloro-4-hydroxyphenyl)-4,5,6,7-tetrahydropyrazolo[1,5-a]pyrazine-5-carbonyl)-2-(trifluoromethyl)pyridin-3-yl)acrylamide;
1-(5-(3-(3,5-dichloro-4-hydroxyphenyl)-4,5,6,7-tetrahydropyrazolo[1,5-a]pyrazine-5-carbonyl)-2,3-dihydro-1H-pyrrolo[2,3-b]pyridin-1-yl)prop-2-en-1-one;
1-(7-((3-(3,5-dichloro-4-hydroxyphenyl)-6,7-dihydropyrazolo[1,5-a]pyrazin-5(4H)-yl)sulfonyl)-2,3-dihydro-4H-benzo[b][1,4]oxazin-4-yl)prop-2-en-1-one;
N-(7-((3-(3,5-dichloro-4-hydroxyphenyl)-6,7-dihydropyrazolo[1,5-a]pyrazin-5(4H)-yl)sulfonyl)naphthalen-2-yl)acrylamide;
N-(4-((3-(3,5-dichloro-4-hydroxyphenyl)-6,7-dihydropyrazolo[1,5-a]pyrazin-5(4H)-yl)sulfonyl)phenyl)acrylamide;
N-(5-((3-(3,5-dichloro-4-hydroxyphenyl)-6,7-dihydropyrazolo[1,5-a]pyrazin-5(4H)-yl)methyl)pyridin-2-yl)acrylamide;
N-(4-((3-(3,5-dichloro-4-hydroxyphenyl)-6,7-dihydropyrazolo[1,5-a]pyrazin-5(4H)-yl)methyl)-2-methoxyphenyl)acrylamide;
1-(5-((3-(3,5-dichloro-4-hydroxyphenyl)-6,7-dihydropyrazolo[1,5-a]pyrazin-5(4H)-yl)methyl)isoindolin-2-yl)prop-2-en-1-one;
N-(2-chloro-4-((3-(3,5-dichloro-4-hydroxyphenyl)-6,7-dihydropyrazolo[1,5-a]pyrazin-5(4H)-yl)methyl)phenyl)acrylamide;
N-(4-(3-(3,5-dichloro-4-hydroxyphenyl)-6-methyl-4,5,6,7-tetrahydropyrazolo[1,5-a]pyrazine-5-carbonyl)phenyl)acrylamide;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(dimethylamino)but-2-enamide;
(E)-N-(5-(3-(3,5-dichloro-4-hydroxyphenyl)-4,5,6,7-tetrahydropyrazolo[1,5-a]pyrazine-5-carbonyl)quinolin-8-yl)-4-(dimethylamino)but-2-enamide;
(E)-N-(4-(3-(3,5-dichloro-4-hydroxyphenyl)-4,5,6,7-tetrahydropyrazolo[1,5-a]pyrazine-5-carbonyl)phenyl)-4-(dimethylamino)but-2-enamide;
(E)-N-(4-(3-(3,5-dichloro-4-hydroxyphenyl)-4,5,6,7-tetrahydropyrazolo[1,5-a]pyrazine-5-carbonyl)-2-fluorophenyl)-4-(dimethylamino)but-2-enamide;
(E)-1-(5-(3-(3,5-dichloro-4-hydroxyphenyl)-4,5,6,7-tetrahydropyrazolo[1,5-a]pyrazine-5-carbonyl)indolin-1-yl)-4-(dimethylamino)but-2-en-1-one;
(E)-N-(4-(3-(3,5-dichloro-4-hydroxyphenyl)-4,5,6,7-tetrahydropyrazolo[1,5-a]pyrazine-5-carbonyl)phenyl)-4-(piperidin-1-yl)but-2-enamide;
(E)-N-(4-(3-(3,5-dichloro-4-hydroxyphenyl)-4,5,6,7-tetrahydropyrazolo[1,5-a]pyrazine-5-carbonyl)phenyl)-4-morpholinobut-2-enamide;
(E)-N-(4-(3-(3,5-dichloro-4-hydroxyphenyl)-4,5,6,7-tetrahydropyrazolo[1,5-a]pyrazine-5-carbonyl)phenyl)-4-(diethylamino)but-2-enamide;
(E)-1-(5-(3-(3,5-dichloro-4-hydroxyphenyl)-4,5,6,7-tetrahydropyrazolo[1,5-a]pyrazine-5-carbonyl)isoindolin-2-yl)-4-(dimethylamino)but-2-en-1-one;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(piperidin-1-yl)but-2-enamide;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(pyrrolidin-1-yl)but-2-enamide;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-morpholinobut-2-enamide;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(diethylamino)but-2-enamide;
(E)-N-(4-(3-(3,5-dichloro-4-hydroxyphenyl)-4,5,6,7-tetrahydropyrazolo[1,5-a]pyrazine-5-carbonyl)-3-methoxyphenyl)-4-(dimethylamino)but-2-enamide;
N-(2-chloro-4-(3-(3,5-dichloro-4-hydroxyphenyl)-4,5,6,7-tetrahydropyrazolo[1,5-a]pyrazine-5-carbonyl)phenyl)acrylamide;
N-(4-(3-(3,5-dichloro-4-hydroxyphenyl)-4,5,6,7-tetrahydropyrazolo[1,5-a]pyrazine-5-carbonyl)-2-fluorophenyl)acrylamide;
N-(3-chloro-4-(3-(3,5-dichloro-4-hydroxyphenyl)-4,5,6,7-tetrahydropyrazolo[1,5-a]pyrazine-5-carbonyl)phenyl)acrylamide;
N-(4-(3-(3,5-dichloro-4-hydroxyphenyl)-4,5,6,7-tetrahydropyrazolo[1,5-a]pyrazine-5-carbonyl)-3-(trifluoromethyl)phenyl)acrylamide;
N-(5-(3-(3,5-dichloro-4-hydroxyphenyl)-4,5,6,7-tetrahydropyrazolo[1,5-a]pyrazine-5-carbonyl)pyridin-2-yl)acrylamide;
N-(4-(3-(3,5-dichloro-4-hydroxyphenyl)-4,5,6,7-tetrahydropyrazolo[1,5-a]pyrazine-5-carbonyl)-3-methoxyphenyl)acrylamide;
N-(4-(3-(3,5-dichloro-4-hydroxyphenyl)-4,5,6,7-tetrahydropyrazolo[1,5-a]pyrazine-5-carbonyl)-2-ethoxyphenyl)acrylamide;
N-(4-(3-(3,5-dichloro-4-hydroxyphenyl)-4,5,6,7-tetrahydropyrazolo[1,5-a]pyrazine-5-carbonyl)-2-isopropoxyphenyl)acrylamide;
N-(4-(3-(3,5-dichloro-4-hydroxyphenyl)-4,5,6,7-tetrahydropyrazolo[1,5-a]pyrazine-5-carbonyl)-2-(trifluoromethoxy)phenyl)acrylamide;
N-(4-(3-(3,5-dichloro-4-hydroxyphenyl)-4,5,6,7-tetrahydropyrazolo[1,5-a]pyrazine-5-carbonyl)-2-(methoxy-d₃)phenyl)acrylamide; and
N-(3-cyano-4-(3-(3,5-dichloro-4-hydroxyphenyl)-4,5,6,7-tetrahydropyrazolo[1,5-a]pyrazine-5-carbonyl)phenyl)acrylamide;

or a pharmaceutically acceptable salt of any of the aforementioned.

In some embodiments, the compound is selected from:
(E)-1-(5-((3-(3,5-dichloro-4-hydroxyphenyl)-6,7-dihydropyrazolo[1,5-a]pyrazin-5(4H)-yl)methyl)-2,3-dihydro-1H-pyrrolo[3,2-b]pyridin-1-yl)-4-(piperidin-1-yl)but-2-en-1-one; and
(E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)indolin-1-yl)-4-morpholinobut-2-en-1-one;

or a pharmaceutically acceptable salt of any of the aforementioned.

In some embodiments, the compound is selected from:
(E)-1-(5-(3′-(2-(2,3-dihydrobenzofuran-7-yl)pyridin-4-yl)-4′,5′-dihydro-7′H-spiro[cyclopropane-1,6′-pyrazolo[1,5-a]pyrazine]-5′-carbonyl)indolin-1-yl)-4-(dimethylamino)but-2-en-1-one;
(E)-4-(dimethylamino)-1-(5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one;
(E)-4-(3-azabicyclo[3.1.1]heptan-3-yl)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)but-2-en-1-one;
(E)-1-(5-(3′-(2-methoxypyridin-4-yl)-4′,5′-dihydro-7′H-spiro[cyclopropane-1,6′-pyrazolo[1,5-a]pyrazine]-5′-carbonyl)indolin-1-yl)-4-(piperidin-1-yl)but-2-en-1-one;
(E)-1-(5-(3′-(3,5-dichloro-4-hydroxyphenyl)-4′,5′-dihydro-7′H-spiro[cyclobutane-1,6′-pyrazolo[1,5-a]pyrazine]-5′-carbonyl)indolin-1-yl)-4-(dimethylamino)but-2-en-1-one;
(E)-4-(3-azabicyclo[3.1.1]heptan-3-yl)-1-(5-((3-(3,5-dichloro-4-hydroxyphenyl)-6,7-dihydropyrazolo[1,5-a]pyrazin-5(4H)-yl)methyl)-2,3-dihydro-1H-pyrrolo[3,2-b]pyridin-1-yl)but-2-en-1-one;
(E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)indolin-1-yl)-4-(dimethylamino)but-2-en-1-one;
(E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)-4-morpholinobut-2-en-1-one;
(E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)-4-(piperidin-1-yl)but-2-en-1-one;
(E)-4-(dimethylamino)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one;
(E)-1-(5-(3′-(3,5-dichloro-4-hydroxyphenyl)-4′,5′-dihydro-7′H-spiro[cyclobutane-1,6′-pyrazolo[1,5-a]pyrazine]-5′-carbonyl)indolin-1-yl)-4-morpholinobut-2-en-1-one;
(R,E)-1-(5-(3-(3,5-dichloro-4-hydroxyphenyl)-6-methyl-4,5,6,7-tetrahydropyrazolo[1,5-a]pyrazine-5-carbonyl)-4-methoxyindolin-1-yl)-4-morpholinobut-2-en-1-one;
(E)-1-(5-(3′-(2-methoxypyrimidin-4-yl)-4′,5′-dihydro-7′H-spiro[cyclopropane-1,6′-pyrazolo[1,5-a]pyrazine]-5′-carbonyl)indolin-1-yl)-4-morpholinobut-2-en-1-one;
(R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-(2,2-dimethylpyrrolidin-1-yl)but-2-en-1-one;
N-(4-(3′-(3,5-dichloro-4-hydroxyphenyl)-4′,5′-dihydro-7′H-spiro[cyclopropane-1,6′-pyrazolo[1,5-a]pyrazine]-5′-carbonyl)-2-methoxyphenyl)acrylamide;
(R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-(piperidin-1-yl)but-2-en-1-one;
(E)-4-(3-fluoroazetidin-1-yl)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one;
(R)-1-(5-(3-(3,5-dichloro-4-hydroxyphenyl)-6-methyl-4,5,6,7-tetrahydropyrazolo[1,5-a]pyrazine-5-carbonyl)isoindolin-2-yl)prop-2-en-1-one;
(R,E)-N-(4-(3-(3,5-dichloro-4-hydroxyphenyl)-6-methyl-4,5,6,7-tetrahydropyrazolo[1,5-a]pyrazine-5-carbonyl)-2-fluorophenyl)-4-morpholinobut-2-enamide;
(R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-morpholinobut-2-en-1-one;
(R,E)-N-(4-(3-(3,5-dichloro-4-hydroxyphenyl)-6-isopropyl-4,5,6,7-tetrahydropyrazolo[1,5-a]pyrazine-5-carbonyl)-2-methoxyphenyl)-4-(dimethylamino)but-2-enamide; and
(E)-4-(3-fluoroazetidin-1-yl)-1-(5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one;

or a pharmaceutically acceptable salt of any of the aforementioned.

In some embodiments, the compound is (E)-4-(3-azabicyclo[3.1.1]heptan-3-yl)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)but-2-en-1-one, or a pharmaceutically acceptable salt thereof.

In some embodiments, the compound is (E)-4-(dimethylamino)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one, or a pharmaceutically acceptable salt thereof.

In some embodiments, the compound is (E)-4-(3-fluoroazetidin-1-yl)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one, or a pharmaceutically acceptable salt thereof.

In some embodiments, the compound is (E)-4-(3-fluoroazetidin-1-yl)-1-(5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one, or a pharmaceutically acceptable salt thereof.

In some embodiments, compounds of Formula (I), or a pharmaceutically acceptable salt thereof, can include one or more hydrogen atoms replaced by deuterium. In some embodiments, 1, 2, 3, 4, 5, 6, 7, or 8 hydrogen atoms, attached to carbon atoms of “alkyl”, “alkenyl”, “alkynyl”, “aryl”, “phenyl”, “cycloalkyl”, “heterocycloalkyl”, or “heteroaryl” substituents or “—C₁₋₄ alkyl-” and “alkylene” linking groups, as described herein, are optionally replaced by deuterium atoms.

It is further appreciated that certain features of the present disclosure, which are, for clarity, described in the context of separate embodiments, can also be provided in combination in a single embodiment (while the embodiments are intended to be combined as if written in multiply dependent form). Conversely, various features of the present disclosure which are, for brevity, described in the context of a single embodiment, can also be provided separately or in any suitable subcombination. Thus, it is contemplated as features described as embodiments of the compounds of Formula (I) can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency, that the designated atom's normal valency is not exceeded, and that the substitution results in a stable compound.

As used herein, the term “independently selected from” means that each occurrence of a variable or substituent are independently selected at each occurrence from the applicable list.

As used herein, the phrase “each ‘variable’ is independently selected from” means substantially the same as wherein “at each occurrence ‘variable’ is selected from.”

When any variable (e.g., R^{G}) occurs more than one time in any constituent or formula for a compound, its definition at each occurrence is independent of its definition at every other occurrence. Thus, for example, if a group is shown to be substituted with 1, 2, 3, or 4 independently selected R^{G} substituents, then said group may optionally be substituted with up to four R^{G} groups and R^{G} at each occurrence is selected independently from the definition of R^{G}.

In some embodiments, when an optionally multiple substituent is designated in the form:

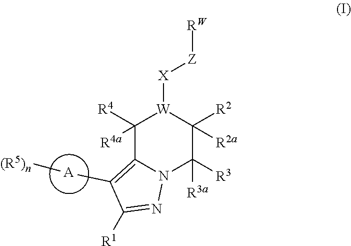

then it is to be understood that substituent R⁵ can occur n number of times on the ring, and R⁵ can be a different moiety at each occurrence. It is to be understood that each R group may replace any hydrogen atom attached to a ring atom.

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (i-Pr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms. As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “C₁₋₄ alkylidene-C₃₋₇ cycloalkyl” refers to a group of formula

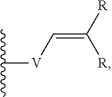

wherein the V is a bond or C₁₋₃ alkylene, and R and R, taken together with the carbon atom to which they are attached, form a C₃₋₇ cycloalkyl ring.

As used herein, “C₁₋₄ alkylidene-4-7-membered heterocycloalkyl” refers to a group of formula

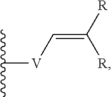

wherein the V is a bond or C₁₋₃ alkylene, and R and R, taken together with the carbon atom to which they are attached, form a 4-7 membered heterocycloalkyl ring.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. In some embodiments, the aryl group has 6 to 10 carbon atoms. In some embodiments, the aryl group is phenyl or naphthyl. In some embodiments, the aryl is phenyl.

As used herein, “halo” refers to F, Cl, Br, or I. In some embodiments, halo is F, Cl, or Br. In some embodiments, halo is F or Cl. In some embodiments, halo is F. In some embodiments, halo is Cl.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group of the haloalkyl has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, the term “Cₙ₋ₘ fluoroalkyl” refers to an alkyl group having from one fluoro atom to 2s+1 fluoro atoms, where “s” is the number of carbon atoms in the alkyl group, wherein the alkyl group has n to m carbon atoms. In some embodiments, the alkyl group of the fluoroalkyl has 1 to 6, 1 to 4, or 1 to 3 carbon atoms. Example fluoroalkyl groups include CF₃, C₂F₅, CHF₂, CH₂F, and the like.

As used herein, the term “thio” refers to a group of formula —SH.

As used herein, the term “Cₙ₋ₘ alkylamino” refers to a group of formula —NH(alkyl), wherein the alkyl group has n to m carbon atoms. In some embodiments, the alkyl group of the alkylamino has 1 to 6, 1 to 4, or 1 to 3 carbon atoms.

As used herein, the term “Cₙ₋ₘ alkoxycarbonyl” refers to a group of formula —C(O)O— alkyl, wherein the alkyl group has n to m carbon atoms. In some embodiments, the alkyl group of the alkoxycarbonyl has 1 to 6, 1 to 4, or 1 to 3 carbon atoms.

As used herein, the term “Cₙ₋ₘ alkylcarbonyl” refers to a group of formula —C(O)— alkyl, wherein the alkyl group has n to m carbon atoms. In some embodiments, the alkyl group of the alkylcarbonyl has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group of the alkylcarbonylamino has 1 to 6, 1 to 4, or 1 to 3 carbon atoms.

As used herein, the term “Cₙ₋ₘ alkoxycarbonylamino” refers to a group of formula —NHC(O)O(Cₙ₋ₘ alkyl), wherein the alkyl group has n to m carbon atoms. In some embodiments, the alkyl group of the alkoxycarbonylamino has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group of the alkylsulfonylamino has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group of the alkylaminosulfonyl has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group of the dialkylaminosulfonyl has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group of the alkylaminosulfonylamino has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group of the dialkylaminosulfonylamino has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group of the alkylaminocarbonylamino has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group of the dialkylaminocarbonylamino has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group of the alkylcarbamyl has 1 to 6, 1 to 4, or 1 to 3 carbon atoms.

As used herein, the term “Cₙ₋ₘ alkylthio” refers to a group of formula —S-alkyl, wherein the alkyl group has n to m carbon atoms. In some embodiments, the alkyl group of the alkylthio has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group of the alkylsulfinyl has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group of the alkylsulfonyl has 1 to 6, 1 to 4, or 1 to 3 carbon atoms.

As used herein, the term “cyano-Cₙ₋ₘ alkyl” refers to a group of formula —(Cₙ₋ₘ alkylene)-CN, wherein the alkylene group has n to m carbon atoms. As used herein, the term “cyano-C₁₋₆ alkyl” refers to a group of formula —(C₁₋₆ alkylene)-CN. As used herein, the term “cyano-C₁₋₃ alkyl” refers to a group of formula —(C₁₋₃ alkylene)-CN.

As used herein, the term “HO—Cₙ₋ₘ alkyl” refers to a group of formula —(Cₙ₋ₘ alkylene)-OH, wherein the alkylene group has n to m carbon atoms. As used herein, the term “HO—C₁₋₃ alkyl” refers to a group of formula —(C₁₋₃ alkylene)-OH.

As used herein, the term “Cₙ₋ₘ alkoxy-Cₒ₋ₚ alkyl” refers to a group of formula —(Cₙ₋ₘ alkylene)-O(Cₒ₋ₚ alkyl), wherein the alkylene group has n to m carbon atoms and the alkyl group has o to p carbon atoms. As used herein, the term “C₁₋₆ alkoxy-C₁₋₆ alkyl” refers to a group of formula —(C₁₋₆ alkylene)-O(C₁₋₆ alkyl). As used herein, the term “C₁₋₃ alkoxy-C₁₋₃ alkyl” refers to a group of formula —(C₁₋₃ alkylene)-O(C₁₋₃ alkyl).

As used herein, the term “carboxy” refers to a group of formula —C(O)OH.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group of the dialkylamino independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group of the dialkylcarbamyl independently has 1 to 6, 1 to 4, or 1 to 3 carbon atoms.

As used herein, the term “Cₙ₋ₘ alkylcarbonyloxy” is a group of formula —OC(O)-alkyl, wherein the alkyl group has n to m carbon atoms. In some embodiments, the alkyl group of the alkylcarbonyloxy has 1 to 6, 1 to 4, or 1 to 3 carbon atoms.

As used herein, “aminocarbonyloxy” is a group of formula —OC(O)—NH₂.

As used herein, “Cₙ₋ₘ alkylaminocarbonyloxy” is a group of formula —OC(O)—NH— alkyl, wherein the alkyl group has n to m carbon atoms. In some embodiments, the alkyl group of the alkylaminocarbonyloxy has 1 to 6, 1 to 4, or 1 to 3 carbon atoms.

As used herein, “di(Cₙ₋ₘalkyl)aminocarbonyloxy” is a group of formula —OC(O)—N(alkyl)₂, wherein each alkyl group has, independently, n to m carbon atoms. In some embodiments, each alkyl group of the dialkylaminocarbonyloxy independently has 1 to 6, 1 to 4, or 1 to 3 carbon atoms.

As used herein “Cₙ₋ₘ alkoxycarbonylamino” refers to a group of formula —NHC(O)—O— alkyl, wherein the alkyl group has n to m carbon atoms.

As used herein, the term “carbamyl” to a group of formula —C(O)NH₂.

As used herein, the term “carbonyl”, employed alone or in combination with other terms, refers to a —C(O)— group.

As used herein, “cycloalkyl” refers to non-aromatic cyclic hydrocarbons including cyclized alkyl and alkenyl groups. Cycloalkyl groups can include mono- or polycyclic (e.g., having 2, 3 or 4 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2, 3, or 4 fused rings) aromatic heterocycle having at least one heteroatom ring member selected from N, O, or S. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, and S. In some embodiments, the heteroaryl group contains 5 to 10 or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridyl, pyrimidinyl, pyrazinyl, pyridazinyl, pyrrolyl, pyrazolyl, azolyl, oxazolyl, isoxazolyl, thiazolyl, isothiazolyl, imidazolyl, furyl, thienyl, triazolyl (e.g., 1,2,3-triazolyl, 1,2,4-triazolyl, 1,3,4-triazolyl), tetrazolyl, thiadiazolyl (e.g., 1,2,3-thiadiazolyl, 1,2,4-thiadiazolyl, 1,3,4-thiadiazolyl), quinolinyl, isoquinolinyl, indolyl, benzothienyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, oxadiazolyl (e.g., 1,2,3-oxadiazolyl, 1,2,4-oxadiazolyl, 1,3,4-oxadiazolyl), 1,2-dihydro-1,2-azoborinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, or S, and wherein the ring-forming carbon atoms and heteroatoms of the heterocycloalkyl group can be optionally substituted by one or more oxo or sulfido (e.g., C(O), S(O), C(S), or S(O)₂, etc.). Heterocycloalkyl groups include monocyclic and polycyclic (e.g., having 2 fused rings) systems. Included in heterocycloalkyl are monocyclic and polycyclic 4-10-, 4-7-, and 5-6-membered heterocycloalkyl groups. Heterocycloalkyl groups can also include spirocycles and bridged rings. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 4 to 10 ring-forming atoms, 4 to 7 ring-forming atoms, 4 to 6 ring-forming atoms or 5 to 6 ring-forming atoms. In some embodiments, the heterocycloalkyl group has 1 to 4 heteroatoms, 1 to 3 heteroatoms, 1 to 2 heteroatoms or 1 heteroatom.

In some embodiments, the heterocycloalkyl is a 4-10 membered monocyclic, bicyclic, or tricyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-10 membered bicyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-7 membered monocyclic heterocycloalkyl having 1 or 2 ring-forming heteroatoms independently selected from N, O, and S, and wherein 1, 2 or 3 ring-forming carbon or heteroatoms can be optionally substituted by one or more oxo or sulfido. In some embodiments, the heterocycloalkyl is a monocyclic 4-6 membered heterocycloalkyl having 1 or 2 heteroatoms independently selected from N, O, S, and B and having one or more oxidized ring members.

Examples of heterocycloalkyl groups include pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, 1,2,3,4-tetrahydroisoquinoline, azabicyclo[3.1.0]hexanyl, diazabicyclo[3.1.0]hexanyl, oxabicyclo[2.1.1]hexanyl, azabicyclo[2.2.1]heptanyl, azabicyclo[2.2.1]heptan-7-yl, azabicyclo[2.2.1]heptan-2-yl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon ring members and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, the term “alkylene” refers a divalent straight chain or branched alkyl linking group. Examples of “alkylene groups” include methylene, ethan-1,1-diyl, ethan-1,2-diyl, propan-1,3-dilyl, propan-1,2-diyl, propan-1,1-diyl and the like.

As used herein, the term “alkenylene” refers a divalent straight chain or branched alkenyl linking group. Examples of “alkenylene groups” include ethen-1,1-diyl, ethen-1,2-diyl, propen-1,3-diyl, 2-buten-1,4-diyl, 3-penten-1,5-diyl, 3-hexen-1,6-diyl, 3-hexen-1,5-diyl, and the like.

As used herein, the term “alkynylene” refers a divalent straight chain or branched alkynyl linking group. Examples of “alkynylene groups” include propyn-1,3-diyl, 2-butyn-1,4-diyl, 3-pentyn-1,5-diyl, 3-hexyn-1,6-diyl, 3-hexyn-1,5-diyl, and the like.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula (I), (II), etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g., hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes, such as those provided in the Schemes below.

Compounds of formula (I) can be prepared using a process illustrated in Scheme 1. In which, compounds of formula (1-1) can react with compounds (1-2) via amide formation (e.g., HATU or T₃P in the presence of DIPEA/TEA) or sulfonamide formation conditions (in the presence of TEA, J. Org. Chem., 2008, 73, 3967-3969), resulting in the formation of the compounds (1-3). Compound (1-3) can then react with the corresponding boronic acids or esters via Suzuki coupling (in the presence of Pd catalyst, e.g. x-phos Pd G2) to afford the final product (I) (certain protection, deprotection, and other modification steps were performed when necessary). Alternatively, the sequence of amide/sulfonamide formation and Suzuki coupling steps can be reversed as depicted in Scheme 1 (via intermediate 1-4).

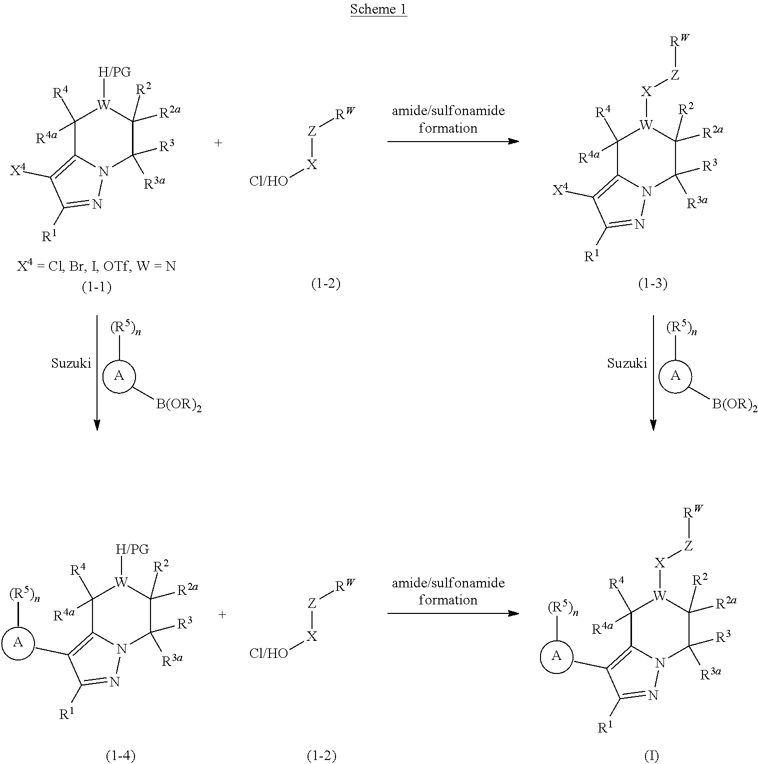

For compounds (1-2), when commercially not available, they can be prepared via reactions shown in Scheme 2. Compounds (2-1), with a free amine available, can react with compounds (2-2) via amide formation (e.g., HATU or T₃P in the presence of DIPEA/TEA) or sulfonamide formation (in the presence of TEA, J. Org. Chem., 2008, 73, 3967-3969), to afford compound (2-3), Compounds (2-3) can then be converted to compounds (1-2) under hydrolysis conditions (e.g., LiOH in MeOH/THF/H₂O), or deprotection conditions (e.g. TFA/DCM, when Rx=^{t}Bu). Along with the steps outlined in Scheme 1, specific protection, deprotection, or other modification steps, as well as reaction sequences, can be adjusted as needed.

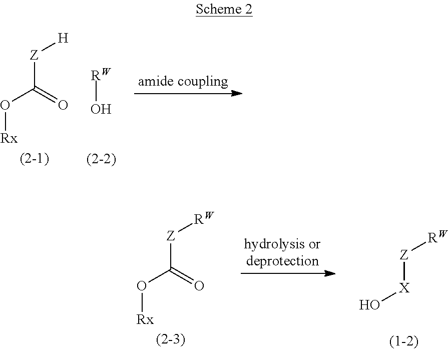

The reactions for preparing compounds described herein can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “r.t.” as used herein, are understood in the art, and refer generally to a temperature, e.g., a reaction temperature, that is about the temperature of the room in which the reaction is carried out, for example, a temperature from about 20° C. to about 30° C.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The present disclosure further provides methods of inhibiting VRK1, comprising contacting the VRK1 with a VRK1 inhibitor, or a pharmaceutically acceptable salt thereof. The present disclosure further provides methods of selectively inhibiting VRK1 over VRK2, comprising contacting VRK1 and VRK2 with a VRK1 inhibitor, or a pharmaceutically acceptable salt thereof.

In some embodiments, the VRK1 inhibitor, or a pharmaceutically acceptable salt thereof, covalently binds with a cysteine in VRK1. In some embodiments, the VRK1 inhibitor, or a pharmaceutically acceptable salt thereof, covalently binds with Cys50 in VRK1.

In some embodiments, the VRK1 inhibitor, or a pharmaceutically acceptable salt thereof, binds with a water in the binding pocket of VRK1. In some embodiments, the water in the binding pocket of VRK1 interacts with one or more of Asp197, Tyr87, and Glu83 of VRK1. In some embodiments, the water in the binding pocket of VRK1 interacts with Asp197, Tyr87, and Glu83 of VRK1. In some embodiments, the water in the binding pocket of VRK1 interacts with Asp197 of VRK1. In some embodiments, the water in the binding pocket of VRK1 interacts with Tyr87 of VRK1. In some embodiments, the water in the binding pocket of VRK1 interacts with Glu83 of VRK1.

In some embodiments, the VRK1 inhibitor, or a pharmaceutically acceptable salt thereof, binds with Phe134 of VRK1.

In some embodiments, the VRK1 inhibitor, or a pharmaceutically acceptable salt thereof, binds with Lys71 of VRK1.

In some embodiments, the VRK1 inhibitor, or a pharmaceutically acceptable salt thereof, binds with Asp132 of VRK1.

In some embodiments, the VRK1 inhibitor, or a pharmaceutically acceptable salt thereof, binds with the phosphate-binding loop of VRK1.

In some embodiments, the VRK1 inhibitor, or a pharmaceutically acceptable salt thereof, covalently binds with a cysteine in VRK1, binds with a water in the binding pocket of VRK1, and binds with Phe134 of VRK1.

In some embodiments, the VRK1 inhibitor, or a pharmaceutically acceptable salt thereof, covalently binds with a cysteine in VRK1, binds with a water in the binding pocket of VRK1, binds with Phe134 of VRK1, binds with Lys71 of VRK1, binds with Asp132 of VRK1, and binds with the phosphate-binding loop of VRK1.

In some embodiments, the VRK1 inhibitor, or a pharmaceutically acceptable salt thereof, covalently bonds with VRK1.

In some embodiments, the VRK1 inhibitor, or a pharmaceutically acceptable salt thereof, binds with VRK1 through one or more strong interactions and one or more weaker interactions.

In some embodiments, the VRK1 inhibitor, or a pharmaceutically acceptable salt thereof, binds with VRK1 through one or more strong interactions. In some embodiments, the strong interactions are selected from hydrogen bond interactions and salt bridge interactions. In some embodiments, the strong interactions are one or more hydrogen bond interactions and one or more salt bridge interactions. In some embodiments, the strong interactions are one or more hydrogen bond interactions. In some embodiments, the strong interactions are one or more salt bridge interactions.

In some embodiments, the VRK1 inhibitor, or a pharmaceutically acceptable salt thereof, binds with VRK1 through one or more weaker interactions. In some embodiments, the weaker interactions are selected from CH-π interactions, C—H hydrogen bonding interactions, and van der Waals interactions. In some embodiments, the weaker interactions are one or more CH-π interactions, one or more C—H hydrogen bonding interactions, and one or more van der Waals interactions. In some embodiments, the weaker interactions are one or more CH-π interactions. In some embodiments, the weaker interactions are one or more C—H hydrogen bonding interactions. In some embodiments, the weaker interactions are one or more van der Waals interactions.

The VRK1 inhibitor can include a compound of the present disclosure, or a pharmaceutically acceptable salt thereof.

Compounds of the present disclosure (and pharmaceutically acceptable salts thereof) can inhibit VRK1 and therefore are useful for treating diseases wherein the underlying pathology is, wholly or partially, mediated by VRK1. In some embodiments, the compounds, and pharmaceutically acceptable salts thereof, inhibit VRK1. In some embodiments, the compounds, and pharmaceutically acceptable salts thereof, are selective for VRK1 over VRK2. In some embodiments, the selectivity referenced in each of the embodiments of this paragraph is at least 10-fold, at least 100-fold selective, at least 500-fold selective, or at least 1000 fold selective.

In some embodiments, R^{W} of Formula (I) is a means for covalently binding with a cysteine (Cys).

In some embodiments, R^{W} of Formula (I) is a means for covalently binding with a Cys in VRK1.

In some embodiments, R^{W} of Formula (I) is a means for covalently binding with Cys50 in VRK1.

In some embodiments, Ring moiety A of Formula (I) is a means for binding with a lyseine (Lys).

In some embodiments, Ring moiety A of Formula (I) is a means for binding with a Lys in VRK1.

In some embodiments, Ring moiety A of Formula (I) is a means for binding with Lys71 in VRK1.

In some embodiments, Ring moiety A of Formula (I) is a means for binding with a water in the binding pocket of VRK1.

In some embodiments, the 4,5,6,7-tetrahydropyrazolo[1,5-a]pyrazine (e.g., when W is N) or 4,5,6,7-tetrahydropyrazolo[1,5-a]pyridine (e.g., when W is CH) of Formula (I) is a means for binding with a phenylalanine (Phe).

In some embodiments, the 4,5,6,7-tetrahydropyrazolo[1,5-a]pyrazine (e.g., when W is N) or 4,5,6,7-tetrahydropyrazolo[1,5-a]pyridine (e.g., when W is CH) of Formula (I) is a means for binding with a Phe in VRK1.

In some embodiments, the 4,5,6,7-tetrahydropyrazolo[1,5-a]pyrazine (e.g., when W is N) or 4,5,6,7-tetrahydropyrazolo[1,5-a]pyridine (e.g., when W is CH) of Formula (I) is a means for binding with Phe134 in VRK1.

In one embodiment, the present disclosure provides a method for inhibiting growth of tumor cells in vitro. The method includes contacting the tumor cells in vitro with a compound of Formula (I) or of any of the formulas as described herein, or of a compound as recited in any of the claims and described herein, or of a salt thereof. The method includes administering to the individual or patient in need thereof a therapeutically effective amount of a compound of Formula (I) or of any of the formulas as described herein, or of a compound as recited in any of the claims and described herein, or a salt or a stereoisomer thereof.

In some embodiments, provided herein is a method of inhibiting VRK1, comprising contacting the VRK1 with a compound of Formula (I) or any of the formulas as described herein, a compound as recited in any of the claims and described herein, or a salt thereof. In some embodiments, provided herein is a method of inhibiting VRK1 in a patient, comprising administering to the patient a compound of Formula (I) or any of the formulas as described herein, a compound as recited in any of the claims and described herein, or a salt thereof.

In some embodiments, the disclosure includes a method of treating a disease or disorder associated with VRK1 in a patient, comprising administering to the patient a therapeutically effective amount of the compound described herein, or pharmaceutically acceptable salt thereof. Such diseases include cancer and other diseases with proliferation disorder. In some embodiments, the present disclosure provides treatment of an individual or a patient in vivo using a compound of Formula (I) or a salt thereof such that growth of cancerous tumors is inhibited. A compound of Formula (I) or of any of the formulas as described herein, or a compound as recited in any of the claims and described herein, or a salt thereof, can be used to inhibit the growth of cancerous tumors with aberrations that activate the VRK1 kinase and/or VRK1 activity.

In some embodiments, provided herein is a method of treating a disease or disorder associated with VRK1 in a patient, comprising administering to the patient a therapeutically effective amount of a compound of Formula (I) or any of the formulas as described herein, a compound as recited in any of the claims and described herein, or a salt thereof. In some embodiments, provided herein is a method for treating cancer. The method includes administering to a patient (in need thereof), a therapeutically effective amount of a compound of Formula (I) or any of the formulas as described herein, a compound as recited in any of the claims and described herein, or a salt thereof. In some embodiments, the cancer is sarcoma, colorectal adenocarcinoma, acute myeloid leukemia, glioblastoma, or neuroblastoma. In some embodiments, the glioblastoma or neuroblastoma has overexpression of VRK1. In some embodiments, the glioblastoma or neuroblastoma has underexpression of VRK2. In some embodiments, the cancer is bone cancer, cancer of the neck and head, rectal cancer, esophageal cancer, sarcoma of soft tissue, neoplasm of the central nervous system (CNS), brain stem, glioma, colon cancer, Ewing's sarcoma, brain cancer, astrocytoma, neuroblastoma, intestinal cancer, nervous system cancers, osteosarcoma, cancer of the small bowel (adenocarcinoma, lymphoma, carcinoid tumors, Kaposi's sarcoma, leiomyoma, hemangioma, lipoma, neurofibroma, fibroma), or cancer of the large bowel (adenocarcinoma, tubular adenoma, villous adenoma, hamartoma, leiomyoma).

Examples of cancers that are treatable using the compounds of the present disclosure include, but are not limited to, glioblastoma and neuroblastoma having underexpression of VRK2, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include melanoma (e.g., metastatic malignant melanoma, BRAF and HSP90 inhibition-resistant melanoma), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer, urothelial cancer (e.g., bladder) and cancers with high microsatellite instability (MSI^{high}). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including follicular lymphoma, including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma, and giant cell tumors.

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre -tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

It is believed that compounds of Formula (I), or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

The terms “individual”, “patient,” and “subject” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

### I. Cancer Therapies

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure for treatment of VRK1-associated diseases, disorders or conditions. Other agents such as therapeutic antibodies can be used in combination with the compounds of the present disclosure for treatment of VRK1-associated diseases, disorders or conditions. The one or more additional pharmaceutical agents can be administered to a patient simultaneously or sequentially.

In some embodiments, the compound or salt is administered or used in combination with a BCL2 inhibitor.

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein. Examples of cancers include solid tumors and non-solid tumors, such as liquid tumors, and blood cancers.

Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-□R, PKA, PKG, PKC, CaM-kinase, phosphorylase kinase, MEKK, ERK, MAPK, mTOR, EGFR, HER2, HER3, HER4, INS—R, IDH2, IGF-1R, IR—R, PDGF□R, PDGF□R, PI3K (alpha, beta, gamma, delta, and multiple or selective), CSF1R, KIT, FLK-II, KDR/FLK-1, FLK-4, fit-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2, e.g., ruxolitinib or baricitinib; JAK1, e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., INCB00928); or combinations thereof.

In some embodiments, the compound or salt described herein is administered with a PI3Kδ inhibitor. In some embodiments, the compound or salt described herein is administered with a JAK inhibitor. In some embodiments, the compound or salt described herein is administered with a JAK1 or JAK2 inhibitor (e.g., baricitinib or ruxolitinib). In some embodiments, the compound or salt described herein is administered with a JAK1 inhibitor. In some embodiments, the compound or salt described herein is administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™, e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide, 17-alpha-ethinylestradiol, diethylstilbestrol, testosterone, prednisone, fluoxymesterone, dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesterone acetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-I agonists, oncolytic virotherapy and immunomodulating small molecules, including thalidomide or JAK1/2 inhibitor, PI3Kδ inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a compound or salt of the present disclosure with an additional agent.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self -antigens. Examples of pathogens for which this therapeutic approach may be particularly useful include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

Viruses causing infections treatable by methods of the present disclosure include, but are not limited to human papillomavirus, influenza, hepatitis A, B, C or D viruses, adenovirus, poxvirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

### II. Immune-Checkpoint Therapies

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, CDK4, CDK12, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, WO 03042402, WO 2008156712, WO 2010089411, WO 2010036959, WO 2011066342, WO 2011159877, WO 2011082400, or WO 2011161699, which are each incorporated herein by reference in its entirety.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012. In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as R^{G}7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1B).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

As provided throughout, the additional compounds, inhibitors, agents, etc. can be combined with the present compound in a single or continuous dosage form, or they can be administered simultaneously or sequentially as separate dosage forms.

### Labeled Compounds and Assay Methods

The compounds of the present disclosure can further be useful in investigations of biological processes in normal and abnormal tissues. Thus, another aspect of the present disclosure relates to labeled compounds of the present disclosure (radio-labeled, fluorescent-labeled, etc.) that would be useful not only in imaging techniques but also in assays, both in vitro and in vivo, for localizing and quantitating VRK1 protein in tissue samples, including human, and for identifying VRK1 ligands by inhibition binding of a labeled compound. Accordingly, the present disclosure includes VRK1 binding assays that contain such labeled compounds.

The present disclosure further includes isotopically-labeled compounds of the disclosure. An “isotopically” or “radio-labeled” compound is a compound of the present disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as -CD₃ being substituted for —CH₃). In some embodiments, alkyl groups in Formula (I) can be perdeuterated.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro VRK1 protein labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the present disclosure can be used in a screening assay to identify and/or evaluate compounds. For example, a newly synthesized or identified compound (i.e., test compound) which is labeled can be evaluated for its ability to bind a VRK1 protein by monitoring its concentration variation when contacting with the VRK1 protein, through tracking of the labeling. For example, a test compound (labeled) can be evaluated for its ability to reduce binding of another compound which is known to bind to a VRK1 protein (i.e., standard compound). Accordingly, the ability of a test compound to compete with the standard compound for binding to the VRK1 protein directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Enumerated Embodiments

Embodiment 1. A compound of Formula (I):

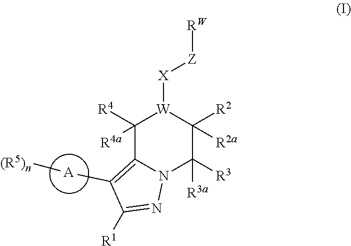

or a pharmaceutically acceptable salt thereof, wherein:
n is 0, 1, 2, 3, 4, 5, or 6;
W is CH or N;
when W is N, then X is CR⁶R^{6a}, C(O), S(O), or S(O)₂;
when W is CH, then X is NR^{6b}, CR⁶R^{6a} C(O), S(O), or S(O)₂;
Z is -Cy¹- or -Cy¹-Cy²-, wherein Cy¹ is bound to X, provided that when Z is -Cy¹-Cy²-, then Cy² is bound to R^{W};
Cy¹ is C₃₋₁₂ cycloalkyl, 6-10 membered aryl, 4-12 membered heterocycloalkyl, and 5-membered heteroaryl, each of which is optionally substituted by 1, 2, 3, 4, 5, or 6 independently selected R⁷ substituents;
Cy² is C₃₋₁₂ cycloalkyl, 6-10 membered aryl, 4-12 membered heterocycloalkyl, and 5-10 membered heteroaryl, each of which is optionally substituted by 1, 2, 3, 4, 5, or 6 independently selected R⁷ substituents;
Ring moiety A is C₃₋₁₀ cycloalkyl, 6-10 membered aryl, 4-10 membered heterocycloalkyl, or 5-10 membered heteroaryl;
R¹ is H, D, halo, CN, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, or C₁₋₃ alkoxy-C₁₋₄ alkyl;
R² and R^{2a} are each independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2} NR^{c2}R^{d2}, NR^{c2}C(O)R^{b2}, NR^{c2}C(O)OR^{a2}, NR^{c2}C(O)NR^{c2}R^{d2} NR^{c2}S(O)NR^{c2}R^{d2} NR^{c2}S(O)R^{b2}, NR^{c2}S(O)₂R^{b2} NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or R² and R^{2a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R² and R^{2a}, taken together with the carbon atom to which they are attached, form a C(O) group;
each R^{a2}, R^{c2}, and R^{d2} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c2} and R^{d2} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
R³ and R^{3a} are each independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3} NR^{c3}S(O)NR^{c3}R^{d3} NR^{c3}S(O)R^{b3}, NR^{c3}S(O)₂R^{b3} NR^{c3}S(O)₂NR^{c3}R^{d3} S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c3} and R^{d3} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents;
each R^{b3} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
R⁴ and R^{4a} are each independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}C(O)R^{b4} NR^{c4}C(O)OR^{a4}, NR^{c4}C(O)NR^{c4}R^{d4} NR^{c4}S(O)NR^{c4}R^{d4} NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c4} and R^{d4} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents;
each R^{b4} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R⁵ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
or, any R^{c5} and R^{d5} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51} NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)NR^{c51}R^{d51}, NR^{c51}S(O)R^{b51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, S(O)(═NR^{e51})R^{b51}, SF₅, P(O)R^{f51}R^{g51}, OP(O)(OR^{h51})(ORⁱ⁵¹), P(O)(OR^{h51})(ORⁱ⁵¹), and BR^{j51}R^{k51}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5B} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5B} substituents;
or, any R^{c51} and R^{d51} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{5B} substituents;
each R^{b51} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{5B} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f51} and R^{g51} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h51} and Rⁱ⁵¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a52}, SR^{a52}, NHOR^{a52}, C(O)R^{b52}, C(O)NR^{c52}R^{d52}, C(O)NR^{c52}(OR^{a52}), C(O)OR^{a52}, OC(O)R^{b52}, OC(O)NR^{c52}R^{d52}, NR^{c52}R^{d52}, NR^{c52}NR^{c52}R^{d52}, NR^{c52}C(O)R^{b52}, NR^{c52}C(O)OR^{a52}, NR^{c52}C(O)NR^{c52}R^{d52}, C(═NR^{e52})R^{b52}, C(═NR^{e52})NR^{c2}R^{d52}, NR^{c52}C(═NR^{e52})NR^{c52}R^{d52}, NR^{c52}C(═NR^{e52})R^{b52}, NR^{c52}S(O)NR^{c52}R^{d52}, NR^{c52}S(O)R^{b52}, NR^{c52}S(O)₂R^{b52}, NR^{c52}S(O)(═NR^{e52})R^{b52}, NR^{c52}S(O)₂NR^{c52}R^{d52}, S(O)R^{b52}, S(O)NR^{c52}R^{d52}, S(O)₂R^{b52}, S(O)₂NR^{c52}R^{d52}, OS(O)(═NR^{e52})R^{b52}, OS(O)₂R^{b52}, S(O)(═NR^{e52})R^{b52}, SF₅, P(O)R^{f52}R^{g52}, OP(O)(OR^{h52})(ORⁱ⁵²), P(O)(OR^{h52})(ORⁱ⁵²), and BR^{j52}R^{k52}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a52}, R^{c52}, and R^{d52} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c52} and R^{d52} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b52} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e52} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f52} and R^{g52} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h52} and Rⁱ⁵² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j52} and R^{k52} is independently selected from OH, C₁₋₆ alkoxy, and C₁₋₆ haloalkoxy;
or any R^{j52} and R^{k52} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁶ and R^{6a} are each independently selected from H, D, halo, CN, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
R^{6b} is selected from H, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R⁷ is independently selected from D, halo, CN, NO₂, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)NR^{c7}R^{d7}, NR^{c7}S(O)R^{b7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, OS(O)(═NR^{e7})R^{b7}, OS(O)₂R^{b7}, S(O)(═NR^{e7})R^{b7}, SF₅, P(O)R^{f7}R^{g7}, OP(O)(OR^{h7})(ORⁱ⁷), P(O)(OR^{h7})(ORⁱ⁷), and BR^{j7}R^{k7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
or, any R^{c7} and R^{d7} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h7} and Rⁱ⁷ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j7} and R^{k7} is independently selected from OH, C₁₋₆ alkoxy, and C₁₋₆ haloalkoxy;
or any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, NHOR^{a71}, C(O)R^{b71}, C(O)NR^{c71}R^{d71}, C(O)NR^{c71}(OR^{a71}), C(O)OR^{a71}, OC(O)R^{b71}, OC(O)NR^{c71}R^{d71}, NR^{c71}R^{d71}, NR^{c71}NR^{c71}R^{d71}, NR^{c71}C(O)R^{b71}, NR^{c71}C(O)OR^{a71}, NR^{c71}C(O)NR^{c71}R^{d71}, C(═NR^{e71})R^{b71}, C(═NR^{e71})NR^{c71}R^{d71}, NR^{c71}C(═NR^{e71})NR^{c71}R^{d71}, NR^{c71}C(═NR^{e71})R^{b71}, NR^{c71}S(O)NR^{c71}R^{d71}, NR^{c71}S(O)R^{b71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, OS(O)₂R^{b71}, S(O)(═NR^{e71})R^{b71}, SF₅, P(O)R^{f71}R^{g71}, OP(O)(OR^{h71})(ORⁱ⁷¹), P(O)(OR^{h71})(ORⁱ⁷¹), and BR^{j71}R^{k71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f71} and R^{g71} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h71} and Rⁱ⁷¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{g71} and R^{k71} is independently selected from OH, C₁₋₆ alkoxy, and C₁₋₆ haloalkoxy;
or any R^{j71} and R^{k71} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a72}, SR^{a72}, NHOR^{a72}, C(O)R^{b72}, C(O)NR^{c72}R^{d72}, C(O)NR^{c72}(OR^{a72}), C(O)OR^{a72}, OC(O)R^{b72}, OC(O)NR^{c72}R^{d72}, NR^{c72}R^{d72}, NR^{c72}NR^{c72}R^{d72}, NR^{c72}C(O)R^{b72}, NR^{c72}C(O)OR^{a72}, NR^{c72}C(O)NR^{c72}R^{d72}, C(═NR^{e72})R^{b72}, C(═NR^{e72})NR^{c72}R^{d72}, NR^{c72}C(═NR^{e72})NR^{c72}R^{d72}, NR^{c72}C(═NR^{e72})R^{b72}, NR^{c72}S(O)NR^{c72}R^{d72}, NR^{c72}S(O)R^{b72}, NR^{c72}S(O)₂R^{b72}, NR^{c72}S(O)(═NR^{e72})R^{b72}, NR^{c72}S(O)₂NR^{c72}R^{d72}, S(O)R^{b72}, S(O)NR^{c72}R^{d72}, S(O)₂R^{b72}, S(O)₂NR^{c72}R^{d72}, OS(O)(═NR^{e72})R^{b72}, OS(O)₂R^{b72}, S(O)(═NR^{e72})R^{b72}, SF₅, P(O)R^{f72}R^{g72}, OP(O)(OR^{h72})(ORⁱ⁷²), P(O)(OR^{h72})(ORⁱ⁷²), and BR^{j72}R^{k72}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a72}, R^{c72}, and R^{d72} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c72} and R^{d72} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b72} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e72} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f72} and R^{g72} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h72} and Rⁱ⁷² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j72} and R^{k72} is independently selected from OH, C₁₋₆ alkoxy, and C₁₋₆ haloalkoxy;
or any R^{j72} and R^{k72} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{W} is a covalent warhead moiety having a reactive functional group capable of covalently binding to one or more cysteine residues in a protein; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 2. The compound of embodiment 1, or a pharmaceutically acceptable salt thereof, wherein R^{W} is a means for covalently binding to cysteine residue in a protein.

Embodiment 3. The compound of embodiment 1 or 2, or a pharmaceutically acceptable salt thereof, wherein R^{W} is:

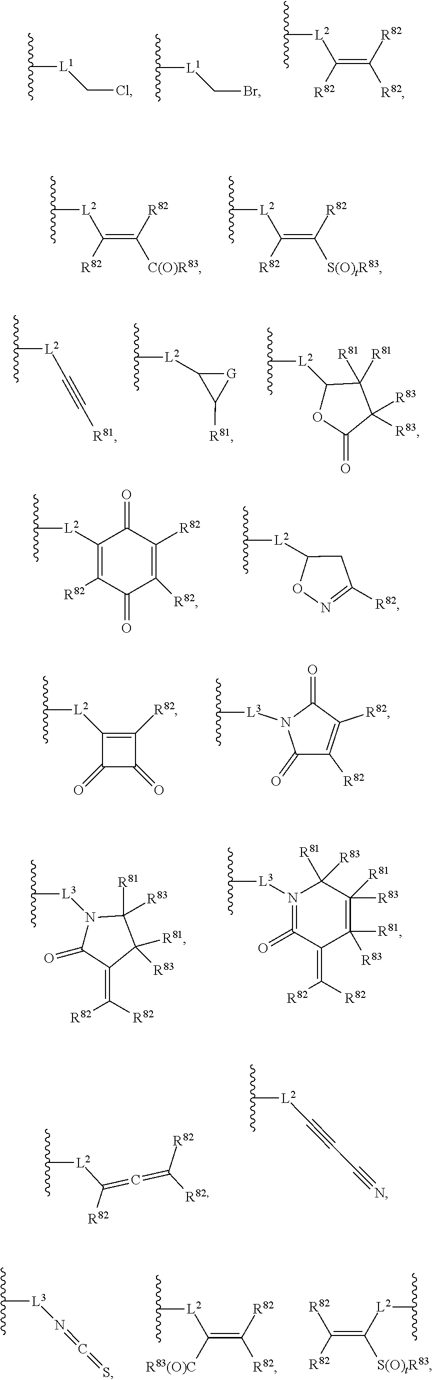

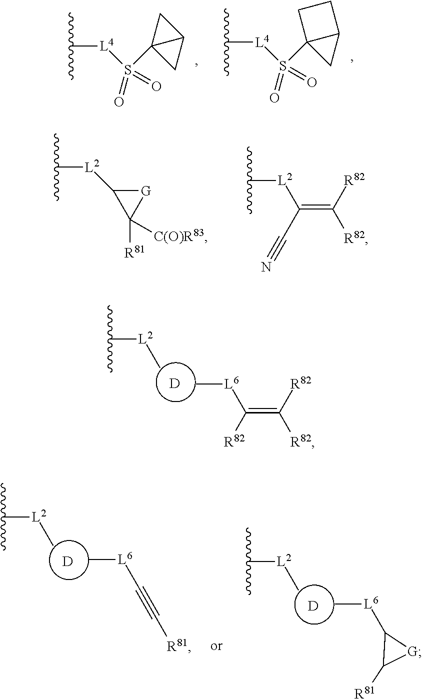

L¹ is -L-C(O)—, -L-NR⁹C(O)—, -L-OC(O)—, -L-S(O)—, -L-S(O)₂—, -L-NRS(O)—, -L-OS(O)—, -L-NR⁹S(O)₂—, or -L-OS(O)₂—, wherein L¹ is attached to Z through the L linking group;
L² is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹S(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L² is attached to Z through the L linking group;
L³ is -L-, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)₂—, or -L-OS(O)₂—, wherein L³ is attached to Z through the L linking group;
L⁴ is -L-, -L-O—, -L-S—, or -L-NR⁹—, wherein L⁴ is attached to Z through the L linking group;
L⁶ is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹S(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L⁶ is attached to Ring moiety D through the L linking group;
L is a bond or C₁₋₆ alkylene, wherein said C₁₋₆ alkylene is optionally substituted by 1, 2, 3, or 4 independently selected R^{G} substituents; or
L is —O—C₁₋₆ alkyl or —N(R^{N})—C₁₋₆ alkyl, wherein L is attached to Ring moiety A via the oxygen of the —O—C₁₋₆ alkyl or the nitrogen atom of the —N(R^{N})—C₁₋₆ alkyl group;
each R^{N} is independently H or C₁₋₆ alkyl;
Ring D is a 4-12 membered heterocycloalkyl, C₃₋₁₂ cycloalkyl, C₆₋₁₀ aryl, or a 5-10 membered heteroaryl, each of which is optionally substituted with 1, 2, 3, or 4 independently selected C₁₋₆ alkyl groups;
G is O or NR⁹;
each t is independently 0, 1, 2, or 3;
each R⁸¹ and R⁸³ are independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{c8}R^{d8}, NR^{c8}R^{d8}, NR^{c8}NR^{c8}R^{d8}, NR^{c8}C(O)R^{b8}, NR^{c8}C(O)OR^{a8}, NR^{c8}C(O)NR^{c8}R^{d8}, C(═NR^{e8})R^{b8}, C(═NR^{e8})NR^{c8}R^{d8}, NR^{c8}C(═NR^{e8})R^{c8}R^{d8}, NR^{c8}C(═NR^{e8})R^{b8}, NR^{c8}S(O)NR^{c8}R^{d8}, NR^{c8}S(O)R^{b8}, NR^{c8}S(O)₂R^{b8}, NR^{c8}S(O)(═NR^{e8})R^{b8}, NR^{c8}S(O)₂NR^{c8}R^{d8}, S(O)R^{b8}, S(O)NR^{c8}R^{d8}, S(O)₂R^{b8}, S(O)₂NR^{c8}R^{d8}, OS(O)(═NR^{e8})R^{b8}, OS(O)₂R^{b8}, S(O)(═NR^{e8})R^{b8}, SF₅, P(O)R^{f8}R^{g8}, OP(O)(OR^{h8})(ORⁱ⁸), P(O)(OR^{h8})(ORⁱ¹), and BR^{j8}R^{k8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R⁸² is independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, C(O)R^{b8}, C(O)NR^{c8}R^{d8}, C(O)OR^{a8}, C(═NR^{e8})R^{b8}, C(═NR^{e8})NR^{c8}R^{d8}, S(O)R^{b8}, S(O)NR^{c8}R^{d8}, S(O)₂R^{b8}, and S(O)₂NR^{c8}R^{d8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b8} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h8} and Rⁱ⁸ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j8} and R^{k8} is independently selected from OH, C₁₋₆ alkoxy, and C₁₋₆ haloalkoxy;
or any R^{j8} and R^{k8} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
or any two R⁸¹ and R⁸² together with the atoms to which they are attached, form C₃₋₇ cycloalkyl, 4-7 membered heterocycloalkyl, phenyl, or 5-6-membered heteroaryl ring, each of which is optionally substituted with 1, 2, 3, or 4 independently selected R^{G} substitutents;
each R⁹ is independently selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, OR^{a9}, SR^{a9}, NHOR^{a9}, C(O)R^{b9}, C(O)NR^{c9}R^{d9}, C(O)NR^{c9}(OR^{a9}), C(O)OR^{a9}, OC(O)R^{b9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, C(═NR^{e9})R^{b9}, C(═NR^{e9})NR^{c9}R^{d9}, NR^{c9}C(═NR^{e9})NR^{c9}R^{d9}, NR^{c9}C(═NR^{e9})R^{b9}, NR^{c9}S(O)NR^{c9}R^{d9}, NR^{c9}S(O)R^{b9}, NR^{c9}S(O)₂R^{b9}, NR^{c9}S(O)(═NR^{e9})R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, OS(O)(═NR^{e9})R^{b9}, OS(O)₂R^{b9}, and S(O)(═NR^{e9})R^{b9}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a9}, R^{c9}, and R^{d9} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c9} and R^{d9} attached to the same N atom, together with the N atom to which they are attached, form a 4-7 membered heterocycloalkyl group, which is optionally substituted with 1, 2, 3, or 4 independently selected R^{G} substituents;
each R^{b9} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents; and
each R^{e9} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl.

Embodiment 4. The compound of embodiment 3, or a pharmaceutically acceptable salt thereof, wherein R^{W} is selected from:

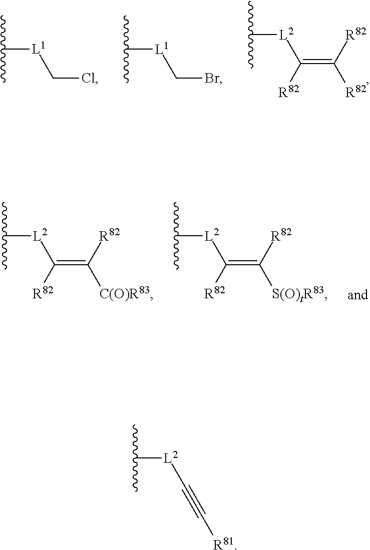

Embodiment 5. The compound of embodiment 3, or a pharmaceutically acceptable salt thereof, wherein R^{W} is selected from:

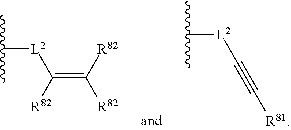

Embodiment 6. The compound of embodiment 3, or a pharmaceutically acceptable salt thereof, wherein R^{W} is

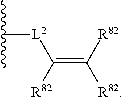

Embodiment 7. The compound of embodiment 3 or 4, or a pharmaceutically acceptable salt thereof, wherein L¹ is -L-C(O)—, -L-NR⁹C(O)—, -L-S(O)₂—, -L-NR⁹S(O)—, or -L-NR⁹S(O)₂—, or wherein L¹ is attached to Z through the L linking group; L is a bond; and R⁹ is selected from H and C₁₋₆ alkyl.

Embodiment 8. The compound of any one of embodiments 3-6, or a pharmaceutically acceptable salt thereof, wherein L² is -L-C(O)—, -L-NR⁹C(O)—, -L-S(O)₂—, or -L-NR⁹S(O)₂—, wherein L² is attached to Z through the L linking group; L is a bond; and R⁹ is selected from H and C₁₋₆ alkyl.

Embodiment 9. The compound of any one of embodiments 3-6, or a pharmaceutically acceptable salt thereof, wherein L² is —C(O)—, —NR⁹C(O)—, —S(O)₂—, or —NR⁹S(O)₂—; and R⁹ is selected from H and C₁₋₃ alkyl.

Embodiment 10. The compound of any one of embodiments 3-6, or a pharmaceutically acceptable salt thereof, wherein when L² is —C(O)— or —S(O)₂— and Z is Cy¹, then L² is attached to a nitrogen ring member of Cy¹; and when L² is —C(O)— or —S(O)₂— and Z is -Cy¹-Cy²-, then then L² is attached to a nitrogen ring member of Cy².

Embodiment 11. The compound of any one of embodiments 3-10, or a pharmaceutically acceptable salt thereof, wherein R⁸¹, R⁸², and R⁸³ are each independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents.

Embodiment 12. The compound of any one of embodiments 3-10, or a pharmaceutically acceptable salt thereof, wherein R⁸¹, R⁸², and R⁸³ are each independently selected from H, halo, CN, C₁₋₆ alkyl, C₁₋₆ haloalkyl, C₃₋₇ cycloalkyl, 4-7 membered heterocycloalkyl, C₃₋₇ cycloalkyl-C₁₋₄ alkyl, and 4-7 membered heterocycloalkyl-C₁₋₄ alkyl, wherein said C₁₋₆ alkyl, C₁₋₆ haloalkyl, C₃₋₇ cycloalkyl, 4-7 membered heterocycloalkyl, C₃₋₇ cycloalkyl-C₁₋₄ alkyl, and 4-7 membered heterocycloalkyl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, amino, C₁₋₃ alkylamino, and di(C₁₋₃ alkyl)amino.

Embodiment 13. The compound of any one of embodiments 3-10, or a pharmaceutically acceptable salt thereof, wherein R⁸¹, R⁸², and R⁸³ are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

Embodiment 14. The compound of any one of embodiments 1-13, or a pharmaceutically acceptable salt thereof, wherein n is 0, 1, 2, 3, or 4.

Embodiment 15. The compound of any one of embodiments 1-13, or a pharmaceutically acceptable salt thereof, wherein n is 1, 2, or 3.

Embodiment 16. The compound of any one of embodiments 1-13, or a pharmaceutically acceptable salt thereof, wherein n is 1.

Embodiment 17. The compound of any one of embodiments 1-13, or a pharmaceutically acceptable salt thereof, wherein n is 2.

Embodiment 18. The compound of any one of embodiments 1-13, or a pharmaceutically acceptable salt thereof, wherein n is 3.

Embodiment 19. The compound of any one of embodiments 1-18, or a pharmaceutically acceptable salt thereof, wherein W is CH.

Embodiment 20. The compound of any one of embodiments 1-18, or a pharmaceutically acceptable salt thereof, wherein W is N.

Embodiment 21. The compound of any one of embodiments 1-20, or a pharmaceutically acceptable salt thereof, wherein R⁶ and R^{6a} are each independently selected from H, D, halo, CN, C₁₋₃ alkyl, and C₁₋₃ haloalkyl; and R^{6b} is selected from H, C₁₋₃ alkyl, and C₁₋₃ haloalkyl.

Embodiment 22. The compound of any one of embodiments 1-20, or a pharmaceutically acceptable salt thereof, wherein R^{6b} is H.

Embodiment 23. The compound of any one of embodiments 1-20, or a pharmaceutically acceptable salt thereof, wherein X is CR⁶R^{6a}, C(O) or S(O)₂.

Embodiment 24. The compound of any one of embodiments 1-20, or a pharmaceutically acceptable salt thereof, wherein R⁶ and R^{6a} are each H.

Embodiment 25. The compound of any one of embodiments 1-24, or a pharmaceutically acceptable salt thereof, wherein X is C(O).

Embodiment 26. The compound of any one of embodiments 1-24, or a pharmaceutically acceptable salt thereof, wherein X is CR⁶R^{6a}.

Embodiment 27. The compound of any one of embodiments 1-24, or a pharmaceutically acceptable salt thereof, wherein X is CH₂.

Embodiment 28. The compound of any one of embodiments 1-24, or a pharmaceutically acceptable salt thereof, wherein X is S(O)₂.

Embodiment 29. The compound of any one of embodiments 1-28, or a pharmaceutically acceptable salt thereof, wherein Z is Cy¹.

Embodiment 30. The compound of any one of embodiments 1-29, or a pharmaceutically acceptable salt thereof, wherein Cy¹ is phenyl, naphthyl, 5-10 membered heteroaryl, or 9-12 membered heterocycloalkyl.

Embodiment 31. The compound of any one of embodiments 1-29, or a pharmaceutically acceptable salt thereof, wherein Cy¹ is phenyl, naphthyl, 5-6 membered heteroaryl, 9-10 membered heteroaryl, or 9-12 membered heterocycloalkyl.

Embodiment 32. The compound of any one of embodiments 1-29, or a pharmaceutically acceptable salt thereof, wherein Cy¹ is selected from phenyl, naphthyl, pyridinyl, pyrazolyl, thiazolyl, indolinyl, isoindolinyl, 2,3-dihydro-1H-pyrrolo[2,3-b]pyridinyl, 1,2,3,4-tetrahydroquinolinyl, 2,3-dihydrobenzo[b][1,4]dioxinyl, 2,3-dihydro-1H-pyrrolo[3,4-c]pyridinyl, 3,4-dihydro-2H-benzo[b][1,4]oxazinyl, and isoquinolinyl.

Embodiment 33. The compound of any one of embodiments 1-29, or a pharmaceutically acceptable salt thereof, wherein Cy¹ is phenyl.

Embodiment 34. The compound of any one of embodiments 1-29, or a pharmaceutically acceptable salt thereof, wherein Cy¹ is naphthyl.

Embodiment 35. The compound of any one of embodiments 1-29, or a pharmaceutically acceptable salt thereof, wherein Cy¹ is 5-6 membered heteroaryl.

Embodiment 36. The compound of any one of embodiments 1-29, or a pharmaceutically acceptable salt thereof, wherein Cy¹ is pyridinyl.

Embodiment 37. The compound of any one of embodiments 1-29, or a pharmaceutically acceptable salt thereof, wherein Cy¹ is 9-10 membered heteroaryl.

Embodiment 38. The compound of any one of embodiments 1-29, or a pharmaceutically acceptable salt thereof, wherein Cy¹ is 9-12 membered heterocycloalkyl.

Embodiment 39. The compound of any one of embodiments 1-29, or a pharmaceutically acceptable salt thereof, wherein Cy¹ is

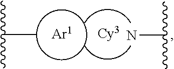

wherein Ar¹ is fused to Cy³, Ar¹ is bonded to X, Ar¹ is phenyl or 5-6 membered heteroaryl, Cy³ is 4-8 membered heterocycloalkyl, and Cy¹ is optionally substituted with 1, 2, 3, or 4 independently selected R⁷ substituents.

Embodiment 40. The compound of any one of embodiments 1-29, or a pharmaceutically acceptable salt thereof, wherein Cy¹ is phenyl, naphthyl, 5-6 membered heteroaryl, 9-10 membered heteroaryl, or

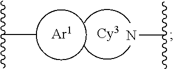

wherein:
Ar¹ is fused to Cy³ and Ar¹ is bonded to X;
Ar¹ is phenyl or 5-6 membered heteroaryl;
Cy³ is 4-8 membered heterocycloalkyl; and
Cy¹ is optionally substituted with 1, 2, 3, or 4 independently selected R⁷ substituents.

Embodiment 41. The compound of any one of embodiments 1-40, or a pharmaceutically acceptable salt thereof, wherein R¹ selected from H, halo, CN, C₁₋₆ alkyl, and C₁₋₆ haloalkyl.

Embodiment 42. The compound of any one of embodiments 1-40, or a pharmaceutically acceptable salt thereof, wherein R¹ selected from H, halo, CN, CH₃ and CF₃.

Embodiment 43. The compound of any one of embodiments 1-40, or a pharmaceutically acceptable salt thereof, wherein R¹ is H.

Embodiment 44. The compound of any one of embodiments 1-43, or a pharmaceutically acceptable salt thereof, wherein Ring moiety A is 6-10 membered aryl or 5-10 membered heteroaryl.

Embodiment 45. The compound of any one of embodiments 1-43, or a pharmaceutically acceptable salt thereof, wherein Ring moiety A is phenyl or 5-10 membered heteroaryl.

Embodiment 46. The compound of any one of embodiments 1-43, or a pharmaceutically acceptable salt thereof, wherein Ring moiety A is 6-10 membered aryl.

Embodiment 47. The compound of any one of embodiments 1-43, or a pharmaceutically acceptable salt thereof, wherein Ring moiety A is phenyl.

Embodiment 48. The compound of any one of embodiments 1-43, or a pharmaceutically acceptable salt thereof, wherein Ring moiety A is 5-10 membered heteroaryl.

Embodiment 49. The compound of any one of embodiments 1-43, or a pharmaceutically acceptable salt thereof, wherein Ring moiety A is 5-6 membered heteroaryl or 9-10 membered heteroaryl.

Embodiment 50. The compound of any one of embodiments 1-43, or a pharmaceutically acceptable salt thereof, wherein Ring moiety A is 5-6 membered heteroaryl.

Embodiment 51. The compound of any one of embodiments 1-43, or a pharmaceutically acceptable salt thereof, wherein Ring moiety A is pyridinyl or pyrimidinyl.

Embodiment 52. The compound of any one of embodiments 1-43, or a pharmaceutically acceptable salt thereof, wherein Ring moiety A is pyridinyl.

Embodiment 53. The compound of any one of embodiments 1-43, or a pharmaceutically acceptable salt thereof, wherein Ring moiety A is pyrimidinyl.

Embodiment 54. The compound of any one of embodiments 1-43, or a pharmaceutically acceptable salt thereof, wherein Ring moiety A is 9-10 membered heteroaryl.

Embodiment 55. The compound of any one of embodiments 1-43, or a pharmaceutically acceptable salt thereof, wherein Ring moiety A is phenyl, pyridinyl, or pyrimidinyl.

Embodiment 56. The compound of any one of embodiments 1-55, or a pharmaceutically acceptable salt thereof, wherein R² and R^{2a} are each independently selected from H, D, halo, CN, C₁₋₆ alkyl, and C₁₋₆ haloalkyl, and OR^{b2}, wherein said C₁₋₆ alkyl and C₁₋₆ haloalkyl are each optionally substituted with 1, 2, 3, or 4 independently selected R^{G} substituents;
or R² and R^{2a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R² and R^{2a}, taken together with the carbon atom to which they are attached, form a C(O) group; and
each R^{b2} is independently selected from C₁₋₆ alkyl and C₁₋₆ haloalkyl, which are each optionally substituted with 1, 2, 3, or 4 independently selected R^{G} substituents.

Embodiment 57. The compound of any one of embodiments 1-55, or a pharmaceutically acceptable salt thereof, wherein R² and R^{2a} are each independently selected from H, D, halo, CN, C₁₋₆ alkyl, and C₁₋₆ haloalkyl, and OR^{b2};
or R² and R^{2a}, taken together with the carbon atom to which they are attached, form a C₃₋₇ cycloalkyl ring or a 3-7 membered heterocycloalkyl ring;
or R² and R^{2a}, taken together with the carbon atom to which they are attached, form a C(O) group; and
each R^{b2} is independently selected from C₁₋₆ alkyl and C₁₋₆ haloalkyl.

Embodiment 58. The compound of any one of embodiments 1-55, or a pharmaceutically acceptable salt thereof, wherein R² and R^{2a} are each independently selected from H, halo, CN, C₁₋₆ alkyl, and C₁₋₆ haloalkyl.

Embodiment 59. The compound of any one of embodiments 1-55, or a pharmaceutically acceptable salt thereof, wherein R² and R^{2a} are each independently selected from H, halo, CN, CH₃ and CF₃.

Embodiment 60. The compound of any one of embodiments 1-55, or a pharmaceutically acceptable salt thereof, wherein R² and R^{2a} are each H.

Embodiment 61. The compound of any one of embodiments 1-55, or a pharmaceutically acceptable salt thereof, wherein R² and R^{2a} are each independently selected from H or C₁₋₃ alkyl;
or R² and R^{2a}, taken together with the carbon atom to which they are attached, form a cyclopropyl ring.

Embodiment 62. The compound of any one of embodiments 1-61, or a pharmaceutically acceptable salt thereof, wherein R³ and R^{3a} are each independently selected from H, D, halo, CN, C₁₋₆ alkyl, and C₁₋₆ haloalkyl, and OR^{b3}, wherein said C₁₋₆ alkyl and C₁₋₆ haloalkyl are each optionally substituted with 1, 2, 3, or 4 independently selected R^{G} substituents;
or R³ and R^{3a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R³ and R^{3a}, taken together with the carbon atom to which they are attached, form a C(O) group; and
each R^{b3} is independently selected from C₁₋₆ alkyl and C₁₋₆ haloalkyl, which are each optionally substituted with 1, 2, 3, or 4 independently selected R^{G} substituents.

Embodiment 63. The compound of any one of embodiments 1-61, or a pharmaceutically acceptable salt thereof, wherein R³ and R^{3a} are each independently selected from H, D, halo, CN, C₁₋₆ alkyl, and C₁₋₆ haloalkyl, and OR^{b3}.
or R³ and R^{3a}, taken together with the carbon atom to which they are attached, form a C₃₋₇ cycloalkyl ring or a 3-7 membered heterocycloalkyl ring;
or R³ and R^{3a}, taken together with the carbon atom to which they are attached, form a C(O) group; and
each R^{b3} is independently selected from C₁₋₆ alkyl and C₁₋₆ haloalkyl.

Embodiment 64. The compound of any one of embodiments 1-61, or a pharmaceutically acceptable salt thereof, wherein R³ and R^{3a} are each independently selected from H, halo, CN, C₁₋₆ alkyl, and C₁₋₆ haloalkyl.

Embodiment 65. The compound of any one of embodiments 1-61, or a pharmaceutically acceptable salt thereof, wherein R³ and R^{3a} are each independently selected from H, halo, CN, CH₃ and CF₃.

Embodiment 66. The compound of any one of embodiments 1-61, or a pharmaceutically acceptable salt thereof, wherein R³ and R^{3a} are each independently selected from H and CN.

Embodiment 67. The compound of any one of embodiments 1-66, or a pharmaceutically acceptable salt thereof, wherein R⁴ and R^{4a} are each independently selected from H, D, halo, CN, C₁₋₆ alkyl, and C₁₋₆ haloalkyl, and OR^{b4}, wherein said C₁₋₆ alkyl and C₁₋₆ haloalkyl are each optionally substituted with 1, 2, 3, or 4 independently selected R^{G} substituents;
or R⁴ and R^{4a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R⁴ and R^{4a}, taken together with the carbon atom to which they are attached, form a C(O) group; and
each R^{b4} is independently selected from C₁₋₆ alkyl and C₁₋₆ haloalkyl, which are each optionally substituted with 1, 2, 3, or 4 independently selected R^{G} substituents.

Embodiment 68. The compound of any one of embodiments 1-66, or a pharmaceutically acceptable salt thereof, wherein R⁴ and R^{4a} are each independently selected from H, D, halo, CN, C₁₋₆ alkyl, and C₁₋₆ haloalkyl;
or R⁴ and R^{4a}, taken together with the carbon atom to which they are attached, form a C₃₋₇ cycloalkyl ring or a 3-7 membered heterocycloalkyl ring;
or R⁴ and R^{4a}, taken together with the carbon atom to which they are attached, form a C(O) group; and
each R^{b4} is independently selected from C₁₋₆ alkyl and C₁₋₆ haloalkyl.

Embodiment 69. The compound of any one of embodiments 1-66, or a pharmaceutically acceptable salt thereof, wherein R⁴ and R^{4a} are each independently selected from H, halo, CN, C₁₋₆ alkyl, and C₁₋₆ haloalkyl.

Embodiment 70. The compound of any one of embodiments 1-66, or a pharmaceutically acceptable salt thereof, wherein R⁴ and R^{4a} are each independently selected from H, halo, CN, CH₃ and CF₃.

Embodiment 71. The compound of any one of embodiments 1-66, or a pharmaceutically acceptable salt thereof, wherein R⁴ and R^{4a} are each H.

Embodiment 72. The compound of any one of embodiments 1-71, or a pharmaceutically acceptable salt thereof, wherein each R⁵ is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-10 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, NHOR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-10 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents.

Embodiment 73. The compound of any one of embodiments 1-71, or a pharmaceutically acceptable salt thereof, wherein each R⁵ is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, NHOR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c}S(O)₂NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents.

Embodiment 74. The compound of any one of embodiments 1-71, or a pharmaceutically acceptable salt thereof, wherein each R⁵ is independently selected from D, halo, CN, C₁₋₆ alkyl, C₂₋₆ alkenyl, C₂₋₆ alkynyl, C₁₋₆ haloalkyl, phenyl, 5-10 membered heteroaryl, OR^{a5} and NR^{c5}R^{d5} wherein said C₁₋₆ alkyl, C₂₋₆ alkenyl, C₂₋₆ alkynyl, C₁₋₆ haloalkyl, phenyl, and 5-10 membered heteroaryl are each optionally substituted with 1, 2, 3, or 4 independently selected R^{5A} substituents.

Embodiment 75. The compound of any one of embodiments 1-71, or a pharmaceutically acceptable salt thereof, wherein each R⁵ is independently selected from D, halo, CN, C₁₋₆ alkyl, C₂₋₆ alkenyl, C₂₋₆ alkynyl, C₁₋₆ haloalkyl, OR^{a5} and NR^{c5}R^{d5}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{5A} substituents.

Embodiment 76. The compound of any one of embodiments 1-75, or a pharmaceutically acceptable salt thereof, wherein:
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl which are each optionally substituted with 1, 2, 3, or 4 independently selected R^{5A} substituents; and
each R^{5A} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 77. The compound of any one of embodiments 1-75, or a pharmaceutically acceptable salt thereof, wherein:
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, wherein said C₁₋₆ alkyl, C₂₋₆ alkenyl, C₂₋₆ alkynyl, C₁₋₆ haloalkyl, C₃₋₄ cycloalkyl, and 4-5 membered heterocycloalkyl are each optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents; and
each R^{5A} is independently selected from OH, CN, halo, C₁₋₃ alkyl, C₁₋₃ haloalkyl, amino, C₁₋₃ alkoxy, C₁₋₃ alkylamino, and di(C₁₋₃ alkyl)amino.

Embodiment 78. The compound of any one of embodiments 1-75, or a pharmaceutically acceptable salt thereof, wherein:
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, wherein said C₁₋₆ alkyl, C₂₋₆ alkenyl, C₂₋₆ alkynyl, C₁₋₆ haloalkyl, C₃₋₄ cycloalkyl, and 4-5 membered heterocycloalkyl are each optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents; and
each R^{5A} is independently selected from OH, CN, halo, C₁₋₃ alkyl, C₁₋₃ haloalkyl, amino, C₁₋₃ alkylamino, and di(C₁₋₃ alkyl)amino.

Embodiment 79. The compound of any one of embodiments 1-71, or a pharmaceutically acceptable salt thereof, wherein each R⁵ is independently selected from halo, CN, C₁₋₃ alkyl, C₁₋₃ haloalkyl, OH, C₁₋₃ alkoxy, C₁₋₃ haloalkoxy, phenyl, pyridinyl, pyrimidinyl, anisolyl, and 2,3-dihydrobenzofuranyl.

Embodiment 80. The compound of any one of embodiments 1-71, or a pharmaceutically acceptable salt thereof, wherein each R⁵ is independently selected from halo, CN, C₁₋₃ alkyl, C₁₋₃ haloalkyl, OH, C₁₋₃ alkoxy, and C₁₋₃ haloalkoxy.

Embodiment 81. The compound of any one of embodiments 1-71, or a pharmaceutically acceptable salt thereof, wherein each R⁵ is independently selected from Cl, F, CN, CH₃, OH, and OCH₃.

Embodiment 82. The compound of any one of embodiments 1-45 and 56-81, or a pharmaceutically acceptable salt thereof, wherein

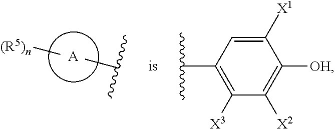

wherein X¹ and X² are each independently Cl or F, and X³ is H, Cl or F.

Embodiment 83. The compound of any one of embodiments 1-45 and 56-81, or a pharmaceutically acceptable salt thereof, wherein

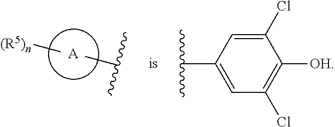

Embodiment 84. The compound of any one of embodiments 1-45 and 56-81, or a pharmaceutically acceptable salt thereof, wherein

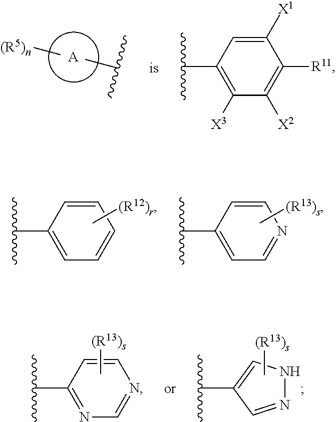

X¹ is H, Cl, F, or OCH₃;
X² is H, Cl, or F;
X³ is H, Cl, F, or OCH₃;
R¹¹ is H, OH, or CN;
r is 2;
s is 0, 1, or 2;
two R¹² substituents, together with the carbon atoms to which they are attached, form a tetrahydrofuranyl, pyrazole, pyrrole, or pyrimidine ring, which is optionally substituted by 1 or 2 independently selected R⁵ substituents selected from halo, CN, and C₁₋₃ alkyl; and
each R¹³ is independently selected from halo, CN, C₁₋₃ alkyl, and OCH₃.

Embodiment 85. The compound of any one of embodiments 1-45 and 56-81, or a pharmaceutically acceptable salt thereof, wherein

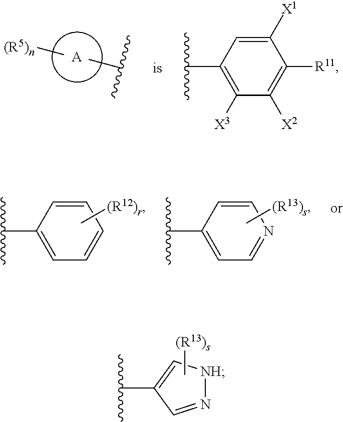

X¹ is Cl, F, or OCH₃;
X² is H, Cl, or F;
X³ is H, Cl or F;
R¹¹ is OH or CN;
r is 2;
s is 0, 1, or 2;
two R¹² substituents, together with the carbon atoms to which they are attached, form a pyrazole, pyrrole, or pyrimidine ring, which is optionally substituted by 1 or 2 independently selected R⁵ substituents selected from halo, CN, and C₁₋₃ alkyl; and
each R¹³ is independently selected from halo, CN, and C₁₋₃ alkyl.

Embodiment 86. The compound of any one of embodiments 1-45 and 56-81, or a pharmaceutically acceptable salt thereof, wherein

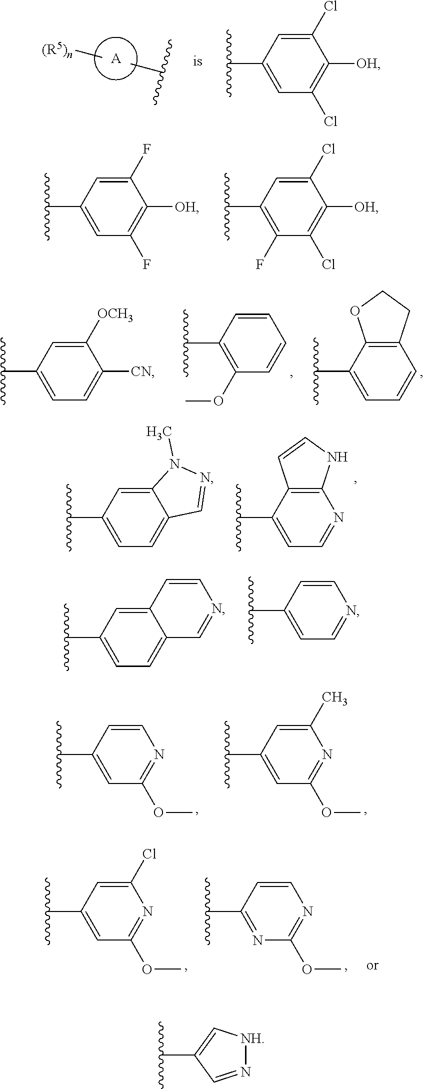

Embodiment 87. The compound of any one of embodiments 1-45 and 56-81, or a pharmaceutically acceptable salt thereof, wherein

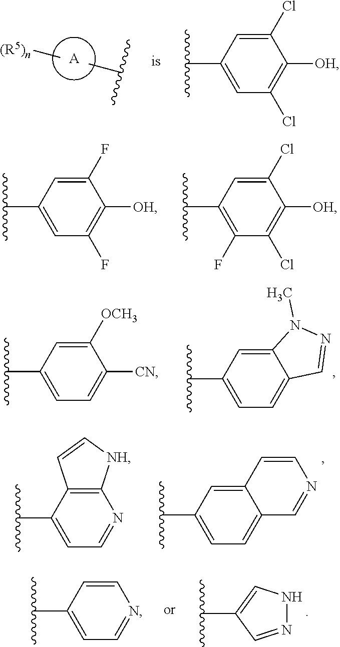

Embodiment 88. The compound of any one of embodiments 1-87, or a pharmaceutically acceptable salt thereof, wherein each R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents.

Embodiment 89. The compound of any one of embodiments 1-87, or a pharmaceutically acceptable salt thereof, wherein each R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents.

Embodiment 90. The compound of any one of embodiments 1-87, or a pharmaceutically acceptable salt thereof, wherein each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents.

Embodiment 91. The compound of any one of embodiments 1-90, or a pharmaceutically acceptable salt thereof, wherein:
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c71}C(O)OR^{a71}, NR^{c71}C(O)NR^{c71}R^{d71}, NR^{c71}S(O)₂R^{b71}, NR^{c71}S(O)₂NR^{c71}R^{d71}, S(O)₂R^{b71}, and S(O)₂NR^{c71}R^{d71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 92. The compound of any one of embodiments 1-90, or a pharmaceutically acceptable salt thereof, wherein:
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, C₃₋₇cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl; and
each R^{b71} is independently selected from C₁₋₆ alkyl and C₁₋₆ haloalkyl.

Embodiment 93. The compound of any one of embodiments 1-90, or a pharmaceutically acceptable salt thereof, wherein:
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents; and
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino.

Embodiment 94. The compound of embodiment 1, or a pharmaceutically acceptable salt thereof, wherein:
n is 0, 1, 2, 3, or 4;
W is N;
X is CR⁶R^{6a}, C(O), or S(O)₂;
Z is Cy¹;
Cy¹ is phenyl, naphthyl, 5-6 membered heteroaryl, 9-10 membered heteroaryl, or

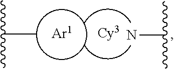

each of which is optionally substituted by 1, 2, 3, 4, 5, or 6 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl or 5-10 membered heteroaryl;
R² and R^{2a} are each independently selected from H, D, halo, CN, C₁₋₆ alkyl, and C₁₋₆ haloalkyl, and OR^{b2}, wherein said C₁₋₆ alkyl and C₁₋₆ haloalkyl are each optionally substituted with 1, 2, 3, or 4 independently selected R^{G} substituents;
or R² and R^{2a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R² and R^{2a}, taken together with the carbon atom to which they are attached, form a C(O) group;
each R^{b2} is independently selected from C₁₋₆ alkyl and C₁₋₆ haloalkyl, which are each optionally substituted with 1, 2, 3, or 4 independently selected R^{G} substituents;
R³ and R^{3a} are each independently selected from H, D, halo, CN, C₁₋₆ alkyl, and C₁₋₆ haloalkyl, and OR^{b3}, wherein said C₁₋₆ alkyl and C₁₋₆ haloalkyl are each optionally substituted with 1, 2, 3, or 4 independently selected R^{G} substituents;
or R³ and R^{3a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R³ and R^{3a}, taken together with the carbon atom to which they are attached, form a C(O) group;
each R^{b3} is independently selected from C₁₋₆ alkyl and C₁₋₆ haloalkyl, which are each optionally substituted with 1, 2, 3, or 4 independently selected R^{G} substituents;
R⁴ and R^{4a} are each independently selected from H, D, halo, CN, C₁₋₆ alkyl, and C₁₋₆ haloalkyl, and OR^{b4}, wherein said C₁₋₆ alkyl and C₁₋₆ haloalkyl are each optionally substituted with 1, 2, 3, or 4 independently selected R^{G} substituents;
or R⁴ and R^{4a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R⁴ and R^{4a}, taken together with the carbon atom to which they are attached, form a C(O) group;
each R^{b4} is independently selected from C₁₋₆ alkyl and C₁₋₆ haloalkyl, which are each optionally substituted with 1, 2, 3, or 4 independently selected R^{G} substituents;
each R⁵ is independently selected from D, halo, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, OR^{a5} and NR^{c5}R^{d5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, and 5-10 membered heteroaryl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, wherein said C₁₋₆ alkyl, C₂₋₆ alkenyl, C₂₋₆ alkynyl, C₁₋₆ haloalkyl, C₃₋₄ cycloalkyl, and 4-5 membered heterocycloalkyl are each optionally substituted with 1, 2, 3, or 4 independently selected R^{5A} substituents;
each R^{5A} is independently selected from OH, OCH₃, CN, halo, C₁₋₃ alkyl, C₁₋₃ haloalkyl, amino, C₁₋₃ alkylamino, and di(C₁₋₃ alkyl)amino;
R⁶ and R^{6a} are each independently selected from H, D, halo, CN, C₁₋₃ alkyl, and C₁₋₃ haloalkyl;
R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c71}C(O)OR^{a71}, NR^{c71}C(O)NR^{c71}R^{d71}, NR^{c71}S(O)₂R^{b71}, NR^{c71}S(O)₂NR^{c71}R^{d71}, S(O)₂R^{b71}, and S(O)₂NR^{c71}R^{d71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{7B} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R^{W} is selected from:

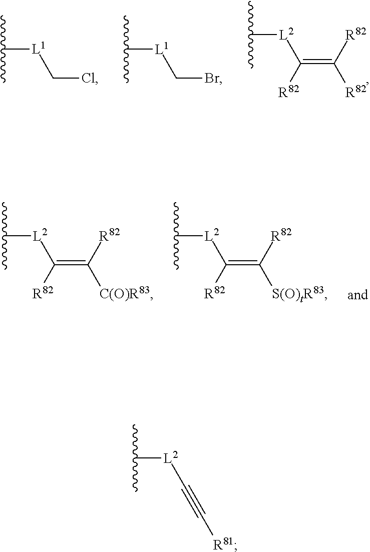

L¹ is -L-C(O)—, -L-NR⁹C(O)—, -L-S(O)₂—, -L-NR⁹S(O)—, or -L-NR⁹S(O)₂—, or wherein L¹ is attached to Z through the L linking group;
L² is -L-C(O)—, -L-NR⁹C(O)—, -L-S(O)₂—, or -L-NR⁹S(O)₂—, wherein L² is attached to Z through the L linking group;
L is a bond;
R⁹ is selected from H and C₁₋₆ alkyl;
R⁸¹, R⁸², and R⁸³ are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each t is independently 0, 1, 2, or 3; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 95. The compound of embodiment 1, or a pharmaceutically acceptable salt thereof, wherein:
n is 0, 1, 2, 3, or 4;
W is N;
X is CR⁶R^{6a}, C(O), or S(O)₂;
Z is Cy¹;
Cy¹ is phenyl, naphthyl, 5-6 membered heteroaryl, 9-10 membered heteroaryl, or

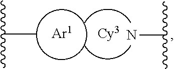

each of which is optionally substituted by 1, 2, 3, 4, 5, or 6 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl or 5-10 membered heteroaryl;
R² and R^{2a} are each independently selected from H, D, halo, CN, C₁₋₆ alkyl, and C₁₋₆ haloalkyl, and OR^{b2}, wherein said C₁₋₆ alkyl and C₁₋₆ haloalkyl are each optionally substituted with 1, 2, 3, or 4 independently selected R^{G} substituents;
or R² and R^{2a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R² and R^{2a}, taken together with the carbon atom to which they are attached, form a C(O) group;
each R^{b2} is independently selected from C₁₋₆ alkyl and C₁₋₆ haloalkyl, which are each optionally substituted with 1, 2, 3, or 4 independently selected R^{G} substituents;
R³ and R^{3a} are each independently selected from H, D, halo, CN, C₁₋₆ alkyl, and C₁₋₆ haloalkyl, and OR^{b3}, wherein said C₁₋₆ alkyl and C₁₋₆ haloalkyl are each optionally substituted with 1, 2, 3, or 4 independently selected R^{G} substituents;
or R³ and R^{3a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R³ and R^{3a}, taken together with the carbon atom to which they are attached, form a C(O) group;
each R^{b3} is independently selected from C₁₋₆ alkyl and C₁₋₆ haloalkyl, which are each optionally substituted with 1, 2, 3, or 4 independently selected R^{G} substituents;
R⁴ and R^{4a} are each independently selected from H, D, halo, CN, C₁₋₆ alkyl, and C₁₋₆ haloalkyl, and OR^{b4}, wherein said C₁₋₆ alkyl and C₁₋₆ haloalkyl are each optionally substituted with 1, 2, 3, or 4 independently selected R^{G} substituents;
or R⁴ and R^{4a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R⁴ and R^{4a}, taken together with the carbon atom to which they are attached, form a C(O) group;
each R^{b4} is independently selected from C₁₋₆ alkyl and C₁₋₆ haloalkyl, which are each optionally substituted with 1, 2, 3, or 4 independently selected R^{G} substituents;
each R⁵ is independently selected from D, halo, CN, C₁₋₆ alkyl, C₂₋₆ alkenyl, C₂₋₆ alkynyl, C₁₋₆ haloalkyl, OR^{a5} and NR^{c5}R^{d5}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, wherein said C₁₋₆ alkyl, C₂₋₆ alkenyl, C₂₋₆ alkynyl, C₁₋₆ haloalkyl, C₃₋₄ cycloalkyl, and 4-5 membered heterocycloalkyl are each optionally substituted with 1, 2, 3, or 4 independently selected R^{5A} substituents;
each R^{5A} is independently selected from OH, CN, halo, C₁₋₃ alkyl, C₁₋₃ haloalkyl, amino, C₁₋₃ alkylamino, and di(C₁₋₃ alkyl)amino;
R⁶ and R^{6a} are each independently selected from H, D, halo, CN, C₁₋₃ alkyl, and C₁₋₃ haloalkyl;
R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c71}C(O)OR^{a71}, NR^{c71}C(O)NR^{c71}R^{d71}, NR^{c71}S(O)₂R^{b71}, NR^{c71}S(O)₂NR^{c71}R^{d71}, S(O)₂R^{b71}, and S(O)₂NR^{c71}R^{d71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{7B} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R^{W} is selected from:

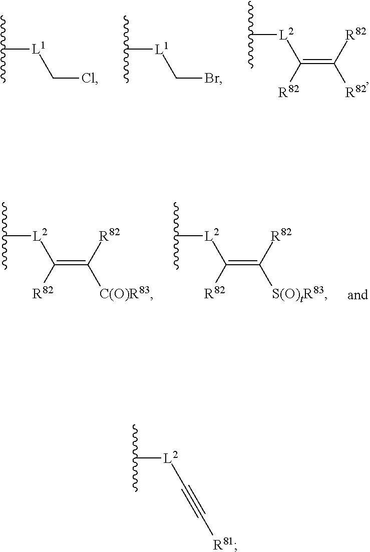

L¹ is -L-C(O)—, -L-NR⁹C(O)—, -L-S(O)₂—, -L-NR⁹S(O)—, or -L-NR⁹S(O)₂—, or wherein L¹ is attached to Z through the L linking group;
L² is -L-C(O)—, -L-NR⁹C(O)—, -L-S(O)₂—, or -L-NR⁹S(O)₂—, wherein L² is attached to Z through the L linking group;
L is a bond;
R⁹ is selected from H and C₁₋₆ alkyl;
R⁸¹, R⁸², and R⁸³ are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each t is independently 0, 1, 2, or 3; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 96. The compound of embodiment 1, or a pharmaceutically acceptable salt thereof, wherein:
n is 0, 1, 2, 3, or 4;
W is N;
X is CR⁶R^{6a}, C(O), or S(O)₂;
Z is Cy¹;
Cy¹ is phenyl, naphthyl, 5-6 membered heteroaryl, 9-10 membered heteroaryl, or

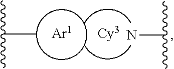

each of which is optionally substituted by 1, 2, 3, 4, 5, or 6 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl;
R¹ is H;
Ring moiety A is phenyl, 5-6 membered heteroaryl, or 9-10 membered heteroaryl;
R² and R^{2a} are each independently selected from H, D, halo, CN, C₁₋₆ alkyl, and C₁₋₆ haloalkyl, and OR^{b2};
or R² and R^{2a}, taken together with the carbon atom to which they are attached, form a C₃₋₇ cycloalkyl ring or a 3-7 membered heterocycloalkyl ring;
each R^{a2} is independently selected from C₁₋₆ alkyl and C₁₋₆ haloalkyl, which are each optionally substituted with 1, 2, 3, or 4 independently selected R^{G} substituents;
R³ and R^{3a} are each independently selected from H, D, halo, CN, C₁₋₆ alkyl, and C₁₋₆ haloalkyl, and OR^{b3};
or R³ and R^{3a}, taken together with the carbon atom to which they are attached, form a C₃₋₇ cycloalkyl ring or a 3-7 membered heterocycloalkyl ring;
R⁴ and R^{4a} are each independently selected from H, D, halo, CN, C₁₋₆ alkyl, and C₁₋₆ haloalkyl, and OR^{b4}, wherein said C₁₋₆ alkyl and C₁₋₆ haloalkyl are each optionally substituted with 1, 2, 3, or 4 independently selected R^{G} substituents;
or R⁴ and R^{4a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
each R^{b4} is independently selected from C₁₋₆ alkyl and C₁₋₆ haloalkyl, which are each optionally substituted with 1, 2, 3, or 4 independently selected R^{G} substituents;
R⁵ is independently selected from D, halo, CN, C₁₋₆ alkyl, C₂₋₆ alkenyl, C₂₋₆ alkynyl, C₁₋₆ haloalkyl, C₃₋₄ cycloalkyl, C₃₋₇ cycloalkyl, phenyl, 4-7 membered heterocycloalkyl, 5-6 membered heteroaryl, OR^{as} and NR^{c5}R^{d5}, wherein said C₁₋₆ alkyl, C₂₋₆ alkenyl, C₂₋₆ alkynyl, C₁₋₆ haloalkyl, C₃₋₄ cycloalkyl C₃₋₇ cycloalkyl, phenyl, 4-7 membered heterocycloalkyl, and 5-6 membered heteroaryl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents;
each R^{5A} is independently selected from OH, OCH₃, CN, halo, C₁₋₃ alkyl, C₁₋₃ haloalkyl, amino, C₁₋₃ alkylamino, and di(C₁₋₃ alkyl)amino;
R⁶ and R^{6a} are each independently selected from H and C₁₋₃ alkyl;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl;
each R^{b71} is independently selected from C₁₋₆ alkyl and C₁₋₆ haloalkyl;
R^{W} is selected from:

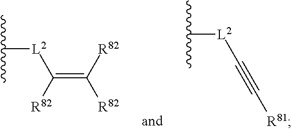

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 97. The compound of embodiment 1, or a pharmaceutically acceptable salt thereof, wherein:
n is 0, 1, 2, 3, or 4;
W is N;
X is CR⁶R^{6a}, C(O), or S(O)₂;
Z is Cy¹;
Cy¹ is phenyl, naphthyl, 5-6 membered heteroaryl, 9-10 membered heteroaryl, or

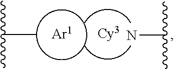

each of which is optionally substituted by 1, 2, 3, 4, 5, or 6 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl;
R¹ is H;
Ring moiety A is phenyl, 5-6 membered heteroaryl, or 9-10 membered heteroaryl;
R² and R^{2a} are each independently selected from H, D, halo, CN, C₁₋₆ alkyl, and C₁₋₆ haloalkyl, and OR^{b2};
or R² and R^{2a}, taken together with the carbon atom to which they are attached, form a C₃₋₇ cycloalkyl ring or a 3-7 membered heterocycloalkyl ring;
each R^{a2} is independently selected from C₁₋₆ alkyl and C₁₋₆ haloalkyl, which are each optionally substituted with 1, 2, 3, or 4 independently selected R^{G} substituents;
R³ and R^{3a} are each independently selected from H, D, halo, CN, C₁₋₆ alkyl, and C₁₋₆ haloalkyl, and OR^{b3};
or R³ and R^{3a}, taken together with the carbon atom to which they are attached, form a C₃₋₇ cycloalkyl ring or a 3-7 membered heterocycloalkyl ring;
R⁴ and R^{4a} are each independently selected from H, D, halo, CN, C₁₋₆ alkyl, and C₁₋₆ haloalkyl, and OR^{b4}, wherein said C₁₋₆ alkyl and C₁₋₆ haloalkyl are each optionally substituted with 1, 2, 3, or 4 independently selected R^{G} substituents;
or R⁴ and R^{4a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
each R^{b4} is independently selected from C₁₋₆ alkyl and C₁₋₆ haloalkyl, which are each optionally substituted with 1, 2, 3, or 4 independently selected R^{G} substituents;
R⁵ is independently selected from D, halo, CN, C₁₋₆ alkyl, C₂₋₆ alkenyl, C₂₋₆ alkynyl, C₁₋₆ haloalkyl, C₃₋₄ cycloalkyl, OR^{a5} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents;
each R^{5A} is independently selected from OH, CN, halo, C₁₋₃ alkyl, C₁₋₃ haloalkyl, amino, C₁₋₃ alkylamino, and di(C₁₋₃ alkyl)amino;
R⁶ and R^{6a} are each independently selected from H and C₁₋₃ alkyl;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d7};
each R^{a71}, R^{c71}, and R^{d71} is independently selected from H, C₁₋₆ alkyl, and C₁₋₆ haloalkyl;
each R^{b71} is independently selected from C₁₋₆ alkyl and C₁₋₆ haloalkyl;
R^{W} is selected from:

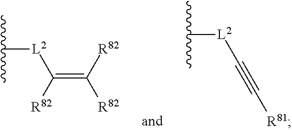

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 98. The compound of embodiment 1, or a pharmaceutically acceptable salt thereof, wherein:
n is 0, 1, 2, 3, or 4;
W is N;
X is CH₂, C(O), or S(O)₂;
Z is Cy¹;
Cy¹ is phenyl, naphthyl, 5-6 membered heteroaryl, 9-10 membered heteroaryl, or

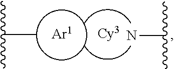

each of which is optionally substituted by 1, 2, or 3 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl, 5-6 membered heteroaryl, or 9-10 membered heteroaryl;
R² and R^{2a} are each independently selected from H, halo, CN, C₁₋₆ alkyl, and C₁₋₆ haloalkyl;
R³ and R^{3a} are each independently selected from H, halo, CN, C₁₋₆ alkyl, and C₁₋₆ haloalkyl;
R⁴ and R^{4a} are each independently selected H, halo, CN, C₁₋₆ alkyl, and C₁₋₆ haloalkyl;
each R⁵ is independently selected from halo, CN, C₁₋₃ alkyl, C₁₋₃ haloalkyl, OH, C₁₋₃ alkoxy, C₁₋₃ haloalkoxy, phenyl, and 5-6 membered heteroaryl, wherein said phenyl and 5-6 membered heteroaryl are each optionally substituted with 1 or 2 independently selected R^{5A} substituents;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂-3 alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from:

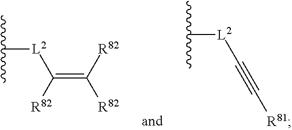

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl; and
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

Embodiment 99. The compound of embodiment 1, or a pharmaceutically acceptable salt thereof, wherein:
n is 0, 1, 2, 3, or 4;
W is N;
X is CH₂, C(O), or S(O)₂;
Z is Cy¹;
Cy¹ is phenyl, naphthyl, 5-6 membered heteroaryl, 9-10 membered heteroaryl, or

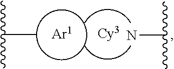

each of which is optionally substituted by 1, 2, or 3 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl, 5-6 membered heteroaryl, or 9-10 membered heteroaryl;
R² and R^{2a} are each independently selected from H, halo, CN, C₁₋₆ alkyl, and C₁₋₆ haloalkyl;
R³ and R^{3a} are each independently selected from H, halo, CN, C₁₋₆ alkyl, and C₁₋₆ haloalkyl;
R⁴ and R^{4a} are each independently selected H, halo, CN, C₁₋₆ alkyl, and C₁₋₆ haloalkyl;
each R⁵ is independently selected from halo, CN, C₁₋₃ alkyl, C₁₋₃ haloalkyl, OH, C₁₋₃ alkoxy, and C₁₋₃ haloalkoxy;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂-3 alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from:

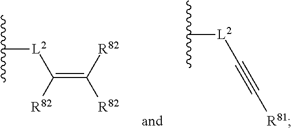

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl; and
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

Embodiment 100. The compound of embodiment 1, or a pharmaceutically acceptable salt thereof, wherein:
n is 0, 1, 2, 3, or 4;
W is N;
X is CH₂, C(O), or S(O)₂;
Z is Cy¹;
Cy¹ is phenyl, naphthyl, 5-6 membered heteroaryl, 9-10 membered heteroaryl, or

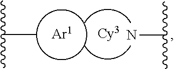

each of which is optionally substituted by 1, 2, or 3 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl, 5-6 membered heteroaryl, or 9-10 membered heteroaryl;
R² and R^{2a} are each independently selected from H, halo, CN, C₁₋₆ alkyl, and C₁₋₆ haloalkyl;
or R² and R^{2a}, taken together with the carbon atom to which they are attached, form a cyclopropyl ring;
R³ and R^{3a} are each independently selected from H, halo, CN, C₁₋₆ alkyl, and C₁₋₆ haloalkyl;
R⁴ and R^{4a} are each independently selected H, halo, CN, C₁₋₆ alkyl, and C₁₋₆ haloalkyl; each R⁵ is independently selected from halo, CN, C₁₋₃ alkyl, C₁₋₃ haloalkyl, OH, C₁₋₃ alkoxy, C₁₋₃ haloalkoxy, phenyl, and 5-6 membered heteroaryl, wherein said phenyl and 5-6 membered heteroaryl are each optionally substituted with 1 or 2 independently selected R^{5A} substituents;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from:

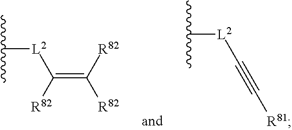

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl; and
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

Embodiment 101. The compound of embodiment 1, or a pharmaceutically acceptable salt thereof, wherein:
n is 0, 1, 2, 3, or 4;
W is N;
X is CH₂, C(O), or S(O)₂;
Z is Cy¹;
Cy¹ is phenyl, naphthyl, 5-6 membered heteroaryl, 9-10 membered heteroaryl, or

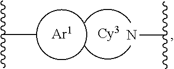

each of which is optionally substituted by 1, 2, or 3 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl, 5-6 membered heteroaryl, or 9-10 membered heteroaryl;
R² and R^{2a} are each independently selected from H, halo, CN, C₁₋₆ alkyl, and C₁₋₆ haloalkyl;
or R² and R^{2a}, taken together with the carbon atom to which they are attached, form a cyclopropyl ring;
R³ and R^{3a} are each independently selected from H, halo, CN, C₁₋₆ alkyl, and C₁₋₆ haloalkyl;
R⁴ and R^{4a} are each independently selected H, halo, CN, C₁₋₆ alkyl, and C₁₋₆ haloalkyl;
each R⁵ is independently selected from halo, CN, C₁₋₃ alkyl, C₁₋₃ haloalkyl, OH, C₁₋₃ alkoxy, and C₁₋₃ haloalkoxy;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂-3 alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from:

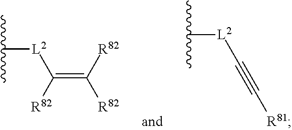

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl; and
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

Embodiment 102. The compound of any one of embodiments 94-101, or a pharmaceutically acceptable salt thereof, wherein

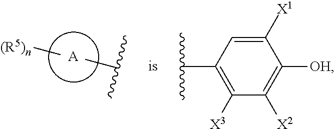

wherein X¹ and X² are each independently Cl or F, and X³ is H, Cl or F.

Embodiment 103. The compound of any one of embodiments 94-101, or a pharmaceutically acceptable salt thereof, wherein

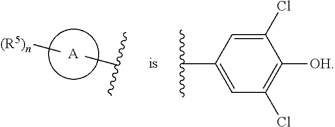

Embodiment 104. The compound of any one of embodiments 94-101, or a pharmaceutically acceptable salt thereof, wherein

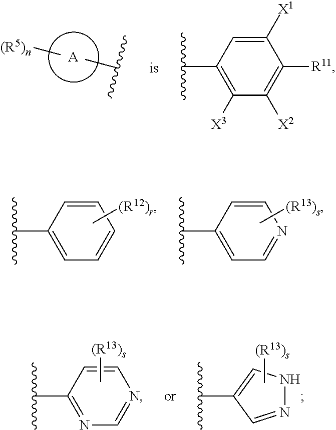

X¹ is H, Cl, F, or OCH₃;
X² is H, Cl, or F;
X³ is H, Cl, F, or OCH₃;
R¹¹ is H, OH, or CN;
r is 2;
s is 0, 1, or 2;
two R¹² substituents, together with the carbon atoms to which they are attached, form a tetrahydrofuranyl, pyrazole, pyrrole, or pyrimidine ring, which is optionally substituted by 1 or 2 independently selected R⁵ substituents selected from halo, CN, and C₁₋₃ alkyl; and
each R¹³ is independently selected from halo, CN, C₁₋₃ alkyl, and OCH₃.

Embodiment 105. The compound of any one of embodiments 94-101, or a pharmaceutically acceptable salt thereof, wherein

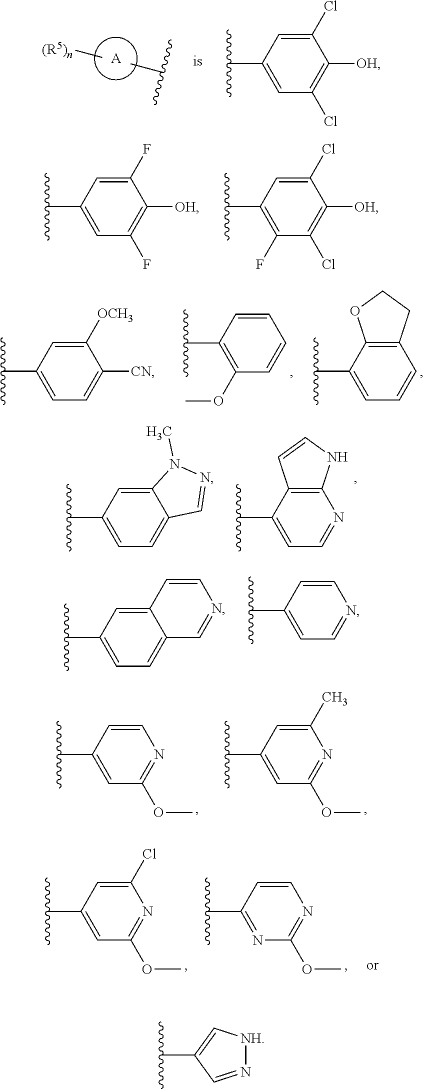

Embodiment 106. The compound of any one of embodiments 94-101, or a pharmaceutically acceptable salt thereof, wherein

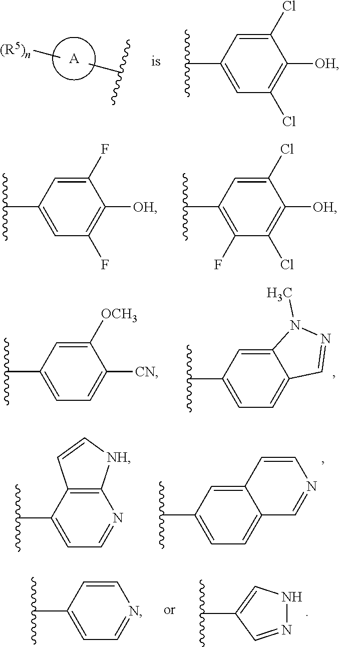

Embodiment 107. The compound of any one of embodiments 94-101, or a pharmaceutically acceptable salt thereof, wherein

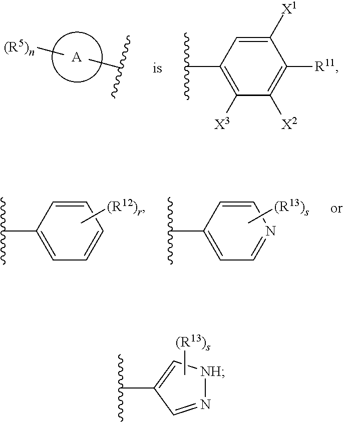

X¹ is Cl, F, or OCH₃;
X² is H, Cl, or F;
X³ is H, Cl or F;
R¹¹ is OH or CN;
r is 2;
s is 0, 1, or 2;
two R¹² substituents, together with the carbon atoms to which they are attached, form a pyrazole, pyrrole, or pyrimidine ring, which is optionally substituted by 1 or 2 independently selected R⁵ substituents selected from halo, CN, and C₁₋₃ alkyl; and
each R¹³ is independently selected from halo, CN, and C₁₋₃ alkyl.

Embodiment 108. The compound of any one of embodiments 94-104, or a pharmaceutically acceptable salt thereof, wherein Z is Cy¹; and Cy¹ is

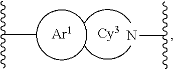

which is optionally substituted by 1, 2, or 3 independently selected R⁷ substituents.

Embodiment 109. The compound of embodiment 1, wherein the compound is selected from:
N-(4-(3-(3,5-dichloro-4-hydroxyphenyl)-4,5,6,7-tetrahydropyrazolo[1,5-a]pyrazine-5-carbonyl)phenyl)acrylamide;
N-(4-(3-(3,5-dichloro-4-hydroxyphenyl)-4,5,6,7-tetrahydropyrazolo[1,5-a]pyrazine-5-carbonyl)-2-methoxyphenyl)acrylamide;
1-(5-(3-(3,5-dichloro-4-hydroxyphenyl)-4,5,6,7-tetrahydropyrazolo[1,5-a]pyrazine-5-carbonyl)isoindolin-2-yl)prop-2-en-1-one;
N-(5-(3-(3,5-dichloro-4-hydroxyphenyl)-4,5,6,7-tetrahydropyrazolo[1,5-a]pyrazine-5-carbonyl)-6-methoxypyridin-2-yl)acrylamide;
N-(6-(3-(3,5-dichloro-4-hydroxyphenyl)-4,5,6,7-tetrahydropyrazolo[1,5-a]pyrazine-5-carbonyl)pyridin-3-yl)acrylamide;
N-(6-(3-(3,5-dichloro-4-hydroxyphenyl)-4,5,6,7-tetrahydropyrazolo[1,5-a]pyrazine-5-carbonyl)-2-methoxypyridin-3-yl)acrylamide;
N-(3-(3-(3,5-dichloro-4-hydroxyphenyl)-4,5,6,7-tetrahydropyrazolo[1,5-a]pyrazine-5-carbonyl)-1-methyl-1H-pyrazol-5-yl)acrylamide;
N-(2-(3-(3,5-dichloro-4-hydroxyphenyl)-4,5,6,7-tetrahydropyrazolo[1,5-a]pyrazine-5-carbonyl)thiazol-5-yl)acrylamide;
N-(5-(3-(3,5-dichloro-4-hydroxyphenyl)-4,5,6,7-tetrahydropyrazolo[1,5-a]pyrazine-5-carbonyl)quinolin-8-yl)acrylamide;
1-(6-(3-(3,5-dichloro-4-hydroxyphenyl)-4,5,6,7-tetrahydropyrazolo[1,5-a]pyrazine-5-carbonyl)-1,3-dihydro-2H-pyrrolo[3,4-c]pyridin-2-yl)prop-2-en-1-one;
1-(5-(3-(3,5-dichloro-4-hydroxyphenyl)-4,5,6,7-tetrahydropyrazolo[1,5-a]pyrazine-5-carbonyl)indolin-1-yl)prop-2-en-1-one;
1-(6-(3-(3,5-dichloro-4-hydroxyphenyl)-4,5,6,7-tetrahydropyrazolo[1,5-a]pyrazine-5-carbonyl)-3,4-dihydroquinolin-1(2H)-yl)prop-2-en-1-one;
N-(4-(3-(3,5-dichloro-4-hydroxyphenyl)-4,5,6,7-tetrahydropyrazolo[1,5-a]pyrazine-5-carbonyl)naphthalen-1-yl)acrylamide;
N-(8-(3-(3,5-dichloro-4-hydroxyphenyl)-4,5,6,7-tetrahydropyrazolo[1,5-a]pyrazine-5-carbonyl)-2,3-dihydrobenzo[b][1,4]dioxin-5-yl)acrylamide;
N-(5-(3-(3,5-dichloro-4-hydroxyphenyl)-4,5,6,7-tetrahydropyrazolo[1,5-a]pyrazine-5-carbonyl)isoquinolin-8-yl)acrylamide;
N-(8-(3-(3,5-dichloro-4-hydroxyphenyl)-4,5,6,7-tetrahydropyrazolo[1,5-a]pyrazine-5-carbonyl)isoquinolin-5-yl)acrylamide;
N-(5-(3-(3,5-dichloro-4-hydroxyphenyl)-4,5,6,7-tetrahydropyrazolo[1,5-a]pyrazine-5-carbonyl)-4-methoxypyridin-2-yl)acrylamide;
N-(6-(3-(3,5-dichloro-4-hydroxyphenyl)-4,5,6,7-tetrahydropyrazolo[1,5-a]pyrazine-5-carbonyl)-2-(trifluoromethyl)pyridin-3-yl)acrylamide;
1-(5-(3-(3,5-dichloro-4-hydroxyphenyl)-4,5,6,7-tetrahydropyrazolo[1,5-a]pyrazine-5-carbonyl)-2,3-dihydro-1H-pyrrolo[2,3-b]pyridin-1-yl)prop-2-en-1-one;
1-(7-((3-(3,5-dichloro-4-hydroxyphenyl)-6,7-dihydropyrazolo[1,5-a]pyrazin-5(4H)-yl)sulfonyl)-2,3-dihydro-4H-benzo[b][1,4]oxazin-4-yl)prop-2-en-1-one;
N-(7-((3-(3,5-dichloro-4-hydroxyphenyl)-6,7-dihydropyrazolo[1,5-a]pyrazin-5(4H)-yl)sulfonyl)naphthalen-2-yl)acrylamide;
N-(4-((3-(3,5-dichloro-4-hydroxyphenyl)-6,7-dihydropyrazolo[1,5-a]pyrazin-5(4H)-yl)sulfonyl)phenyl)acrylamide;
N-(5-((3-(3,5-dichloro-4-hydroxyphenyl)-6,7-dihydropyrazolo[1,5-a]pyrazin-5(4H)-yl)methyl)pyridin-2-yl)acrylamide;
N-(4-((3-(3,5-dichloro-4-hydroxyphenyl)-6,7-dihydropyrazolo[1,5-a]pyrazin-5(4H)-yl)methyl)-2-methoxyphenyl)acrylamide;
1-(5-((3-(3,5-dichloro-4-hydroxyphenyl)-6,7-dihydropyrazolo[1,5-a]pyrazin-5(4H)-yl)methyl)isoindolin-2-yl)prop-2-en-1-one;
N-(2-chloro-4-((3-(3,5-dichloro-4-hydroxyphenyl)-6,7-dihydropyrazolo[1,5-a]pyrazin-5(4H)-yl)methyl)phenyl)acrylamide;
N-(4-(3-(3,5-dichloro-4-hydroxyphenyl)-6-methyl-4,5,6,7-tetrahydropyrazolo[1,5-a]pyrazine-5-carbonyl)phenyl)acrylamide;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(dimethylamino)but-2-enamide;
(E)-N-(5-(3-(3,5-dichloro-4-hydroxyphenyl)-4,5,6,7-tetrahydropyrazolo[1,5-a]pyrazine-5-carbonyl)quinolin-8-yl)-4-(dimethylamino)but-2-enamide;
(E)-N-(4-(3-(3,5-dichloro-4-hydroxyphenyl)-4,5,6,7-tetrahydropyrazolo[1,5-a]pyrazine-5-carbonyl)phenyl)-4-(dimethylamino)but-2-enamide;
(E)-N-(4-(3-(3,5-dichloro-4-hydroxyphenyl)-4,5,6,7-tetrahydropyrazolo[1,5-a]pyrazine-5-carbonyl)-2-fluorophenyl)-4-(dimethylamino)but-2-enamide;
(E)-1-(5-(3-(3,5-dichloro-4-hydroxyphenyl)-4,5,6,7-tetrahydropyrazolo[1,5-a]pyrazine-5-carbonyl)indolin-1-yl)-4-(dimethylamino)but-2-en-1-one;
(E)-N-(4-(3-(3,5-dichloro-4-hydroxyphenyl)-4,5,6,7-tetrahydropyrazolo[1,5-a]pyrazine-5-carbonyl)phenyl)-4-(piperidin-1-yl)but-2-enamide;
(E)-N-(4-(3-(3,5-dichloro-4-hydroxyphenyl)-4,5,6,7-tetrahydropyrazolo[1,5-a]pyrazine-5-carbonyl)phenyl)-4-morpholinobut-2-enamide;
(E)-N-(4-(3-(3,5-dichloro-4-hydroxyphenyl)-4,5,6,7-tetrahydropyrazolo[1,5-a]pyrazine-5-carbonyl)phenyl)-4-(diethylamino)but-2-enamide;
(E)-1-(5-(3-(3,5-dichloro-4-hydroxyphenyl)-4,5,6,7-tetrahydropyrazolo[1,5-a]pyrazine-5-carbonyl)isoindolin-2-yl)-4-(dimethylamino)but-2-en-1-one;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(piperidin-1-yl)but-2-enamide;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(pyrrolidin-1-yl)but-2-enamide;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-morpholinobut-2-enamide;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(diethylamino)but-2-enamide;
(E)-N-(4-(3-(3,5-dichloro-4-hydroxyphenyl)-4,5,6,7-tetrahydropyrazolo[1,5-a]pyrazine-5-carbonyl)-3-methoxyphenyl)-4-(dimethylamino)but-2-enamide;
N-(2-chloro-4-(3-(3,5-dichloro-4-hydroxyphenyl)-4,5,6,7-tetrahydropyrazolo[1,5-a]pyrazine-5-carbonyl)phenyl)acrylamide;
N-(4-(3-(3,5-dichloro-4-hydroxyphenyl)-4,5,6,7-tetrahydropyrazolo[1,5-a]pyrazine-5-carbonyl)-2-fluorophenyl)acrylamide;
N-(3-chloro-4-(3-(3,5-dichloro-4-hydroxyphenyl)-4,5,6,7-tetrahydropyrazolo[1,5-a]pyrazine-5-carbonyl)phenyl)acrylamide;
N-(4-(3-(3,5-dichloro-4-hydroxyphenyl)-4,5,6,7-tetrahydropyrazolo[1,5-a]pyrazine-5-carbonyl)-3-(trifluoromethyl)phenyl)acrylamide;
N-(5-(3-(3,5-dichloro-4-hydroxyphenyl)-4,5,6,7-tetrahydropyrazolo[1,5-a]pyrazine-5-carbonyl)pyridin-2-yl)acrylamide;
N-(4-(3-(3,5-dichloro-4-hydroxyphenyl)-4,5,6,7-tetrahydropyrazolo[1,5-a]pyrazine-5-carbonyl)-3-methoxyphenyl)acrylamide;
N-(4-(3-(3,5-dichloro-4-hydroxyphenyl)-4,5,6,7-tetrahydropyrazolo[1,5-a]pyrazine-5-carbonyl)-2-ethoxyphenyl)acrylamide;
N-(4-(3-(3,5-dichloro-4-hydroxyphenyl)-4,5,6,7-tetrahydropyrazolo[1,5-a]pyrazine-5-carbonyl)-2-isopropoxyphenyl)acrylamide;
N-(4-(3-(3,5-dichloro-4-hydroxyphenyl)-4,5,6,7-tetrahydropyrazolo[1,5-a]pyrazine-5-carbonyl)-2-(trifluoromethoxy)phenyl)acrylamide;
N-(4-(3-(3,5-dichloro-4-hydroxyphenyl)-4,5,6,7-tetrahydropyrazolo[1,5-a]pyrazine-5-carbonyl)-2-(methoxy-d₃)phenyl)acrylamide; and
N-(3-cyano-4-(3-(3,5-dichloro-4-hydroxyphenyl)-4,5,6,7-tetrahydropyrazolo[1,5-a]pyrazine-5-carbonyl)phenyl)acrylamide;

or a pharmaceutically acceptable salt of any of the aforementioned.

Embodiment 110. The compound of embodiment 1, wherein the compound is selected from:
(E)-1-(5-((3-(3,5-dichloro-4-hydroxyphenyl)-6,7-dihydropyrazolo[1,5-a]pyrazin-5(4H)-yl)methyl)-2,3-dihydro-1H-pyrrolo[3,2-b]pyridin-1-yl)-4-(piperidin-1-yl)but-2-en-1-one; and
(E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)indolin-1-yl)-4-morpholinobut-2-en-1-one;

or a pharmaceutically acceptable salt of any of the aforementioned.

Embodiment 111. The compound of embodiment 1, wherein the compound is selected from:
(E)-1-(5-(3′-(2-(2,3-dihydrobenzofuran-7-yl)pyridin-4-yl)-4′,5′-dihydro-7′H-spiro[cyclopropane-1,6′-pyrazolo[1,5-a]pyrazine]-5′-carbonyl)indolin-1-yl)-4-(dimethylamino)but-2-en-1-one;
(E)-4-(dimethylamino)-1-(5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one;
(E)-4-(3-azabicyclo[3.1.1]heptan-3-yl)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)but-2-en-1-one;
(E)-1-(5-(3′-(2-methoxypyridin-4-yl)-4′,5′-dihydro-7′H-spiro[cyclopropane-1,6′-pyrazolo[1,5-a]pyrazine]-5′-carbonyl)indolin-1-yl)-4-(piperidin-1-yl)but-2-en-1-one;
(E)-1-(5-(3′-(3,5-dichloro-4-hydroxyphenyl)-4′,5′-dihydro-7′H-spiro[cyclobutane-1,6′-pyrazolo[1,5-a]pyrazine]-5′-carbonyl)indolin-1-yl)-4-(dimethylamino)but-2-en-1-one;
(E)-4-(3-azabicyclo[3.1.1]heptan-3-yl)-1-(5-((3-(3,5-dichloro-4-hydroxyphenyl)-6,7-dihydropyrazolo[1,5-a]pyrazin-5(4H)-yl)methyl)-2,3-dihydro-1H-pyrrolo[3,2-b]pyridin-1-yl)but-2-en-1-one;
(E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)indolin-1-yl)-4-(dimethylamino)but-2-en-1-one;
(E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)-4-morpholinobut-2-en-1-one;
(E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)-4-(piperidin-1-yl)but-2-en-1-one;
(E)-4-(dimethylamino)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one;
(E)-1-(5-(3′-(3,5-dichloro-4-hydroxyphenyl)-4′,5′-dihydro-7′H-spiro[cyclobutane-1,6′-pyrazolo[1,5-a]pyrazine]-5′-carbonyl)indolin-1-yl)-4-morpholinobut-2-en-1-one;
(R,E)-1-(5-(3-(3,5-dichloro-4-hydroxyphenyl)-6-methyl-4,5,6,7-tetrahydropyrazolo[1,5-a]pyrazine-5-carbonyl)-4-methoxyindolin-1-yl)-4-morpholinobut-2-en-1-one;
(E)-1-(5-(3′-(2-methoxypyrimidin-4-yl)-4′,5′-dihydro-7′H-spiro[cyclopropane-1,6′-pyrazolo[1,5-a]pyrazine]-5′-carbonyl)indolin-1-yl)-4-morpholinobut-2-en-1-one;
(R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-(2,2-dimethylpyrrolidin-1-yl)but-2-en-1-one;
N-(4-(3′-(3,5-dichloro-4-hydroxyphenyl)-4′,5′-dihydro-7′H-spiro[cyclopropane-1,6′-pyrazolo[1,5-a]pyrazine]-5′-carbonyl)-2-methoxyphenyl)acrylamide;
(R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-(piperidin-1-yl)but-2-en-1-one;
(E)-4-(3-fluoroazetidin-1-yl)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one;
(R)-1-(5-(3-(3,5-dichloro-4-hydroxyphenyl)-6-methyl-4,5,6,7-tetrahydropyrazolo[1,5-a]pyrazine-5-carbonyl)isoindolin-2-yl)prop-2-en-1-one;
(R,E)-N-(4-(3-(3,5-dichloro-4-hydroxyphenyl)-6-methyl-4,5,6,7-tetrahydropyrazolo[1,5-a]pyrazine-5-carbonyl)-2-fluorophenyl)-4-morpholinobut-2-enamide;
(R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-morpholinobut-2-en-1-one;
(R,E)-N-(4-(3-(3,5-dichloro-4-hydroxyphenyl)-6-isopropyl-4,5,6,7-tetrahydropyrazolo[1,5-a]pyrazine-5-carbonyl)-2-methoxyphenyl)-4-(dimethylamino)but-2-enamide; and
(E)-4-(3-fluoroazetidin-1-yl)-1-(5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one;

or a pharmaceutically acceptable salt of any of the aforementioned.

Embodiment 112. The compound of embodiment 1, wherein the compound is (E)-4-(3-azabicyclo[3.1.1]heptan-3-yl)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)but-2-en-1-one, or a pharmaceutically acceptable salt thereof.

Embodiment 113. The compound of embodiment 1, wherein the compound is (E)-4-(dimethylamino)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one, or a pharmaceutically acceptable salt thereof.

Embodiment 114. The compound of embodiment 1, wherein the compound is (E)-4-(3-fluoroazetidin-1-yl)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one, or a pharmaceutically acceptable salt thereof.

Embodiment 115. The compound of embodiment 1, wherein the compound is (E)-4-(3-fluoroazetidin-1-yl)-1-(5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one, or a pharmaceutically acceptable salt thereof.

Embodiment 116. The compound of any one of any one of embodiments 1 to 115, or a pharmaceutically acceptable salt thereof, wherein the compound is deuterated.

Embodiment 117. A pharmaceutical composition comprising the compound of any one of embodiments 1-116, or a pharmaceutically acceptable salt thereof, and a pharmaceutically acceptable carrier.

Embodiment 118. A method of inhibiting VRK1, comprising contacting the VRK1 with the compound of any one of embodiments 1-116, or a pharmaceutically acceptable salt thereof.

Embodiment 119. A method of inhibiting VRK1 in a patient, comprising administering to the patient a compound of any one of embodiments 1-116, or a pharmaceutically acceptable salt thereof.

Embodiment 120. A method of selectively inhibiting VRK1 over VRK2, comprising contacting the VRK1 and VRK2 with a VRK1 inhibitor, or a pharmaceutically acceptable salt thereof.

Embodiment 121. A method of covalently binding a cysteine in VRK1 with a VRK1 inhibitor, or a pharmaceutically acceptable salt thereof.

Embodiment 122. The method of embodiment 121, further comprising binding with a water in the binding pocket of VRK1.

Embodiment 123. The method of embodiment 121 or 122, further comprising binding with Phe134 of VRK1.

Embodiment 124. The method of any one of embodiments 121-123, further comprising binding with Lys71 of VRK1.

Embodiment 125. The method of any one of embodiments 121-124, further comprising binding with Asp132 of VRK1.

Embodiment 126. The method of any one of embodiments 121-125, further comprising binding with the phosphate-binding loop of VRK1.

Embodiment 127. A method of covalently binding with a cysteine in VRK1, binding with a water in the binding pocket of VRK1, and binding with Phe134 of VRK1 with a VRK1 inhibitor, or a pharmaceutically acceptable salt thereof.

Embodiment 128. A method of covalently binding with a cysteine in VRK1, binding with a water in the binding pocket of VRK1, binding with Phe134 of VRK1, and binding with Lys71 of VRK1 with a VRK1 inhibitor, or a pharmaceutically acceptable salt thereof.

Embodiment 129. A method of covalently binding with a cysteine in VRK1, binding with a water in the binding pocket of VRK1, binding with Phe134 of VRK1, binding with Lys71 of VRK1, and binding with the phosphate-binding loop of VRK1 with a VRK1 inhibitor, or a pharmaceutically acceptable salt thereof.

Embodiment 130. A method of covalently binding with a cysteine in VRK1, binding with a water in the binding pocket of VRK1, binding with Phe134 of VRK1, binding with Asp132 of VRK1, and binding with the phosphate-binding loop of VRK1 with a VRK1 inhibitor, or a pharmaceutically acceptable salt thereof.

Embodiment 131. A method of covalently binding with a cysteine in VRK1, binding with a water in the binding pocket of VRK1, binding with Phe134 of VRK1, binding with Lys71 of VRK1, and binding with Asp132 of VRK1 with a VRK1 inhibitor, or a pharmaceutically acceptable salt thereof.

Embodiment 132. A method of covalently binding with a cysteine in VRK1, binding with a water in the binding pocket of VRK1, binding with Phe134 of VRK1, and binding with Asp132 of VRK1 with a VRK1 inhibitor, or a pharmaceutically acceptable salt thereof.

Embodiment 133. A method of covalently binding with a cysteine in VRK1, binding with a water in the binding pocket of VRK1, binding with Phe134 of VRK1, binding with Lys71 of VRK1, binding with Asp132 of VRK1, and binding with the phosphate-binding loop of VRK1 with a VRK1 inhibitor, or a pharmaceutically acceptable salt thereof.

Embodiment 134. The method of any one of embodiments 120-133, wherein the VRK1 inhibitor, or a pharmaceutically acceptable salt thereof, is a compound of any one of claims 1-116, or a pharmaceutically acceptable salt thereof.

Embodiment 135. A method of treating a disease or disorder associated with VRK1 in a patient, comprising administering to the patient a therapeutically effective amount of the compound of any one of embodiments 1-116, or pharmaceutically acceptable salt thereof.

Embodiment 136. The method of embodiment 135, wherein the disease or disorder is cancer.

Embodiment 137. The method of embodiment 136, wherein the cancer is sarcoma, colorectal adenocarcinoma, acute myeloid leukemia, glioblastoma, and neuroblastoma.

Embodiment 138. The method of embodiment 137, wherein the glioblastoma or neuroblastoma have overexpression of VRK1.

Embodiment 139. The method of embodiment 137, wherein the glioblastoma or neuroblastoma have underexpression of VRK2.

Embodiment 140. The method of embodiment 136, wherein the cancer is bone cancer, cancer of the neck and head, rectal cancer, esophageal cancer, sarcoma of soft tissue, neoplasm of the central nervous system (CNS), brain stem, glioma, colon cancer, Ewing's sarcoma, brain cancer, astrocytoma, neuroblastoma, intestinal cancer, nervous system cancers, osteosarcoma, cancer of the small bowel, adenocarcinoma of the small bowel, lymphoma of the small bowel, carcinoid tumors of the small bowel, Kaposi's sarcoma of the small bowel, leiomyoma of the small bowel, hemangioma of the small bowel, lipoma of the small bowel, neurofibroma of the small bowel, fibroma of the small bowel, cancer of the large bowel, adenocarcinoma of the large bowel, tubular adenoma of the large bowel, villous adenoma of the large bowel, hamartoma of the large bowel, or leiomyoma of the large bowel.

### EXAMPLES

Certain embodiments of the present disclosure will be described in greater detail by way of specific examples. The following examples are offered for illustrative purposes and are not intended to limit the present disclosure in any manner. Those of skill in the art will readily recognize a variety of non-critical parameters which can be changed or modified to yield essentially the same results. The compounds of the Examples have been found to inhibit the activity of VRK1 according to at least one assay described herein.

Experimental procedures for compounds of the present disclosure are provided below. Open Access Preparative LCMS Purification of some of the compounds prepared was performed on Waters mass directed fractionation systems. The basic equipment setup, protocols and control software for the operation of these systems have been described in detail in literature. See, e.g., Blom, “Two-Pump At Column Dilution Configuration for Preparative LC-MS”, K. Blom, J. Combi. Chem., 2002, 4, 295-301; Blom et al., “Optimizing Preparative LC-MS Configurations and Methods for Parallel Synthesis Purification”, J. Combi. Chem., 2003, 5, 670-83; and Blom et al., “Preparative LC-MS Purification: Improved Compound Specific Method Optimization”, J. Combi. Chem., 2004, 6, 874-883.

The compounds separated were typically subjected to analytical liquid chromatography mass spectrometry (LCMS) for purity analysis under the following conditions: Instrument=Agilent 1100 series, LC/MSD; Column: Waters Sunfire™ C₁₈ 5 μm, 2.1×50 mm, Buffers: mobile phase A: 0.025% TFA in water and mobile phase B: acetonitrile; gradient 2% to 80% B in 3 min with flow rate 2.0 mL/min.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm, 30×100 mm or Waters XBridge™ C₁₈ 5 μm, 30×100 mm column, eluting with mobile phase A: 0.1% TFA (trifluoroacetic acid) in water and mobile phase B: acetonitrile; the flow rate was 60 mL/min, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

pH=10 purifications: Waters XBridge™ C₁₈ 5 μm, 30×100 mm column, eluting with mobile phase A: 0.1% NH₄OH in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

The following abbreviations may be used herein: aq. (aqueous); br (broad); d (doublet); dd (doublet of doublets); DCM (dichloromethane); DMF (N, N-dimethylformamide); Et (ethyl); EtOAc (ethyl acetate); g (gram(s)); h (hrs)); HPLC (high performance liquid chromatography); Hz (hertz); J (coupling constant); LCMS (liquid chromatography-mass spectrometry); m (multiplet); M (molar); MS (Mass spectrometry); Me (methyl); MeCN (acetonitrile); MeOH (methanol); mg (milligram(s)); min. (minutes(s)); mL (milliliter(s)); mmol (millimole(s)); nM (nanomolar); NMR (nuclear magnetic resonance spectroscopy); Ph (phenyl); r.t. (room temperature), s (singlet); t (triplet or tertiary); TBS (tert-butyldimethylsilyl); tert (tertiary); tt (triplet of triplets); TFA (trifluoroacetic acid); THF (tetrahydrofuran); μg (microgram(s)); μL (microliter(s)); μM (micromolar); wt % (weight percent).

### Intermediate I. 2,6-dichloro-4-(4,5,6,7-tetrahydropyrazolo[1,5-a]pyrazin-3-yl)phenol

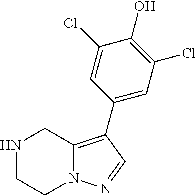 [SMILES: Oc1c(Cl)cc(-c2cnn3c2CNCC3)cc1Cl]

A mixture of tert-butyl 3-bromo-6,7-dihydropyrazolo[1,5-a]pyrazine-5(4H)-carboxylate (455 mg, 1.5 mmol), 2,6-dichloro-4-(4,4,5,5-tetramethyl-1,3,2-dioxaborolan-2-yl)phenol (522 mg, 1.8 mmol), [1,1′-bis(diphenylphosphino)ferrocene]dichloropalladium(II), complex with dichloromethane (123 mg, 0.15 mmol) and K₃PO₄ (1279 mg, 6.0 mmol) in 1,4-dioxane (4 mL) and water (1.5 mL) was heated to 100° C. for 1 hour. Upon completion, the resulting mixture was cooled and diluted with water and extracted with ethyl acetate (50 mL). The organic layer was dried with MgSO₄, filtered, and concentrated in vacuo. The obtained crude product was dissolved in DCM (2.0 mL), before 2,2,2-trifluoroacetic acid (1.0 mL) was added and stirred for 20 minutes. Upon completion, the reaction mixture was diluted with DCM, washed with NaHCO₃ (aq.), and the organic layer was dried and concentrated in vacuo. The residue was used in the next step without further purification. LCMS calculated for C₁₂H₁₂Cl₂N₃O (M+H)⁺: m/z=284.0; found: 284.0.

### Example 1. N-(4-(3-(3,5-dichloro-4-hydroxyphenyl)-4,5,6,7-tetrahydropyrazolo[1,5-a]pyrazine-5-carbonyl)phenyl)acrylamide

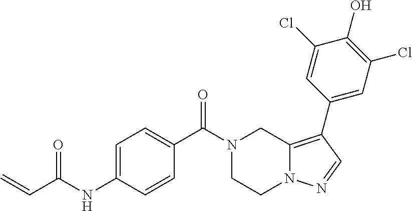 [SMILES: C=CC(=O)Nc1ccc(C(=O)N2CCn3ncc(-c4cc(Cl)c(O)c(Cl)c4)c3C2)cc1]

A solution of Intermediate I (30 mg, 0.1 mmol), 4-acrylamidobenzoic acid (60 mg, 0.32 mmol), HATU (120 mg, 0.16 mmol), and DIPEA (0.18 mL, 1.0 mmol) in DMF (1 mL) was stirred at room temperature for 2 hours. Upon completion, the reaction mixture was diluted with MeOH and purified by prep-LCMS (XBridge C18 column, eluting with a gradient of acetonitrile/water containing 0.1% TFA, at flow rate of 60 mL/min) to afford the desired product. LCMS calculated for C₂₂H₁₉Cl₂N₄O₃ (M+H)⁺: m/z=457.1; found: 457.2, 459.2. ¹H NMR (600 MHz, DMSO-d₆) δ 10.37 (s, 1H), 10.05 (br, 1H), 7.86 (s, 1H), 7.78 (d, J=8.4 Hz, 2H), 7.55-7.49 (m, 2H), 7.45-7.29 (m, 2H), 6.46 (dd, J=17.0, 10.2 Hz, 1H), 6.30 (dd, J=17.0, 1.9 Hz, 1H), 5.80 (dd, J=10.1, 1.9 Hz, 1H), 4.96 (s, 2H), 4.25 (t, J=5.5 Hz, 2H), 3.93 (s, 2H).

### Example 2. N-(4-(3-(3,5-dichloro-4-hydroxyphenyl)-4,5,6,7-tetrahydropyrazolo[1,5-a]pyrazine-5-carbonyl)-2-methoxyphenyl)acrylamide

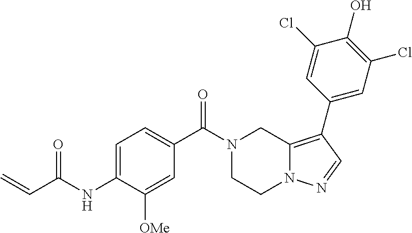 [SMILES: C=CC(=O)Nc1ccc(C(=O)N2CCn3ncc(-c4cc(Cl)c(O)c(Cl)c4)c3C2)cc1OC]

### Step 1. tert-butyl 4-acrylamido-3-methoxybenzoate

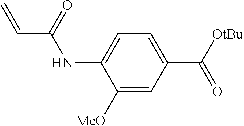 [SMILES: C=CC(=O)Nc1ccc(C(=O)OC(C)(C)C)cc1OC]

To a 40 mL scintillation vial equipped with a magnetic stir bar was added tert-butyl 4-amino-3-methoxybenzoate (95 mg, 0.425 mmol), triethylamine (0.119 mL, 0.851 mmol), and DCM (2 mL). At 0° C., acryloyl chloride (41 mg, 0.447 mmol) dissolved in DCM (5 mL) was added to the stirring mixture dropwise via an addition funnel over 15 minutes. The reaction mixture was stirred and warmed up from 0° C. to room temperature for 20 minutes. Upon completion, the mixture was quenched with water and extracted with DCM for 3 times. The combined organic layer was dried over MgSO₄, concentrated, and purified by silica gel column chromatography (25%-60% ethyl acetate/hexanes) to afford the desired product in quantitative yield. LCMS calculated for C₁₅H₂₀NO₄ (M+H)⁺: m/z=278.1; found: 278.1.

### Step 2. 4-acrylamido-3-methoxybenzoic acid

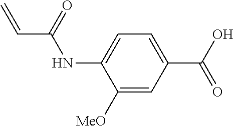 [SMILES: C=CC(=O)Nc1ccc(C(=O)O)cc1OC]

To a 40 mL scintillation vial containing a magnetic stir bar and tert-butyl 4-acrylamido-3-methoxybenzoate (118 mg, 0.425 mmol) was added DCM (2 mL) and trifluoroacetic acid (1 mL). The mixture was stirred at room temperature until analysis by LCMS indicated full conversion of the reactant. The mixture was then concentrated in vacuo and carried forward to the next step without further purification. LCMS calculated for C₁₁H₁₂NO₄ (M+H)⁺: m/z=222.1; found: 222.1.

### Step 3. N-(4-(3-(3,5-dichloro-4-hydroxyphenyl)-4,5,6,7-tetrahydropyrazolo[1,5-a]pyrazine-5-carbonyl)-2-methoxyphenyl)acrylamide

To a 40 mL scintillation vial containing a magnetic stir bar was added 2,6-dichloro-4-(4,5,6,7-tetrahydropyrazolo[1,5-a]pyrazin-3-yl)phenol (30 mg, 0.11 mmol), 4-acrylamido-3-methoxybenzoic acid (35 mg, 0.16 mmol), HATU (72 mg, 0.19 mmol), DMF (1 mL), and DIPEA (0.065 mL, 0.37 mmol). The mixture was stirred at room temperature for 15 minutes. Upon full consumption of the starting material, lithium hydroxide monohydrate (14 mg, 0.33 mmol) and water (0.2 mL) were added, and the mixture was stirred at 50° C. for 10 minutes. The reaction mixture was then diluted with acetonitrile, filtered, and purified by prep LCMS (pH 10) to afford the desired product. LCMS calculated for C₂₃H₂₁Cl₂N₄O₄ (M+H)⁺: m/z=487.1; found: 487.1. ¹H NMR (500 MHz, DMSO-d₆) δ 10.13 (s, 1H), 9.52 (s, 1H), 8.22 (d, J=8.2 Hz, 1H), 7.88 (s, 1H), 7.51-7.26 (m, 2H), 7.20 (d, J=1.8 Hz, 1H), 7.12 (dd, J=8.2, 1.8 Hz, 1H), 6.76 (dd, J=17.0, 10.2 Hz, 1H), 6.27 (dd, J=17.0, 2.1 Hz, 1H), 5.75 (dd, J=10.1, 2.1 Hz, 1H), 4.99 (s, 2H), 4.25 (t, J=5.5 Hz, 2H), 3.90 (m, 5H).

### Example 3. 1-(5-(3-(3,5-dichloro-4-hydroxyphenyl)-4,5,6,7-tetrahydropyrazolo[1,5-a]pyrazine-5-carbonyl)isoindolin-2-yl)prop-2-en-1-one

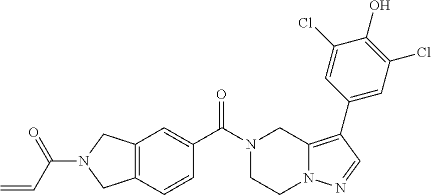 [SMILES: C=CC(=O)N1Cc2ccc(C(=O)N3CCn4ncc(-c5cc(Cl)c(O)c(Cl)c5)c4C3)cc2C1]

### Step 1. Methyl 2-acryloylisoindoline-5-carboxylate

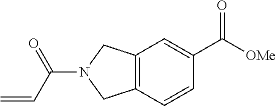 [SMILES: C=CC(=O)N1Cc2ccc(C(=O)OC)cc2C1]

To a 40 mL scintillation vial equipped with a magnetic stir bar was added methyl isoindoline-5-carboxylate (75 mg, 0.35 mmol), triethylamine (0.12 mL, 0.88 mmol), and DCM (2 mL). At 0° C., acryloyl chloride (38 mg, 0.42 mmol) dissolved in DCM (5 mL) was added to the stirring mixture dropwise via an addition funnel over 15 minutes. The reaction mixture was stirred from 0° C. to room temperature for 20 minutes. Upon completion, the mixture was quenched with water and extracted with DCM for 3 times. The combined organic layer was dried over MgSO₄, concentrated, and purified by silica gel column chromatography (25%-70% ethyl acetate/hexanes) to afford the desired product in 90% yield. LCMS calculated for C₁₃H₁₄NO₃ (M+H)⁺: m/z=232.1; found: 232.1.

### Step 2. 2-Acryloylisoindoline-5-carboxylic acid

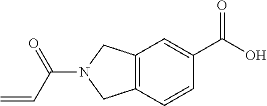 [SMILES: C=CC(=O)N1Cc2ccc(C(=O)O)cc2C1]

To a 40 mL scintillation vial containing a magnetic stir bar and methyl 2-acryloylisoindoline-5-carboxylate (73 mg, 0.32 mmol) was added lithium hydroxide monohydrate (33 mg, 0.79 mmol), THF (1.6 mL) and water (0.33 mL). The mixture was stirred at 40° C. for 1 hour. Upon completion, the reaction mixture was quenched with concentrated HCl (0.024 mL, 12.1 M, 0.79 mmol), concentrated in vacuo, and carried forward to the next step without further purification. LCMS calculated for C₁₂H₁₂NO₃ (M+H)⁺: m/z=218.1; found: 218.1.

### Step 3. 1-(5-(3-(3,5-dichloro-4-hydroxyphenyl)-4,5,6,7-tetrahydropyrazolo[1,5-a]pyrazine-5-carbonyl)isoindolin-2-yl)prop-2-en-1-one

To a 40 mL scintillation vial containing a magnetic stir bar was added 2,6-dichloro-4-(4,5,6,7-tetrahydropyrazolo[1,5-a]pyrazin-3-yl)phenol (36 mg, 0.13 mmol), 2-acryloylisoindoline-5-carboxylic acid (33 mg, 0.15 mmol), HATU (81 mg, 0.21 mmol), DMF (1 mL), and DIPEA (0.088 mL, 0.50 mmol). The mixture was stirred at room temperature for 15 minutes. Upon full consumption of the starting material, lithium hydroxide monohydrate (16 mg, 0.39 mmol) and water (0.2 mL) were added, and the mixture was stirred at 50° C. for 10 minutes. The reaction mixture was then diluted with acetonitrile, filtered, and purified by prep LCMS (pH 10) to afford the desired product. LCMS calculated for C₂₄H₂₁Cl₂N₄O₃ (M+H)⁺: m/z=483.1; found: 483.1. ¹H NMR (600 MHz, DMSO-d₆) δ 7.81 (s, 1H), 7.54-7.41 (m, 3H), 7.35 (m, 2H), 6.68 (t, J=14.0 Hz, 1H), 6.52 (s, 1H), 6.24 (d, J=16.7 Hz, 1H), 5.77 (d, J=10.6 Hz, 1H), 5.00 (d, J=9.1 Hz, 4H), 4.76 (s, 2H), 4.22 (t, J=5.6 Hz, 2H), 3.82 (br, 2H).

### Example 4. N-(5-(3-(3,5-dichloro-4-hydroxyphenyl)-4,5,6,7-tetrahydropyrazolo[1,5-a]pyrazine-5-carbonyl)-6-methoxypyridin-2-yl)acrylamide

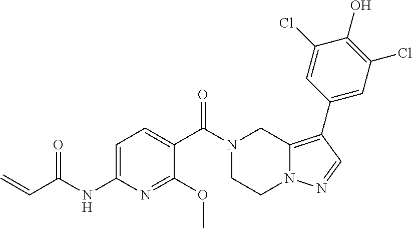 [SMILES: C=CC(=O)Nc1ccc(C(=O)N2CCn3ncc(-c4cc(Cl)c(O)c(Cl)c4)c3C2)c(OC)n1]

This compound was prepared according to the procedures described in Example 3, with methyl 6-amino-2-methoxynicotinate replacing methyl isoindoline-5-carboxylate in step 1. LCMS calculated for C₂₂H₂₀Cl₂N₅O₄ (M+H)⁺: m/z=488.1, found: 488.1. ¹H NMR (600 MHz, DMSO-d₆) δ 10.70 (s, 1H), 7.93-7.75 (m, 2H), 7.44 (s, 2H), 7.23 (s, 1H), 6.67 (dd, J=17.0, 10.1 Hz, 1H), 6.52 (s, 1H), 6.33 (d, J=16.6 Hz, 1H), 5.81 (d, J=10.7 Hz, 1H), 5.02 (s, 2H), 4.27-4.06 (m, 2H), 3.93 (s, 3H), 3.75 (d, J=17.4 Hz, 2H).

### Example 5. N-(6-(3-(3,5-dichloro-4-hydroxyphenyl)-4,5,6,7-tetrahydropyrazolo[1,5-a]pyrazine-5-carbonyl)pyridin-3-yl)acrylamide

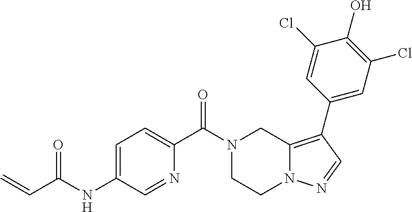 [SMILES: C=CC(=O)Nc1ccc(C(=O)N2CCn3ncc(-c4cc(Cl)c(O)c(Cl)c4)c3C2)nc1]

This compound was prepared according to the procedures described in Example 3, with methyl 5-aminopicolinate replacing methyl isoindoline-5-carboxylate in step 1. LCMS calculated for C₂₁H₁₈Cl₂N₅O₃ (M+H)⁺: m/z=458.1, found: 458.2. ¹H NMR (600 MHz, DMSO-d₆) δ 10.62 (s, 1H), 8.93-8.76 (m, 1H), 8.26 (d, J=8.4 Hz, 1H), 7.93-7.68 (m, 2H), 7.44 (s, 2H), 7.29 (s, 1H), 6.47 (dd, J=17.0, 10.2 Hz, 1H), 6.34 (d, J=16.8 Hz, 1H), 5.86 (d, J=10.0 Hz, 1H), 5.23 (s, 1H), 5.05 (s, 2H), 4.29-4.02 (m, 3H).

### Example 6. N-(6-(3-(3,5-dichloro-4-hydroxyphenyl)-4,5,6,7-tetrahydropyrazolo[1,5-a]pyrazine-5-carbonyl)-2-methoxypyridin-3-yl)acrylamide

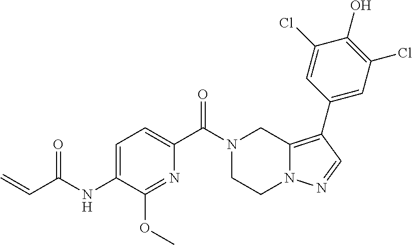 [SMILES: C=CC(=O)Nc1ccc(C(=O)N2CCn3ncc(-c4cc(Cl)c(O)c(Cl)c4)c3C2)nc1OC]

This compound was prepared according to the procedures described in Example 3, with methyl 5-amino-6-methoxypicolinate replacing methyl isoindoline-5-carboxylate in step 1. LCMS calculated for C₂₂H₂₀Cl₂N₅O₄ (M+H)⁺: m/z=488.1, found: 488.2. ¹H NMR (600 MHz, DMSO-d₆) δ 10.27-10.09 (m, 1H), 9.82 (s, 1H), 8.61 (d, J=7.8 Hz, 1H), 7.92-7.74 (m, 1H), 7.55-7.23 (m, 3H), 6.79 (dd, J=17.5, 9.9 Hz, 1H), 6.52 (s, 1H), 6.29 (t, J=14.5 Hz, 1H), 5.79 (d, J=10.4 Hz, 1H), 5.41 (s, 1H), 5.03 (s, 1H), 4.31-3.82 (m, 6H).

### Example 7. N-(3-(3-(3,5-dichloro-4-hydroxyphenyl)-4,5,6,7-tetrahydropyrazolo[1,5-a]pyrazine-5-carbonyl)-1-methyl-1H-pyrazol-5-yl)acrylamide

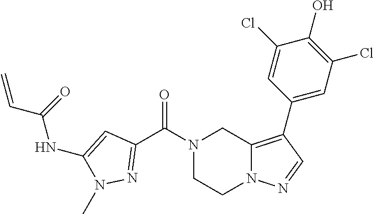 [SMILES: C=CC(=O)Nc1cc(C(=O)N2CCn3ncc(-c4cc(Cl)c(O)c(Cl)c4)c3C2)nn1C]

This compound was prepared according to the procedures described in Example 3, with methyl 5-amino-1-methyl-1H-pyrazole-3-carboxylate replacing methyl isoindoline-5-carboxylate in step 1. LCMS calculated for C₂₀H₁₉Cl₂N₆O₃ (M+H)⁺: m/z=461.1, found: 461.2. ¹H NMR (600 MHz, DMSO-d₆) δ 10.35 (s, 1H), 7.87 (s, 1H), 7.42 (s, 2H), 6.76-6.64 (m, 1H), 6.51 (s, 1H), 6.32 (d, J=17.1 Hz, 1H), 5.85 (d, J=10.3 Hz, 1H), 5.65 (s, 1H), 5.01 (s, 1H), 4.50 (s, 1H), 4.30-4.01 (m, 4H), 3.79 (s, 3H).

### Example 8. N-(2-(3-(3,5-dichloro-4-hydroxyphenyl)-4,5,6,7-tetrahydropyrazolo[1,5-a]pyrazine-5-carbonyl)thiazol-5-yl)acrylamide

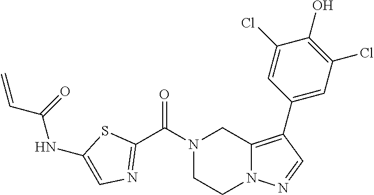 [SMILES: C=CC(=O)Nc1cnc(C(=O)N2CCn3ncc(-c4cc(Cl)c(O)c(Cl)c4)c3C2)s1]

This compound was prepared according to the procedures described in Example 3, with methyl 5-aminothiazole-2-carboxylate replacing methyl isoindoline-5-carboxylate in step 1. LCMS calculated for C₁₉H₁₆Cl₂N₅O₃S (M+H)⁺: m/z=464.0, found: 464.1. ¹H NMR (600 MHz, DMSO-d₆) δ 12.00 (s, 1H), 10.19 (s, 1H), 7.86 (s, 1H), 7.76 (s, 1H), 7.43 (d, J=8.5 Hz, 2H), 6.47-6.35 (m, 2H), 5.93 (d, J=9.9 Hz, 1H), 5.89 (s, 1H), 5.05 (s, 1H), 4.85 (s, 1H), 4.32-4.07 (m, 3H).

### Example 9. N-(5-(3-(3,5-dichloro-4-hydroxyphenyl)-4,5,6,7-tetrahydropyrazolo[1,5-a]pyrazine-5-carbonyl)quinolin-8-yl)acrylamide

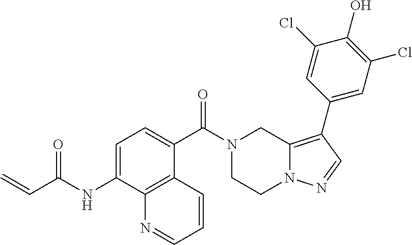 [SMILES: C=CC(=O)Nc1ccc(C(=O)N2CCn3ncc(-c4cc(Cl)c(O)c(Cl)c4)c3C2)c2cccnc12]

This compound was prepared according to the procedures described in Example 3, with methyl 8-aminoquinoline-5-carboxylate replacing methyl isoindoline-5-carboxylate in step 1. LCMS calculated for C₂₅H₂₀Cl₂N₅O₃ (M+H)⁺: m/z=508.1, found: 508.1. ¹H NMR (600 MHz, DMSO-d₆) δ 10.53 (s, 1H), 9.02 (s, 1H), 8.79 (d, J=7.9 Hz, 1H), 7.95-7.82 (m, 1H), 7.74 (d, J=8.5 Hz, 2H), 7.48 (s, 2H), 7.21-6.92 (m, 2H), 6.52 (s, 1H), 6.37 (d, J=16.9 Hz, 1H), 5.85 (d, J=10.3 Hz, 1H), 5.18 (s, 2H), 4.29-4.04 (m, 2H), 3.72 (s, 2H).

### Example 10. 1-(6-(3-(3,5-dichloro-4-hydroxyphenyl)-4,5,6,7-tetrahydropyrazolo[1,5-a]pyrazine-5-carbonyl)-1,3-dihydro-2H-pyrrolo[3,4-c]pyridin-2-yl)prop-2-en-1-one

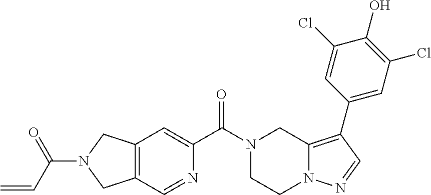 [SMILES: C=CC(=O)N1Cc2cnc(C(=O)N3CCn4ncc(-c5cc(Cl)c(O)c(Cl)c5)c4C3)cc2C1]

This compound was prepared according to the procedures described in Example 3, with 2,3-dihydro-1H-pyrrolo[3,4-c]pyridine-6-carboxylate replacing methyl isoindoline-5-carboxylate in step 1. LCMS calculated for C₂₃H₂₀Cl₂N₅O₃ (M+H)⁺: m/z=484.1, found: 484.2. ¹H NMR (600 MHz, DMSO-d₆) δ 10.23-10.10 (m, 1H), 8.69-8.55 (m, 1H), 7.89 (s, 1H), 7.81-7.71 (m, 1H), 7.45 (s, 2H), 7.26 (s, 1H), 6.77-6.59 (m, 1H), 6.25 (d, J=14.9 Hz, 1H), 5.79 (s, 1H), 5.12-4.97 (m, 2H), 4.97-4.69 (m, 3H), 4.31-3.93 (m, 4H).

### Example 11. 1-(5-(3-(3,5-dichloro-4-hydroxyphenyl)-4,5,6,7-tetrahydropyrazolo[1,5-a]pyrazine-5-carbonyl)indolin-1-yl)prop-2-en-1-one

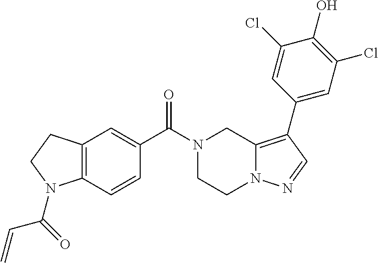 [SMILES: C=CC(=O)N1CCc2cc(C(=O)N3CCn4ncc(-c5cc(Cl)c(O)c(Cl)c5)c4C3)ccc21]

This compound was prepared according to the procedures described in Example 3, with methyl indoline-5-carboxylate replacing methyl isoindoline-5-carboxylate in step 1. LCMS calculated for C₂₄H₂₁Cl₂N₄O₃ (M+H)⁺: m/z=483.1, found: 483.1. ¹H NMR (600 MHz, DMSO-d₆) δ 10.16 (s, 1H), 8.19 (s, 1H), 7.86 (s, 1H), 7.45-7.30 (m, 3H), 6.76 (dd, J=16.7, 10.3 Hz, 1H), 6.33 (d, J=16.6 Hz, 1H), 5.86 (d, J=10.4 Hz, 1H), 4.96 (s, 2H), 4.25 (dt, J=11.0, 6.8 Hz, 5H), 3.94 (s, 2H), 3.21 (t, J=8.6 Hz, 2H).

### Example 12. 1-(6-(3-(3,5-dichloro-4-hydroxyphenyl)-4,5,6,7-tetrahydropyrazolo[1,5-a]pyrazine-5-carbonyl)-3,4-dihydroquinolin-1(2H)-yl)prop-2-en-1-one

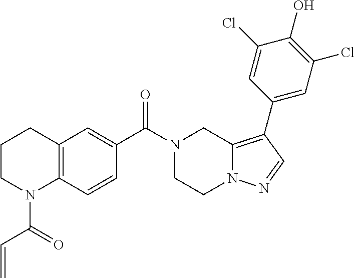 [SMILES: C=CC(=O)N1CCCc2cc(C(=O)N3CCn4ncc(-c5cc(Cl)c(O)c(Cl)c5)c4C3)ccc21]

This compound was prepared according to the procedures described in Example 3, with methyl 1,2,3,4-tetrahydroquinoline-6-carboxylate replacing methyl isoindoline-5-carboxylate in step 1. LCMS calculated for C₂₅H₂₃Cl₂N₄O₃ (M+H)⁺: m/z=497.1, found: 497.1. ¹H NMR (600 MHz, DMSO-d₆) δ 7.87 (s, 1H), 7.38 (s, 1H), 7.35 (d, J=8.4 Hz, 1H), 7.25 (d, J=8.4 Hz, 1H), 6.59 (s, 1H), 6.25 (d, J=16.7 Hz, 1H), 5.74 (d, J=10.4 Hz, 1H), 4.96 (s, 2H), 4.23 (d, J=5.6 Hz, 3H), 3.94 (s, 2H), 3.76 (t, J=6.5 Hz, 3H), 2.75 (d, J=6.5 Hz, 2H), 1.90 (p, J=6.7 Hz, 3H).

### Example 13. N-(4-(3-(3,5-dichloro-4-hydroxyphenyl)-4,5,6,7-tetrahydropyrazolo[1,5-a]pyrazine-5-carbonyl)naphthalen-1-yl)acrylamide

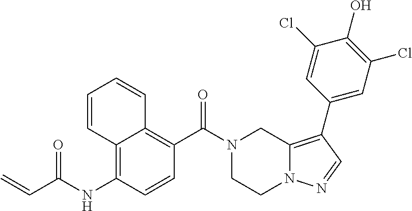 [SMILES: C=CC(=O)Nc1ccc(C(=O)N2CCn3ncc(-c4cc(Cl)c(O)c(Cl)c4)c3C2)c2ccccc12]

### Step 1. methyl 4-acrylamido-1-naphthoate

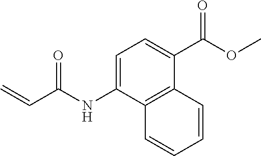 [SMILES: C=CC(=O)Nc1ccc(C(=O)OC)c2ccccc12]

To a mixture of acrylic acid (23.78 mg, 0.330 mmol), methyl 4-amino-1-naphthoate (0.060 g, 0.3 mmol), and triethylamine (209 μL, 1.500 mmol) in DMF (2 mL) was added propane phosphonic acid anhydride (210 mg, 0.330 mmol). The reaction was stirred at 60° C. for 1 hour. The reaction was diluted with ethyl acetate (2 mL) and washed with water (6 mL). The organic layer was dried and concentrated. The residue was purified with flash column chromatography (0-100% hexanes/EtOAc) to give 42 mg product (55% yield). LCMS calculated for C₁₅H₁₄NO₃ (M+H)⁺: m/z=256.1, found: 256.1.

### Step 2. 4-acrylamido-1-naphthoic acid

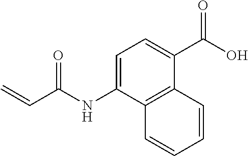 [SMILES: C=CC(=O)Nc1ccc(C(=O)O)c2ccccc12]

To a solution of methyl 4-acrylamido-1-naphthoate (35 mg, 0.137 mmol) in THF (1.500 mL) and water (1 mL) was added lithium hydroxide monohydrate (0.5 mL, 0.750 mmol). The reaction was stirred overnight at 40° C. Upon completion, the reaction was neutralized with 1N HCl (0.75 mL, 0.750 mmol), concentrated under vacuum and used in the next step directly. LCMS calculated for C₁₄H₁₂NO₃ (M+H)⁺: m/z=242.1, found: 242.1.

### Step 3. N-(4-(3-(3,5-dichloro-4-hydroxyphenyl)-4,5,6,7-tetrahydropyrazolo[1,5-a]pyrazine-5-carbonyl)naphthalen-1-yl)acrylamide

To a mixture of Intermediate I (0.028 g, 0.100 mmol), 4-acrylamido-1-naphthoic acid (0.024 g, 0.1 mmol), and DIPEA (0.087 mL, 0.500 mmol) in DMF (2 mL) was added HATU (0.057 g, 0.150 mmol) in one portion. The reaction was stirred at 60° C. for 1 hour. Upon completion, the reaction mixture was acidified with TFA, diluted with MeOH, and purified by prep-LCMS (XBridge C18 column, eluting with a gradient of acetonitrile/water containing 0.1% TFA, at flow rate of 60 mL/min) to afford desired product (2.4 mg, 47% yield). LCMS calculated for C₂₆H₂₁Cl₂N₄O₃ (M+H)⁺: m/z=507.1, found: 507.1. ¹H NMR (600 MHz, DMSO-d₆) δ 10.29-10.17 (m, 1H), 8.20 (d, J=8.1 Hz, 1H), 7.95-7.81 (m, 2H), 7.67-7.56 (m, 2H), 7.49 (s, 2H), 6.74 (s, 1H), 6.52 (s, 5H), 6.35 (d, J=17.2 Hz, 1H), 5.85 (d, J=10.5 Hz, 1H), 5.33-5.11 (m, 1H), 4.03 (s, 1H), 3.70-3.57 (m, 2H).

### Example 14. N-(8-(3-(3,5-dichloro-4-hydroxyphenyl)-4,5,6,7-tetrahydropyrazolo[1,5-a]pyrazine-5-carbonyl)-2,3-dihydrobenzo[b][1,4]dioxin-5-yl)acrylamide

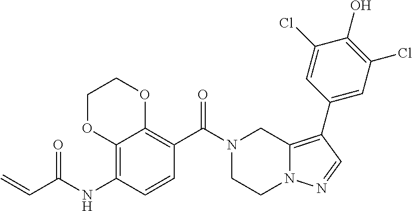 [SMILES: C=CC(=O)Nc1ccc(C(=O)N2CCn3ncc(-c4cc(Cl)c(O)c(Cl)c4)c3C2)c2c1OCCO2]

This compound was prepared according to the procedures described in Example 13, with methyl 8-amino-2,3-dihydrobenzo[b][1,4]dioxine-5-carboxylate replacing methyl 4-amino-1-naphthoate in step 1. LCMS calculated for C₂₄H₂₁Cl₂N₄O₅ (M+H)⁺: m/z=515.1, found: 515.1. ¹H NMR (600 MHz, DMSO-d₆) δ 9.63-9.36 (m, 1H), 7.92-7.82 (m, 1H), 7.73 (d, J=8.4 Hz, 1H), 7.44 (s, 2H), 7.24 (s, 1H), 6.84 (d, J=8.4 Hz, 1H), 6.79-6.61 (m, 1H), 6.24 (t, J=16.5 Hz, 1H), 5.74 (d, J=10.8 Hz, 1H), 5.07-4.92 (m, 2H), 4.37 (s, 3H), 4.24-4.10 (m, 3H), 3.86-3.67 (m, 2H).

### Example 15. N-(5-(3-(3,5-dichloro-4-hydroxyphenyl)-4,5,6,7-tetrahydropyrazolo[1,5-a]pyrazine-5-carbonyl)isoquinolin-8-yl)acrylamide

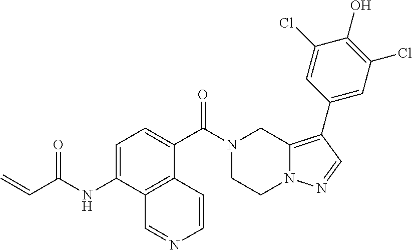 [SMILES: C=CC(=O)Nc1ccc(C(=O)N2CCn3ncc(-c4cc(Cl)c(O)c(Cl)c4)c3C2)c2ccncc12]

This compound was prepared according to the procedures described in Example 13, with methyl 8-aminoisoquinoline-5-carboxylate replacing methyl 4-amino-1-naphthoate in step 1. LCMS calculated for C₂₅H₂₀Cl₂N₅O₃ (M+H)⁺: m/z=508.1, found: 508.2. ¹H NMR (600 MHz, DMSO-d₆) δ 10.57 (s, 1H), 10.21 (s, 1H), 9.62 (s, 1H), 8.60 (s, 1H), 8.13 (d, J=7.9 Hz, 1H), 7.90 (d, J=10.0 Hz, 2H), 7.76 (s, 1H), 7.48 (s, 2H), 6.76 (dd, J=17.0, 10.2 Hz, 1H), 6.39 (d, J=17.2 Hz, 1H), 5.90 (d, J=10.1 Hz, 1H), 5.20 (s, 2H), 4.12 (s, 2H), 3.68 (s, 2H).

### Example 16. N-(8-(3-(3,5-dichloro-4-hydroxyphenyl)-4,5,6,7-tetrahydropyrazolo[1,5-a]pyrazine-5-carbonyl)isoquinolin-5-yl)acrylamide

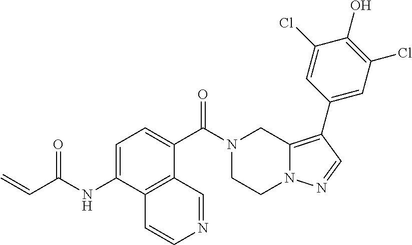 [SMILES: C=CC(=O)Nc1ccc(C(=O)N2CCn3ncc(-c4cc(Cl)c(O)c(Cl)c4)c3C2)c2cnccc12]

This compound was prepared according to the procedures described in Example 13, with methyl 5-aminoisoquinoline-8-carboxylate replacing methyl 4-amino-1-naphthoate in step 1. LCMS calculated for C₂₅H₂₀Cl₂N₅O₃ (M+H)⁺: m/z=508.1, found: 508.1. ¹H NMR (600 MHz, DMSO-d₆) δ 10.37 (s, 1H), 9.30 (s, 1H), 8.66 (s, 1H), 8.28 (d, J=7.7 Hz, 1H), 8.14 (d, J=6.3 Hz, 1H), 7.91 (s, 1H), 7.80 (d, J=8.0 Hz, 1H), 7.49 (s, 2H), 6.76 (dd, J=16.9, 10.3 Hz, 1H), 6.38 (d, J=16.9 Hz, 1H), 5.89 (d, J=10.1 Hz, 2H), 5.22 (s, 2H), 4.11 (s, 2H), 3.73 (s, 2H).

### Example 17. N-(5-(3-(3,5-dichloro-4-hydroxyphenyl)-4,5,6,7-tetrahydropyrazolo[1,5-a]pyrazine-5-carbonyl)-4-methoxypyridin-2-yl)acrylamide

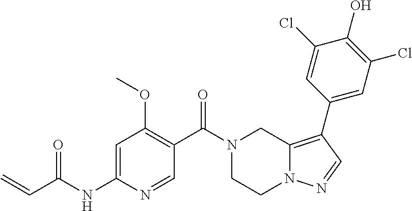 [SMILES: C=CC(=O)Nc1cc(OC)c(C(=O)N2CCn3ncc(-c4cc(Cl)c(O)c(Cl)c4)c3C2)cn1]

This compound was prepared according to the procedures described in Example 13, with methyl 6-amino-4-methoxynicotinate replacing methyl 4-amino-1-naphthoate in step 1. LCMS calculated for C₂₂H₂₀Cl₂N₅O₄ (M+H)⁺: m/z=488.1, found: 488.1.

### Example 18. N-(6-(3-(3,5-dichloro-4-hydroxyphenyl)-4,5,6,7-tetrahydropyrazolo[1,5-a]pyrazine-5-carbonyl)-2-(trifluoromethyl)pyridin-3-yl)acrylamide

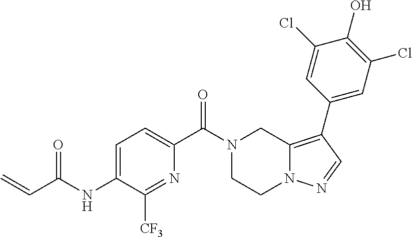 [SMILES: C=CC(=O)Nc1ccc(C(=O)N2CCn3ncc(-c4cc(Cl)c(O)c(Cl)c4)c3C2)nc1C(F)(F)F]

### Step 1. 5-acrylamido-6-(trifluoromethyl)picolinic acid

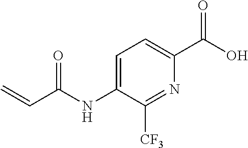 [SMILES: C=CC(=O)Nc1ccc(C(=O)O)nc1C(F)(F)F]

To a mixture of acrylic acid (23.78 mg, 0.330 mmol), 5-amino-6-(trifluoromethyl)picolinic acid (61.8 mg, 0.3 mmol), and triethylamine (209 μL, 1.500 mmol) in DMF (2 mL) was added propane phosphonic acid anhydride (210 mg, 0.330 mmol). The reaction was stirred at 60° C. for 1 hour. Upon completion, the reaction mixture was acidified with TFA, diluted with MeOH, and purified with HPLC to give desired product (23.5 mg, 30% yield). LCMS calculated for C₁₀H₈F₃N₂O₃(M+H)⁺: m/z=261.1, found: 261.0.

### Step 2. N-(6-(3-(3,5-dichloro-4-hydroxyphenyl)-4,5,6,7-tetrahydropyrazolo[1,5-a]pyrazine-5-carbonyl)-2-(trifluoromethyl)pyridin-3-yl)acrylamide

To a mixture of Intermediate I (0.028 g, 0.100 mmol), 5-acrylamido-6-(trifluoromethyl)picolinic acid (0.023 g, 0.09 mmol), and DIPEA (0.087 mL, 0.500 mmol) in DMF (2 mL) was added HATU (0.057 g, 0.150 mmol) in one portion. The reaction was stirred at 60° C. for 1 hour. Upon completion, the reaction mixture was acidified with TFA, diluted with MeOH, and purified by prep-LCMS (XBridge C18 column, eluting with a gradient of acetonitrile/water containing 0.1% TFA, at flow rate of 60 mL/min) to afford desired product (2.9 mg, 55% yield). LCMS calculated for C₂₂H₁₇Cl₂F₃N₅O₃(M+H)⁺: m/z=526.1, found: 526.0. ¹H NMR (600 MHz, DMSO-d₆) δ 10.21-10.09 (m, 1H), 8.33-8.25 (m, 1H), 8.16-8.03 (m, 1H), 7.92-7.83 (m, 1H), 7.44 (s, 2H), 7.27 (s, 1H), 6.66-6.54 (m, 1H), 6.32 (dd, J=17.2, 9.7 Hz, 1H), 5.87 (t, J=9.3 Hz, 1H), 5.27 (s, 1H), 5.08 (s, 1H), 4.37-4.14 (m, 3H), 4.04 (d, J=5.8 Hz, 1H).

### Example 19. 1-(5-(3-(3,5-dichloro-4-hydroxyphenyl)-4,5,6,7-tetrahydropyrazolo[1,5-a]pyrazine-5-carbonyl)-2,3-dihydro-1H-pyrrolo[2,3-b]pyridin-1-yl)prop-2-en-1-one

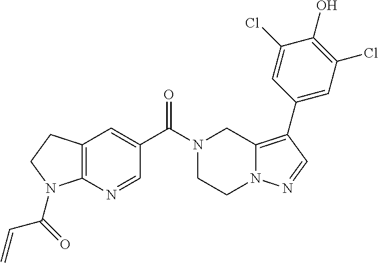 [SMILES: C=CC(=O)N1CCc2cc(C(=O)N3CCn4ncc(-c5cc(Cl)c(O)c(Cl)c5)c4C3)cnc21]

### Step 1. (3-bromo-6,7-dihydropyrazolo[1,5-a]pyrazin-5(4H)-yl)(2,3-dihydro-1H-pyrrolo[2,3-b]pyridin-5-yl)methanone

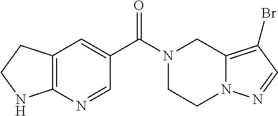 [SMILES: O=C(c1cnc2c(c1)CCN2)N1CCn2ncc(Br)c2C1]

To a solution of methyl 2,3-dihydro-1H-pyrrolo[2,3-b]pyridine-5-carboxylate (53.5 mg, 0.3 mmol) in THF (1 mL) and water (1.5 mL) was added lithium hydroxide monohydrate (0.5 mL, 0.750 mmol). The reaction was stirred for 24 hours at 60° C. Upon completion, the reaction was quenched with 1 N HCl (0.750 mL, 0.750 mmol) and concentrated under vacuum. The residue was dissolved in DMF (2 mL), then 3-bromo-4,5,6,7-tetrahydropyrazolo[1,5-a]pyrazine HCl salt (107 mg, 0.450 mmol), DIPEA (0.267 mL, 1.500 mmol), and HATU (171 mg, 0.450 mmol) were added. The mixture was stirred at 60° C. for 1 hour. Upon completion, the reaction was diluted with ethyl acetate (2 mL) and washed with water (6 mL). The organic layer was dried and concentrated. The residue was purified with flash column chromatography (0-100% hexanes/EtOAc) to give 59 mg product (57% yield). LCMS calculated for C₁₄H₁₅BrN₅O (M+H)⁺: m/z=348.1, found: 348.1.

### Step 2. 1-(5-(3-bromo-4,5,6,7-tetrahydropyrazolo[1,5-a]pyrazine-5-carbonyl)-2,3-dihydro-1H-pyrrolo[2,3-b]pyridin-1-yl)prop-2-en-1-one

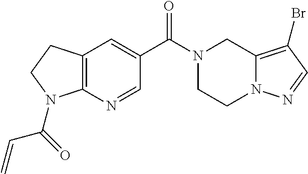 [SMILES: C=CC(=O)N1CCc2cc(C(=O)N3CCn4ncc(Br)c4C3)cnc21]

To a mixture of acrylic acid (13.48 mg, 0.187 mmol), (3-bromo-6,7-dihydropyrazolo[1,5-a]pyrazin-5(4H)-yl)(2,3-dihydro-1H-pyrrolo[2,3-b]pyridin-5-yl)methanone (59.2 mg, 0.170 mmol), and triethylamine (0.118 mL, 0.850 mmol) in DMF (1 mL) was added propane phosphonic acid anhydride (119 mg, 0.187 mmol). The reaction was stirred at 60° C. and monitored by LCMS. Upon completion, the reaction was diluted with ethyl acetate (1 mL) and washed with water (4 mL). The organic layer was dried and concentrated. The residue was purified with flash column chromatography (0-100% hexanes/EtOAc) to give 28 mg product (41% yield). LCMS calculated for C₁₇H₁₇BrN₅O₂ (M+H)⁺: m/z=402.1, found: 402.0.

### Step 3. 1-(5-(3-(3,5-dichloro-4-hydroxyphenyl)-4,5,6,7-tetrahydropyrazolo[1,5-a]pyrazine-5-carbonyl)-2,3-dihydro-1H-pyrrolo[2,3-b]pyridin-1-yl)prop-2-en-1-one

To a solution of 1-(5-(3-bromo-4,5,6,7-tetrahydropyrazolo[1,5-a]pyrazine-5-carbonyl)-2,3-dihydro-1H-pyrrolo[2,3-b]pyridin-1-yl)prop-2-en-1-one (27.7 mg, 0.069 mmol) in 1,4-dioxane (1.4 mL) and water (0.200 mL) was added 2,6-dichloro-4-(4,4,5,5-tetramethyl-1,3,2-dioxaborolan-2-yl)phenol (29.8 mg, 0.103 mmol), tripotassium phosphate (43.9 mg, 0.207 mmol), and XPhos Pd G4 (5.93 mg, 6.89 μmol). The reaction mixture was then degassed for 5 minutes before heating to 90° C. The reaction was stirred for 1 hour. Upon completion, the reaction mixture was acidified with TFA, diluted with MeOH, purified by prep-LCMS (XBridge C18 column, eluting with a gradient of acetonitrile/water containing 0.1% TFA, at flow rate of 60 mL/min) to afford desired product (4.2 mg, 13% yield). LCMS calculated for C₂₃H₂₀Cl₂N₅O₃ (M+H)⁺: m/z=484.1, found: 484.1. ¹H NMR (600 MHz, DMSO-d₆) δ 8.30 (d, J=13.2 Hz, 1H), 8.00 (dd, J=17.0, 10.3 Hz, 1H), 7.87 (s, 1H), 7.82 (s, 1H), 7.41 (s, 2H), 6.37 (dd, J=17.1, 2.2 Hz, 1H), 5.87 (dd, J=10.4, 2.4 Hz, 1H), 5.01 (s, 1H), 4.98 (s, 2H), 4.75 (t, J=7.3 Hz, 1H), 4.26 (t, J=5.6 Hz, 2H), 4.17 (t, J=8.3 Hz, 1H), 4.09 (t, J=8.4 Hz, 2H), 3.11 (t, J=8.5 Hz, 2H).

### Example 20. 1-(7-((3-(3,5-dichloro-4-hydroxyphenyl)-6,7-dihydropyrazolo[1,5-a]pyrazin-5(4H)-yl)sulfonyl)-2,3-dihydro-4H-benzo[b][1,4]oxazin-4-yl)prop-2-en-1-one

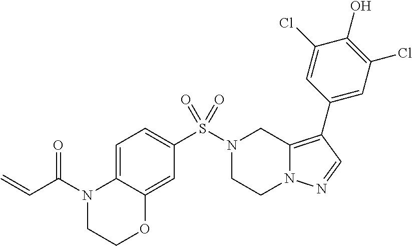 [SMILES: C=CC(=O)N1CCOc2cc(S(=O)(=O)N3CCn4ncc(-c5cc(Cl)c(O)c(Cl)c5)c4C3)ccc21]

### Step 1. tert-butyl 7-((3-(3,5-dichloro-4-hydroxyphenyl)-6,7-dihydropyrazolo[1,5-a]pyrazin-5(4H)-yl)sulfonyl)-2,3-dihydro-4H-benzo[b][1,4]oxazine-4-carboxylate

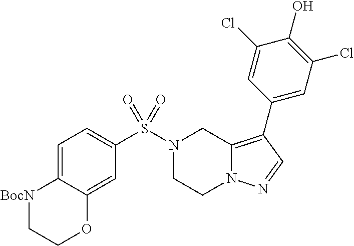 [SMILES: CC(C)(C)OC(=O)N1CCOc2cc(S(=O)(=O)N3CCn4ncc(-c5cc(Cl)c(O)c(Cl)c5)c4C3)ccc21]

To a solution of Intermediate I (25 mg, 0.070 mmol) in DMF (0.70 mL) at 25° C. was added triethylamine (49 μL, 0.35 mmol) and tert-butyl 7-(chlorosulfonyl)-2,3-dihydro-4H-benzo[b][1,4]oxazine-4-carboxylate (25.7 mg, 0.077 mmol). The reaction mixture was then allowed to stir for 5 minutes. Upon completion, the reaction mixture was diluted with EtOAc (15 mL), washed three times with H₂O (10 mL), dried over Na₂SO₄, and concentrated in vacuo. The crude residue was then purified by flash column chromatography (0-20% DCM/MeOH) to provide the desired product (39.0 mg, 0.069 mmol, 97% yield). LCMS calculated for C₂₅H₂₇Cl₂N₄O₆S (M+H)⁺: m/z=581.1; found: 581.1.

### Step 2. 2,6-dichloro-4-(5-((3,4-dihydro-2H-benzo[b][1,4]oxazin-7-yl)sulfonyl)-4,5,6,7-tetrahydropyrazolo[1,5-a]pyrazin-3-yl)phenol

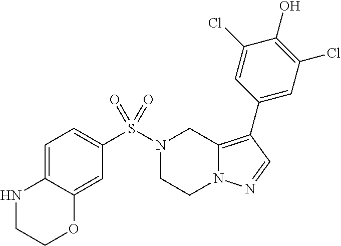 [SMILES: O=S(=O)(c1ccc2c(c1)OCCN2)N1CCn2ncc(-c3cc(Cl)c(O)c(Cl)c3)c2C1]

tert-Butyl 7-((3-(3,5-dichloro-4-hydroxyphenyl)-6,7-dihydropyrazolo[1,5-a]pyrazin-5(4H)-yl)sulfonyl)-2,3-dihydro-4H-benzo[b][1,4]oxazine-4-carboxylate (39 mg, 0.067 mmol) was dissolved in a solution of HCl (172 μL, 4.0 M in 1,4-dioxane, 0.690 mmol). The reaction mixture was allowed to stir for 1 hour. Upon completion, the reaction contents were concentrated in vacuo directly to produce the desired product, and the crude material was used directly in the next step (33.9 mg, 0.069 mmol, 91%). LCMS calculated for C₂₀H₁₉Cl₂N₄O₄S (M+H)⁺: m/z=481.1; found: 481.1.

### Step 3. 1-(7-((3-(3,5-dichloro-4-hydroxyphenyl)-6,7-dihydropyrazolo[1,5-a]pyrazin-5(4H)-yl)sulfonyl)-2,3-dihydro-4H-benzo[b][1,4]oxazin-4-yl)prop-2-en-1-one

To a solution of 2,6-dichloro-4-(5-((3,4-dihydro-2H-benzo[b][1,4]oxazin-7-yl)sulfonyl)-4,5,6,7-tetrahydropyrazolo[1,5-a]pyrazin-3-yl)phenol (16.0 mg, 0.030 mmol) in DMF (0.50 mL) was added DIPEA (30 μL, 0.18 mmol), acrylic acid (2.0 μL, 0.03 mmol), and HATU (11.3 mg, 0.030 mmol). The reaction mixture was then heated to 50° C. and was allowed to stir for 30 minutes. Upon completion, the reaction contents were quenched with TFA, diluted with MeCN, and purified by prep-LCMS (XBridge C18 column, eluting with a gradient of acetonitrile/water containing 0.1% TFA, at flow rate of 60 mL/min) to afford the desired product as a TFA salt. LCMS calculated for C₂₃H₂₁Cl₂N₄O₅S (M+H)⁺: m/z=535.1; found: 535.1. ¹H NMR (600 MHz, DMSO-d₆) δ 10.08 (s, 1H), 7.67 (s, 1H), 7.61 (s, 1H), 7.25 (d, J=2.1 Hz, 1H), 7.23 (s, 2H), 7.18 (d, J=2.2 Hz, 1H), 6.66 (dd, J=16.8, 10.4 Hz, 1H), 6.20 (d, J=16.8 Hz, 1H), 5.78 (d, J=10.5 Hz, 1H), 4.45 (s, 2H), 4.24 (d, J=4.6 Hz, 2H), 4.01 (t, J=5.8 Hz, 2H), 3.85 (t, J=4.6 Hz, 2H), 3.57 (t, J=5.7 Hz, 2H).

### Example 21. N-(7-((3-(3,5-dichloro-4-hydroxyphenyl)-6,7-dihydropyrazolo[1,5-a]pyrazin-5(4H)-yl)sulfonyl)naphthalen-2-yl)acrylamide

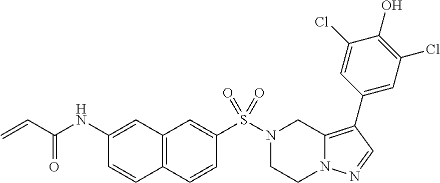 [SMILES: C=CC(=O)Nc1ccc2ccc(S(=O)(=O)N3CCn4ncc(-c5cc(Cl)c(O)c(Cl)c5)c4C3)cc2c1]

This compound was prepared according to the procedures described in Example 20, with tert-butyl (7-(chlorosulfonyl)naphthalen-2-yl)carbamate replacing tert-butyl 7-(chlorosulfonyl)-2,3-dihydro-4H-benzo[b][1,4]oxazine-4-carboxylate in step 1. LCMS calculated for C₂₅H₂₁Cl₂N₄O₄S (M+H)⁺: m/z=543.1; found: 543.1.

### Example 22. N-(4-((3-(3,5-dichloro-4-hydroxyphenyl)-6,7-dihydropyrazolo[1,5-a]pyrazin-5(4H)-yl)sulfonyl)phenyl)acrylamide

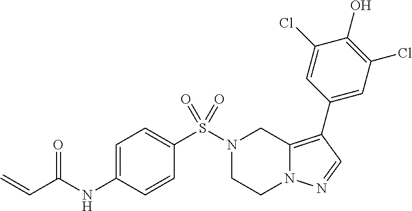 [SMILES: C=CC(=O)Nc1ccc(S(=O)(=O)N2CCn3ncc(-c4cc(Cl)c(O)c(Cl)c4)c3C2)cc1]

### Step 1. tert-butyl (4-((3-bromo-6,7-dihydropyrazolo[1,5-a]pyrazin-5(4H)-yl)sulfonyl)phenyl)carbamate

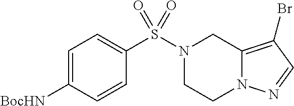 [SMILES: CC(C)(C)OC(=O)Nc1ccc(S(=O)(=O)N2CCn3ncc(Br)c3C2)cc1]

To a solution of 3-bromo-4,5,6,7-tetrahydropyrazolo[1,5-a]pyrazine hydrochloride (25 mg, 0.11 mmol) in DMF (1.0 mL) at 25° C. was added Et₃N (73 μL, 0.52 mmol) and tert-butyl (4-(chlorosulfonyl)phenyl)carbamate (36.7 mg, 0.126 mmol). The reaction mixture was then allowed to stir for 5 minutes. Upon completion, the reaction mixture was diluted with EtOAc (15 mL), washed three times with H₂O (10 mL), dried over Na₂SO₄, and concentrated in vacuo. The crude residue was then purified by flash column chromatography (0-20% DCM/MeOH) to provide the desired product (48 mg, 0.11 mmol, 100%). LCMS calculated for C₁₇H₂₂BrN₄O₄S (M+H)⁺: m/z=457.1; found: 457.1.

### Step 2. 4-((3-bromo-6,7-dihydropyrazolo[1,5-a]pyrazin-5(4H)-yl)sulfonyl)aniline hydrochloride

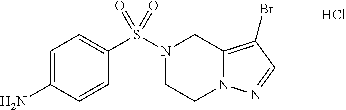 [SMILES: Nc1ccc(S(=O)(=O)N2CCn3ncc(Br)c3C2)cc1 ; Cl]

tert-Butyl (4-((3-bromo-6,7-dihydropyrazolo[1,5-a]pyrazin-5(4H)-yl)sulfonyl)phenyl)carbamate (47.9 mg, 0.105 mmol) was dissolved in a solution of HCl (157 μL, 4.0M in 1,4-Dioxane, 0.628 mmol). The reaction mixture was allowed to stir for 1 hour. Upon completion, the reaction contents were concentrated in vacuo directly to produce the desired product, and the crude material was used directly in the next step (41.2 mg, 0.105 mmol, 100%). LCMS calculated for C₁₂H₁₄BrN₄O_{2S} (M+H)⁺: m/z=357.0; found: 357.0.

### Step 3. N-(4-((3-bromo-6,7-dihydropyrazolo[1,5-a]pyrazin-5(4H)-yl)sulfonyl)phenyl)acrylamide

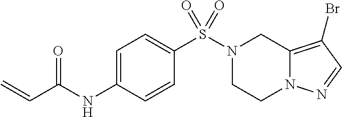 [SMILES: C=CC(=O)Nc1ccc(S(=O)(=O)N2CCn3ncc(Br)c3C2)cc1]

To a solution of 4-((3-bromo-6,7-dihydropyrazolo[1,5-a]pyrazin-5(4H)-yl)sulfonyl)aniline hydrochloride (41.2 mg, 0.105 mmol) in DMF (1047 μL) at 25° C. was added Et₃N (72.9 μL, 0.523 mmol) and acryloyl chloride (10.99 μL, 0.136 mmol). The reaction contents were allowed to stir for 5 minutes. Upon completion, the reaction mixture was diluted with EtOAc (15 mL), washed three times with H₂O (10 mL), dried over Na₂SO₄, and concentrated in vacuo. The crude residue was then purified by flash column chromatography (0-20% DCM/MeOH) to provide the desired product (30 mg, 0.073 mmol, 70%). LCMS calculated for C₁₅H₁₆BrN₄O₃S (M+H)⁺: m/z=411.0; found: 411.0.

### Step 4. N-(4-((3-(3,5-dichloro-4-hydroxyphenyl)-6,7-dihydropyrazolo[1,5-a]pyrazin-5(4H)-yl)sulfonyl)phenyl)acrylamide

To a solution of N-(4-((3-bromo-6,7-dihydropyrazolo[1,5-a]pyrazin-5(4H)-yl)sulfonyl)phenyl)acrylamide (18.0 mg, 0.044 mmol) in 1,4-dioxane (1.0 mL) and water (0.14 mL) was added 2,6-dichloro-4-(4,4,5,5-tetramethyl-1,3,2-dioxaborolan-2-yl)phenol (19.0 mg, 0.066 mmol), tripotassium phosphate (27.9 mg, 0.131 mmol), and XPhos Pd G4 (7.5 mg, 8.8 μmol). The reaction mixture was then degassed for 5 minutes before heating to 90° C. The reaction mixture was stirred for 1 hour. Upon completion, the reaction contents were quenched with TFA, diluted with MeCN, and purified by prep-LCMS (XBridge C18 column, eluting with a gradient of acetonitrile/water containing 0.1% TFA, at flow rate of 60 mL/min) to afford the desired product as a TFA salt. LCMS calculated for C₂₁H₁₉Cl₂N₄O₄S (M+H)⁺: m/z=493.0; found: 493.1. ¹H NMR (600 MHz, DMSO-d₆) δ 10.61 (s, 1H), 10.21 (s, 1H), 7.92 (d, J=8.6 Hz, 2H), 7.84 (d, J=8.5 Hz, 2H), 7.80 (s, 1H), 7.37 (s, 2H), 6.48 (dd, J=16.9, 10.1 Hz, 1H), 6.35 (dd, J=16.9, 1.8 Hz, 1H), 5.86 (dd, J=10.2, 1.8 Hz, 1H), 4.55 (s, 2H), 4.14 (t, J=5.7 Hz, 2H), 3.65 (t, J=5.8 Hz, 2H).

### Example 23. N-(5-((3-(3,5-dichloro-4-hydroxyphenyl)-6,7-dihydropyrazolo[1,5-a]pyrazin-5(4H)-yl)methyl)pyridin-2-yl)acrylamide

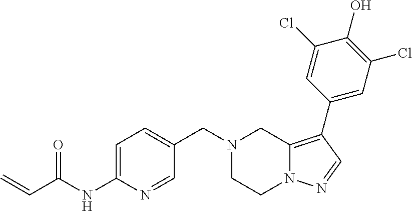 [SMILES: C=CC(=O)Nc1ccc(CN2CCn3ncc(-c4cc(Cl)c(O)c(Cl)c4)c3C2)cn1]

### Step 1. tert-butyl (5-((3-bromo-6,7-dihydropyrazolo[1,5-a]pyrazin-5(4H)-yl)methyl)pyridin-2-yl)carbamate

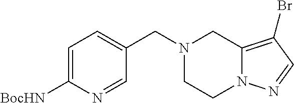 [SMILES: CC(C)(C)OC(=O)Nc1ccc(CN2CCn3ncc(Br)c3C2)cn1]

To a solution of tert-butyl (5-formylpyridin-2-yl)carbamate (55.9 mg, 0.252 mmol) in MeCN (0.84 mL) at 25° C. was added acetic acid (14.4 μL, 0.252 mmol) and 3-bromo-4,5,6,7-tetrahydropyrazolo[1,5-a]pyrazine hydrochloride (20.0 mg, 0.084 mmol). The reaction was stirred for 30 minutes before the addition of sodium triacetoxyborohydride (53.3 mg, 0.252 mmol). The reaction mixture was then allowed to stir for 5 minutes. Upon completion, the reaction mixture was quenched with water (10 mL), extracted three times with DCM, dried over Na₂SO₄, and concentrated in vacuo. The crude residue was then purified by flash column chromatography (0-20% DCM/MeOH) to provide the desired product (19.2 mg, 0.047 mmol, 56%). LCMS calculated for C₁₇H₂₃BrN₅O₂(M+H)⁺: m/z=407.1; found: 407.1.

### Step 2. 5-((3-bromo-6,7-dihydropyrazolo[1,5-a]pyrazin-5(4H)-yl)methyl)pyridin-2-amine dihydrochloride

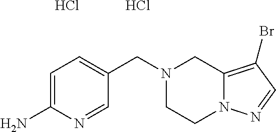 [SMILES: Cl ; Cl ; Nc1ccc(CN2CCn3ncc(Br)c3C2)cn1]

tert-Butyl (5-((3-bromo-6,7-dihydropyrazolo[1,5-a]pyrazin-5(4H)-yl)methyl)pyridin-2-yl)carbamate (19.2 mg, 0.047 mmol) was dissolved in a solution of HCl (70.5 μL, 4.0 M in 1,4-dioxane, 0.282 mmol). The reaction mixture was allowed to stir for 1 hour. Upon completion, the reaction contents were concentrated in vacuo directly to produce the desired product, and the crude material was used directly in the next step (17.9 mg, 0.047 mmol, 100%). LCMS calculated for C₁₂H₁₅BrN₅ (M+H)⁺: m/z=307.0; found: 307.0.

### Step 3. N-(5-((3-bromo-6,7-dihydropyrazolo[1,5-a]pyrazin-5(4H)-yl)methyl)pyridin-2-yl)acrylamide

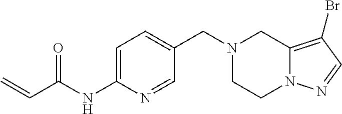 [SMILES: C=CC(=O)Nc1ccc(CN2CCn3ncc(Br)c3C2)cn1]

To a solution of 5-((3-bromo-6,7-dihydropyrazolo[1,5-a]pyrazin-5(4H)-yl)methyl)pyridin-2-amine dihydrochloride (17.9 mg, 0.047 mmol) in DMF (0.47 mL) at 25° C. was added Et₃N (32.7 μL, 0.235 mmol) and acryloyl chloride (4.6 μL, 0.06 mmol). The reaction contents were allowed to stir for 5 minutes. Upon completion, the reaction mixture was diluted with EtOAc (15 mL), washed three times with water (10 mL), dried over Na₂SO₄, and concentrated in vacuo. The crude residue was then purified by flash column chromatography (0-20% DCM/MeOH) to provide the desired product (5.0 mg, 0.014 mmol, 29%). LCMS calculated for C₁₅H₁₇BrN₅O (M+H)⁺: m/z=362.1; found: 362.1.

### Step 4. N-(5-((3-(3,5-dichloro-4-hydroxyphenyl)-6,7-dihydropyrazolo[1,5-a]pyrazin-5(4H)-yl)methyl)pyridin-2-yl)acrylamide

To a solution of N-(5-((3-bromo-6,7-dihydropyrazolo[1,5-a]pyrazin-5(4H)-yl)methyl)pyridin-2-yl)acrylamide (5.0 mg, 0.014 mmol) in 1,4-dioxane (1.0 mL) and water (0.14 mL) was added 2,6-dichloro-4-(4,4,5,5-tetramethyl-1,3,2-dioxaborolan-2-yl)phenol (6.0 mg, 0.021 mmol), tripotassium phosphate (8.8 mg, 0.041 mmol), and XPhos Pd G4 (2.4 mg, 2.8 μmol). The reaction mixture was then degassed for 5 minutes before heating to 90° C. The reaction mixture was stirred for 1 hour. Upon completion, the reaction contents were quenched with TFA, diluted with MeCN, and purified by prep-LCMS (XBridge C18 column, eluting with a gradient of acetonitrile/water containing 0.1% TFA, at flow rate of 60 mL/min) to afford the desired product as a TFA salt. LCMS calculated for C₂₁H₂₀Cl₂N₅O₂ (M+H)⁺: m/z=444.1; found: 444.2. ¹H NMR (600 MHz, DMSO-d₆) δ 10.75 (s, 1H), 10.11 (s, 1H), 8.34 (s, 1H), 8.20 (d, J=8.4 Hz, 1H), 7.82 (s, 2H), 7.34 (s, 2H), 6.61 (dd, J=17.0, 10.3 Hz, 1H), 6.32 (d, J=17.1 Hz, 1H), 5.79 (d, J=10.3 Hz, 1H), 4.13 (s, 2H), 3.91 (s, 2H), 3.81 (s, 2H), 2.94 (s, 2H).

### Example 24. N-(4-((3-(3,5-dichloro-4-hydroxyphenyl)-6,7-dihydropyrazolo[1,5-a]pyrazin-5(4H)-yl)methyl)-2-methoxyphenyl)acrylamide

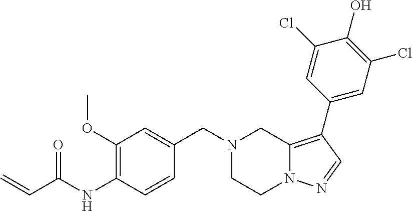 [SMILES: C=CC(=O)Nc1ccc(CN2CCn3ncc(-c4cc(Cl)c(O)c(Cl)c4)c3C2)cc1OC]

This compound was prepared according to the procedures described in Example 23, with tert-butyl (4-formyl-2-methoxyphenyl)carbamate replacing tert-butyl (5-formylpyridin-2-yl)carbamate in step 1. LCMS calculated for C₂₃H₂₃Cl₂N₄O₃ (M+H)⁺: m/z=473.1; found: 473.1.

### Example 25. 1-(5-((3-(3,5-dichloro-4-hydroxyphenyl)-6,7-dihydropyrazolo[1,5-a]pyrazin-5(4H)-yl)methyl)isoindolin-2-yl)prop-2-en-1-one

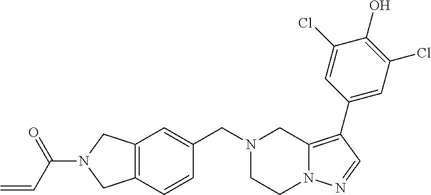 [SMILES: C=CC(=O)N1Cc2ccc(CN3CCn4ncc(-c5cc(Cl)c(O)c(Cl)c5)c4C3)cc2C1]

This compound was prepared according to the procedures described in Example 23, with tert-butyl 5-formylisoindoline-2-carboxylate replacing tert-butyl (5-formylpyridin-2-yl)carbamate in step 1. LCMS calculated for C₂₄H₂₃Cl₂N₄O₂ (M+H)⁺: m/z=469.1; found: 469.1. ¹H NMR (600 MHz, DMSO-d₆) δ 7.77 (s, 1H), 7.45-7.32 (m, 3H), 7.29 (s, 2H), 6.67 (dd, J=16.7 Hz, 10.3 Hz, 1H), 6.22 (d, J=16.7 Hz, 1H), 5.74 (d, J=10.3 Hz, 1H), 4.94 (s, 2H), 4.74 (s, 2H), 4.19 (t, J=4.7 Hz, 2H), 4.04 (s, 2H), 3.98 (s, 2H), 3.15 (s, 2H).

### Example 26. N-(2-chloro-4-((3-(3,5-dichloro-4-hydroxyphenyl)-6,7-dihydropyrazolo[1,5-a]pyrazin-5(4H)-yl)methyl)phenyl)acrylamide

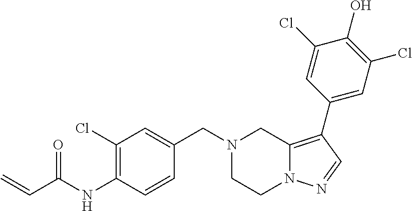 [SMILES: C=CC(=O)Nc1ccc(CN2CCn3ncc(-c4cc(Cl)c(O)c(Cl)c4)c3C2)cc1Cl]

This compound was prepared according to the procedures described in Example 23, with tert-butyl (2-chloro-4-formylphenyl)carbamate replacing tert-butyl (5-formylpyridin-2-yl)carbamate in step 1. LCMS calculated for C₂₂H₂₀Cl₃N₄O₂ (M+H)⁺: m/z=477.1; found: 477.1.

### Example 27. N-(4-(3-(3,5-dichloro-4-hydroxyphenyl)-6-methyl-4,5,6,7-tetrahydropyrazolo[1,5-a]pyrazine-5-carbonyl)phenyl)acrylamide

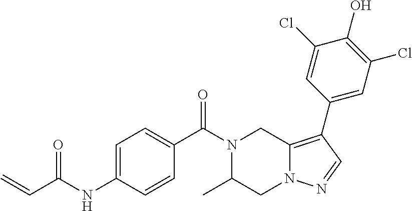 [SMILES: C=CC(=O)Nc1ccc(C(=O)N2Cc3c(-c4cc(Cl)c(O)c(Cl)c4)cnn3CC2C)cc1]

This compound was prepared according to the procedures described in Example 1, with tert-butyl 3-bromo-6-methyl-6,7-dihydropyrazolo[1,5-a]pyrazine-5(4H)-carboxylate replacing tert-butyl 3-bromo-6,7-dihydropyrazolo[1,5-a]pyrazine-5(4H)-carboxylate in the Intermediate I preparation. LC-MS calculated for C₂₃H₂₁Cl₂N₄O₃ (M+H)⁺: m/z=471.1; found 471.1. ¹H NMR (600 MHz, DMSO-d₆) δ 10.15 (s, 1H), 7.83 (s, 1H), 7.77 (d, J=8.4 Hz, 2H), 7.48 (d, J=8.4 Hz, 2H), 7.40 (brs, 2H), 6.45 (dd, J=16.4 Hz, 10.3 Hz, 1H), 6.30 (d, J=16.4 Hz, 1H), 5.78 (d, J=10.3 Hz, 1H), 5.24-5.16 (m, 1H), 4.72 (s, 1H), 4.69 (brs, 1H), 4.31 (dd, J=11.9 Hz, 4.4 Hz, 1H), 4.10 (d, J=11.9 Hz, 1H), 1.24 (d, J=7.2 Hz, 3H).

### Example 28. (E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(dimethylamino)but-2-enamide

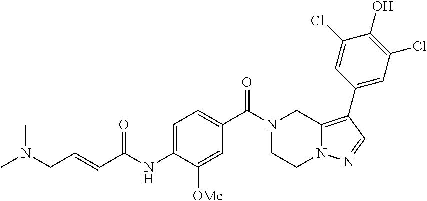 [SMILES: COc1cc(C(=O)N2CCn3ncc(-c4cc(Cl)c(O)c(Cl)c4)c3C2)ccc1NC(=O)/C=C/CN(C)C]

### Step 1. methyl (E)-4-(4-(dimethylamino)but-2-enamido)-3-methoxybenzoate

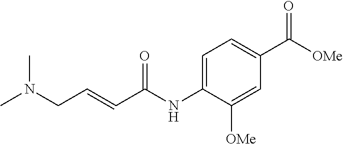 [SMILES: COC(=O)c1ccc(NC(=O)/C=C/CN(C)C)c(OC)c1]

To a suspension of (E)-4-(dimethylamino)but-2-enoic acid hydrochloride (260 mg, 1.57 mmol) in MeCN (20 mL) was added a few drops of DMF. Oxalyl chloride (0.180 mL, 2.04 mmol) was then added slowly and the mixture was stirred at room temperature for 2 hours. The mixture was then concentrated and redissolved in DCM (5 mL). Methyl 4-amino-3-methoxybenzoate (313 mg, 1.73 mmol) was dissolved in DCM (5 mL) and subsequently added to the stirring reaction mixture at room temperature. Upon completion, the mixture was quenched with saturated aqueous NaHCO₃, extracted with DCM and ethyl acetate. The combined organic layer was dried over MgSO₄, concentrated, and purified by silica gel column chromatography (0-20% MeOH/DCM) to afford the desired product (115 mg, 25% yield). LCMS calculated for C₁₅H₂₁N₂O₄ (M+H)⁺: m/z=293.1; found: 293.1.

### Step 2. (E)-4-(4-(dimethylamino)but-2-enamido)-3-methoxybenzoic acid

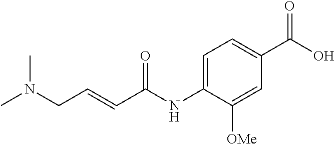 [SMILES: COc1cc(C(=O)O)ccc1NC(=O)/C=C/CN(C)C]

To a 40 mL scintillation vial containing a magnetic stir bar and methyl (E)-4-(4-(dimethylamino)but-2-enamido)-3-methoxybenzoate (115 mg, 0.393 mmol) was added lithium hydroxide monohydrate (41.3 mg, 0.983 mmol), THF (1.6 mL) and water (0.33 mL). The mixture was stirred at 50° C. for 1 hour. Upon completion, the reaction mixture was quenched with concentrated HCl (0.036 mL, 12.1 M, 1.18 mmol), concentrated in vacuo, and carried forward to the next step without further purification. LCMS calculated for C₁₄H₁₉N₂O₄ (M+H)⁺: m/z=279.1; found: 279.2.

### Step 3. (E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(dimethylamino)but-2-enamide

To a 40 mL scintillation vial containing a magnetic stir bar was added 2,6-dichloro-4-(4,5,6,7-tetrahydropyrazolo[1,5-a]pyrazin-3-yl)phenol (63 mg, 0.22 mmol), (E)-4-(4-(dimethylamino)but-2-enamido)-3-methoxybenzoic acid (110 mg, 0.40 mmol), HATU (209 mg, 0.55 mmol), DMF (1.1 mL), and DIPEA (0.192 mL, 1.10 mmol). The mixture was stirred at room temperature for 15 minutes. Upon full consumption of the starting material, lithium hydroxide monohydrate (28 mg, 0.66 mmol) and water (0.3 mL) were added and the mixture was stirred at 50° C. for 10 minutes. The reaction mixture was then diluted with acetonitrile, filtered, and purified by prep LCMS (pH 10) to afford the desired product. LCMS calculated for C₂₆H₂₈Cl₂N₅O₄ (M+H)⁺: 544.1; found: 544.1. ¹H NMR (600 MHz, DMSO-d₆) δ 9.70 (s, 1H), 8.18 (d, J=8.3 Hz, 1H), 7.87 (s, 1H), 7.39 (s, 2H), 7.21 (s, 1H), 7.12 (d, J=8.4 Hz, 1H), 6.80-6.69 (m, 2H), 4.98 (s, 2H), 4.24 (d, J=5.6 Hz, 3H), 3.91 (d, J=17.4 Hz, 6H), 3.50 (s, 1H), 2.81 (d, J=3.3 Hz, 6H).

### Example 29. (E)-N-(5-(3-(3,5-dichloro-4-hydroxyphenyl)-4,5,6,7-tetrahydropyrazolo[1,5-a]pyrazine-5-carbonyl)quinolin-8-yl)-4-(dimethylamino)but-2-enamide

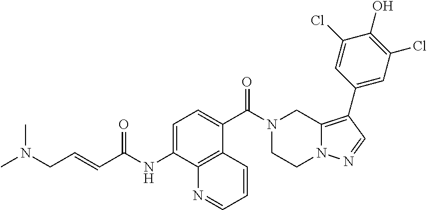 [SMILES: CN(C)C/C=C/C(=O)Nc1ccc(C(=O)N2CCn3ncc(-c4cc(Cl)c(O)c(Cl)c4)c3C2)c2cccnc12]

This compound was prepared according to the procedures described in Example 28, with methyl 8-aminoquinoline-5-carboxylate replacing methyl 4-amino-3-methoxybenzoate in step 1. LCMS calculated for C₂₈H₂₇Cl₂N₆O₃ (M+H)⁺: m/z=565.2, found: 565.2. ¹H NMR (600 MHz, DMSO-d₆) δ 10.65 (s, 1H), 9.67 (s, 1H), 9.03 (s, 1H), 8.77 (d, J=7.9 Hz, 1H), 8.45-8.28 (m, 1H), 7.97-7.86 (m, 1H), 7.75 (s, 2H), 7.48 (s, 2H), 7.03 (t, J=16.1 Hz, 1H), 6.90-6.79 (m, 1H), 5.19 (s, 2H), 3.97 (d, J=6.7 Hz, 3H), 3.72 (s, 3H), 2.85 (d, J=4.2 Hz, 6H).

### Example 30. (E)-N-(4-(3-(3,5-dichloro-4-hydroxyphenyl)-4,5,6,7-tetrahydropyrazolo[1,5-a]pyrazine-5-carbonyl)phenyl)-4-(dimethylamino)but-2-enamide

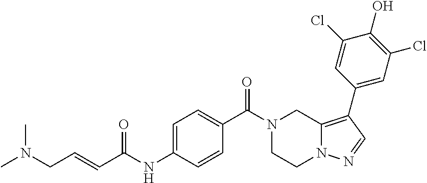 [SMILES: CN(C)C/C=C/C(=O)Nc1ccc(C(=O)N2CCn3ncc(-c4cc(Cl)c(O)c(Cl)c4)c3C2)cc1]

This compound was prepared according to the procedures described in Example 28, with methyl 4-aminobenzoate replacing methyl 4-amino-3-methoxybenzoate in step 1. LCMS calculated for C₂₅H₂₆Cl₂N₅O₃ (M+H)⁺: m/z=514.1; found: 514.1. ¹H NMR (600 MHz, DMSO-d₆) δ 10.51 (s, 1H), 7.87 (s, 1H), 7.77 (d, J=8.3 Hz, 2H), 7.53 (d, J=8.3 Hz, 2H), 7.38 (s, 2H), 6.77 (dt, J=14.7, 7.0 Hz, 1H), 6.56-6.40 (m, 1H), 4.95 (s, 2H), 4.24 (t, J=5.6 Hz, 3H), 3.93 (s, 4H), 2.79 (s, 6H).

### Example 31. (E)-N-(4-(3-(3,5-dichloro-4-hydroxyphenyl)-4,5,6,7-tetrahydropyrazolo[1,5-a]pyrazine-5-carbonyl)-2-fluorophenyl)-4-(dimethylamino)but-2-enamide

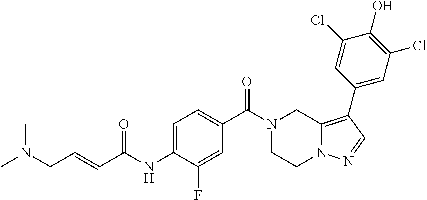 [SMILES: CN(C)C/C=C/C(=O)Nc1ccc(C(=O)N2CCn3ncc(-c4cc(Cl)c(O)c(Cl)c4)c3C2)cc1F]

This compound was prepared according to the procedures described in Example 28, with methyl 4-amino-3-fluorobenzoate replacing methyl 4-amino-3-methoxybenzoate in step 1. LCMS calculated for C₂₅H₂₅Cl₂FN₅O₃(M+H)⁺: m/z=532.1; found: 532.1. ¹H NMR (600 MHz, DMSO-d₆) δ 10.30 (s, 1H), 8.16 (t, J=7.9 Hz, 1H), 7.86 (d, J=16.9 Hz, 1H), 7.50 (d, J=11.0 Hz, 1H), 7.38 (d, J=8.4 Hz, 2H), 6.79 (dt, J=14.7, 7.0 Hz, 1H), 6.68 (d, J=15.3 Hz, 1H), 4.97 (s, 2H), 4.25 (t, J=5.4 Hz, 2H), 3.95 (d, J=7.0 Hz, 3H), 2.82 (d, J=3.5 Hz, 8H).

### Example 32. (E)-1-(5-(3-(3,5-dichloro-4-hydroxyphenyl)-4,5,6,7-tetrahydropyrazolo[1,5-a]pyrazine-5-carbonyl)indolin-1-yl)-4-(dimethylamino)but-2-en-1-one

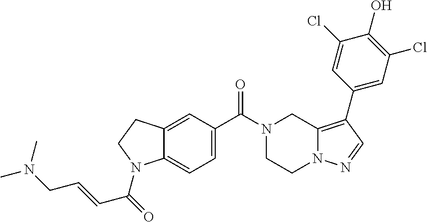 [SMILES: CN(C)C/C=C/C(=O)N1CCc2cc(C(=O)N3CCn4ncc(-c5cc(Cl)c(O)c(Cl)c5)c4C3)ccc21]

This compound was prepared according to the procedures described in Example 28, with methyl indoline-5-carboxylate replacing methyl 4-amino-3-methoxybenzoate in step 1. LCMS calculated for C₂₇H₂₈Cl₂N₅O₃ (M+H)⁺: m/z=540.2, found: 540.1. ¹H NMR (600 MHz, DMSO-d₆) δ 8.19 (d, J=8.3 Hz, 1H), 7.86 (s, 1H), 7.44 (s, 1H), 7.44-7.36 (m, 2H), 6.87-6.75 (m, 2H), 4.96 (s, 3H), 4.30-4.21 (m, 4H), 3.96 (s, 3H), 3.23 (s, 2H), 2.81 (s, 8H).

### Example 33. (E)-N-(4-(3-(3,5-dichloro-4-hydroxyphenyl)-4,5,6,7-tetrahydropyrazolo[1,5-a]pyrazine-5-carbonyl)phenyl)-4-(piperidin-1-yl)but-2-enamide

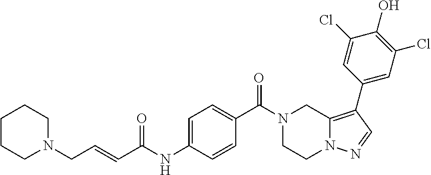 [SMILES: O=C(/C=C/CN1CCCCC1)Nc1ccc(C(=O)N2CCn3ncc(-c4cc(Cl)c(O)c(Cl)c4)c3C2)cc1]

This compound was prepared according to the procedures described in Example 30, with (E)-4-(piperidin-1-yl)but-2-enoic acid replacing (E)-4-(dimethylamino)but-2-enoic acid in step 1. LCMS calculated for C₂₈H₃₀Cl₂N₅O₃ (M+H)⁺: m/z=554.2; found: 554.2.

### Example 34. (E)-N-(4-(3-(3,5-dichloro-4-hydroxyphenyl)-4,5,6,7-tetrahydropyrazolo[1,5-a]pyrazine-5-carbonyl)phenyl)-4-morpholinobut-2-enamide

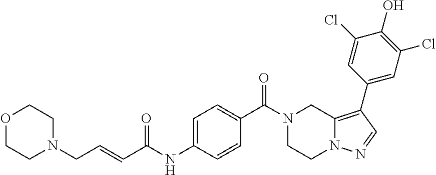 [SMILES: O=C(/C=C/CN1CCOCC1)Nc1ccc(C(=O)N2CCn3ncc(-c4cc(Cl)c(O)c(Cl)c4)c3C2)cc1]

This compound was prepared according to the procedures described in Example 30, with (E)-4-morpholinobut-2-enoic acid replacing (E)-4-(dimethylamino)but-2-enoic acid in step 1. LCMS calculated for C₂₇H₂₈Cl₂N₅O₄ (M+H)⁺: m/z=556.1; found: 556.1. ¹H NMR (600 MHz, DMSO-d₆) δ 10.28 (s, 1H), 7.75 (d, J=8.3 Hz, 2H), 7.49 (d, J=8.2 Hz, 2H), 7.28 (s, 2H), 6.76 (dt, J=15.5, 5.9 Hz, 1H), 6.52 (d, J=2.4 Hz, 1H), 6.30 (d, J=15.4 Hz, 1H), 4.92 (s, 2H), 4.22 (d, J=5.6 Hz, 2H), 3.91 (s, 2H), 3.61 (t, J=4.7 Hz, 4H), 3.13 (d, J=5.9 Hz, 2H), 2.40 (s, 4H).

### Example 35. (E)-N-(4-(3-(3,5-dichloro-4-hydroxyphenyl)-4,5,6,7-tetrahydropyrazolo[1,5-a]pyrazine-5-carbonyl)phenyl)-4-(diethylamino)but-2-enamide

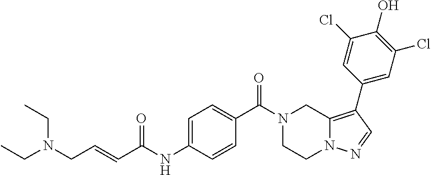 [SMILES: CCN(CC)C/C=C/C(=O)Nc1ccc(C(=O)N2CCn3ncc(-c4cc(Cl)c(O)c(Cl)c4)c3C2)cc1]

This compound was prepared according to the procedures described in Example 30, with (E)-4-(diethylamino)but-2-enoic acid replacing (E)-4-(dimethylamino)but-2-enoic acid in step 1. LCMS calculated for C₂₇H₃₀Cl₂N₅O₃ (M+H)⁺: m/z=542.2; found: 542.2. ¹H NMR (600 MHz, DMSO-d₆) δ 10.25 (s, 1H), 7.75 (d, J=8.3 Hz, 2H), 7.49 (d, J=8.2 Hz, 2H), 7.24 (s, 2H), 6.85-6.77 (m, 1H), 6.52 (d, J=2.5 Hz, 1H), 6.30 (d, J=15.3 Hz, 1H), 4.93 (d, J=17.2 Hz, 2H), 4.22 (s, 2H), 3.91 (s, 2H), 3.22 (d, J=5.7 Hz, 2H), 2.47 (m, 4H), 0.99 (t, J=7.1 Hz, 6H).

### Example 36. (E)-1-(5-(3-(3,5-dichloro-4-hydroxyphenyl)-4,5,6,7-tetrahydropyrazolo[1,5-a]pyrazine-5-carbonyl)isoindolin-2-yl)-4-(dimethylamino)but-2-en-1-one

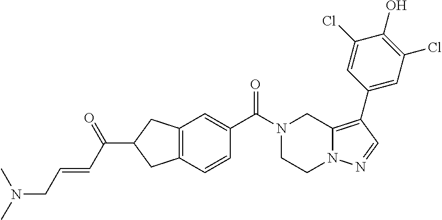 [SMILES: CN(C)C/C=C/C(=O)C1Cc2ccc(C(=O)N3CCn4ncc(-c5cc(Cl)c(O)c(Cl)c5)c4C3)cc2C1]

This compound was prepared according to the procedures described in Example 28, with methyl isoindoline-5-carboxylate replacing methyl 4-amino-3-methoxybenzoate in step 1. LCMS calculated for C₂₇H₂₈Cl₂N₅O₃ (M+H)⁺: m/z=540.2; found: 540.2. ¹H NMR (600 MHz, DMSO-d₆) δ 7.82 (s, 1H), 7.54 (s, 1H), 7.50-7.43 (m, 2H), 7.36 (s, 1H), 6.72 (dt, J=13.3, 6.0 Hz, 1H), 6.53-6.45 (m, 2H), 5.09-4.86 (m, 4H), 4.75 (s, 2H), 4.22 (t, J=5.5 Hz, 2H), 3.84 (s, 1H), 3.08 (t, J=4.6 Hz, 3H), 2.18 (s, 6H).

### Example 37. (E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(piperidin-1-yl)but-2-enamide

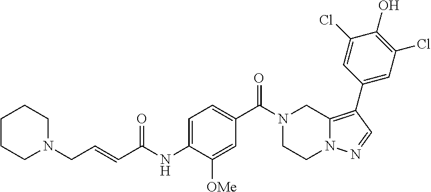 [SMILES: COc1cc(C(=O)N2CCn3ncc(-c4cc(Cl)c(O)c(Cl)c4)c3C2)ccc1NC(=O)/C=C/CN1CCCCC1]

### Step 1. tert-butyl (E)-3-methoxy-4-(4-(piperidin-1-yl)but-2-enamido)benzoate

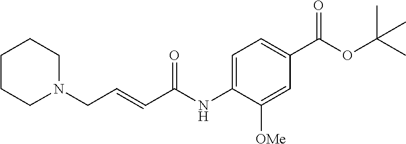 [SMILES: COc1cc(C(=O)OC(C)(C)C)ccc1NC(=O)/C=C/CN1CCCCC1]

To a 40 mL scintillation vial equipped with a stir bar was added tert-butyl 4-amino-3-methoxybenzoate (155 mg, 0.694 mmol), (E)-4-(piperidin-1-yl)but-2-enoic acid hydrochloride (286 mg, 1.39 mmol), THF (3.5 mL), and DIPEA (0.73 mL, 4.17 mmol). To this stirring solution at room temperature was added propane phosphonic acid anhydride (1.24 mL, 2.08 mmol, 50% solution in THF) dropwise. The mixture was stirred at room temperature for 1 hour. After the reaction was complete, water and saturated brine were added, and the mixture was extracted with ethyl acetate 3 times. The combined organic layer was dried over anhydrous MgSO₄, concentrated in vacuo, and purified by silica gel column chromatography (0-20% MeOH/DCM) to afford the desired product (222 mg, 0.593 mmol, 85% yield) as dark oil. LCMS calculated for C₂₁H₃₁N₂O₄ (M+H)⁺: m/z=375.2; found: 375.3.

### Step 2. (E)-3-methoxy-4-(4-(piperidin-1-yl)but-2-enamido)benzoic acid

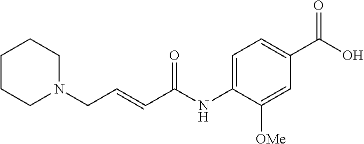 [SMILES: COc1cc(C(=O)O)ccc1NC(=O)/C=C/CN1CCCCC1]

To a 40 mL scintillation vial equipped with a stir bar was added tert-butyl (E)-3-methoxy-4-(4-(piperidin-1-yl)but-2-enamido)benzoate (222 mg, 0.593 mmol), DCM (3 mL), and trifluoroacetic acid (1 mL). The mixture was stirred at room temperature until analysis by LCMS indicated full conversion of the reactant. The mixture was then evaporated in vacuo and carried forward to the next step without further purification. LCMS calculated for C₁₇H₂₃N₂O₄ (M+H)⁺: m/z=319.2; found: 319.2.

### Step 3. (E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(piperidin-1-yl)but-2-enamide

To a 40 mL scintillation vial containing a magnetic stir bar was added 2,6-dichloro-4-(4,5,6,7-tetrahydropyrazolo[1,5-a]pyrazin-3-yl)phenol (50 mg, 0.18 mmol), (E)-3-methoxy-4-(4-(piperidin-1-yl)but-2-enamido)benzoic acid (73 mg, 0.23 mmol), HATU (100 mg, 0.26 mmol), DMF (1.8 mL), and DIPEA (0.092 mL, 0.53 mmol). The mixture was stirred at room temperature for 15 minutes. Upon full consumption of the starting material, lithium hydroxide monohydrate (22 mg, 0.53 mmol) and water (0.3 mL) were added and the mixture was stirred at 50° C. for 10 minutes. The reaction mixture was then diluted with acetonitrile, filtered, and purified by prep LCMS (pH 10) to afford the desired product. LCMS calculated for C₂₉H₃₂Cl₂N₅O₄ (M+H)⁺: m/z=584.2; found: 584.2. ¹H NMR (600 MHz, DMSO-d₆) δ 9.41 (s, 1H), 8.19 (d, J=8.1 Hz, 1H), 7.85 (s, 1H), 7.37 (s, 2H), 7.17 (s, 1H), 7.10 (d, J=8.3 Hz, 1H), 6.74 (dt, J=15.4, 6.1 Hz, 1H), 6.54 (d, J=15.3 Hz, 1H), 4.98 (s, 2H), 4.24 (t, J=5.4 Hz, 2H), 3.88 (br, 5H), 3.08 (s, 2H), 2.35 (s, 4H), 1.52 (m, 5H), 1.39 (d, J=8.4 Hz, 2H).

### Example 38. (E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(pyrrolidin-1-yl)but-2-enamide

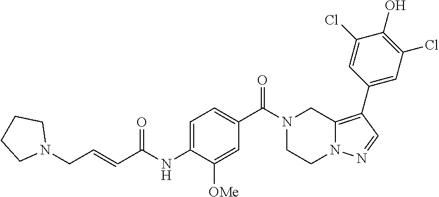 [SMILES: COc1cc(C(=O)N2CCn3ncc(-c4cc(Cl)c(O)c(Cl)c4)c3C2)ccc1NC(=O)/C=C/CN1CCCC1]

This compound was prepared according to the procedures described in Example 37, with (E)-4-(pyrrolidin-1-yl)but-2-enoic acid replacing (E)-4-(piperidin-1-yl)but-2-enoic acid in step 1. LCMS calculated for C₂₈H₃₀Cl₂N₅O₄ (M+H)⁺: m/z=570.2; found: 570.2. ¹H NMR (600 MHz, DMSO-d₆) δ 9.67 (s, 1H), 8.22-8.09 (m, 1H), 7.87 (s, 1H), 7.49-7.29 (m, 2H), 7.20 (s, 1H), 7.12 (d, J=8.3 Hz, 1H), 6.85-6.71 (m, 2H), 4.98 (s, 2H), 4.24 (t, J=5.4 Hz, 2H), 4.10-3.78 (m, 7H), 3.64-3.47 (m, 2H), 3.11-3.06 (m, 2H), 2.11-1.94 (m, 2H), 1.89 (m, 2H).

### Example 39. (E)-N-(4-(3-(3,5-dichloro-4-hydroxyphenyl)-4,5,6,7-tetrahydropyrazolo[1,5-a]pyrazine-5-carbonyl)-2-methoxyphenyl)-4-morpholinobut-2-enamide

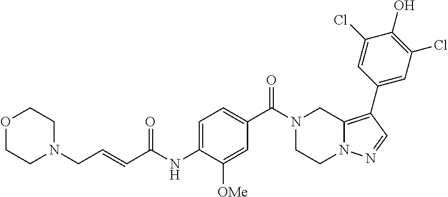 [SMILES: COc1cc(C(=O)N2CCn3ncc(-c4cc(Cl)c(O)c(Cl)c4)c3C2)ccc1NC(=O)/C=C/CN1CCOCC1]

This compound was prepared according to the procedures described in Example 37, with (E)-4-morpholinobut-2-enoic acid replacing (E)-4-(piperidin-1-yl)but-2-enoic acid in step 1. LCMS calculated for C₂₈H₃₀Cl₂N₅O₅ (M+H)⁺: m/z=586.2; found: 586.2. ¹H NMR (600 MHz, DMSO-d₆) δ 9.69 (s, 1H), 8.17 (d, J=8.2 Hz, 1H), 7.87 (s, 1H), 7.50-7.27 (br, 2H), 7.20 (s, 1H), 7.12 (d, J=8.4 Hz, 1H), 6.76 (s, 3H), 4.98 (s, 3H), 4.24 (t, J=5.6 Hz, 3H), 4.12-3.76 (m, 10H), 3.64 (s, 2H).

### Example 40. (E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(diethylamino)but-2-enamide

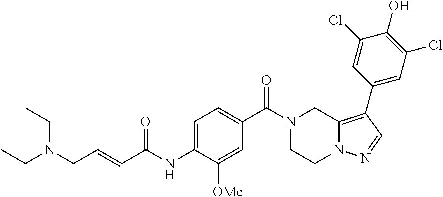 [SMILES: CCN(CC)C/C=C/C(=O)Nc1ccc(C(=O)N2CCn3ncc(-c4cc(Cl)c(O)c(Cl)c4)c3C2)cc1OC]

This compound was prepared according to the procedures described in Example 37, with (E)-4-(diethylamino)but-2-enoic acid replacing (E)-4-(piperidin-1-yl)but-2-enoic acid in step 1. LCMS calculated for C₂₈H₃₂Cl₂N₅O₄ (M+H)⁺: m/z=572.2; found: 572.2. ¹H NMR (600 MHz, DMSO-d₆) δ 9.40 (s, 1H), 8.19 (d, J=8.3 Hz, 1H), 7.85 (s, 1H), 7.53-7.23 (br, 2H), 7.17 (d, J=1.7 Hz, 1H), 7.10 (d, J=8.2 Hz, 1H), 6.77 (dt, J=15.3, 6.1 Hz, 1H), 6.57 (d, J=15.3 Hz, 1H), 4.98 (s, 2H), 4.24 (t, J=5.4 Hz, 2H), 4.05-3.77 (m, 5H), 3.21 (d, J=6.0 Hz, 2H), 2.47 (m, 4H), 0.99 (t, J=7.1 Hz, 6H).

### Example 41. (E)-N-(4-(3-(3,5-dichloro-4-hydroxyphenyl)-4,5,6,7-tetrahydropyrazolo[1,5-a]pyrazine-5-carbonyl)-3-methoxyphenyl)-4-(dimethylamino)but-2-enamide

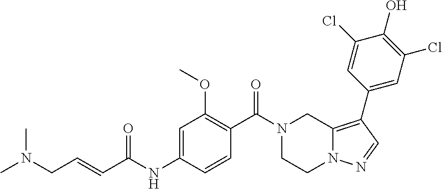 [SMILES: COc1cc(NC(=O)/C=C/CN(C)C)ccc1C(=O)N1CCn2ncc(-c3cc(Cl)c(O)c(Cl)c3)c2C1]

This compound was prepared according to the procedures described in Example 37, with (E)-4-(dimethylamino)but-2-enoic acid replacing (E)-4-(piperidin-1-yl)but-2-enoic acid and tert-butyl 4-amino-2-methoxybenzoate replacing tert-butyl 4-amino-3-methoxybenzoate in step 1. LCMS calculated for C₂₆H₂₈Cl₂N₅O₄ (M+H)⁺: m/z=544.1; found: 544.1. ¹H NMR (600 MHz, DMSO-d₆) δ 10.26 (s, 1H), 7.84 (s, 1H), 7.59 (s, 1H), 7.40 (s, 1H), 7.24 (p, J=9.2 Hz, 2H), 7.13 (s, 1H), 6.77 (dt, J=15.6, 5.9 Hz, 1H), 6.28 (d, J=15.4 Hz, 1H), 5.00 (s, 2H), 4.11 (t, J=5.6 Hz, 2H), 3.79 (s, 3H), 3.67 (s, 2H), 3.07 (d, J=5.9 Hz, 2H), 2.18 (d, J=6.5 Hz, 6H).

### Example 42. N-(2-chloro-4-(3-(3,5-dichloro-4-hydroxyphenyl)-4,5,6,7-tetrahydropyrazolo[1,5-a]pyrazine-5-carbonyl)phenyl)acrylamide

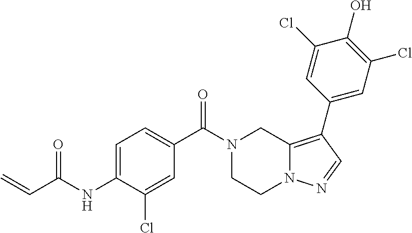 [SMILES: C=CC(=O)Nc1ccc(C(=O)N2CCn3ncc(-c4cc(Cl)c(O)c(Cl)c4)c3C2)cc1Cl]

This compound was prepared according to the procedures described in Example 3, with methyl 4-amino-3-chlorobenzoate replacing methyl isoindoline-5-carboxylate in step 1. LCMS calculated for C₂₂H₁₈Cl₃N₄O₃ (M+H)⁺: m/z=491.0; found: 491.0. ¹H NMR (600 MHz, DMSO-d₆) δ 9.83 (s, 1H), 7.98 (d, J=8.3 Hz, 1H), 7.81 (s, 1H), 7.68 (d, J=1.9 Hz, 1H), 7.51 (d, J=8.3 Hz, 1H), 7.33 (s, 2H), 6.67 (dd, J=17.0, 10.2 Hz, 1H), 6.52 (d, J=1.8 Hz, 1H), 6.31 (d, J=17.2 Hz, 1H), 5.82 (d, J=10.5 Hz, 1H), 4.95 (s, 2H), 4.23 (t, J=5.5 Hz, 2H), 3.88 (br, 2H).

### Example 43. N-(4-(3-(3,5-dichloro-4-hydroxyphenyl)-4,5,6,7-tetrahydropyrazolo[1,5-a]pyrazine-5-carbonyl)-2-fluorophenyl)acrylamide

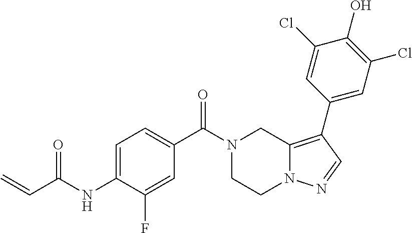 [SMILES: C=CC(=O)Nc1ccc(C(=O)N2CCn3ncc(-c4cc(Cl)c(O)c(Cl)c4)c3C2)cc1F]

This compound was prepared according to the procedures described in Example 3, with methyl 4-amino-3-fluorobenzoate replacing methyl isoindoline-5-carboxylate in step 1. LCMS calculated for C₂₂H₁₈Cl₂FN₄O₃(M+H)⁺: m/z=475.1; found: 475.1. ¹H NMR (600 MHz, DMSO-d₆) δ 10.13 (s, 1H), 8.20 (t, J=8.1 Hz, 1H), 7.83 (s, 1H), 7.48 (d, J=10.9 Hz, 1H), 7.36 (m, J=8.1, 1.9 Hz, 3H), 6.66 (dd, J=17.0, 10.2 Hz, 1H), 6.31 (dd, J=17.0, 1.9 Hz, 1H), 5.81 (d, J=10.3 Hz, 1H), 4.95 (s, 2H), 4.24 (t, J=5.5 Hz, 2H), 3.90 (s, 2H).

### Example 44. N-(3-chloro-4-(3-(3,5-dichloro-4-hydroxyphenyl)-4,5,6,7-tetrahydropyrazolo[1,5-a]pyrazine-5-carbonyl)phenyl)acrylamide

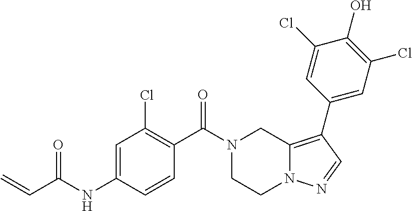 [SMILES: C=CC(=O)Nc1ccc(C(=O)N2CCn3ncc(-c4cc(Cl)c(O)c(Cl)c4)c3C2)c(Cl)c1]

This compound was prepared according to the procedures described in Example 3, with methyl 4-amino-2-chlorobenzoate replacing methyl isoindoline-5-carboxylate in step 1. LCMS calculated for C₂₂H₁₈Cl₃N₄O₃ (M+H)⁺: m/z=491.0; found: 491.0. ¹H NMR (600 MHz, DMSO-d₆) δ 10.49 (s, 1H), 8.01 (d, J=2.0 Hz, 1H), 7.86 (s, 1H), 7.62 (dd, J=8.2, 2.0 Hz, 1H), 7.47 (d, J=8.3 Hz, 1H), 7.41 (d, J=7.8 Hz, 2H), 6.43 (dd, J=16.9, 10.2 Hz, 1H), 6.35-6.26 (m, 1H), 5.86-5.79 (m, 1H), 5.12 (s, 1H), 5.04-4.91 (m, 1H), 4.59 (s, 1H), 4.14 (t, J=5.6 Hz, 2H), 3.70 (s, 2H).

### Example 45. N-(4-(3-(3,5-dichloro-4-hydroxyphenyl)-4,5,6,7-tetrahydropyrazolo[1,5-a]pyrazine-5-carbonyl)-3-(trifluoromethyl)phenyl)acrylamide

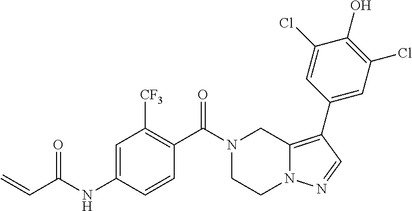 [SMILES: C=CC(=O)Nc1ccc(C(=O)N2CCn3ncc(-c4cc(Cl)c(O)c(Cl)c4)c3C2)c(C(F)(F)F)c1]

This compound was prepared according to the procedures described in Example 3, with methyl 4-amino-2-(trifluoromethyl)benzoate replacing methyl isoindoline-5-carboxylate in step 1. LCMS calculated for C₂₃H₁₈Cl₂F₃N₄O₃(M+H)⁺: m/z=525.1; found: 525.1. ¹H NMR (600 MHz, DMSO-d₆) δ 10.64 (s, 1H), 8.25 (s, 1H), 7.96 (d, J=8.3 Hz, 1H), 7.87 (s, 1H), 7.61 (d, J=8.3 Hz, 1H), 7.42 (s, 2H), 6.43 (td, J=16.4, 10.0 Hz, 1H), 6.33 (dd, J=17.0, 13.1 Hz, 1H), 5.85 (t, J=11.0 Hz, 2H), 5.16 (d, J=16.8 Hz, 1H), 4.92 (d, J=17.1 Hz, 1H), 4.16 (s, 1H), 4.05 (s, 1H), 3.66 (d, J=5.6 Hz, 2H).

### Example 46. N-(5-(3-(3,5-dichloro-4-hydroxyphenyl)-4,5,6,7-tetrahydropyrazolo[1,5-a]pyrazine-5-carbonyl)pyridin-2-yl)acrylamide

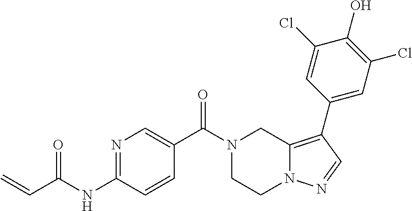 [SMILES: C=CC(=O)Nc1ccc(C(=O)N2CCn3ncc(-c4cc(Cl)c(O)c(Cl)c4)c3C2)cn1]

This compound was prepared according to the procedures described in Example 3, with methyl 6-aminonicotinate replacing methyl isoindoline-5-carboxylate in step 1. LCMS calculated for C₂₁H₁₈Cl₂N₅O₃ (M+H)⁺: m/z=458.1; found: 458.1. ¹H NMR (600 MHz, DMSO-d₆) δ 10.97 (s, 1H), 8.51 (d, J=2.3 Hz, 1H), 8.28 (d, J=8.7 Hz, 1H), 8.00 (dd, J=8.6, 2.3 Hz, 1H), 7.81 (s, 1H), 7.32 (s, 2H), 6.63 (dd, J=17.0, 10.2 Hz, 1H), 6.35 (d, J=17.0 Hz, 1H), 5.83 (d, J=10.2 Hz, 1H), 4.97 (s, 2H), 4.24 (d, J=5.5 Hz, 2H), 3.94 (s, 2H).

### Example 47. N-(4-(3-(3,5-dichloro-4-hydroxyphenyl)-4,5,6,7-tetrahydropyrazolo[1,5-a]pyrazine-5-carbonyl)-3-methoxyphenyl)acrylamide

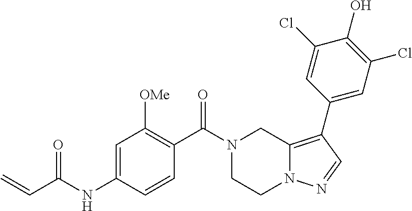 [SMILES: C=CC(=O)Nc1ccc(C(=O)N2CCn3ncc(-c4cc(Cl)c(O)c(Cl)c4)c3C2)c(OC)c1]

This compound was prepared according to the procedures described in Example 3, with methyl 4-amino-2-methoxybenzoate replacing methyl isoindoline-5-carboxylate in step 1. LCMS calculated for C₂₃H₂₁Cl₂N₄O₄ (M+H)⁺: m/z=487.1; found: 487.1. ¹H NMR (600 MHz, DMSO-d₆) δ 10.41-10.23 (m, 1H), 7.88 (s, 1H), 7.58 (s, 1H), 7.44 (s, 1H), 7.32-7.21 (m, 2H), 7.19 (s, 1H), 6.49-6.37 (m, 1H), 6.28 (t, J=16.4 Hz, 1H), 5.79 (t, J=11.5 Hz, 1H), 5.02 (s, 2H), 4.19 (s, 1H), 4.12 (t, J=5.3 Hz, 2H), 3.80 (s, 2H), 3.62 (s, 2H).

### Example 48. N-(4-(3-(3,5-dichloro-4-hydroxyphenyl)-4,5,6,7-tetrahydropyrazolo[1,5-a]pyrazine-5-carbonyl)-2-ethoxyphenyl)acrylamide

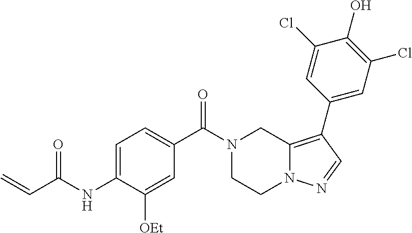 [SMILES: C=CC(=O)Nc1ccc(C(=O)N2CCn3ncc(-c4cc(Cl)c(O)c(Cl)c4)c3C2)cc1OCC]

This compound was prepared according to the procedures described in Example 3, with methyl 4-amino-3-ethoxybenzoate replacing methyl isoindoline-5-carboxylate in step 1. LCMS calculated for C₂₄H₂₃Cl₂N₄O₄ (M+H)⁺: m/z=501.1; found: 501.1. ¹H NMR (600 MHz, DMSO-d₆) δ 10.17 (s, 1H), 9.33 (s, 1H), 8.18 (d, J=8.3 Hz, 1H), 7.87 (s, 1H), 7.39 (s, 2H), 7.16 (s, 1H), 7.09 (d, J=8.3 Hz, 1H), 6.73 (dd, J=17.0, 10.1 Hz, 1H), 6.26 (d, J=17.0 Hz, 1H), 5.76 (d, J=10.3 Hz, 1H), 4.97 (s, 2H), 4.24 (t, J=5.5 Hz, 2H), 4.15 (d, J=8.4 Hz, 2H), 3.92 (s, 2H), 1.38 (t, J=6.9 Hz, 3H).

### Example 49. N-(4-(3-(3,5-dichloro-4-hydroxyphenyl)-4,5,6,7-tetrahydropyrazolo[1,5-a]pyrazine-5-carbonyl)-2-isopropoxyphenyl)acrylamide

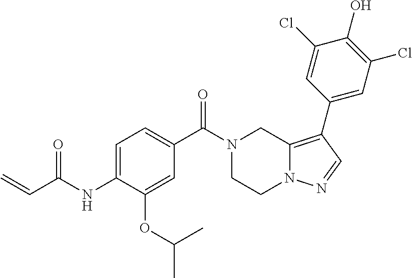 [SMILES: C=CC(=O)Nc1ccc(C(=O)N2CCn3ncc(-c4cc(Cl)c(O)c(Cl)c4)c3C2)cc1OC(C)C]

This compound was prepared according to the procedures described in Example 3, with methyl 4-amino-3-isopropoxybenzoate replacing methyl isoindoline-5-carboxylate in step 1. LCMS calculated for C₂₅H₂₅Cl₂N₄O₄ (M+H)⁺: m/z=515.1; found: 515.1. ¹H NMR (600 MHz, DMSO-d₆) δ 9.19 (s, 1H), 8.19 (d, J=8.3 Hz, 1H), 7.86 (s, 1H), 7.38 (s, 2H), 7.17 (s, 1H), 7.07 (d, J=8.5 Hz, 1H), 6.73 (dd, J=16.9, 10.3 Hz, 1H), 6.26 (d, J=17.0 Hz, 1H), 5.76 (d, J=10.3 Hz, 1H), 4.96 (s, 2H), 4.68 (s, 1H), 4.24 (t, J=5.5 Hz, 2H), 3.91 (s, 2H), 1.31 (d, J=6.0 Hz, 6H).

### Example 50. N-(4-(3-(3,5-dichloro-4-hydroxyphenyl)-4,5,6,7-tetrahydropyrazolo[1,5-a]pyrazine-5-carbonyl)-2-(trifluoromethoxy)phenyl)acrylamide

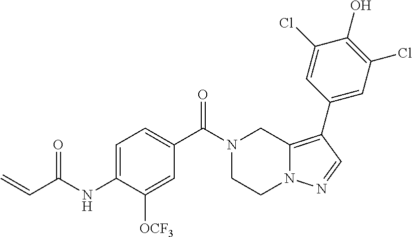 [SMILES: C=CC(=O)Nc1ccc(C(=O)N2CCn3ncc(-c4cc(Cl)c(O)c(Cl)c4)c3C2)cc1OC(F)(F)F]

This compound was prepared according to the procedures described in Example 3, with methyl 4-amino-3-(trifluoromethoxy)benzoate replacing methyl isoindoline-5-carboxylate in step 1. LCMS calculated for C₂₃H₁₈Cl₂F₃N₄O₄(M+H)⁺: m/z=541.1; found: 541.1. ¹H NMR (600 MHz, DMSO-d₆) δ 10.09 (s, 1H), 8.18 (d, J=8.4 Hz, 1H), 7.84 (s, 1H), 7.61-7.55 (m, 2H), 7.36 (s, 3H), 6.68 (dd, J=17.0, 10.2 Hz, 1H), 6.32 (d, J=17.0 Hz, 1H), 5.83 (d, J=10.5 Hz, 1H), 4.96 (s, 2H), 4.24 (t, J=5.4 Hz, 2H), 3.90 (s, 2H).

### Example 51. N-(4-(3-(3,5-dichloro-4-hydroxyphenyl)-4,5,6,7-tetrahydropyrazolo[1,5-a]pyrazine-5-carbonyl)-2-(methoxy-d₃)phenyl)acrylamide

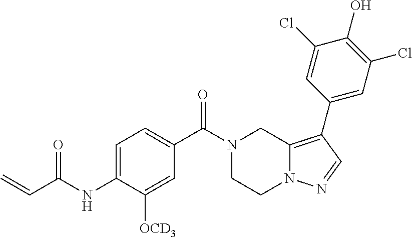

This compound was prepared according to the procedures described in Example 3, with methyl 4-amino-3-(methoxy-d₃)benzoate replacing methyl isoindoline-5-carboxylate in step 1. LCMS calculated for C₂₃H₁₈D₃Cl₂N₄O₄ (M+H)⁺: m/z=490.1; found: 490.1. ¹H NMR (600 MHz, DMSO-d₆) δ 9.52 (s, 1H), 8.21 (d, J=8.2 Hz, 1H), 7.87 (s, 1H), 7.39 (s, 2H), 7.18 (s, 1H), 7.11 (d, J=8.3 Hz, 1H), 6.74 (dd, J=17.1, 10.3 Hz, 1H), 6.26 (d, J=17.0 Hz, 1H), 5.75 (d, J=10.3 Hz, 1H), 4.98 (s, 2H), 4.24 (t, J=5.5 Hz, 2H), 3.93 (s, 2H).

### Example 52. N-(3-cyano-4-(3-(3,5-dichloro-4-hydroxyphenyl)-4,5,6,7-tetrahydropyrazolo[1,5-a]pyrazine-5-carbonyl)phenyl)acrylamide

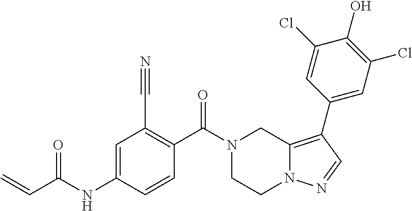 [SMILES: C=CC(=O)Nc1ccc(C(=O)N2CCn3ncc(-c4cc(Cl)c(O)c(Cl)c4)c3C2)c(C#N)c1]

This compound was prepared according to the procedures described in Example 3, with methyl 4-amino-2-cyanobenzoate replacing methyl isoindoline-5-carboxylate in step 1. LCMS calculated for C₂₃H₁₈Cl₂N₅O₃ (M+H)⁺: m/z=482.1; found: 482.1. ¹H NMR (600 MHz, DMSO-d₆) δ 10.67 (s, 1H), 8.27 (s, 1H), 8.02-7.84 (m, 2H), 7.70 (d, J=8.5 Hz, 1H), 7.46 (s, 2H), 6.44 (dd, J=17.0, 10.2 Hz, 1H), 6.34 (d, J=16.8 Hz, 1H), 5.87 (d, J=10.3 Hz, 1H), 5.07 (s, 2H), 4.22 (s, 2H), 3.81 (s, 2H).

### Example 53. (E)-1-(5-((3-(3,5-dichloro-4-hydroxyphenyl)-6,7-dihydropyrazolo[1,5-a]pyrazin-5(4H)-yl)methyl)-2,3-dihydro-1H-pyrrolo[3,2-b]pyridin-1-yl)-4-(piperidin-1-yl)but-2-en-1-one

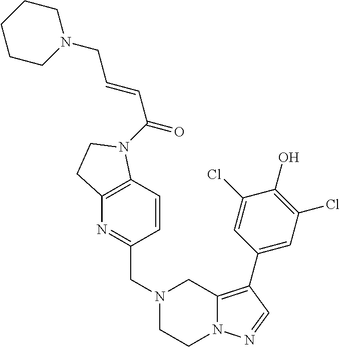 [SMILES: O=C(/C=C/CN1CCCCC1)N1CCc2nc(CN3CCn4ncc(-c5cc(Cl)c(O)c(Cl)c5)c4C3)ccc21]

### Step 1. tert-butyl 5-((3-bromo-6,7-dihydropyrazolo[1,5-a]pyrazin-5(4H)-yl)methyl)-2,3-dihydro-1H-pyrrolo[3,2-b]pyridine-1-carboxylate

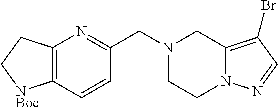 [SMILES: CC(C)(C)OC(=O)N1CCc2nc(CN3CCn4ncc(Br)c4C3)ccc21]

To a solution of 3-bromo-4,5,6,7-tetrahydropyrazolo[1,5-a]pyrazine (248 mg, 1.23 mmol) in DMF (5.60 mL) was added tert-butyl 5-(chloromethyl)-2,3-dihydro-1H-pyrrolo[3,2-b]pyridine-1-carboxylate (300 mg, 1.12 mmol) and DIPEA (585 μL, 3.35 mmol). The mixture was heated to 60° C. for 5 hours. Upon completion, the reaction mixture was diluted with EtOAc, and the mixture was washed with water and brine. The organic layer was then dried over Na₂SO₄, filtered and concentrated. The resulting residue was then purified by flash column (20-100% hexanes/EtOAc) to give desired product. LCMS calculated for C₁₉H₂₅BrN₅O₂(M+H)⁺: m/z=434.1; found: 434.1.

### Step 2. tert-butyl 5-((3-(3,5-dichloro-4-hydroxyphenyl)-6,7-dihydropyrazolo[1,5-a]pyrazin-5(4H)-yl)methyl)-2,3-dihydro-1H-pyrrolo[3,2-b]pyridine-1-carboxylate

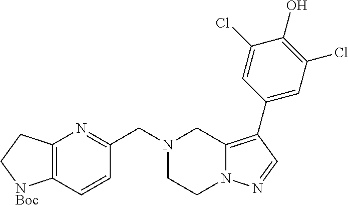 [SMILES: CC(C)(C)OC(=O)N1CCc2nc(CN3CCn4ncc(-c5cc(Cl)c(O)c(Cl)c5)c4C3)ccc21]

To a 40 mL vial equipped with a stir bar was added tert-butyl 5-((3-bromo-6,7-dihydropyrazolo[1,5-a]pyrazin-5(4H)-yl)methyl)-2,3-dihydro-1H-pyrrolo[3,2-b]pyridine-1-carboxylate (450 mg, 1.04 mmol), potassium phosphate (396 mg, 1.86 mmol), (3,5-dichloro-4-hydroxyphenyl)boronic acid (321 mg, 1.55 mmol), in dioxane (9.10 mL) and water (1.29 mL). The mixture was sparged with N₂ for 10 mins, then (dicyclohexyl(2′,4′,6′-triisopropyl-[1,1′-biphenyl]-2-yl)-15-phosphaneyl)(2′-(methylamino)-[1,1′-biphenyl]-2-yl)palladium(II) methanesulfonate (89.0 mg, 0.104 mmol) was added. The resulting mixture was heated at 100° C. for 3 hours. Upon completion, the reaction was quenched with sat. NH₄Cl (aq.), extracted with EtOAc. The combined organic layers were dried over MgSO₄, concentrated, and purified with flash column (0-20% MeOH/DCM) to afford the desired product as a yellow oil. LCMS calculated for C₂₅H₂₈Cl₂N₅O₃ (M+H)⁺: m/z=516.2; found: 516.2.

### Step 3. 2,6-dichloro-4-(5-((2,3-dihydro-1H-pyrrolo[3,2-b]pyridin-5-yl)methyl)-4,5,6,7-tetrahydropyrazolo[1,5-a]pyrazin-3-yl)phenol

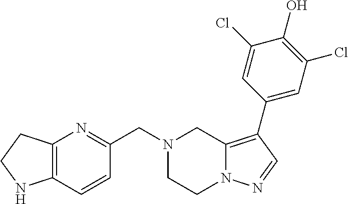 [SMILES: Oc1c(Cl)cc(-c2cnn3c2CN(Cc2ccc4c(n2)CCN4)CC3)cc1Cl]

To a 40 mL vial equipped with a stir bar, tert-butyl 5-((3-bromo-6,7-dihydropyrazolo[1,5-a]pyrazin-5(4H)-yl)methyl)-2,3-dihydro-1H-pyrrolo[3,2-b]pyridine-1-carboxylate (450 mg, 1.04 mmol), was dissolved in MeOH (1.04 mL). The mixture was added hydrogen chloride (2.07 mL, 8.29 mmol, 4 M in dioxane), then the mixture was stirred at rt for 3 hours. Upon completion, the reaction mixture was concentrated under N₂ flow to afford the desired product as HCl salt which was used for next step without further purification. LCMS calculated for C₂₀H₂₀Cl₂N₅O (M+H)⁺: m/z=416.1; found: 416.1.

### Step 4. (E)-1-(5-((3-(3,5-dichloro-4-hydroxyphenyl)-6,7-dihydropyrazolo[1,5-a]pyrazin-5(4H)-yl)methyl)-2,3-dihydro-1H-pyrrolo[3,2-b]pyridin-1-yl)-4-(piperidin-1-yl)but-2-en-1-one

To a solution of 2,6-dichloro-4-(5-((2,3-dihydro-1H-pyrrolo[3,2-b]pyridin-5-yl)methyl)-4,5,6,7-tetrahydropyrazolo[1,5-a]pyrazin-3-yl)phenol (50.0 mg, 0.12 mmol) and (E)-4-(piperidin-1-yl)but-2-enoic acid hydrochloride (54.3 mg, 0.264 mmol) in DMF (601 μL) was added propane phosphonic acid anhydride (84 mg, 0.264 mmol), and DIPEA (105 μL, 0.601 mmol). The reaction mixture was then heated to 60° C. for 6 hours. Upon completion, the reaction mixture was quenched with 50 μL TFA and diluted with MeOH and purified by prep-LCMS (XBridge C18 column, eluting with a gradient of acetonitrile/water containing 0.1% TFA, at flow rate of 60 mL/min) to afford the desired product. LCMS calculated for C₂₉H₃₃Cl₂N₆O₂ (M+H)⁺: m/z=567.2; found: 567.2. ¹H NMR (600 MHz, DMSO-d₆) δ 10.16 (s, 1H), 9.64 (s, 1H), 8.34 (d, J=8.2 Hz, 1H), 7.86 (s, 1H), 7.37 (d, J=8.3 Hz, 1H), 7.34 (s, 2H), 6.93-6.78 (m, 2H), 4.35-4.23 (m, 6H), 4.19 (s, 2H), 3.98 (t, J=5.3 Hz, 2H), 3.43 (d, J=12.2 Hz, 2H), 3.32 (q, J=13.2 Hz, 3H), 3.01-2.82 (m, 2H), 1.84 (dd, J=10.9, 7.2 Hz, 2H), 1.77-1.68 (m, 1H), 1.64 (t, J=13.4 Hz, 2H), 1.50-1.29 (m, 1H).

### Example 54. (E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)indolin-1-yl)-4-morpholinobut-2-en-1-one

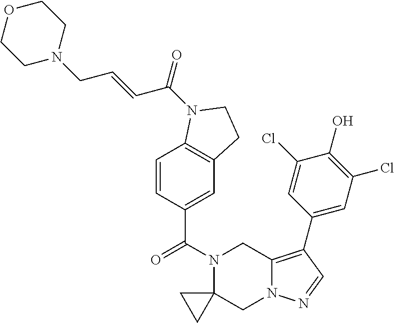 [SMILES: O=C(/C=C/CN1CCOCC1)N1CCc2cc(C(=O)N3Cc4c(-c5cc(Cl)c(O)c(Cl)c5)cnn4CC34CC4)ccc21]

### Step 1. tert-butyl 7′H-spiro[cyclopropane-1,6′-pyrazolo[1,5-a]pyrazine]-5′(4′H)-carboxylate

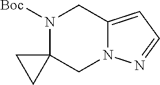 [SMILES: CC(C)(C)OC(=O)N1Cc2ccnn2CC12CC2]

This intermediate was synthesized based on reported literature: Lv et al. European Journal of Medicinal Chemistry 2022, 228, 113974, which is incorporated herein by reference in its entirety.

### Step 2. tert-butyl 3′-bromo-7′H-spiro[cyclopropane-1,6′-pyrazolo[1,5-a]pyrazine]-5′(4′H)-carboxylate

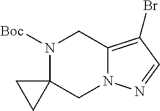 [SMILES: CC(C)(C)OC(=O)N1Cc2c(Br)cnn2CC12CC2]

To a 100 mL round bottom flask equipped with a magnetic stir bar was added tert-butyl 7′H-spiro[cyclopropane-1,6′-pyrazolo[1,5-a]pyrazine]-5′(4′H)-carboxylate (709 mg, 2.84 mmol) and DCM (28.4 mL). To this stirring mixture was added N-bromosuccinimide (607 mg, 3.41 mmol, 1.20 equiv) portionwise and the mixture was stirred at room temperature for 4 hours. After full conversion of the starting material, the solvent was evaporated in vacuo and the mixture was purified by silica gel column chromatography (0-40% EtOAc/hexanes) to give the desired product (623 mg, 1.90 mmol, 67% yield). LCMS calculated for C₁₃H₁₉BrN₃O₂(M+H)⁺: m/z=328.1; found: 328.1.

### Step 3. 3′-bromo-4′,5′-dihydro-7′H-spiro[cyclopropane-1,6′-pyrazolo[1,5-a]pyrazine]

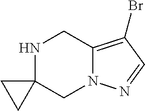 [SMILES: Brc1cnn2c1CNC1(CC1)C2]

To a 40 mL vial equipped with a magnetic stir bar was added tert-butyl 3′-bromo-7′H-spiro[cyclopropane-1,6′-pyrazolo[1,5-a]pyrazine]-5′(4′H)-carboxylate (623 mg, 1.90 mmol), DCM (6 mL) and TFA (2 mL). The mixture was stirred at room temperature overnight. Upon full conversion of the starting material, the solvent was evaporated in vacuo and the mixture was purified by silica gel column chromatography (0-15% MeOH/DCM) to give the desired product as a white powder (433 mg, quantitative yield). LCMS calculated for C₈H₁₁BrN₃ (M+H)⁺: m/z=228.0; found: 228.0.

### Step 4. (E)-1-(5-(3′-bromo-4′,5′-dihydro-7′H-spiro[cyclopropane-1,6′-pyrazolo[1,5-a]pyrazine]-5′-carbonyl)indolin-1-yl)-4-morpholinobut-2-en-1-one

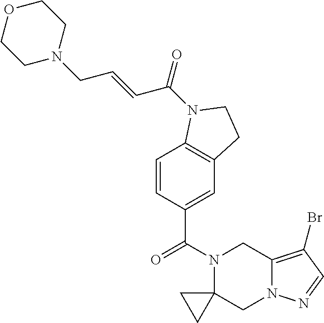 [SMILES: O=C(/C=C/CN1CCOCC1)N1CCc2cc(C(=O)N3Cc4c(Br)cnn4CC34CC4)ccc21]

To a 40 mL vial equipped with a magnetic stir bar was added 3′-bromo-4′,5′-dihydro-7′H-spiro[cyclopropane-1,6′-pyrazolo[1,5-a]pyrazine](50 mg, 0.219 mmol), (E)-1-(4-morpholinobut-2-enoyl)indoline-5-carboxylic acid (83 mg, 0.263 mmol), HATU (133 mg, 0.351 mmol), DMF (2 mL) and DIPEA (0.69 mL, 3.95 mmol). The mixture stirred at 40° C. overnight. The mixture was then diluted with ethyl acetate and washed with water and saturated brine for 3 times. The organic layer was separated, dried over MgSO₄, concentrated in vacuo, and purified by silica gel column chromatography (0-15% MeOH/DCM) to afford the desired product (80 mg, 0.152 mmol, 69% yield). LCMS calculated for C₂₅H₂₉BrN₅O₃ (M+H)⁺: m/z=526.1; found: 526.1.

### Step 5. (E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)indolin-1-yl)-4-morpholinobut-2-en-1-one

To a 40 mL scintillation vial equipped with a magnetic stir bar was added (E)-1-(5-(3′-bromo-4′,5′-dihydro-7′H-spiro[cyclopropane-1,6′-pyrazolo[1,5-a]pyrazine]-5′-carbonyl)indolin-1-yl)-4-morpholinobut-2-en-1-one (80.0 mg, 0.152 mmol), 2,6-dichloro-4-(4,4,5,5-tetramethyl-1,3,2-dioxaborolan-2-yl)phenol (57.1 mg, 0.198 mmol), potassium phosphate (129 mg, 0.608 mmol), (1,1′-bis(diphenylphosphino)ferrocene)-dichloropalladium(II) (24.8 mg, 0.030 mmol), 1,4-dioxane (1.4 mL) and water (0.2 mL). The mixture was sparged with N₂ for 10 min and then stirred at 90° C. for 2 hours. After full conversion, the reaction mixture was diluted with MeCN (3 mL) and MeOH (1 mL), filtered through a syringe filter, and purified by prep-HPLC (pH=10.0, then pH=2.0) to afford the desired product as a white powder. LCMS calculated for C₃₁H₃₂Cl₂N₅O₄ (M+H)⁺: m/z=608.2; found: 608.2. ¹H NMR (600 MHz, DMSO-d₆) δ 10.13 (s, 1H), 8.17 (d, J=8.3 Hz, 1H), 7.86 (s, 1H), 7.45 (s, 1H), 7.43-7.39 (m, 1H), 7.37 (s, 2H), 6.80 (dd, J=13.4, 7.1 Hz, 2H), 5.06-4.94 (br, 2H), 4.27 (t, J=8.6 Hz, 3H), 4.08-3.97 (m, 4H), 3.74-3.63 (m, 3H), 3.22 (m, 3H), 3.16-3.04 (m, 2H), 0.96 (s, 4H).

### Example 55. (E)-1-(5-(3′-(2-(2,3-dihydrobenzofuran-7-yl)pyridin-4-yl)-4′,5′-dihydro-7′H-spiro[cyclopropane-1,6′-pyrazolo[1,5-a]pyrazine]-5′-carbonyl)indolin-1-yl)-4-(dimethylamino)but-2-en-1-one

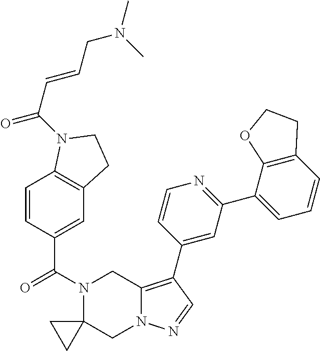 [SMILES: CN(C)C/C=C/C(=O)N1CCc2cc(C(=O)N3Cc4c(-c5ccnc(-c6cccc7c6OCC7)c5)cnn4CC34CC4)ccc21]

### Step 1. tert-butyl 5-(3′-bromo-4′,5′-dihydro-7′H-spiro[cyclopropane-1,6′-pyrazolo[1,5-a]pyrazine]-5′-carbonyl)indoline-1-carboxylate

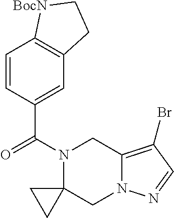 [SMILES: CC(C)(C)OC(=O)N1CCc2cc(C(=O)N3Cc4c(Br)cnn4CC34CC4)ccc21]

To a solution of 3′-bromo-4′,5′-dihydro-7′H-spiro[cyclopropane-1,6′-pyrazolo[1,5-a]pyrazine](400 mg, 1.75 mmol) in DMF (8.8 mL) was added DIPEA (1.8 mL, 10.52 mmol), 1-(tert-butoxycarbonyl)indoline-5-carboxylic acid (554 mg, 2.10 mmol), and HATU (1.33 g, 3.51 mmol). The reaction mixture was then heated to 60° C. and was allowed to stir for 16 h. Upon completion, the reaction contents were diluted with EtOAc (50 mL), washed three times with H₂O (60 mL), dried over Na₂SO₄, and concentrated in vacuo. The crude residue was then purified by flash column chromatography (0-20% CH₂Cl₂/MeOH) to provide the desired product (775 mg, 1.64 mmol, 93%). LCMS calculated for C₂₂H₂₆BrN₄O₃ (M+H)⁺: m/z=473.1; found: 473.1.

### Step 2. tert-butyl 5-(3′-(2-chloropyridin-4-yl)-4′,5′-dihydro-7′H-spiro[cyclopropane-1,6′-pyrazolo[1,5-a]pyrazine]-5′-carbonyl)indoline-1-carboxylate

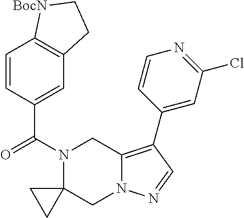 [SMILES: CC(C)(C)OC(=O)N1CCc2cc(C(=O)N3Cc4c(-c5ccnc(Cl)c5)cnn4CC34CC4)ccc21]

To a solution of tert-butyl 5-(3′-bromo-4′,5′-dihydro-7′H-spiro[cyclopropane-1,6′-pyrazolo[1,5-a]pyrazine]-5′-carbonyl)indoline-1-carboxylate (690 mg, 1.46 mmol) in 1,4-dioxane (12.8 mL) and water (1.8 mL) was added (2-chloropyridin-4-yl)boronic acid (275 mg, 1.75 mmol), tripotassium phosphate (1.86 g, 8.75 mmol), and [1,1′-bis(diphenylphosphino)ferrocene]dichloropalladium(II), complex with dichloromethane (238 mg, 0.29 mmol). The reaction mixture was then degassed for 5 min before heating to 90° C. and was allowed to stir for 1 h. Upon completion, the reaction mixture was filtered through Celite, quenched with NH₄Cl_{(aq)} (20 mL), extracted three times with EtOAc (25 mL), dried over Na₂SO₄, and concentrated in vacuo. The crude residue was then purified by flash column chromatography (0-100% hexanes/EtOAc) to provide the desired product (271 mg, 0.54 mmol, 37%). LCMS calculated for C₂₇H₂₉ClN₅O₃(M+H)⁺: m/z=506.2; found: 506.2.

### Step 3. tert-butyl 5-(3′-(2-(2,3-dihydrobenzofuran-7-yl)pyridin-4-yl)-4′,5′-dihydro-7′H-spiro[cyclopropane-1,6′-pyrazolo[1,5-a]pyrazine]-5′-carbonyl)indoline-1-carboxylate

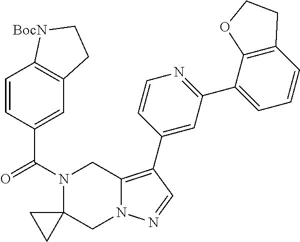 [SMILES: CC(C)(C)OC(=O)N1CCc2cc(C(=O)N3Cc4c(-c5ccnc(-c6cccc7c6OCC7)c5)cnn4CC34CC4)ccc21]

To a solution of tert-butyl 5-(3′-(2-chloropyridin-4-yl)-4′,5′-dihydro-7′H-spiro[cyclopropane-1,6′-pyrazolo[1,5-a]pyrazine]-5′-carbonyl)indoline-1-carboxylate (150 mg, 0.30 mmol) in 1,4-dioxane (2.6 mL) and water (0.4 mL) was added (2,3-dihydrobenzofuran-7-yl)boronic acid (97 mg, 0.59 mmol), tripotassium phosphate (378 mg, 1.78 mmol), and [1,1′-bis(diphenylphosphino)ferrocene]dichloropalladium(II), complex with dichloromethane (48.4 mg, 0.06 mmol). The reaction mixture was then degassed for 5 min before heating to 90° C. and was allowed to stir for 1 h. Upon completion, the reaction mixture was filtered through Celite, quenched with NH₄Cl_{(aq)} (15 mL), extracted three times with EtOAc (15 mL), dried over Na₂SO₄, and concentrated in vacuo. The crude residue was then purified by flash column chromatography (0-100% hexanes/EtOAc) to provide the desired product (140 mg, 0.24 mmol, 81%). LCMS calculated for C₃₅H₃₆N₅O₄ (M+H)⁺: m/z=590.3; found: 590.3.

### Step 4. (3′-(2-(2,3-dihydrobenzofuran-7-yl)pyridin-4-yl)-7′H-spiro[cyclopropane-1,6′-pyrazolo[1,5-a]pyrazin]-5′(4′H)-yl)(indolin-5-yl)methanone dihydrochloride

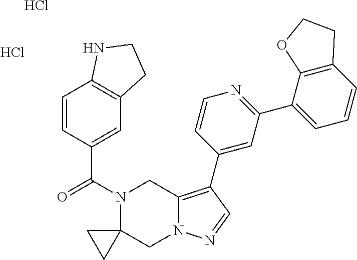 [SMILES: Cl ; Cl ; O=C(c1ccc2c(c1)CCN2)N1Cc2c(-c3ccnc(-c4cccc5c4OCC5)c3)cnn2CC12CC2]

Tert-butyl 5-(3′-(2-(2,3-dihydrobenzofuran-7-yl)pyridin-4-yl)-4′,5′-dihydro-7′H-spiro[cyclopropane-1,6′-pyrazolo[1,5-a]pyrazine]-5′-carbonyl)indoline-1-carboxylate (140 mg, 0.24 mmol) was dissolved in a solution of HCl (1.5 mL, 4.0M in 1,4-Dioxane, 6.00 mmol). The reaction mixture was allowed to stir for 1 h. Upon completion, the reaction contents were concentrated in vacuo directly to produce the desired product, and the crude material was used directly in the next step (134 mg, 0.24 mmol, 100%). LCMS calculated for C₃₀H₂₈N₅O₂ (M+H)⁺: m/z=490.2; found: 490.2.

### Step 5. (E)-1-(5-(3′-(2-(2,3-dihydrobenzofuran-7-yl)pyridin-4-yl)-4′,5′-dihydro-7′H-spiro[cyclopropane-1,6′-pyrazolo[1,5-a]pyrazine]-5′-carbonyl)indolin-1-yl)-4-(dimethylamino)but-2-en-1-one

To a solution of (3′-(2-(2,3-dihydrobenzofuran-7-yl)pyridin-4-yl)-7′H-spiro[cyclopropane-1,6′-pyrazolo[1,5-a]pyrazin]-5′(4′H)-yl)(indolin-5-yl)methanone dihydrochloride (60 mg, 0.11 mmol) in THF (1.1 mL) was added (E)-4-(dimethylamino)but-2-enoic acid hydrochloride (35.3 mg, 0.21 mmol), DIPEA (0.11 mL, 0.64 mmol), and n-propylphosphonic acid anhydride, cyclic trimer (0.13 mL, 0.21 mmol). The reaction mixture was then allowed to stir for 12 h. Upon completion, the reaction contents were diluted with MeCN, and purified by prep-LCMS (XBridge C18 column, eluting with a gradient of acetonitrile/water containing 0.1% TFA, at flow rate of 60 mL/min) to afford the desired product as a TFA salt. LCMS calculated for C₃₆H₃₇N₆O₃ (M+H)⁺: m/z=601.3; found: 601.3. ¹H NMR (600 MHz, DMSO_d6) δ 9.96 (s, 1H), 8.68 (d, J=5.6 Hz, 1H), 8.27-8.08 (m, 3H), 7.82 (d, J=7.9 Hz, 1H), 7.61 (s, 1H), 7.49-7.44 (m, 2H), 7.40 (d, J=7.2 Hz, 1H), 7.03 (t, J=7.5 Hz, 1H), 6.86-6.74 (m, 2H), 5.16 (br s, 4H), 4.75 (t, J=8.7 Hz, 2H), 4.25 (t, J=8.5 Hz, 2H), 3.96 (d, J=6.5 Hz, 2H), 3.30 (t, J=8.7 Hz, 2H), 3.20 (t, J=8.6 Hz, 2H), 2.81 (s, 6H), 0.97 (s, 4H).

### Example 56. (E)-4-(dimethylamino)-1-(5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one

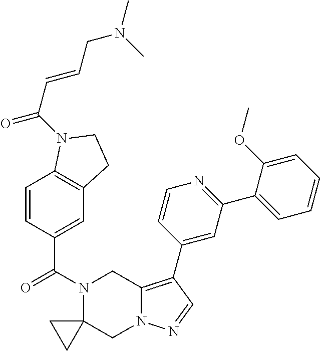 [SMILES: COc1ccccc1-c1cc(-c2cnn3c2CN(C(=O)c2ccc4c(c2)CCN4C(=O)/C=C/CN(C)C)C2(CC2)C3)ccn1]

Example 56 was prepared according to the procedures described in Example 55 as a TFA salt, using 2-(2-methoxyphenyl)-4,4,5,5-tetramethyl-1,3,2-dioxaborolane instead of (2,3-dihydrobenzofuran-7-yl)boronic acid in Step 3. LCMS calculated for C₃₅H₃₇N₆O₃ (M+H)⁺: m/z=589.3; found: 589.3. ¹H NMR (600 MHz, DMSO_d6) δ 9.89 (s, 1H), 8.70 (d, J=5.7 Hz, 1H), 8.27 (s, 1H), 8.18 (d, J=8.3 Hz, 1H), 7.99 (s, 1H), 7.73 (d, J=7.6 Hz, 1H), 7.67 (s, 1H), 7.54 (t, J=8.0 Hz, 1H), 7.49-7.40 (m, 2H), 7.25 (d, J=8.4 Hz, 1H), 7.15 (t, J=7.5 Hz, 1H), 6.87-6.73 (m, 2H), 5.19 (s, 2H), 4.38 (s, 2H), 4.26 (t, J=8.5 Hz, 2H), 3.97 (d, J=6.3 Hz, 2H), 3.91 (s, 3H), 3.21 (d, J=16.9 Hz, 2H), 2.82 (s, 6H), 0.95 (d, J=14.3 Hz, 4H).

### Example 57. (E)-4-(3-azabicyclo[3.1.1]heptan-3-yl)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)but-2-en-1-one

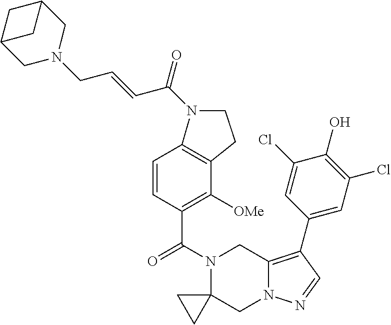 [SMILES: COc1c(C(=O)N2Cc3c(-c4cc(Cl)c(O)c(Cl)c4)cnn3CC23CC3)ccc2c1CCN2C(=O)/C=C/CN1CC2CC(C2)C1]

### Step 1. tert-butyl 5-(3′-bromo-4′,5′-dihydro-7′H-spiro[cyclopropane-1,6′-pyrazolo[l, 5-a]pyrazine]-5′-carbonyl)-4-methoxyindoline-1-carboxylate

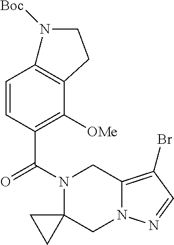 [SMILES: COc1c(C(=O)N2Cc3c(Br)cnn3CC23CC3)ccc2c1CCN2C(=O)OC(C)(C)C]

To a 500 mL round bottom flask containing a magnetic stir bar was added 3′-bromo-4′,5′-dihydro-7′H-spiro[cyclopropane-1,6′-pyrazolo[1,5-a]pyrazine](5.00 g, 21.92 mmol), 1-(tert-butoxycarbonyl)-4-methoxyindoline-5-carboxylic acid (6.43 g, 21.92 mmol), HATU (12.50 g, 32.90 mmol), DMF (150 mL), and DIPEA (65 mL, 373 mmol). The mixture was stirred at 40° C. overnight. After the reaction reached full conversion, the mixture was diluted with ethyl acetate (600 mL), washed with water (150 mL) twice and saturated brine (150 mL) once. The organic layer was separated, dried over MgSO₄, concentrated in vacuo, and purified by silica gel column chromatography using 0-30% ethyl acetate/hexanes to afford the product as a brown oil (9.64 g, 19.14 mmol, 87% yield). LCMS calculated for C₂₃H₂₈BrN₄O₄(M+H)⁺: m/z=503.1; found: 503.1.

### Step 2. (3′-bromo-7′H-spiro[cyclopropane-1,6′-pyrazolo[1,5-a]pyrazin]-5′(4′H)-yl)(4-methoxyindolin-5-yl)methanone

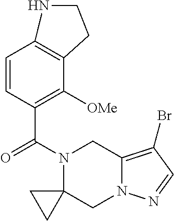 [SMILES: COc1c(C(=O)N2Cc3c(Br)cnn3CC23CC3)ccc2c1CCN2]

To a 100 mL round bottom flask containing a magnetic stir bar was added tert-butyl 5-(3′-bromo-4′,5′-dihydro-7′H-spiro[cyclopropane-1,6′-pyrazolo[1,5-a]pyrazine]-5′-carbonyl)-4-methoxyindoline-1-carboxylate (3.09 g, 6.14 mmol), DCM (12.28 mL) and HCl (11.51 mL, 46.1 mmol, 4.0 M solution in dioxane). The mixture was stirred at room temperature overnight. Upon completion, the reaction mixture was concentrated in vacuo. The precipitates were collected by filtration and washed with diethyl ether (20 mL) three times to afford the HCl salt of the product as a light brown powder (2.19 g, 6.14 mmol, 89% yield). LCMS calculated for C₁₈H₂₀BrN₄O₂(M+H)⁺: m/z=403.1; found: 403.1.

### Step 3. (E)-4-(3-azabicyclo[3.1.1]heptan-3-yl)-1-(5-(3′-bromo-4′,5′-dihydro-7′H-spiro[cyclopropane-1,6′-pyrazolo[1,5-a]pyrazine]-5′-carbonyl)-4-methoxyindolin-1-yl)but-2-en-1-one

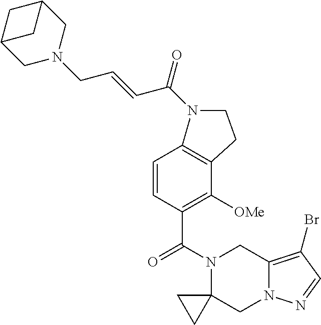 [SMILES: COc1c(C(=O)N2Cc3c(Br)cnn3CC23CC3)ccc2c1CCN2C(=O)/C=C/CN1CC2CC(C2)C1]

To a 100 mL round bottom flask containing a magnetic stir bar was added (3′-bromo-7′H-spiro[cyclopropane-1,6′-pyrazolo[1,5-a]pyrazin]-5′(4′H)-yl)(4-methoxyindolin-5-yl)methanone (2.2 g, 5.46 mmol), (E)-4-(3-azabicyclo[3.1.1]heptan-3-yl)but-2-enoic acid hydrochloride (1.188 g, 5.46 mmol), THF (27.3 mL), DIPEA (7.62 mL, 43.6 mmol), and propane phosphonic acid anhydride (8.12 mL, 13.64 mmol). The mixture was stirred at 40° C. overnight. Upon completion, the mixture was quenched with saturated aqueous solution of NaHCO₃ (50 mL), extracted with a mixture of ethyl acetate and methanol (20:1 v/v, 30 mL) three times. The organic layers were combined, dried over MgSO₄, concentrated in vacuo, and purified by silica gel column chromatography (MeOH/DCM 0-20%) to afford the product as a brown oil (2.23 g, 3.94 mmol, 72.2% yield). LCMS calculated for C₂₈H₃₃BrN₅O₃(M+H)⁺: m/z=566.2; found: 566.2.

### Step 4. (E)-4-(3-azabicyclo[3.1.1]heptan-3-yl)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)but-2-en-1-one

To a 40 mL scintillation vial equipped with a magnetic stir bar was added (E)-4-(3-azabicyclo[3.1.1]heptan-3-yl)-1-(5-(3′-bromo-4′,5′-dihydro-7′H-spiro[cyclopropane-1,6′-pyrazolo[1,5-a]pyrazine]-5′-carbonyl)-4-methoxyindolin-1-yl)but-2-en-1-one (1.116 g, 1.970 mmol), 2,6-dichloro-4-(4,4,5,5-tetramethyl-1,3,2-dioxaborolan-2-yl)phenol (0.740 g, 2.56 mmol), (1,1′-Bis(diphenylphosphino)ferrocene)-dichloropalladium(II) (0.322 g, 0.394 mmol), potassium phosphate (1.673 g, 7.88 mmol), dioxane (17.24 mL) and water (2.462 mL). The mixture was sparged with N₂ for 10 min and subsequently stirred at 90° C. for 1 h. After completion, the reaction mixture was loaded directly onto silica gel and purified by column chromatography (0-25% MeOH/DCM). The crude product was further purified by prep-HPLC (pH=2) to afford the product as a white powder. LCMS calculated for C₃₄H₃₆Cl₂N₅O₄ (M+H)⁺: m/z=648.2; found: 648.2. ¹H NMR (600 MHz, DMSO-d6) δ 10.21-10.07 (m, 2H), 7.97-7.74 (m, 2H), 7.51-7.47 (m, 1H), 7.25-7.11 (m, 1H), 6.93-6.85 (m, 1H), 6.88-6.81 (m, 1H), 4.74-4.64 (m, 1H), 4.33-4.15 (m, 2H), 4.10 (t, J=6.1 Hz, 2H), 3.72 (m, 8H), 3.61-3.56 (m, 1H), 3.38 (dd, J=12.1, 7.5 Hz, 2H), 3.35-3.30 (m, 1H), 2.47 (q, J=4.6 Hz, 2H), 2.26 (q, J=7.5 Hz, 1H), 2.16 (dt, J=11.3, 6.0 Hz, 1H), 1.82 (t, J=9.3 Hz, 1H), 1.50 (t, J=8.9 Hz, 1H), 1.29-1.19 (m, 1H), 1.18-1.07 (m, 1H), 0.92-0.85 (m, 1H), 0.84-0.72 (m, 1H).

### Example 58. (E)-1-(5-(3′-(2-methoxypyridin-4-yl)-4′,5′-dihydro-7′H-spiro[cyclopropane-1,6′-pyrazolo[1,5-a]pyrazine]-5′-carbonyl)indolin-1-yl)-4-(piperidin-1-yl)but-2-en-1-one

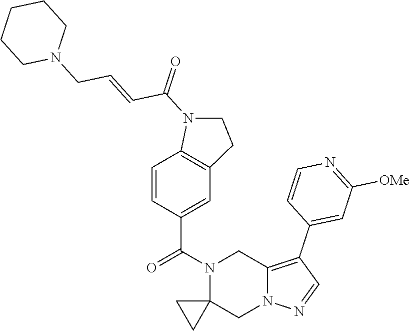 [SMILES: COc1cc(-c2cnn3c2CN(C(=O)c2ccc4c(c2)CCN4C(=O)/C=C/CN2CCCCC2)C2(CC2)C3)ccn1]

This compound was prepared according to the procedures described in Example 57, with 1-(tert-butoxycarbonyl)indoline-5-carboxylic acid replacing 1-(tert-butoxycarbonyl)-4-methoxyindoline-5-carboxylic acid in step 1, (E)-4-(piperidin-1-yl)but-2-enoic acid hydrochloride replacing (E)-4-(3-azabicyclo[3.1.1]heptan-3-yl)but-2-enoic acid hydrochloride in step 3, and (2-methoxypyridin-4-yl)boronic acid replacing 2,6-dichloro-4-(4,4,5,5-tetramethyl-1,3,2-dioxaborolan-2-yl)phenol in step 4. LCMS calculated for C₃₂H₃₇N₆O₃ (M+H)⁺: m/z=553.3; found: 553.3. ¹H NMR (500 MHz, DMSO-d6) δ 8.21-8.10 (m, 2H), 8.04 (s, 1H), 7.45-7.38 (m, 2H), 7.08-7.02 (m, 1H), 6.87-6.75 (m, 2H), 6.57-6.50 (m, 1H), 5.12-4.98 (m, 2H), 4.44-4.17 (m, 2H), 4.23 (t, J=8.6 Hz, 2H), 3.86 (s, 3H), 3.18 (t, J=8.6 Hz, 2H), 3.13 (dd, J=6.2, 1.6 Hz, 2H), 2.46-2.34 (m, 4H), 1.52 (p, J=5.7 Hz, 4H), 1.40 (q, J=6.0 Hz, 2H), 1.04-0.90 (m, 4H).

### Example 59. (E)-1-(5-(3′-(3,5-dichloro-4-hydroxyphenyl)-4′,5′-dihydro-7′H-spiro[cyclobutane-1,6′-pyrazolo[1,5-a]pyrazine]-5′-carbonyl)indolin-1-yl)-4-(dimethylamino)but-2-en-1-one

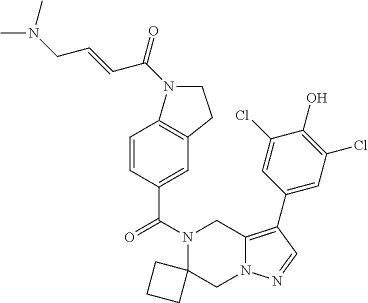 [SMILES: CN(C)C/C=C/C(=O)N1CCc2cc(C(=O)N3Cc4c(-c5cc(Cl)c(O)c(Cl)c5)cnn4CC34CCC4)ccc21]

### Step 1. tert-butyl ((1H-pyrazol-5-yl)methyl)(1-(hydroxymethyl)cyclobutyl)carbamate

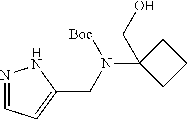 [SMILES: CC(C)(C)OC(=O)N(Cc1ccn[nH]1)C1(CO)CCC1]

To a 100 mL round bottom flask equipped with a magnetic stir bar was added 1H-pyrazole-5-carbaldehyde (1 g, 10.41 mmol), (1-aminocyclobutyl)methanol hydrochloride (1.432 g, 10.41 mmol), MeOH (20.24 mL), and TEA (1.523 mL, 10.93 mmol). The mixture was stirred at room temperature for 2 h before NaBH₄ (0.787 g, 20.81 mmol) was added portion-wise at 0° C. The reaction was stirred from 0° C. to room temperature over 2 h. Subsequently, water (14.45 mL) was added, followed by Boc₂O (4.54 g, 20.81 mmol) and the mixture was stirred at room temperature overnight. Upon completion, the reaction mixture was diluted by water (15 mL) and saturated brine (15 mL), extracted with ethyl acetate (20 mL) three times. The organic layers were combined, dried over MgSO₄, concentrated in vacuo, and purified by silica gel column chromatography (0-5% MeOH/DCM) to afford the product as a yellow oil (1.014 g, 3.60 mmol, 35% yield). LCMS calculated for C₁₄H₂₄N₃O₃ (M+H)⁺: m/z=282.2; found: 282.2.

### Step 2. (1-(((1H-pyrazol-5-yl)methyl)(tert-butoxycarbonyl)amino)cyclobutyl)methyl 4-methylbenzenesulfonate

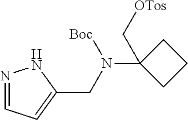

To a 40 mL scintillation vial equipped with a magnetic stir bar was added tert-butyl ((1H-pyrazol-5-yl)methyl)(1-(hydroxymethyl)cyclobutyl)carbamate (1.014 g, 3.60 mmol), DCM (7.2 mL) and TEA (5.02 mL, 36.0 mmol). The solution was cooled to 0° C. before 4-toluenesulfonyl chloride (2.061 g, 10.81 mmol) was added portion-wise. The reaction was then stirred from 0° C. to room temperature over 2 h. Upon completion, the reaction mixture was concentrated in vacuo and purified by silica gel column chromatography (0-40% ethyl acetate/hexanes) to afford the product (0.92 g, 2.112 mmol, 59% yield). LCMS calculated for C₂₁H₃₀N₃O₅S (M+H)⁺: m/z=436.2; found: 436.2.

### Step 3. tert-butyl 7′H-spiro[cyclobutane-1,6′-pyrazolo[1,5-a]pyrazine]-5′(4′H)-carboxylate

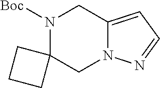 [SMILES: CC(C)(C)OC(=O)N1Cc2ccnn2CC12CCC2]

To a 40 mL scintillation vial equipped with a magnetic stir bar was added (1-(((1H-pyrazol-5-yl)methyl)(tert-butoxycarbonyl)amino)cyclobutyl)methyl 4-methylbenzenesulfonate (920 mg, 2.112 mmol) and anhydrous DMF (10.6 mL). While stirring, NaH (253 mg, 6.34 mmol, 60% in mineral oil) was added portion-wise. The reaction was stirred at room temperature for 2 h. After completion, the reaction was quenched with water (10 mL) and saturated aqueous solution of NH₄Cl (10 mL) and then diluted with ethyl acetate (30 mL). The organic layer was washed with water (10 mL) twice and saturated brine (10 mL) once, dried over MgSO₄, concentrated in vacuo, and purified by silica gel column chromatography (0-40% ethyl acetate/hexanes) to afford the product as a colorless oil (508 mg, 1.929 mmol, 91% yield). LCMS calculated for C₁₄H₂₂N₃O₂ (M+H)⁺: m/z=264.2; found: 264.2.

### Step 4. tert-butyl 3′-bromo-7′H-spiro[cyclobutane-1,6′-pyrazolo[1,5-a]pyrazine]-5′(4′H)-carboxylate

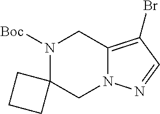 [SMILES: CC(C)(C)OC(=O)N1Cc2c(Br)cnn2CC12CCC2]

To a 40 mL scintillation vial containing a magnetic stir bar and tert-butyl 7′H-spiro[cyclobutane-1,6′-pyrazolo[1,5-a]pyrazine]-5′(4′H)-carboxylate (508 mg, 1.929 mmol) was added DCM (19.3 mL). While stirring, NBS (378 mg, 2.122 mmol) was added portion-wise and the mixture was stirred at room temperature for 1.5 h. After completion, DCM was partially evaporated by N₂ flow at room temperature. The remaining mixture was then purified by silica gel column chromatography (0-40% ethyl acetate/hexanes) to afford the product as a beige solid (599 mg, 1.75 mmol, 91% yield). LCMS calculated for C₁₄H₂₁BrN₃O₂(M+H)⁺: m/z=342.1; found: 342.1.

### Step 5. 3′-bromo-4′,5′-dihydro-7′H-spiro[cyclobutane-1,6′-pyrazolo[1,5-a]pyrazine]

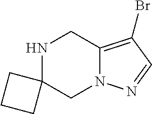 [SMILES: Brc1cnn2c1CNC1(CCC1)C2]

To a 40 mL scintillation vial containing a magnetic stir bar and tert-butyl 3′-bromo-7′H-spiro[cyclobutane-1,6′-pyrazolo[1,5-a]pyrazine]-5′(4′H)-carboxylate (599 mg, 1.75 mmol) was added DCM (8 mL) and TFA (3 mL). The mixture was stirred at room temperature overnight. After completion, volatiles were removed in vacuo and the remaining mixture was purified by silica gel column chromatography (0-15% MeOH/DCM) to afford the desired product in quantitative yield. LCMS calculated for C₉H₁₃BrN₃ (M+H)⁺: m/z=242.0; found: 242.0.

### Step 6. tert-butyl 5-(3′-bromo-4′,5′-dihydro-7′H-spiro[cyclobutane-1,6′-pyrazolo[1,5-a]pyrazine]-5′-carbonyl)indoline-1-carboxylate

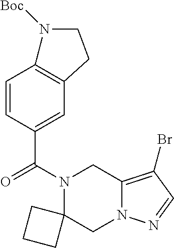 [SMILES: CC(C)(C)OC(=O)N1CCc2cc(C(=O)N3Cc4c(Br)cnn4CC34CCC4)ccc21]

This intermediate was prepared according to the procedure described in Step 1 of Example 57, with 3′-bromo-4′,5′-dihydro-7′H-spiro[cyclobutane-1,6′-pyrazolo[1,5-a]pyrazine] replacing 3′-bromo-4′,5′-dihydro-7′H-spiro[cyclopropane-1,6′-pyrazolo[1,5-a]pyrazine] and 1-(tert-butoxycarbonyl)indoline-5-carboxylic acid replacing 1-(tert-butoxycarbonyl)-4-methoxyindoline-5-carboxylic acid. LCMS calculated for C₂₃H₂₈BrN₄O₃(M+H)⁺: m/z=487.1; found: 487.1.

### Step 7. (3′-bromo-7′H-spiro[cyclobutane-1,6′-pyrazolo[1,5-a]pyrazin]-5′(4′H)-yl)(indolin-5-yl)methanone

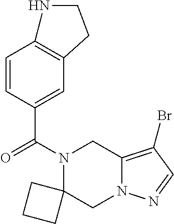 [SMILES: O=C(c1ccc2c(c1)CCN2)N1Cc2c(Br)cnn2CC12CCC2]

This intermediate was prepared according to the procedure described in Step 2 of Example 57, with tert-butyl 5-(3′-bromo-4′,5′-dihydro-7′H-spiro[cyclobutane-1,6′-pyrazolo[1,5-a]pyrazine]-5′-carbonyl)indoline-1-carboxylate replacing tert-butyl 5-(3′-bromo-4′,5′-dihydro-7′H-spiro[cyclopropane-1,6′-pyrazolo[1,5-a]pyrazine]-5′-carbonyl)-4-methoxyindoline-1-carboxylate. LCMS calculated for C₁₈H₂₀BrN₄O (M+H)⁺: m/z=387.1; found: 387.1.

### Step 8. (E)-1-(5-(3′-bromo-4′,5′-dihydro-7′H-spiro[cyclobutane-1,6′-pyrazolo[1,5-a]pyrazine]-5′-carbonyl)indolin-1-yl)-4-(dimethylamino)but-2-en-1-one

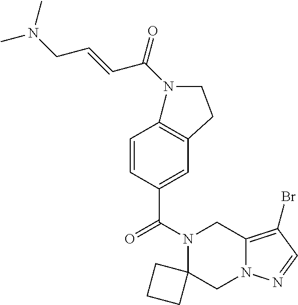 [SMILES: CN(C)C/C=C/C(=O)N1CCc2cc(C(=O)N3Cc4c(Br)cnn4CC34CCC4)ccc21]

This intermediate was prepared according to the procedure described in Step 3 of Example 57, with (E)-4-(dimethylamino)but-2-enoic acid hydrochloride replacing (E)-4-(3-azabicyclo[3.1.1]heptan-3-yl)but-2-enoic acid hydrochloride. LCMS calculated for C₂₄H₂₉BrN₅O₂(M+H)⁺: m/z=498.1; found: 498.1.

### Step 9. (E)-1-(5-(3′-(3,5-dichloro-4-hydroxyphenyl)-4′,5′-dihydro-7′H-spiro[cyclobutane-1,6′-pyrazolo[1,5-a]pyrazine]-5′-carbonyl)indolin-1-yl)-4-(dimethylamino)but-2-en-1-one

This compound was prepared according to the procedure described in Step 4 of Example 57, with (E)-1-(5-(3′-bromo-4′,5′-dihydro-7′H-spiro[cyclobutane-1,6′-pyrazolo[1,5-a]pyrazine]-5′-carbonyl)indolin-1-yl)-4-(dimethylamino)but-2-en-1-one replacing (E)-4-(3-azabicyclo[3.1.1]heptan-3-yl)-1-(5-(3′-bromo-4′,5′-dihydro-7′H-spiro[cyclopropane-1,6′-pyrazolo[1,5-a]pyrazine]-5′-carbonyl)-4-methoxyindolin-1-yl)but-2-en-1-one. LCMS calculated for C₃₀H₃₂Cl₂N₅O₃ (M+H)⁺: m/z=580.2; found: 580.2. ¹H NMR (600 MHz, DMSO-d6) δ 10.09 (s, 1H), 9.89 (s, 1H), 8.16 (d, J=8.2 Hz, 1H), 7.81 (s, 1H), 7.47-7.42 (m, 2H), 7.26 (s, 2H), 6.88-6.74 (m, 2H), 4.85 (s, 2H), 4.46 (s, 2H), 4.25 (t, J=8.5 Hz, 2H), 3.96 (d, J=6.3 Hz, 2H), 3.19 (t, J=8.5 Hz, 2H), 2.81 (s, 6H), 2.42 (d, J=10.7 Hz, 2H), 2.09-2.04 (m, 2H), 2.00 (q, J=9.2 Hz, 1H), 1.85 (t, J=10.1 Hz, 1H).

### Example 60. (E)-4-(3-azabicyclo[3.1.1]heptan-3-yl)-1-(5-((3-(3,5-dichloro-4-hydroxyphenyl)-6,7-dihydropyrazolo[1,5-a]pyrazin-5(4H)-yl)methyl)-2,3-dihydro-1H-pyrrolo[3,2-b]pyridin-1-yl)but-2-en-1-one

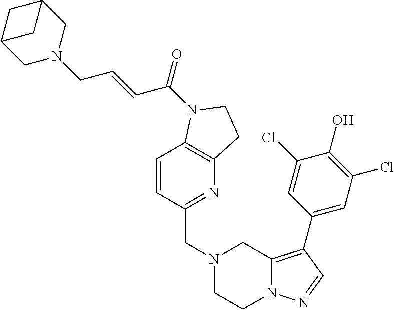 [SMILES: O=C(/C=C/CN1CC2CC(C2)C1)N1CCc2nc(CN3CCn4ncc(-c5cc(Cl)c(O)c(Cl)c5)c4C3)ccc21]

### Step 1. tert-butyl 5-((3-bromo-6,7-dihydropyrazolo[1,5-a]pyrazin-5(4H)-yl)methyl)-2,3-dihydro-1H-pyrrolo[3,2-b]pyridine-1-carboxylate

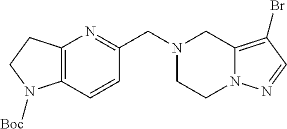 [SMILES: CC(C)(C)OC(=O)N1CCc2nc(CN3CCn4ncc(Br)c4C3)ccc21]

To a 60° C. mixture of 3-bromo-4,5,6,7-tetrahydropyrazolo[1,5-a]pyrazine (120 mg, 0.594 mmol) in DMF (3 mL) and DIPEA (0.471 mL, 2.70 mmol) was added a solution of tert-butyl 5-(chloromethyl)-2,3-dihydro-1H-pyrrolo[3,2-b]pyridine-1-carboxylate (145 mg, 0.540 mmol) in DCM (1 mL) dropwise. The reaction was stirred at 60° C. and monitored by LCMS. After the reaction, ethyl acetate was added to dilute the reaction, and the mixture was then washed three times with water before a final wash by brine. The organic layer was then dried with Na₂SO₄, filtered and concentrated. The residue was purified by silica gel column chromatography (0-100% ethyl acetate/hexanes) to give the desired product. LCMS calculated for C₁₉H₂₅BrN₅O₂(M+H)⁺: m/z=434.1, 436.1; found: 434.1, 436.1.

### Step 2. 2,6-dichloro-4-(5-((2,3-dihydro-1H-pyrrolo[3,2-b]pyridin-5-yl)methyl)-4,5,6,7-tetrahydropyrazolo[1,5-a]pyrazin-3-yl)phenol

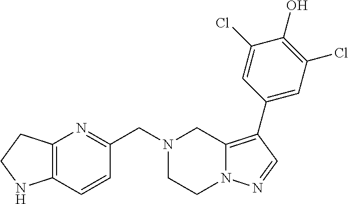 [SMILES: Oc1c(Cl)cc(-c2cnn3c2CN(Cc2ccc4c(n2)CCN4)CC3)cc1Cl]

To a solution of tert-butyl 5-((3-bromo-6,7-dihydropyrazolo[1,5-a]pyrazin-5(4H)-yl)methyl)-2,3-dihydro-1H-pyrrolo[3,2-b]pyridine-1-carboxylate (489 mg, 1.126 mmol) in 1,4-dioxane (10.5 mL) and water (1.5 mL) was added 2,6-dichloro-4-(4,4,5,5-tetramethyl-1,3,2-dioxaborolan-2-yl)phenol (390 mg, 1.351 mmol), potassium phosphate (717 mg, 3.38 mmol), and XPhos Pd G4 (97 mg, 0.113 mmol). The reaction mixture was degassed with N₂ for 5 minutes before heating to 90° C. The reaction was stirred for 2 hours and cooled down. The reaction mixture was then quenched by 1 N HCl and extracted with ethyl acetate. The combined organic layer was dried with Na₂SO₄, concentrated, and dissolved in DCM (10 mL). TFA (2.5 mL) was then added to the solution at 25° C. The reaction was stirred and monitored by LCMS. Upon completion, the reaction was concentrated and purified by silica gel column chromatography (0-100% methanol/DCM) to give the desired product. LCMS calculated for C₂₀H₂₀Cl₂N₅O (M+H)⁺: m/z=416.1; found: 416.1.

### Step 3. (E)-4-(3-azabicyclo[3.1.1]heptan-3-yl)-1-(5-((3-(3,5-dichloro-4-hydroxyphenyl)-6,7-dihydropyrazolo[1,5-a]pyrazin-5(4H)-yl)methyl)-2,3-dihydro-1H-pyrrolo[3,2-b]pyridin-1-yl)but-2-en-1-one

To a solution of (E)-4-(3-azabicyclo[3.1.1]heptan-3-yl)but-2-enoic acid (18.10 mg, 0.100 mmol) in DCM (1 mL) was added oxalyl chloride (2 M in DCM) (0.150 ml, 0.300 mmol) and DMF (0.02 mL). The reaction was stirred at 25° C. until no gas evolution was observed. The reaction mixture was then concentrated under vacuum. To the residue was added a solution of 2,6-dichloro-4-(5-((2,3-dihydro-1H-pyrrolo[3,2-b]pyridin-5-yl)methyl)-4,5,6,7-tetrahydropyrazolo[1,5-a]pyrazin-3-yl)phenol (15 mg, 0.036 mmol) in DMF (1 mL). The reaction was stirred at 25° C. and monitored by LCMS. Upon completion, the reaction mixture was diluted with methanol, filtered, and purified by HPLC to afford the desired product. LCMS calculated for C₃₀H₃₃Cl₂N₆O₂ (M+H)⁺: m/z=579.2; found: 579.4. ¹H NMR (600 MHz, DMSO-d₆) δ 8.32 (d, J=8.2 Hz, 1H), 7.82 (s, 1H), 7.33 (d, J=9.3 Hz, 3H), 6.92-6.78 (m, 2H), 4.33-4.25 (m, 3H), 4.24-4.14 (m, 2H), 4.15-3.91 (m, 8H), 3.74-3.64 (m, 2H), 3.29 (t, J=8.6 Hz, 2H), 3.21-3.05 (m, 2H), 2.28-2.08 (m, 4H), 1.79 (t, J=9.5 Hz, 1H), 1.48 (t, J=8.9 Hz, 1H).

### Example 61. (E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)indolin-1-yl)-4-(dimethylamino)but-2-en-1-one

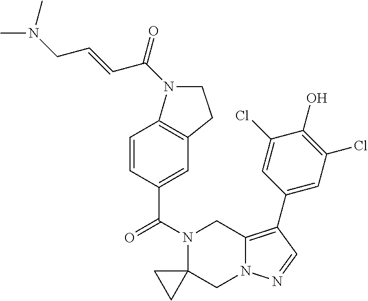 [SMILES: CN(C)C/C=C/C(=O)N1CCc2cc(C(=O)N3Cc4c(-c5cc(Cl)c(O)c(Cl)c5)cnn4CC34CC4)ccc21]

This compound was prepared according to the procedures described in Example 57, with 1-(tert-butoxycarbonyl)indoline-5-carboxylic acid replacing 1-(tert-butoxycarbonyl)-4-methoxyindoline-5-carboxylic acid in step 1 and (E)-4-(dimethylamino)but-2-enoic acid hydrochloride replacing (E)-4-(3-azabicyclo[3.1.1]heptan-3-yl)but-2-enoic acid hydrochloride in step 3. LCMS calculated for C₂₉H₃₀Cl₂N₅O₃ (M+H)⁺: m/z=566.2; found: 566.2. ¹H NMR (500 MHz, DMSO-d6) δ 10.12 (s, 1H), 9.88 (s, 1H), 8.17 (d, J=8.3 Hz, 1H), 7.86 (s, 1H), 7.47-7.39 (m, 2H), 7.37 (s, 2H), 6.88-6.74 (m, 2H), 6.52 (s, 1H), 4.99 (s, 2H), 4.27 (t, J=8.5 Hz, 2H), 3.97 (d, J=6.3 Hz, 2H), 3.22 (t, J=8.4 Hz, 2H), 2.82 (s, 6H), 0.97 (s, 4H).

### Example 62. (E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)-4-morpholinobut-2-en-1-one

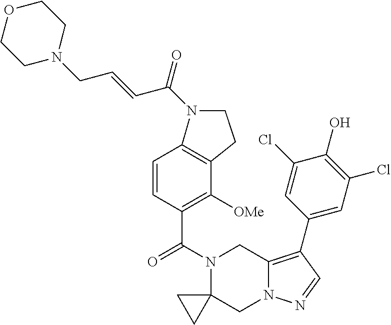 [SMILES: COc1c(C(=O)N2Cc3c(-c4cc(Cl)c(O)c(Cl)c4)cnn3CC23CC3)ccc2c1CCN2C(=O)/C=C/CN1CCOCC1]

This compound was prepared according to the procedures described in Example 57, with (E)-4-morpholinobut-2-enoic acid hydrochloride replacing (E)-4-(3-azabicyclo[3.1.1]heptan-3-yl)but-2-enoic acid hydrochloride in step 3. LCMS calculated for C₃₂H₃₄Cl₂N₅O₅ (M+H)⁺: m/z=638.2; found: 638.2.

### Example 63. (E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)-4-(piperidin-1-yl)but-2-en-1-one

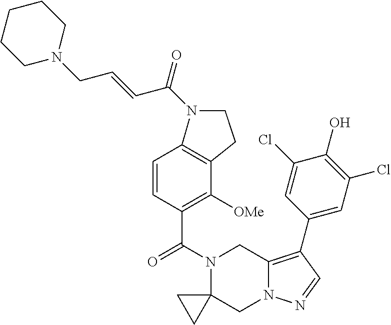 [SMILES: COc1c(C(=O)N2Cc3c(-c4cc(Cl)c(O)c(Cl)c4)cnn3CC23CC3)ccc2c1CCN2C(=O)/C=C/CN1CCCCC1]

This compound was prepared according to the procedures described in Example 57, with (E)-4-(piperidin-1-yl)but-2-enoic acid hydrochloride replacing (E)-4-(3-azabicyclo[3.1.1]heptan-3-yl)but-2-enoic acid hydrochloride in step 3. LCMS calculated for C₃₃H₃₆Cl₂N₅O₄ (M+H)⁺: m/z=636.2; found: 636.2. ¹H NMR (500 MHz, DMF-d7) δ 8.01 (s, 1H), 7.93 (s, 1H), 7.80 (s, 1H), 7.36 (s, 2H), 7.18 (d, J=8.3 Hz, 1H), 6.99-6.94 (m, 2H), 4.99 (s, 2H), 4.31 (t, J=8.4 Hz, 2H), 4.25 (s, 2H), 4.08 (d, J=5.1 Hz, 2H), 3.74 (s, 3H), 3.29 (t, J=8.4 Hz, 2H), 2.92-2.88 (m, 3H), 2.74 (d, J=2.0 Hz, 1H), 1.91 (p, J=5.9 Hz, 4H), 1.66 (s, 2H), 1.18 (s, 2H), 1.01 (s, 2H).

### Example 64. (E)-4-(dimethylamino)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one

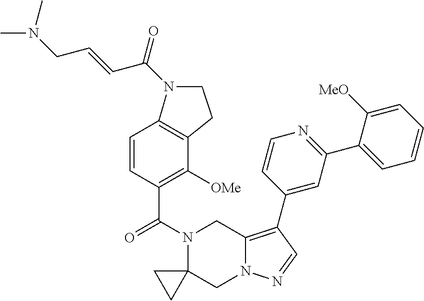 [SMILES: COc1ccccc1-c1cc(-c2cnn3c2CN(C(=O)c2ccc4c(c2OC)CCN4C(=O)/C=C/CN(C)C)C2(CC2)C3)ccn1]

This compound was prepared according to the procedures described in Example 57, with (E)-4-(dimethylamino)but-2-enoic acid hydrochloride replacing (E)-4-(3-azabicyclo[3.1.1]heptan-3-yl)but-2-enoic acid hydrochloride in step 3 and (2-(2-methoxyphenyl)pyridin-4-yl)boronic acid replacing 2,6-dichloro-4-(4,4,5,5-tetramethyl-1,3,2-dioxaborolan-2-yl)phenol in step 4. LCMS calculated for C₃₆H₃₉N₆O₄ (M+H)⁺: m/z=619.3; found: 619.3. ¹H NMR (500 MHz, DMSO-d6) δ 8.66 (d, J=5.5 Hz, 1H), 8.15 (s, 1H), 7.96-7.83 (m, 2H), 7.78-7.73 (m, 1H), 7.62-7.53 (m, 1H), 7.49 (t, J=8.1 Hz, 1H), 7.19 (dd, J=14.6, 8.2 Hz, 2H), 7.12 (t, J=7.5 Hz, 1H), 6.87-6.74 (m, 2H), 5.16-5.07 (m, 2H), 4.26 (s, 2H), 4.24-4.17 (m, 1H), 3.98 (d, J=5.8 Hz, 2H), 3.88-3.83 (m, 3H), 3.68 (s, 3H), 3.28-3.11 (m, 2H), 2.85 (s, 3H), 2.51 (h, J=1.7 Hz, 4H), 1.14-1.02 (m, 2H), 1.03-0.91 (m, 2H).

### Example 65. (E)-1-(5-(3′-(3,5-dichloro-4-hydroxyphenyl)-4′,5′-dihydro-7′H-spiro[cyclobutane-1,6′-pyrazolo[1,5-a]pyrazine]-5′-carbonyl)indolin-1-yl)-4-morpholinobut-2-en-1-one

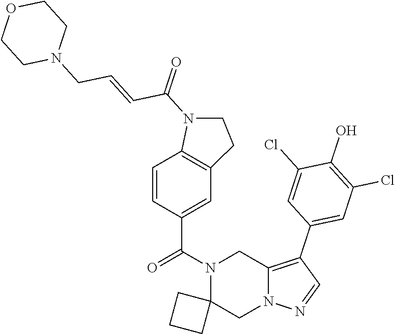 [SMILES: O=C(/C=C/CN1CCOCC1)N1CCc2cc(C(=O)N3Cc4c(-c5cc(Cl)c(O)c(Cl)c5)cnn4CC34CCC4)ccc21]

This compound was prepared according to the procedures described in Example 59, with (E)-4-morpholinobut-2-enoic acid hydrochloride replacing (E)-4-(dimethylamino)but-2-enoic acid hydrochloride in step 8. LCMS calculated for C₃₂H₃₄Cl₂N₅O₄ (M+H)⁺: m/z=622.2; found: 622.2. ¹H NMR (600 MHz, DMSO-d6) δ 10.09 (s, 1H), 8.16 (d, J=8.3 Hz, 1H), 7.81 (s, 1H), 7.47-7.42 (m, 2H), 7.26 (s, 2H), 6.79 (dd, J=12.8, 6.6 Hz, 2H), 4.85 (s, 2H), 4.46 (s, 2H), 4.25 (t, J=8.6 Hz, 2H), 4.01 (s, 4H), 3.68-3.64 (m, 3H), 3.19 (t, J=8.5 Hz, 2H), 3.10 (s, 3H), 2.46-2.39 (m, 2H), 2.09-1.96 (m, 3H), 1.84 (q, J=10.1 Hz, 1H).

### Example 66. (R,E)-1-(5-(3-(3,5-dichloro-4-hydroxyphenyl)-6-methyl-4,5,6,7-tetrahydropyrazolo[1,5-a]pyrazine-5-carbonyl)-4-methoxyindolin-1-yl)-4-morpholinobut-2-en-1-one

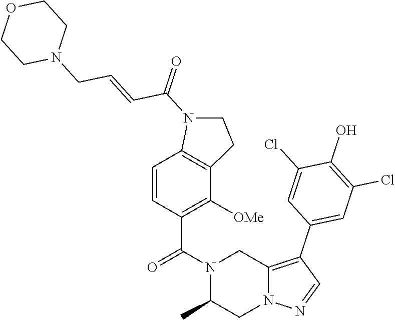 [SMILES: COc1c(C(=O)N2Cc3c(-c4cc(Cl)c(O)c(Cl)c4)cnn3C[C@H]2C)ccc2c1CCN2C(=O)/C=C/CN1CCOCC1]

This compound was prepared according to the procedures described in Example 57, with (R)-3-bromo-6-methyl-4,5,6,7-tetrahydropyrazolo[1,5-a]pyrazine replacing 3′-bromo-4′,5′-dihydro-7′H-spiro[cyclopropane-1,6′-pyrazolo[1,5-a]pyrazine] in step 1 and (E)-4-morpholinobut-2-enoic acid hydrochloride replacing (E)-4-(3-azabicyclo[3.1.1]heptan-3-yl)but-2-enoic acid hydrochloride in step 3. LCMS calculated for C₃₁H₃₄Cl₂N₅O₅ (M+H)⁺: m/z=626.2; found: 626.2.

### Example 67. (E)-1-(5-(3′-(2-methoxypyrimidin-4-yl)-4′,5′-dihydro-7′H-spiro[cyclopropane-1,6′-pyrazolo[1,5-a]pyrazine]-5′-carbonyl)indolin-1-yl)-4-morpholinobut-2-en-1-one

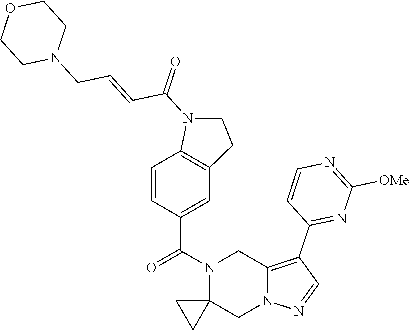 [SMILES: COc1nccc(-c2cnn3c2CN(C(=O)c2ccc4c(c2)CCN4C(=O)/C=C/CN2CCOCC2)C2(CC2)C3)n1]

### Step 1. tert-butyl 3′-(4,4,5,5-tetramethyl-1,3,2-dioxaborolan-2-yl)-7′H-spiro[cyclopropane-1,6′-pyrazolo[1,5-a]pyrazine]-5′(4′H)-carboxylate

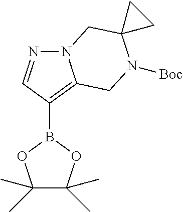 [SMILES: CC(C)(C)OC(=O)N1Cc2c(B3OC(C)(C)C(C)(C)O3)cnn2CC12CC2]

To a 40 mL scintillation vial equipped with a magnetic stir bar was added tert-butyl 7′H-spiro[cyclopropane-1,6′-pyrazolo[1,5-a]pyrazine]-5′(4′H)-carboxylate (300 mg, 1.203 mmol), 4,4,4′,4′,5,5,5′,5′-octamethyl-2,2′-bi(1,3,2-dioxaborolane) (611 mg, 2.407 mmol), di-mu-methoxobis(1,5-cyclooctadiene)diiridium (I) (19.94 mg, 0.030 mmol), 2-methyl-1,10-phenanthroline (11.69 mg, 0.060 mmol) and cyclooctane (12 mL) as the solvent. The vial was degassed with N₂ for 10 min and heated at 110° C. overnight. After full conversion, the reaction mixture was loaded directly onto silica gel and purified by column chromatography (0-50% ethyl acetate/hexanes) to afford the product as a yellow oil (454 mg, 1.20 mmol, quantitative yield). LCMS calculated for C₁₉H₃₁BN₃O₄(M+H)⁺: m/z=376.2; found: 376.2.

### Step 2. tert-butyl 3′-(2-methoxypyrimidin-4-yl)-7′H-spiro[cyclopropane-1,6′-pyrazolo[1,5-a]pyrazine]-5′(4′H)-carboxylate

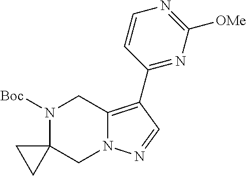 [SMILES: COc1nccc(-c2cnn3c2CN(C(=O)OC(C)(C)C)C2(CC2)C3)n1]

To a 100 mL round bottom flask equipped with a magnetic stir bar was added tert-butyl 3′-(4,4,5,5-tetramethyl-1,3,2-dioxaborolan-2-yl)-7′H-spiro[cyclopropane-1,6′-pyrazolo[1,5-a]pyrazine]-5′(4′H)-carboxylate (1.3627 g, 3.63 mmol), 4-chloro-2-methoxypyrimidine (0.630 g, 4.36 mmol), (1,1′-Bis(diphenylphosphino)ferrocene)-dichloropalladium(II) (0.297 g, 0.363 mmol), potassium phosphate (3.08 g, 14.52 mmol), dioxane (31.8 mL), and water (4.54 mL). The vessel was degassed with N₂ for 10 min before being heated at 90° C. for 15 h. After completion, the reaction mixture was diluted with water (30 mL), extracted with ethyl acetate (30 mL) three times. The organic layers were combined, dried over MgSO₄, concentrated in vacuo, and purified by silica gel column chromatography (0-85% ethyl acetate/hexanes) to afford the product (276 mg, 0.772 mmol, 21% yield). LCMS calculated for C₁₈H₂₄N₅O₃ (M+H)⁺: m/z=358.2; found: 358.2.

### Step 3. 3′-(2-methoxypyrimidin-4-yl)-4′,5′-dihydro-7′H-spiro[cyclopropane-1,6′-pyrazolo[1,5-a]pyrazine]

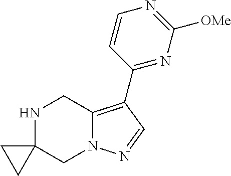 [SMILES: COc1nccc(-c2cnn3c2CNC2(CC2)C3)n1]

To a 40 mL scintillation vial equipped with a magnetic stir bar was added tert-butyl 3′-(2-methoxypyrimidin-4-yl)-7′H-spiro[cyclopropane-1,6′-pyrazolo[1,5-a]pyrazine]-5′(4′H)-carboxylate (276 mg, 0.772 mmol), DCM (3 mL), and TFA (1.5 mL). The mixture was stirred at room temperature overnight. After completion, volatiles were removed in vacuo and the remaining mixture was purified by silica gel column chromatography (0-10% MeOH/DCM) to afford the desired product in quantitative yield. LCMS calculated for C₁₃H₁₆N₅O (M+H)⁺: m/z=258.1; found: 258.1.

### Step 4. (E)-1-(5-(3′-(2-methoxypyrimidin-4-yl)-4′,5′-dihydro-7′H-spiro[cyclopropane-1,6′-pyrazolo[1,5-a]pyrazine]-5′-carbonyl)indolin-1-yl)-4-morpholinobut-2-en-1-one

To a 40 mL scintillation vial equipped with a magnetic stir bar was added 3′-(2-methoxypyrimidin-4-yl)-4′,5′-dihydro-7′H-spiro[cyclopropane-1,6′-pyrazolo[1,5-a]pyrazine](240 mg, 0.933 mmol), (E)-1-(4-morpholinobut-2-enoyl)indoline-5-carboxylic acid (354 mg, 1.119 mmol), HATU (567 mg, 1.492 mmol), DMF (9.3 mL), and DIPEA (2.93 mL, 16.8 mmol). The mixture was stirred at 40° C. overnight. After completion, the reaction mixture was quenched by TFA (0.72 mL), diluted with acetonitrile, filtered, purified by prep-HPLC (pH=10), and further purified by prep-HPLC (pH=2) to afford the product. LCMS calculated for C₃₀H₃₄N₇O₄ (M+H)⁺: m/z=556.3; found: 556.3. ¹H NMR (500 MHz, DMSO-d6) δ 8.46 (d, J=5.2 Hz, 1H), 8.27 (s, 1H), 8.14 (s, 1H), 7.46 (s, 1H), 7.45-7.37 (m, 2H), 6.83 (dt, J=15.2, 6.1 Hz, 1H), 6.58 (d, J=15.1 Hz, 1H), 5.25 (s, 2H), 4.26 (t, J=8.5 Hz, 2H), 3.80 (s, 3H), 3.61 (t, J=4.6 Hz, 4H), 3.29 (s, 2H), 3.21-3.14 (m, 4H), 2.42 (t, J=4.6 Hz, 4H), 1.12-0.93 (m, 4H).

### Example 68. (R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-(2,2-dimethylpyrrolidin-1-yl)but-2-en-1-one

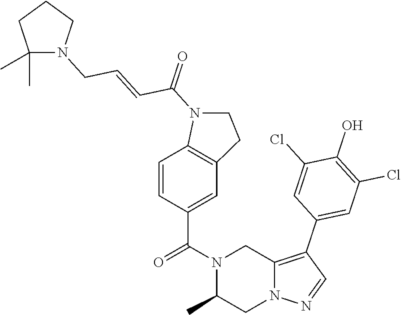 [SMILES: C[C@@H]1Cn2ncc(-c3cc(Cl)c(O)c(Cl)c3)c2CN1C(=O)c1ccc2c(c1)CCN2C(=O)/C=C/CN1CCCC1(C)C]

This compound was prepared according to the procedures described in Example 57, with (R)-3-bromo-6-methyl-4,5,6,7-tetrahydropyrazolo[1,5-a]pyrazine replacing 3′-bromo-4′,5′-dihydro-7′H-spiro[cyclopropane-1,6′-pyrazolo[1,5-a]pyrazine], 1-(tert-butoxycarbonyl)indoline-5-carboxylic acid replacing 1-(tert-butoxycarbonyl)-4-methoxyindoline-5-carboxylic acid in step 1 and (E)-4-(2,2-dimethylpyrrolidin-1-yl)but-2-enoic acid hydrochloride replacing (E)-4-(3-azabicyclo[3.1.1]heptan-3-yl)but-2-enoic acid hydrochloride in step 3. LCMS calculated for C₃₂H₃₆Cl₂N₅O₃ (M+H)⁺: m/z=608.2; found: 608.2. ¹H NMR (500 MHz, DMSO-d6) δ 10.12 (s, 1H), 9.60 (s, 1H), 8.21 (d, J=8.2 Hz, 1H), 7.89 (s, 1H), 7.44 (d, J=4.8 Hz, 3H), 7.37 (dd, J=8.2, 1.8 Hz, 1H), 6.93 (d, J=15.1 Hz, 1H), 6.83 (dt, J=14.6, 6.8 Hz, 1H), 4.77 (s, 2H), 4.35-4.24 (m, 3H), 4.13 (dd, J=13.2, 6.0 Hz, 2H), 3.77 (s, 1H), 3.58 (s, 1H), 3.25 (t, J=8.7 Hz, 3H), 2.05-1.98 (m, 2H), 1.95-1.81 (m, 2H), 1.47 (s, 3H), 1.28-1.21 (m, 6H).

### Example 69. N-(4-(3′-(3,5-dichloro-4-hydroxyphenyl)-4′,5′-dihydro-7′H-spiro[cyclopropane-1,6′-pyrazolo[1,5-a]pyrazine]-5′-carbonyl)-2-methoxyphenyl)acrylamide

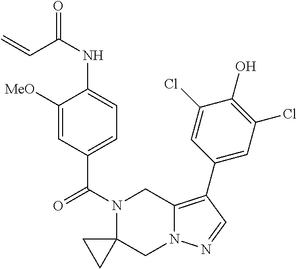 [SMILES: C=CC(=O)Nc1ccc(C(=O)N2Cc3c(-c4cc(Cl)c(O)c(Cl)c4)cnn3CC23CC3)cc1OC]

### Step 1. tert-butyl 4-acrylamido-3-methoxybenzoate

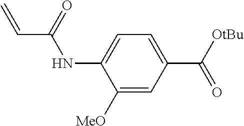 [SMILES: C=CC(=O)Nc1ccc(C(=O)OC(C)(C)C)cc1OC]

To a 40 mL scintillation vial equipped with a magnetic stir bar was added tert-butyl 4-amino-3-methoxybenzoate (95 mg, 0.425 mmol), triethylamine (0.119 mL, 0.851 mmol), and DCM (2 mL). At 0° C., acryloyl chloride (41 mg, 0.447 mmol) dissolved in DCM (5 mL) was added to the stirring mixture dropwise via an addition funnel over 15 minutes. The reaction mixture was stirred and warmed up from 0° C. to room temperature for 20 minutes. Upon completion, the mixture was quenched with water and extracted with DCM. The combined organic layer was dried over MgSO₄, concentrated, and purified by silica gel column chromatography (25%-60% ethyl acetate/hexanes) to afford the desired product in quantitative yield. LCMS calculated for C₁₅H₂₀NO₄ (M+H)⁺: m/z=278.1; found: 278.1.

### Step 2. 4-acrylamido-3-methoxybenzoic acid

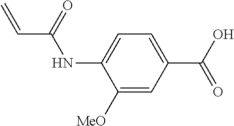 [SMILES: C=CC(=O)Nc1ccc(C(=O)O)cc1OC]

To a 40 mL scintillation vial containing a magnetic stir bar and tert-butyl 4-acrylamido-3-methoxybenzoate (118 mg, 0.425 mmol) was added DCM (2 mL) and trifluoroacetic acid (1 mL). The mixture was stirred at room temperature until analysis by LCMS indicated full conversion of the reactant. The mixture was then concentrated in vacuo and carried forward to the next step without further purification. LCMS calculated for C₁₁H₁₂NO₄ (M+H)⁺: m/z=222.1; found: 222.1.

### Step 3. N-(4-(3′-bromo-4′,5′-dihydro-7′H-spiro[cyclopropane-1,6′-pyrazolo[1,5-a]pyrazine]-5′-carbonyl)-2-methoxyphenyl)acrylamide

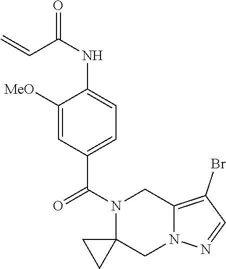 [SMILES: C=CC(=O)Nc1ccc(C(=O)N2Cc3c(Br)cnn3CC23CC3)cc1OC]

To a 40 mL scintillation vial containing a magnetic stir bar was added 3′-bromo-4′,5′-dihydro-7′H-spiro[cyclopropane-1,6′-pyrazolo[1,5-a]pyrazine](15 mg, 0.066 mmol), 4-acrylamido-3-methoxybenzoic acid (21.82 mg, 0.099 mmol), HATU (50.0 mg, 0.132 mmol), DMF (658 μl), and DIPEA (68.9 μl, 0.395 mmol). The mixture was stirred at 40° C. overnight. After completion, the reaction mixture was diluted with ethyl acetate (10 mL), washed with water (5 mL) twice and saturated brine (5 mL) once. The organic layer was separated, dried over MgSO₄, concentrated in vacuo, and purified by silica gel column chromatography (0-5% MeOH/DCM) to afford the product in quantitative yield. LCMS calculated for C₁₉H₂₀BrN₄O₃(M+H)⁺: m/z=431.1; found: 431.1.

### Step 4. N-(4-(3′-(3,5-dichloro-4-hydroxyphenyl)-4′,5′-dihydro-7′H-spiro[cyclopropane-1,6′-pyrazolo[1,5-a]pyrazine]-5′-carbonyl)-2-methoxyphenyl)acrylamide

To a 40 mL scintillation vial containing a magnetic stir bar was added N-(4-(3′-bromo-4′,5′-dihydro-7′H-spiro[cyclopropane-1,6′-pyrazolo[1,5-a]pyrazine]-5′-carbonyl)-2-methoxyphenyl)acrylamide (0.094 g, 0.219 mmol), 2,6-dichloro-4-(4,4,5,5-tetramethyl-1,3,2-dioxaborolan-2-yl)phenol (0.127 g, 0.438 mmol), methanesulfonato(2-dicyclohexylphosphino-2′,4′,6′-tri-1-propyl-1,1′-biphenyl)(2′-methylamino-1,1′-biphenyl-2-yl)palladium(II) (0.028 g, 0.033 mmol), potassium phosphate (0.186 g, 0.876 mmol), dioxane (1.916 mL), and water (0.274 mL). The vessel was degassed with N₂ for 10 min before being heated at 90° C. for 70 min. After completion, the reaction was quenched with a few drops of TFA, diluted with acetonitrile, filtered, and purified by prep-HPLC (pH=2) to afford the product. LCMS calculated for C₂₅H₂₃Cl₂N₄O₄ (M+H)⁺: m/z=513.1; found: 513.1. ¹H NMR (500 MHz, DMSO-d6) δ 10.11 (s, 1H), 9.50 (s, 1H), 8.23 (d, J=8.3 Hz, 1H), 7.87 (s, 1H), 7.39 (s, 2H), 7.19 (d, J=1.8 Hz, 1H), 7.14 (dd, J=8.3, 1.8 Hz, 1H), 6.76 (dd, J=17.0, 10.2 Hz, 1H), 6.27 (dd, J=17.0, 2.0 Hz, 1H), 5.75 (dd, J=10.1, 2.0 Hz, 1H), 5.09-4.94 (m, 2H), 4.45-4.12 (m, 2H), 3.91 (s, 3H), 1.04-0.93 (m, 4H).

### Example 70. (R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-(piperidin-1-yl)but-2-en-1-one

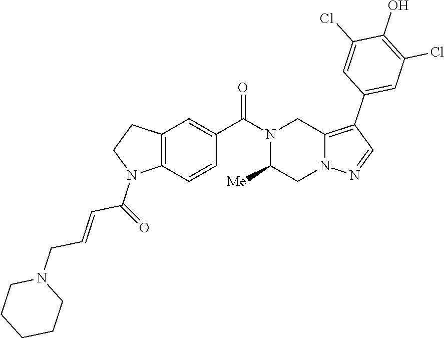 [SMILES: C[C@@H]1Cn2ncc(-c3cc(Cl)c(O)c(Cl)c3)c2CN1C(=O)c1ccc2c(c1)CCN2C(=O)/C=C/CN1CCCCC1]

### Step 1. (R)-2,6-dichloro-4-(6-methyl-4,5,6,7-tetrahydropyrazolo[1,5-a]pyrazin-3-yl)phenol

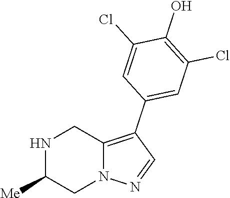 [SMILES: C[C@@H]1Cn2ncc(-c3cc(Cl)c(O)c(Cl)c3)c2CN1]

To a solution of tert-butyl (R)-3-bromo-6-methyl-6,7-dihydropyrazolo[1,5-a]pyrazine-5(4H)-carboxylate (474 mg, 1.5 mmol) in 1,4-dioxane (14 mL) and water (2 mL) was added 2,6-dichloro-4-(4,4,5,5-tetramethyl-1,3,2-dioxaborolan-2-yl)phenol (563 mg, 1.950 mmol), potassium phosphate (955 mg, 4.50 mmol), and XPhos Pd G4 (129 mg, 0.150 mmol). then degassed with N₂ for 5 minutes before heated to 90° C. The reaction was stirred for 2 hours and cooled down. The reaction mixture was then quenched by 1 N HCl and extracted with ethyl acetate. The combined organic layer was dried with Na₂SO₄ and concentrated. The residue was redissolved in DCM (8 ml) and mixed with TFA (2 mL). The mixture was then stirred at 40° C. until LCMS showed complete deprotection of Boc group. The reaction mixture was concentrated under vacuum and purified by silica gel column chromatography (0-100% methanol/DCM) to give the desired product. LCMS calculated for C₁₃H₁₄Cl₂N₃O (M+H)⁺: m/z=298.1; found: 298.1.

### Step 2. (R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-(piperidin-1-yl)but-2-en-1-one

To a mixture of (R)-2,6-dichloro-4-(6-methyl-4,5,6,7-tetrahydropyrazolo[1,5-a]pyrazin-3-yl)phenol (7.45 mg, 0.025 mmol), (E)-1-(4-(piperidin-1-yl)but-2-enoyl)indoline-5-carboxylic acid (7.86 mg, 0.025 mmol), DIPEA (0.017 ml, 0.100 mmol) in DMF (1 mL) was added HATU (0.014 g, 0.038 mmol) in one portion. The reaction was stirred at 60° C. monitored by LCMS. Upon completion, the reaction mixture was diluted with methanol, filtered, and purified by HPLC to afford the desired product. LCMS calculated for C₃₁H₃₄Cl₂N₅O₃ (M+H)⁺: m/z=594.2; found: 594.4. ¹H NMR (600 MHz, DMSO-d₆) δ 8.20 (d, J=8.3 Hz, 1H), 7.89 (s, 1H), 7.55-7.32 (m, 4H), 6.91-6.75 (m, 2H), 4.29 (dt, J=17.2, 10.7 Hz, 4H), 4.11 (d, 1H), 3.96 (d, J=6.2 Hz, 2H), 3.24 (s, 2H), 2.92 (q, J=11.1 Hz, 2H), 1.85 (d, J=14.2 Hz, 3H), 1.75-1.58 (m, 5H), 1.40 (d, J=13.0 Hz, 1H), 1.28-1.14 (m, 5H).

### Example 71. (E)-4-(3-fluoroazetidin-1-yl)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one

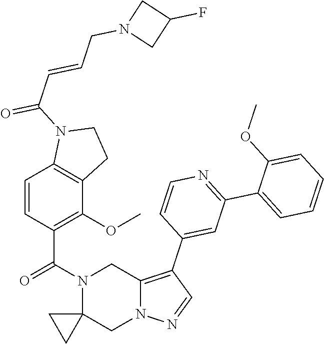 [SMILES: COc1ccccc1-c1cc(-c2cnn3c2CN(C(=O)c2ccc4c(c2OC)CCN4C(=O)/C=C/CN2CC(F)C2)C2(CC2)C3)ccn1]

Example 71 was prepared according to the procedures described in Example 76 as a TFA salt, using tert-butyl 5-(3′-bromo-4′,5′-dihydro-7′H-spiro[cyclopropane-1,6′-pyrazolo[1,5-a]pyrazine]-5′-carbonyl)-4-methoxyindoline-1-carboxylate instead of tert-butyl 5-(3′-bromo-4′,5′-dihydro-7′H-spiro[cyclopropane-1,6′-pyrazolo[1,5-a]pyrazine]-5′-carbonyl)indoline-1-carboxylate in Step 1. LCMS calculated for C₃₇H₃₈FN₆O₄(M+H)⁺: m/z=649.3; found: 649.3.

### Example 72. (R)-1-(5-(3-(3,5-dichloro-4-hydroxyphenyl)-6-methyl-4,5,6,7-tetrahydropyrazolo[1,5-a]pyrazine-5-carbonyl)isoindolin-2-yl)prop-2-en-1-one

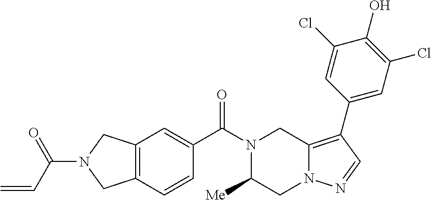 [SMILES: C=CC(=O)N1Cc2ccc(C(=O)N3Cc4c(-c5cc(Cl)c(O)c(Cl)c5)cnn4C[C@H]3C)cc2C1]

### Step 1. tert-butyl (R)-5-(3-bromo-6-methyl-4,5,6,7-tetrahydropyrazolo[1,5-a]pyrazine-5-carbonyl)isoindoline-2-carboxylate

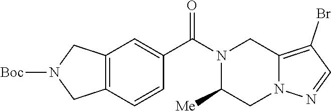 [SMILES: C[C@@H]1Cn2ncc(Br)c2CN1C(=O)c1ccc2c(c1)CN(C(=O)OC(C)(C)C)C2]

To a mixture of (R)-3-bromo-6-methyl-4,5,6,7-tetrahydropyrazolo[1,5-a]pyrazine hydrochloride (30.3 mg, 0.12 mmol), 2-(tert-butoxycarbonyl)isoindoline-5-carboxylic acid (31.6 mg, 0.120 mmol), and DIPEA (0.105 mL, 0.600 mmol) in DMF (2.5 mL) was added HATU (91 mg, 0.240 mmol) in one portion. The reaction was stirred at 60° C. monitored by LCMS. Upon completion, the reaction mixture was quenched with water and extracted with ethyl acetate. The combined organic layer was dried with Na₂SO₄ and concentrated. The residue was purified by silica gel column chromatography (0%-100% ethyl acetate/hexanes) to give the desired product. LCMS calculated for C₂₁H₂₆BrN₄O₃(M+H)⁺: m/z=461.1, 463.1; found: 461.1, 463.1.

### Step 2. (R)-(3-bromo-6-methyl-6,7-dihydropyrazolo[1,5-a]pyrazin-5(4H)-yl)(isoindolin-5-yl)methanone

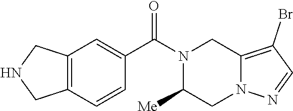 [SMILES: C[C@@H]1Cn2ncc(Br)c2CN1C(=O)c1ccc2c(c1)CNC2]

To a solution of tert-butyl (R)-5-(3-bromo-6-methyl-4,5,6,7-tetrahydropyrazolo[1,5-a]pyrazine-5-carbonyl)isoindoline-2-carboxylate (0.055 g, 0.12 mmol) in DCM (3 mL) was added HCl (4.0 M in dioxane) (0.300 mL, 1.200 mmol). The mixture was stirred at 40° C. until LCMS showed complete transformation. The reaction mixture was concentrated and used in the next step without purification. LCMS calculated for C₁₆H₁₈BrN₄O (M+H)⁺: m/z=361.1, 363.1; found: 361.0, 363.0.

### Step 3. (R)-1-(5-(3-bromo-6-methyl-4,5,6,7-tetrahydropyrazolo[1,5-a]pyrazine-5-carbonyl)isoindolin-2-yl)prop-2-en-1-one

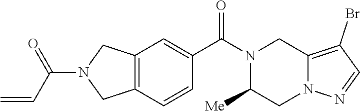 [SMILES: C=CC(=O)N1Cc2ccc(C(=O)N3Cc4c(Br)cnn4C[C@H]3C)cc2C1]

To a solution of (R)-(3-bromo-6-methyl-6,7-dihydropyrazolo[1,5-a]pyrazin-5(4H)-yl)(isoindolin-5-yl)methanone (25.3 mg, 0.07 mmol) at 0° C. in DCM (1 mL) was added DIPEA (0.024 mL, 0.140 mmol) and acryloyl chloride (6.26 μl, 0.077 mmol). The reaction mixture was stirred for 20 minutes and quenched with saturated NH₄Cl solution. The mixture was extracted three times with ethyl acetate, and the combined organic layer was dried with Na₂SO₄ and concentrated. The residue was purified by silica gel column chromatography (0%-100% ethyl acetate/hexanes, then 0-50% methanol/ethyl acetate) to give the desired product. LCMS calculated for C₁₉H₂₀BrN₄O₂(M+H)⁺: m/z=415.1, 417.1; found: 415.0, 417.0.

### Step 4. (R)-1-(5-(3-(3,5-dichloro-4-hydroxyphenyl)-6-methyl-4,5,6,7-tetrahydropyrazolo[1,5-a]pyrazine-5-carbonyl)isoindolin-2-yl)prop-2-en-1-one

To a solution of (R)-1-(5-(3-bromo-6-methyl-4,5,6,7-tetrahydropyrazolo[1,5-a]pyrazine-5-carbonyl)isoindolin-2-yl)prop-2-en-1-one (16.1 mg, 0.039 mmol) in 1,4-dioxane (0.7 mL) and water (0.100 mL) was added 2,6-dichloro-4-(4,4,5,5-tetramethyl-1,3,2-dioxaborolan-2-yl)phenol (16.80 mg, 0.058 mmol), potassium phosphate (24.69 mg, 0.116 mmol), and XPhos Pd G4 (3.34 mg, 3.88 μmol). The reaction mixture was then degassed with N₂ for 5 minutes before heated to 90° C. The reaction was stirred for 2 hours and cooled down. The reaction mixture was then diluted with methanol, filtered, and purified by HPLC to afford the desired product. LCMS calculated for C₂₅H₂₃Cl₂N₄O₃ (M+H)⁺: m/z=497.1; found: 497.3. ¹H NMR (600 MHz, DMSO-d₆) δ 7.88 (s, 1H), 7.58-7.35 (m, 6H), 6.69 (ddd, J=16.1, 10.3, 5.2 Hz, 1H), 6.24 (d, J=16.8 Hz, 1H), 5.77 (dd, J=10.3, 2.3 Hz, 1H), 5.00 (d, J=6.5 Hz, 3H), 4.77 (d, J=4.4 Hz, 4H), 4.37-4.27 (m, 1H), 4.15-4.01 (m, 1H), 1.30-1.17 (m, 3H).

### Example 73. (R,E)-N-(4-(3-(3,5-dichloro-4-hydroxyphenyl)-6-methyl-4,5,6,7-tetrahydropyrazolo[1,5-a]pyrazine-5-carbonyl)-2-fluorophenyl)-4-morpholinobut-2-enamide

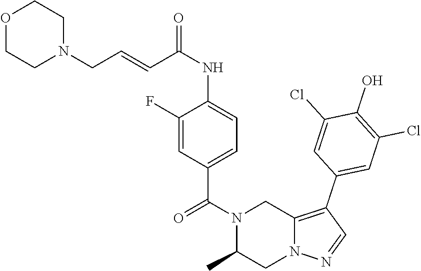 [SMILES: C[C@@H]1Cn2ncc(-c3cc(Cl)c(O)c(Cl)c3)c2CN1C(=O)c1ccc(NC(=O)/C=C/CN2CCOCC2)c(F)c1]

This compound was prepared according to the procedures described in Example 28, with methyl 4-amino-3-fluorobenzoate replacing methyl 4-amino-3-methoxybenzoate and (E)-4-morpholinobut-2-enoic acid hydrochloride replacing (E)-4-(dimethylamino)but-2-enoic acid hydrochloride in step 1, and (R)-2,6-dichloro-4-(6-methyl-4,5,6,7-tetrahydropyrazolo[1,5-a]pyrazin-3-yl)phenol replacing 2,6-dichloro-4-(4,5,6,7-tetrahydropyrazolo[1,5-a]pyrazin-3-yl)phenol in step 3. LCMS calculated for C₂₈H₂₉Cl₂FN₅O₄(M+H)⁺: m/z=588.2; found: 588.2. ¹H NMR (500 MHz, DMSO-d₆): δ 10.03 (s, 1H), 9.87 (s, 1H), 8.14 (q, J=7.7 Hz, 1H), 7.84 (s, 1H), 7.52-7.42 (m, 1H), 7.41 (s, 2H), 7.35 (dd, J=8.2, 1.8 Hz, 1H), 6.83 (dt, J=14.2, 6.8 Hz, 1H), 6.66 (d, J=15.5 Hz, 1H), 5.20 (d, J=17.2 Hz, 1H), 4.74 (d, J=17.2 Hz, 1H), 4.66 (s, 1H), 4.34 (dd, J=12.8, 4.7 Hz, 1H), 4.11 (d, J=12.8 Hz, 1H), 3.81 (s, 6H), 3.10 (s, 4H), 1.26 (d, J=6.9 Hz, 3H).

### Example 74. (R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-morpholinobut-2-en-1-one

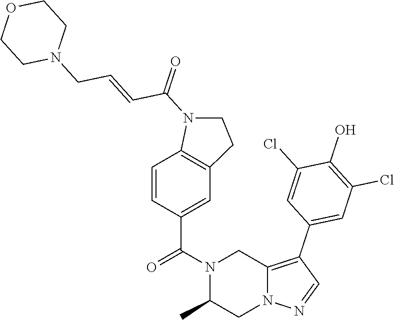 [SMILES: C[C@@H]1Cn2ncc(-c3cc(Cl)c(O)c(Cl)c3)c2CN1C(=O)c1ccc2c(c1)CCN2C(=O)/C=C/CN1CCOCC1]

This compound was prepared according to the procedures described in Example 57, with (R)-3-bromo-6-methyl-4,5,6,7-tetrahydropyrazolo[1,5-a]pyrazine replacing 3′-bromo-4′,5′-dihydro-7′H-spiro[cyclopropane-1,6′-pyrazolo[1,5-a]pyrazine], 1-(tert-butoxycarbonyl)indoline-5-carboxylic acid replacing 1-(tert-butoxycarbonyl)-4-methoxyindoline-5-carboxylic acid in step 1 and (E)-4-morpholinobut-2-enoic acid hydrochloride replacing (E)-4-(3-azabicyclo[3.1.1]heptan-3-yl)but-2-enoic acid hydrochloride in step 3. LCMS calculated for C₃₀H₃₂Cl₂N₅O₄ (M+H)⁺: m/z=596.2; found: 596.2. ¹H NMR (500 MHz, DMSO-d6) δ 9.87 (s, 1H), 8.15 (s, 1H), 7.84 (s, 1H), 7.41 (d, J=9.0 Hz, 3H), 7.35 (dd, J=8.2, 1.8 Hz, 1H), 6.82 (d, J=3.0 Hz, 2H), 5.18 (d, J=17.1 Hz, 1H), 4.74 (d, J=17.2 Hz, 2H), 4.32 (dd, J=12.9, 4.8 Hz, 1H), 4.27 (t, J=8.5 Hz, 2H), 4.12 (d, J=12.8 Hz, 1H), 3.92 (s, 2H), 3.83 (s, 4H), 3.24 (t, J=8.6 Hz, 2H), 3.16 (s, 4H), 1.26 (d, J=6.9 Hz, 3H).

### Example 75. (R,E)-N-(4-(3-(3,5-dichloro-4-hydroxyphenyl)-6-isopropyl-4,5,6,7-tetrahydropyrazolo[1,5-a]pyrazine-5-carbonyl)-2-methoxyphenyl)-4-(dimethylamino)but-2-enamide

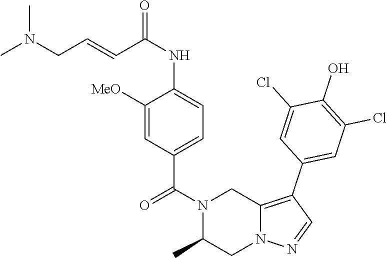 [SMILES: COc1cc(C(=O)N2Cc3c(-c4cc(Cl)c(O)c(Cl)c4)cnn3C[C@H]2C)ccc1NC(=O)/C=C/CN(C)C]

### Step 1. (R,E)-N-(4-(3-bromo-6-isopropyl-4, 5, 6, 7-tetrahydropyrazolo[1,5-a]pyrazine-5-carbonyl)-2-methoxyphenyl)-4-(dimethylamino)but-2-enamide

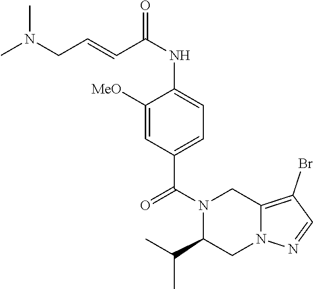 [SMILES: COc1cc(C(=O)N2Cc3c(Br)cnn3C[C@H]2C(C)C)ccc1NC(=O)/C=C/CN(C)C]

To a 40 mL scintillation vial containing a magnetic stir bar was added (R)-3-bromo-6-isopropyl-4,5,6,7-tetrahydropyrazolo[1,5-a]pyrazine (100 mg, 0.410 mmol), (E)-4-(4-(dimethylamino)but-2-enamido)-3-methoxybenzoic acid (125 mg, 0.451 mmol), HATU (234 mg, 0.614 mmol), DMF (4.1 mL), and DIPEA (215 μL, 1.229 mmol). The mixture was stirred at 60° C. overnight. After completion, the reaction mixture was diluted with ethyl acetate (10 mL), washed with water (5 mL) twice and brine (5 mL) once. The organic layer was separated, dried over MgSO₄, concentrated in vacuo and purified by column chromatography (0-15% MeOH/DCM) afford the product (54 mg, 0.107 mmol, 26% yield). LCMS calculated for C₂₃H₃₁BrN₅O₃(M+H)⁺: m/z=504.2; found: 504.2.

### Step 2. (R,E)-N-(4-(3-(3,5-dichloro-4-hydroxyphenyl)-6-isopropyl-4,5,6,7-tetrahydropyrazolo[1,5-a]pyrazine-5-carbonyl)-2-methoxyphenyl)-4-(dimethylamino)but-2-enamide

To a 40 mL scintillation vial containing a magnetic stir bar was added (R,E)-N-(4-(3-bromo-6-isopropyl-4,5,6,7-tetrahydropyrazolo[1,5-a]pyrazine-5-carbonyl)-2-methoxyphenyl)-4-(dimethylamino)but-2-enamide (54 mg, 0.107 mmol), (3,5-dichloro-4-hydroxyphenyl)boronic acid (28.8 mg, 0.139 mmol), methanesulfonato(2-dicyclohexylphosphino-2′,4′,6′-tri-1-propyl-1,1′-biphenyl)(2′-methylamino-1,1′-biphenyl-2-yl)palladium(II) (9.22 mg, 10.71 μmol), potassium phosphate (56.8 mg, 0.268 mmol), dioxane (937 μl), and water (134 μl). The vessel was degassed with N₂ for 10 min before being heated at 90° C. for 1 hour. After completion, the reaction was quenched with a few drops of TFA, diluted with acetonitrile, filtered, and purified by prep-HPLC (pH=2) to afford the product. LCMS calculated for C₂₉H₃₄Cl₂N₅O₄ (M+H)⁺: m/z=586.2; found: 586.2. ¹H NMR (500 MHz, DMSO-d6) δ 10.12 (s, 1H), 9.69 (s, 1H), 8.19 (d, J=8.2 Hz, 1H), 7.90 (s, 1H), 7.52 (s, 1H), 7.34 (s, 1H), 7.18 (d, J=1.7 Hz, 1H), 7.09 (d, J=8.1 Hz, 1H), 6.83-6.70 (m, 2H), 6.52 (s, 1H), 5.33 (d, J=17.5 Hz, 1H), 4.59 (d, J=17.4 Hz, 1H), 4.47 (s, 1H), 4.38 (d, J=13.1 Hz, 1H), 3.93 (d, J=6.0 Hz, 2H), 3.91 (s, 3H), 2.82 (s, 6H), 1.87 (s, 1H), 1.05 (s, 2H), 0.86-0.79 (m, 4H).

### Example 76. (E)-4-(3-fluoroazetidin-1-yl)-1-(5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one

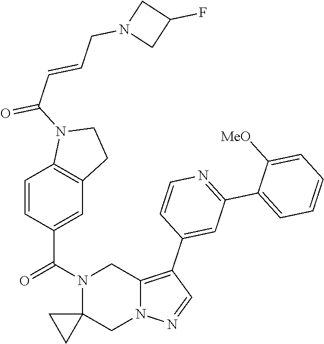 [SMILES: COc1ccccc1-c1cc(-c2cnn3c2CN(C(=O)c2ccc4c(c2)CCN4C(=O)/C=C/CN2CC(F)C2)C2(CC2)C3)ccn1]

### Step 1. (3′-bromo-7′H-spiro[cyclopropane-1,6′-pyrazolo[1,5-a]pyrazin]-5′(4′H)-yl)(indolin-5-yl)methanone dihydrochloride

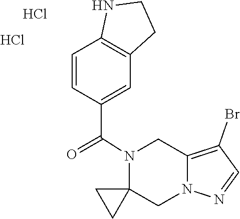 [SMILES: Cl ; Cl ; O=C(c1ccc2c(c1)CCN2)N1Cc2c(Br)cnn2CC12CC2]

Tert-butyl 5-(3′-bromo-4′,5′-dihydro-7′H-spiro[cyclopropane-1,6′-pyrazolo[1,5-a]pyrazine]-5′-carbonyl)indoline-1-carboxylate (150 mg, 0.32 mmol) was dissolved in a solution of HCl (3.0 mL, 4.0M in 1,4-Dioxane, 12.00 mmol). The reaction mixture was allowed to stir for 1 h. Upon completion, the reaction contents were concentrated in vacuo directly to produce the desired product, and the crude material was used directly in the next step (141 mg, 0.32 mmol, 100%). LCMS calculated for C₁₇H₁₈BrN₄O (M+H)⁺: m/z=373.1; found: 373.1.

### Step 2. (E)-4-bromo-1-(5-(3′-bromo-4′,5′-dihydro-7′H-spiro[cyclopropane-1,6′-pyrazolo[1,5-a]pyrazine]-5′-carbonyl)indolin-1-yl)but-2-en-1-one

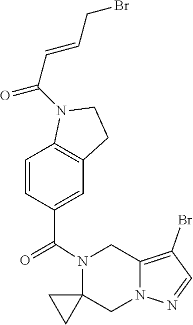 [SMILES: O=C(/C=C/CBr)N1CCc2cc(C(=O)N3Cc4c(Br)cnn4CC34CC4)ccc21]

To a solution of (3′-bromo-7′H-spiro[cyclopropane-1,6′-pyrazolo[1,5-a]pyrazin]-5′(4′H)-yl)(indolin-5-yl)methanone dihydrochloride (141 mg, 0.32 mmol) in DMF (2.1 mL) was added (E)-4-bromobut-2-enoic acid (156 mg, 0.95 mmol), DIPEA (0.33 mL, 1.90 mmol), and n-propylphosphonic acid anhydride, cyclic trimer (0.37 mL, 0.63 mmol). The reaction mixture was allowed to stir for 12 h. Upon completion, the reaction contents were diluted with EtOAc (20 mL), washed subsequently with NaHCO_{3(aq)} (30 mL), H₂O (30 mL), and brine (30 mL), dried over Na₂SO₄. The organic layer was then concentrated in vacuo directly to produce the desired product, and the crude material was used directly in the next step (160 mg, 0.31 mmol, 97%). LCMS calculated for C₂₁H₂₁Br₂N₄O₂ (M+H)⁺: m/z=519.0; found: 519.0.

### Step 3. (E)-1-(5-(3′-bromo-4′,5′-dihydro-7′H-spiro[cyclopropane-1,6′-pyrazolo[1,5-a]pyrazine]-5′-carbonyl)indolin-1-yl)-4-(3-fluoroazetidin-1-yl)but-2-en-1-one

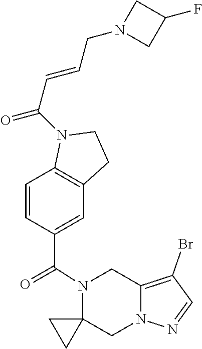 [SMILES: O=C(/C=C/CN1CC(F)C1)N1CCc2cc(C(=O)N3Cc4c(Br)cnn4CC34CC4)ccc21]

To a solution of (E)-4-bromo-1-(5-(3′-bromo-4′,5′-dihydro-7′H-spiro[cyclopropane-1,6′-pyrazolo[1,5-a]pyrazine]-5′-carbonyl)indolin-1-yl)but-2-en-1-one (160 mg, 0.31 mmol) in DMF (2.1 mL) was added 3-fluoroazetidine hydrochloride (51.5 mg, 0.46 mmol) and tripotassium phosphate (653 mg, 3.08 mmol). The reaction mixture was allowed to stir for 3 h. Upon completion, the reaction contents were diluted with EtOAc (15 mL), then washed subsequently with NH₄Cl_{(aq)} (20 mL), H₂O (20 mL), and brine (20 mL). The organic layer was then dried over Na₂SO₄, and concentrated in vacuo before being purified by flash column chromatography (0-100% hexanes/EtOAc) to provide the desired product (63.2 mg, 0.12 mmol, 40%). LCMS calculated for C₂₄H₂₆BrFN₅O₂(M+H)⁺: m/z=514.1; found: 514.1.

### Step 4. (E)-4-(3-fluoroazetidin-1-yl)-1-(5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one

To a solution of (E)-1-(5-(3′-bromo-4′,5′-dihydro-7′H-spiro[cyclopropane-1,6′-pyrazolo[1,5-a]pyrazine]-5′-carbonyl)indolin-1-yl)-4-(3-fluoroazetidin-1-yl)but-2-en-1-one (63.2 mg, 0.12 mmol) in 1,4-Dioxane (1.1 mL) and H₂O (0.15 mL) was added (2-(2-methoxyphenyl)pyridin-4-yl)boronic acid (42.2 mg, 0.18 mmol), tripotassium phosphate (156 mg, 0.74 mmol), and [1,1′-bis(diphenylphosphino)ferrocene]dichloropalladium(II), complex with dichloromethane (20.1 mg, 0.03 mmol). The reaction mixture was then degassed for 5 min before heating to 90° C. The reaction was stirred for 1 h. Upon completion, reaction contents were filtered through Celite, quenched with NH₄Cl_{(aq)} (15 mL), extracted three times with EtOAc (10 mL), and concentrated in vacuo. The crude residue was purified by FCC (0-10% CH₂Cl₂/MeOH) before further purification by prep-LCMS (XBridge C18 column, eluting with a gradient of acetonitrile/water containing 0.1% TFA, at flow rate of 60 mL/min) to afford the desired product as a TFA salt. LCMS calculated for C₃₆H₃₆FN₆O₃(M+H)⁺: m/z=619.3; found: 619.3. ¹H NMR (600 MHz, DMSO_d6) δ 8.70 (d, J=5.7 Hz, 1H), 8.26 (s, 1H), 8.17 (d, J=8.3 Hz, 1H), 7.99 (s, 1H), 7.73 (d, J=7.5 Hz, 1H), 7.66 (s, 1H), 7.53 (t, J=7.9 Hz, 1H), 7.48-7.41 (m, 2H), 7.24 (d, J=8.4 Hz, 1H), 7.15 (t, J=7.5 Hz, 1H), 6.87-6.72 (m, 1H), 6.69 (dt, J=15.1, 6.2 Hz, 1H), 5.54-5.35 (m, 1H), 5.19 (s, 2H), 4.61-4.30 (m, 6H), 4.26 (t, J=8.6 Hz, 2H), 4.12 (d, J=6.0 Hz, 2H), 3.91 (s, 3H), 3.21 (t, 2H), 0.95 (d, J=13.1 Hz, 4H).

### Example A. VRK1 and VRK2 Biochemical Activity Assay

Inhibitor potency was determined by luminescence-based ADP-Glo activity assay (Promega), which quantifies the ADP product of kinase reactions. Purified recombinant VRK proteins were custom-ordered from Proteos, Inc. (Kalamazoo, MI), with Baculovirus (Sf9) expression and containing 6×His tag and TEV-cleavage site at the N-terminus. Full-length construct was used for VRK1 (amino acids 1-396, Uniprot Q99986), while “Isoform 2” sequence was used for VRK2 (amino acids 2-394, Uniprot Q86Y07-2). ADP-Glo assay buffer contained 40 mM HEPES, pH 7.5, 0.02% CHAPS, 10 mM NaCl, 10 mM MgCl2, 1 mM EGTA, 0.05 mg/mL BSA, 0.5 mM TCEP, and 10% Glycerol. ATP substrate mixtures were prepared at 2× final concentrations containing ATP at 1×Km concentrations, determined experimentally to be 45 μM for VRK1 and 75 μM for VRK2. Enzyme was diluted in assay buffer to 2× final concentration immediately prior to initiating assay. Final enzyme concentrations were 20 nM VRK1 and 4 nM VRK2. DMSO or test compound in 100% DMSO at 100× concentration was added to white 384-well low-volume assay plates at 40 nL/well volume using an Echo® acoustic dispenser (Labcyte). A Tempest® liquid dispenser (Formulatrix) was used to add 2 μL/well 2× enzyme mix, followed by 30 minute pre-incubation at ambient temperature. After pre-incubation, 2 μL/well 2×ATP mix was added by MultiDrop Combi (ThermoFisher) to initiate reactions. After 180 min incubation, 2 μL/well ADP-Glo reagent was added by Combi to quench the kinase reaction, and to deplete remaining ATP from the reactions. After 60 min incubation with ADP-Glo reagent, 2 μL/well detection reagent was added by Combi to convert ADP product to luminescence signal. Luminescence was measured on a PHERAstar® FSX plate reader (BMG Labtech). Enzyme titrations were used to determine reaction rates and linearity, and to choose the concentration used in inhibitor studies for each enzyme. Chosen concentrations yielded robust assay statistics and reproducibility, with enzyme reactions and rate vs enzyme concentration relationships both in a linear range.

### Example B. VRK1 Competition Binding Assay

Inhibitor binding affinity was determined by competition Binding TR-FRET assay using a Fluorescein-labeled ATP-competitive ligand and LanthaScreen® Tb-Streptavidin detection reagent (ThermoFisher, Carlsbad, CA). Purified recombinant biotinylated VRK1 protein was custom ordered from Proteos, Inc. (Kalamazoo, MI), with Baculovirus (Sf9) expression and containing n-terminal avi-tag in addition to 6×His-tag, with in vitro biotinylation by co-expression with BirA. Full-length construct was used for VRK1 (amino acids 1-396, Uniprot Q99986). Binding assay buffer contained 40 mM HEPES, pH 7.5, 10 mM MgCl2, 10 mM NaCl, 1 mM EGTA, 0.02% CHAPS, 0.01% Brij-35, 0.1 mg/mL BSA, 2 mM DTT, and 5% Glycerol. DMSO or test compound in 100% DMSO at 200× concentration was added to white 384-well low-volume assay plates at 25 nL/well volume using an Echo® acoustic dispenser (Labcyte). Enzyme was diluted in assay buffer to 1.67× final concentration immediately prior to adding to assay. Final enzyme concentration was 0.15 nM VRK1. A Tempest® liquid dispenser (Formulatrix) was used to add 3 l/well 1.67× Enzyme mix, followed by 120 minutes pre-incubation at ambient temperature. Ligand and detection mixture was prepared at 2.5× final concentrations containing custom Fluorescein-labeled ATP-competitive ligand synthesized internally at Incyte (final concentration 20 nM, 1×Kd concentration), and LanthaScreen® Tb-Streptavidin detection reagent (final concentration 0.4 nM). After pre-incubation, 2 μL/well 2.5× ligand and detection mix was added by MultiDrop Combi (ThermoFisher). After 120 min incubation, LanthaScreen signal (337 nm emission, 520/490 nm dual emission) was measured on a PHERAstar® FSX plate reader (BMG Labtech). Positive signal is generated by TR-FRET between fluorescein ligand bound to enzyme in close proximity to Terbium-Streptavidin, which binds with high affinity to enzyme via Biotin tag. Displacement of ligand (competition) by test compound causes reduction of signal.

### Example C. IC₅₀ Data Analysis

The IC₅₀s for the assays in Example A and B are calculated by the following procedure. The data analysis tools integrated in Genedata Screener® software were used to analyze the inhibition data. For LanthaScreen® TR-FRET assay with dual emission, Ratiometric data were calculated and used for data fitting (“A” Channel, 520 nm emission counts, divided by “B” Channel, 490 nm emission counts, then multiplied by 10,000). Data were normalized to percent of vehicle control, after subtracting background (reference inhibitor control at 100×IC₅₀ to fully inhibit assay signal). The IC₅₀ values were derived by fitting the data to a 4-parameter logistic equation producing a sigmoidal dose-response curve using the following equation: Y=Bottom+(Top−Bottom)/(1+10\hat{ }([Log IC₅₀−X] Hill slope)), where X is the logarithm of inhibitor concentration and Y is the response.

### Potency and Selectivity Data

Table 1 presents the IC₅₀ data at VRK1 and the selectivity ratio of VRK2/VRK1 calculated by dividing the IC₅₀ value for VRK2 by the IC₅₀ value for VRK1.

In Table 1, the VRK1 IC₅₀ was measured as described in Example B, while the VRK2 IC₅₀ was measured as described in Example A.

In Table 1, + means an IC₅₀ of <10 nM; ++ means an IC₅₀ of 10 nM to <100 nM; +++ means an IC₅₀ of 100 nM to <1000 nM; and ++++ means an IC₅₀ of 1000 nM to 2000 nM.

TABLE 1

| Example No. | VRK1 IC₅₀ (nM) | Selectivity (VRK2/VRK1) |
|---|---|---|
| 1 | + | >100 |
| 2 | + | >100 |
| 3 | + | >100 |
| 4 | + | >100 |
| 5 | + | >100 |
| 6 | + | >100 |
| 7 | ++ | >100 |
| 8 | + | >100 |
| 9 | + | >100 |
| 10 | + | >100 |
| 11 | + | >100 |
| 12 | + | >100 |
| 13 | ++ | >100 |
| 14 | + | >100 |
| 15 | + | >100 |
| 16 | ++ | >100 |
| 17 | + | >100 |
| 18 | + | >100 |
| 19 | + | >100 |
| 20 | +++ | >10 |
| 21 | ++ | >100 |
| 22 | + | >100 |
| 23 | ++ | >100 |
| 24 | ++ | >100 |
| 25 | ++ | >100 |
| 26 | ++ | >100 |
| 27 | + | >100 |
| 28 | + | >100 |
| 29 | + | >100 |
| 30 | + | >100 |
| 31 | + | >100 |
| 32 | + | >100 |
| 33 | + | >100 |
| 34 | + | >100 |
| 35 | + | >100 |
| 36 | + | >100 |
| 37 | + | >100 |
| 38 | + | >100 |
| 39 | + | >100 |
| 40 | + | >100 |
| 41 | + | >100 |
| 42 | + | >100 |
| 43 | + | >100 |
| 44 | + | >100 |
| 45 | + | >100 |
| 46 | + | >100 |
| 47 | + | >100 |
| 48 | + | >100 |
| 49 | + | >100 |
| 50 | + | >100 |
| 51 | + | >100 |
| 52 | + | >100 |
| 53 | + | >100 |
| 54 | + | >100 |
| 55 | + | >100 |
| 56 | + | >100 |
| 57 | + | >100 |
| 58 | + | >100 |
| 59 | + | >100 |
| 60 | + | >100 |
| 61 | + | >100 |
| 62 | + | >100 |
| 63 | + | >100 |
| 64 | + | >100 |
| 65 | + | >100 |
| 66 | + | >100 |
| 67 | + | >100 |
| 68 | + | >100 |
| 69 | + | >100 |
| 70 | + | >100 |
| 71 | + | >100 |
| 72 | + | >100 |
| 73 | + | >100 |
| 74 | + | >100 |
| 75 | + | >100 |
| 76 | + | >100 |

### Example D. VRK1 Inhibitor Cellular Activity Assays

Human brain cancer cell lines were obtained from ATCC which included VRK1-dependent HS683 (HTB-138), U118MG (HTB-15) and VRK1-independent U87MG (HTB-14). A VRK1-independent line was engineered by transfecting HS683 cells to express VRK2 (termed HS683VRK2); HS683 cells were transduced with a lentiviral VRK2 expression construct from VectorBuilder, Inc and pooled transduced cells were selected for two weeks with 1 microgram per milliliter of puromycin (Gibco cat #A1113803) followed by confirmation of VRK2 expression via western blotting. All cells are maintained in growth medium DMEM (Gibco cat #1195065) supplemented with 10% FBS (Gibco cat #16140071) and are kept at 37° C. in a humidified 5% CO₂ incubator.

### Cellular Assay—VRK1 Inhibitor 6 Day Viability Assays

Compounds are diluted at 1:3 ratios for dose-response determination in DMSO prior to dispensation to assay plates, with all wells receiving an equal amount of DMSO. DMSO alone serves as no treatment control (100% viability) and 20 micromolar Staurosporine (Selleck Chemicals LLC, Cat #S1421) serves as a positive control (0% viability). Compounds are dispensed to assay plates prior to addition of cells and growth medium at volumes indicated below. All cells are disassociated with TrypLE (Gibco cat #12604-013), counted, and resuspended in growth medium DMEM with 10% FBS. Cell viability assays are performed in either 96- or 384-well format. For 96-well assays, 100 microliters containing 800 cells and growth medium are seeded into black 96 well plates (Greiner cat #655090) containing 400 nanoliters per well of compounds diluted in DMSO. For 384-well assays, 50 microliters containing 400 cells and growth medium are seeded into white 384-well plates (Corning cat #3765) containing 200 nanoliters per well of compounds diluted in DMSO. Plates are incubated for six days at 37° C. in a humidified 5% CO₂ incubator. Following incubation, 50 microliters (96-well) or 25 microliters (384-well) of CellTiter Glo reagent (Promega cat #G7573) is added and plates are read on the Pherastar FSX (BMG Labtech) for luminescence. Data analyses are carried out using Genedata Screener SmartFit (Danaher Corporation), with CellTiter Glo raw data (luminescence) normalized to controls (DMSO 100% viability, Staurosporine, 0% viability). Fitting parameters can include dose response IC₅₀, Hill slope (n), maximum response (S_{∞}), and minimum response (S₀).

Various modifications of the contents of the present disclosure, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

What is claimed is:

1. A compound of Formula (I): 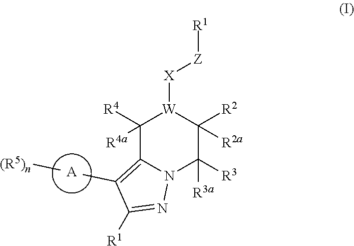
or a pharmaceutically acceptable salt thereof, wherein:
n is 0, 1, 2, 3, 4, 5, or 6;
W is CH or N;
when W is N, then X is CR⁶R^{6a}, C(O), S(O), or S(O)₂;
when W is CH, then X is NR^{6b}, CR⁶R^{6a} C(O), S(O), or S(O)₂;
Z is -Cy¹- or -Cy¹-Cy²-, wherein Cy¹ is bound to X, provided that when Z is -Cy¹-Cy²-, then Cy² is bound to R^{W};
Cy¹ is C₃₋₁₂ cycloalkyl, 6-10 membered aryl, 4-12 membered heterocycloalkyl, and 5-10 membered heteroaryl, each of which is optionally substituted by 1, 2, 3, 4, 5, or 6 independently selected R⁷ substituents;
Cy² is C₃₋₁₂ cycloalkyl, 6-10 membered aryl, 4-12 membered heterocycloalkyl, and 5-10 membered heteroaryl, each of which is optionally substituted by 1, 2, 3, 4, 5, or 6 independently selected R⁷ substituents;
Ring moiety A is C₃₋₁₀ cycloalkyl, 6-10 membered aryl, 4-10 membered heterocycloalkyl, or 5-10 membered heteroaryl;
R¹ is H, D, halo, CN, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, or C₁₋₃ alkoxy-C₁₋₄ alkyl;
R² and R^{2a} are each independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or R² and R^{2a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R² and R^{2a}, taken together with the carbon atom to which they are attached, form a C(O) group;
each R^{a2}, R^{c2}, and R^{d2} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c2} and R^{d2} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
R³ and R^{3a} are each independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c3} and R^{d3} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents;
each R^{b3} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
R⁴ and R^{4a} are each independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a4}, SR^{a4}, NHOR^{a4}, C(O)R^{c4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c4} and R^{d4} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents;
each R^{b4} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R⁵ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
or, any R^{c5} and R^{d5} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)₂NR^{c51}R^{d51}, NR^{c51}S(O)R^{b51}, NR^{c51}S(O)₂R^{b51}, NR^{c51}S(O)(═NR^{e51})R^{b51}, NR^{c51}S(O)₂NR^{c51}R^{d51}, S(O)R⁵¹, S(O)NR^{c51}R^{d51}, S(O)₂R^{b51}, S(O)₂NR^{c51}R^{d51}, OS(O)(═NR^{e51})R^{b51}, OS(O)₂R^{b51}, S(O)(═NR^{e51})R^{b51}, SF₅, P(O)R^{f51}R^{g51}, OP(O)(OR^{h51})(ORⁱ⁵¹), P(O)(OR^{h51})(ORⁱ⁵¹), and BR^{j51}R^{k51}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5B} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5B} substituents;
or, any R^{c51} and R^{d51} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{5B} substituents;
each R^{b51} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{5B} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f51} and R^{g51} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h51} and Rⁱ⁵¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a52}, SR^{a52}, NHOR^{a52}, C(O)R^{b52}, C(O)NR^{c52}R^{d52}, C(O)NR^{c52}(OR^{a52}), C(O)OR^{a52}, OC(O)R^{b52}, OC(O)NR^{c52}R^{d52}, NR^{c52}R^{d52}, NR^{c52}NR^{c52}R^{d52}, NR^{c52}C(O)R^{b52}, NR^{c52}C(O)OR^{a52}, NR^{c52}C(O)NR^{c52}R^{d52}, C(═NR^{e52})R^{b52}, C(═NR^{e52})NR^{c2}R^{d52}, NR^{c52}C(═NR^{e52})NR^{c52}R^{d52}, NR^{c52}C(═NR^{e52})R^{b52}, NR^{c52}S(O)NR^{c52}R^{d52}, NR^{c52}S(O)R^{b52}, NR^{c52}S(O)₂R^{b52}, NR^{c52}S(O)(═NR^{e52})R^{b52}, NR^{c52}S(O)₂NR^{c52}R^{d52}, S(O)R^{b52}, S(O)NR^{c52}R^{d52}, S(O)₂R^{b52}, S(O)₂NR^{c52}R^{d52}, OS(O)(═NR^{e52})R^{b52}, OS(O)₂R^{b52}, S(O)(═NR^{e52})R^{b52}, SF₅, P(O)R^{f52}R^{g52}, OP(O)(OR^{h52})(ORⁱ⁵²), P(O)(OR^{h52})(ORⁱ⁵²), and BR^{j52}R^{k52}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a52}, R^{c52}, and R^{d52} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c52} and R^{d52} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b52} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e52} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f52} and R^{g52} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h52} and Rⁱ⁵² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j52} and R^{k52} is independently selected from OH, C₁₋₆ alkoxy, and C₁₋₆ haloalkoxy;
or any R^{j52} and R^{k52} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁶ and R^{6a} are each independently selected from H, D, halo, CN, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
R^{6b} is selected from H, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R⁷ is independently selected from D, halo, CN, NO₂, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)NR^{c7}R^{d7}, NR^{c7}S(O)R^{b7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, OS(O)(═NR^{e7})R^{b7}, OS(O)₂R^{b7}, S(O)(═NR^{e7})R^{b7}, SF₅, P(O)R^{f7}R^{g7}, OP(O)(OR^{h7})(ORⁱ⁷), P(O)(OR^{h7})(ORⁱ⁷), and BR^{j7}R^{k7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
or, any R^{c7} and R^{d7} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h7} and Rⁱ⁷ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j7} and R^{k7} is independently selected from OH, C₁₋₆ alkoxy, and C₁₋₆ haloalkoxy;
or any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, NHOR^{a71}, C(O)R^{b71}, C(O)NR^{c71}R^{d71}, C(O)NR^{c71}(OR^{a71}), C(O)OR^{a71}, OC(O)R^{b71}, OC(O)NR^{c71}R^{d71}, NR^{c71}R^{d71}, NR^{c71}NR^{c71}R^{d71}, NR^{c71}C(O)R^{b71}, NR^{c71}C(O)OR^{a71}, NR^{c71}C(O)NR^{c71}R^{d71}, C(═NR^{e71})R^{b71}, C(═NR^{e71})NR^{c71}R^{d71}, NR^{c71}C(═NR^{e71})NR^{c71}R^{d71}, NR^{c71}C(═NR^{e71})R^{b71}, NR^{c71}S(O)NR^{c71}R^{d71}, NR^{c71}, S(O)R^{b71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, OS(O)₂R^{b71}, S(O)(═NR^{e71})R^{b71}, SF₅, P(O)R^{f71}R^{g71}, OP(O)(OR^{h71})(ORⁱ⁷¹), P(O)(OR^{h71})(ORⁱ⁷¹), and BR^{j71}R^{k71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f71} and R^{g71} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h71} and Rⁱ⁷¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j71} and R^{k71} is independently selected from OH, C₁₋₆ alkoxy, and C₁₋₆ haloalkoxy;
or any R^{j71} and R^{k71} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a72}, SR^{a72}, NHOR^{a72}, C(O)R^{b72}, C(O)NR^{c72}R^{d72}, C(O)NR^{c72}(OR^{a72}), C(O)OR^{a72}, OC(O)R^{b72}, OC(O)NR^{c72}R^{d72}, NR^{c72}R^{d72}, NR^{c72}NR^{c72}R^{d72}, NR^{c72}C(O)R^{b72}, NR^{c72}C(O)OR^{a72}, NR^{c72}C(O)NR^{c72}R^{d72}, C(═NR^{e72})R^{b72}, C(═NR^{e72})NR^{c72}R^{d72}, NR^{c72}C(═NR^{e72})NR^{c72}R^{d72}, NR^{c72}C(═NR^{e72})R^{b72}, NR^{c72}S(O)NR^{c72}R^{d72}, NR^{c72}S(O)R^{b72}, NR^{c72}S(O)₂R^{b72}, NR^{c72}S(O)(═NR^{e72})R^{b72}, NR^{c72}S(O)₂NR^{c72}R^{d72}, S(O)R^{b72}, S(O)NR^{c72}R^{d72}, S(O)₂R^{b72}, S(O)₂NR^{c72}R^{d72}, OS(O)(═NR^{e72})R^{b72}, OS(O)₂R^{b72}, S(O)(═NR^{e72})R^{b72}, SF₅, P(O)R^{f72}R^{g72}, OP(O)(OR^{h72})(ORⁱ⁷²), P(O)(OR^{h72})(ORⁱ⁷²), and BR^{j72}R^{k72}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a72}, R^{c72}, and R^{d72} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c72} and R^{d72} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b72} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e72} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f72} and R^{g72} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h72} and Rⁱ⁷² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j72} and R^{k72} is independently selected from OH, C₁₋₆ alkoxy, and C₁₋₆ haloalkoxy;
or any R^{j72} and R^{k72} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{W} is a covalent warhead moiety having a reactive functional group capable of covalently binding to one or more cysteine residues in a protein; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

2. The compound of claim 1, or a pharmaceutically acceptable salt thereof, wherein R^{W} is a means for covalently binding to cysteine residue in a protein.

3. The compound of claim 1, or a pharmaceutically acceptable salt thereof, wherein R^{W} is: 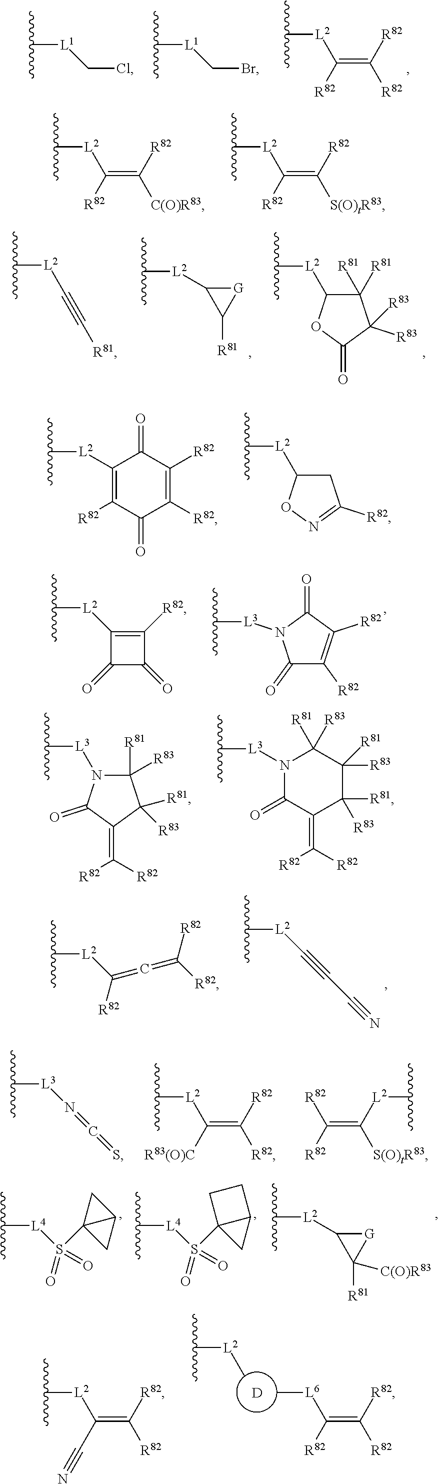 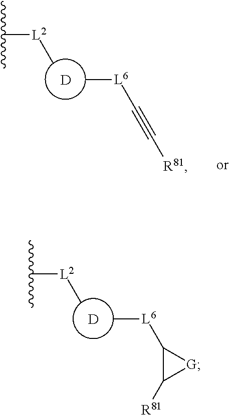
L¹ is -L-C(O)—, -L-NR⁹C(O)—, -L-OC(O)—, -L-S(O)—, -L-S(O)₂—, -L-NR⁹S(O)—, -L-OS(O)—, -L-NR⁹S(O)₂—, or -L-OS(O)₂—, wherein L¹ is attached to Z through the L linking group;
L² is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NRS(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L² is attached to Z through the L linking group;
L³ is -L-, -L-C(O)—, -L-NR⁹C(O)—, -L-OC(O)—, -L-S(O)—, -L-S(O)₂—, -L-NRS(O)—, -L-OS(O)—, -L-NR⁹S(O)₂—, or -L-OS(O)₂—, wherein L³ is attached to Z through the L linking group;
L⁴ is -L-, -L-O—, -L-S—, or -L-NR⁹—, wherein L⁴ is attached to Z through the L linking group;
L⁶ is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹S(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L⁶ is attached to Ring moiety D through the L linking group;
L is a bond or C₁₋₆ alkylene, wherein said C₁₋₆ alkylene is optionally substituted by 1, 2, 3, or 4 independently selected R^{G} substituents; or
L is —O—C₁₋₆ alkyl or —N(R^{N})—C₁₋₆ alkyl, wherein L is attached to Ring moiety A via the oxygen of the —O—C₁₋₆ alkyl or the nitrogen atom of the —N(R^{N})—C₁₋₆ alkyl group;
each R^{N} is independently H or C₁₋₆ alkyl;
Ring D is a 4-12 membered heterocycloalkyl, C₃₋₁₂ cycloalkyl, C₆₋₁₀ aryl, or a 5-10 membered heteroaryl, each of which is optionally substituted with 1, 2, 3, or 4 independently selected C₁₋₆ alkyl groups;
G is O or NR⁹;
each t is independently 0, 1, 2, or 3;
each R⁸¹ and R⁸³ are independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{b8}R^{d8}, NR^{c8}R^{d8}, NR^{c8}NR^{c8}R^{d8}, NR^{c8}C(O)R^{b8}, NR^{c8}C(O)OR^{a8}, NR^{c8}C(O)NR^{c8}R^{d8}, C(═NR^{e8})R^{b8}, C(═NR^{e8})NR^{c8}R^{d8}, NR^{c8}C(═NR^{e8})NR^{c8}R^{d8}, NR^{c8}C(═NR^{e8})R^{b8}, NR^{c8}S(O)NR^{c8}R^{d8}, NR^{c8}(O)R^{b8}, NR^{c8}S(O)₂R^{b8}, NR^{c8}S(O)(═NR^{e8})R^{b8}, NR^{c8}S(O)₂NR^{c8}R^{d8}, S(O)R^{b8}, S(O)NR^{c8}R^{d8}, S(O)₂R^{b8}, S(O)₂NR^{c8}R^{d8}, OS(O)(═NR^{e8})R^{b8}, OS(O)₂R^{b8}, S(O)(═NR^{e8})R^{b8}, SF₅, P(O)R^{f8}R^{g8}, OP(O)(OR^{h8})(ORⁱ⁸), P(O)(OR^{h8})(ORⁱ⁸), and BR^{j8}R^{k8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R⁸² is independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, C(O)R^{b8}, C(O)NR^{c8}R^{d8}, C(O)OR^{a8}, C(═NR^{e8})R^{b8}, C(═NR^{e8})NR^{c8}R^{d8}, S(O)R^{b8}, S(O)NR^{c8}R^{d8}, S(O)₂R^{b8}, and S(O)₂NR^{c8}R^{d8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b8} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h8} and Rⁱ⁸ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j8} and R^{k8} is independently selected from OH, C₁₋₆ alkoxy, and C₁₋₆ haloalkoxy;
or any R^{j8} and R^{k8} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
or any two R⁸¹ and R⁸² together with the atoms to which they are attached, form C₃₋₇ cycloalkyl, 4-7 membered heterocycloalkyl, phenyl, or 5-6-membered heteroaryl ring, each of which is optionally substituted with 1, 2, 3, or 4 independently selected R^{G} substitutents;
each R⁹ is independently selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, OR^{a9}, SR^{a9}, NHOR^{a9}, C(O)R^{b9}, C(O)NR^{c9}R^{d9}, C(O)NR^{c9}(OR^{a9}), C(O)OR^{a9}, OC(O)R^{b9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, C(═NR^{e9})R^{b9}, C(═NR^{e9})NR^{c9}R^{d9}, NR^{c9}C(═NR^{e9})NR^{c9}R^{d9}, NR^{c9}C(═NR^{e9})R^{b9}, NR^{c9}S(O)NR^{c9}R^{d9}, NR^{c9}S(O)R^{b9}, NR^{c9}S(O)₂R^{b9}, NR^{c9}S(O)(═NR^{e9})R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, OS(O)(═NR^{e9})R^{b9}, OS(O)₂R^{b9}, and S(O)(═NR^{e9})R^{b9}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a9}, R^{c9}, and R^{d9} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c9} and R^{d9} attached to the same N atom, together with the N atom to which they are attached, form a 4-7 membered heterocycloalkyl group, which is optionally substituted with 1, 2, 3, or 4 independently selected R^{G} substituents;
each R^{b9} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents; and
each R^{e9} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl.

4. The compound of claim 3, or a pharmaceutically acceptable salt thereof, wherein R^{W} is selected from:

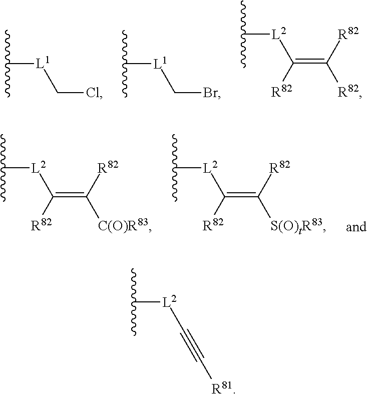

5. The compound of claim 3, or a pharmaceutically acceptable salt thereof, wherein R^{W} is

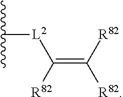

6. The compound of claim 3, or a pharmaceutically acceptable salt thereof, wherein L² is —C(O)—, —NR⁹C(O)—, —S(O)₂—, or —NR⁹S(O)₂—; and R⁹ is selected from H and C₁₋₃ alkyl.

7. The compound of claim 3, or a pharmaceutically acceptable salt thereof, wherein R⁸¹, R⁸², and R⁸³ are each independently selected from H, halo, CN, C₁₋₆ alkyl, C₁₋₆ haloalkyl, C₃₋₇ cycloalkyl, 4-7 membered heterocycloalkyl, C₃₋₇ cycloalkyl-C₁₋₄ alkyl, and 4-7 membered heterocycloalkyl-C₁₋₄ alkyl, wherein said C₁₋₆ alkyl, C₁₋₆ haloalkyl, C₃₋₇ cycloalkyl, 4-7 membered heterocycloalkyl, C₃₋₇ cycloalkyl-C₁₋₄ alkyl, and 4-7 membered heterocycloalkyl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, amino, C₁₋₃ alkylamino, and di(C₁₋₃ alkyl)amino.

8. The compound of claim 1, or a pharmaceutically acceptable salt thereof, wherein n is 0, 1, 2, 3, or 4.

9. The compound of claim 1, or a pharmaceutically acceptable salt thereof, wherein W is N.

10. The compound of claim 1, or a pharmaceutically acceptable salt thereof, wherein R⁶ and R^{6a} are each independently selected from H, D, halo, CN, C₁₋₃ alkyl, and C₁₋₃ haloalkyl; and R^{6b} is selected from H, C₁₋₃ alkyl, and C₁₋₃ haloalkyl.

11. The compound of claim 1, or a pharmaceutically acceptable salt thereof, wherein X is CR⁶R^{6a}, C(O) or S(O)₂.

12. The compound of claim 1, or a pharmaceutically acceptable salt thereof, wherein Z is Cy¹.

13. The compound of claim 1, or a pharmaceutically acceptable salt thereof, wherein Cy¹ is phenyl, naphthyl, 5-6 membered heteroaryl, 9-10 membered heteroaryl, or 9-12 membered heterocycloalkyl.

14. The compound of claim 1, or a pharmaceutically acceptable salt thereof, wherein Cy¹ is selected from phenyl, naphthyl, pyridinyl, pyrazolyl, thiazolyl, indolinyl, isoindolinyl, 2,3-dihydro-1H-pyrrolo[2,3-b]pyridinyl, 1,2,3,4-tetrahydroquinolinyl, 2,3-dihydrobenzo[b][1,4]dioxinyl, 2,3-dihydro-1H-pyrrolo[3,4-c]pyridinyl, 3,4-dihydro-2H-benzo[b][1,4]oxazinyl, and isoquinolinyl.

15. The compound of claim 1, or a pharmaceutically acceptable salt thereof, wherein Cy¹ is phenyl, naphthyl, 5-6 membered heteroaryl, 9-10 membered heteroaryl, or 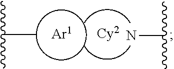
wherein:
Ar¹ is fused to Cy³ and Ar¹ is bonded to X;
Ar¹ is phenyl or 5-6 membered heteroaryl;
Cy³ is 4-8 membered heterocycloalkyl; and
Cy¹ is optionally substituted with 1, 2, 3, or 4 independently selected R⁷ substituents.

16. The compound of claim 1, or a pharmaceutically acceptable salt thereof, wherein R¹ selected from H, halo, CN, CH₃ and CF₃.

17. The compound of claim 1, or a pharmaceutically acceptable salt thereof, wherein Ring moiety A is phenyl or 5-10 membered heteroaryl.

18. The compound of claim 1, or a pharmaceutically acceptable salt thereof, wherein Ring moiety A is 5-10 membered heteroaryl.

19. The compound of claim 1, or a pharmaceutically acceptable salt thereof, wherein Ring moiety A is phenyl, pyridinyl, or pyrimidinyl.

20. The compound of claim 1, or a pharmaceutically acceptable salt thereof, wherein R² and R^{2a} are each independently selected from H, D, halo, CN, C₁₋₆ alkyl, and C₁₋₆ haloalkyl, and OR^{b2};
or R² and R^{2a}, taken together with the carbon atom to which they are attached, form a C₃₋₇ cycloalkyl ring or a 3-7 membered heterocycloalkyl ring;
or R² and R^{2a}, taken together with the carbon atom to which they are attached, form a C(O) group; and
each R^{b2} is independently selected from C₁₋₆ alkyl and C₁₋₆ haloalkyl.

21. The compound of claim 1, or a pharmaceutically acceptable salt thereof, wherein R² and R^{2a} are each independently selected from H, halo, CN, CH₃ and CF₃.

22. The compound of claim 1, or a pharmaceutically acceptable salt thereof, wherein R³ and R^{3a} are each independently selected from H, D, halo, CN, C₁₋₆ alkyl, and C₁₋₆ haloalkyl, and OR^{b3};
or R³ and R^{3a}, taken together with the carbon atom to which they are attached, form a C₃₋₇ cycloalkyl ring or a 3-7 membered heterocycloalkyl ring;
or R³ and R^{3a}, taken together with the carbon atom to which they are attached, form a C(O) group; and
each R^{b3} is independently selected from C₁₋₆ alkyl and C₁₋₆ haloalkyl.

23. The compound of claim 1, or a pharmaceutically acceptable salt thereof, wherein R³ and R^{3a} are each independently selected from H, halo, CN, CH₃ and CF₃.

24. The compound of claim 1, or a pharmaceutically acceptable salt thereof, wherein R⁴ and R^{4a} are each independently selected from H, D, halo, CN, C₁₋₆ alkyl, and C₁₋₆ haloalkyl;
or R⁴ and R^{4a}, taken together with the carbon atom to which they are attached, form a C₃₋₇ cycloalkyl ring or a 3-7 membered heterocycloalkyl ring;
or R⁴ and R^{4a}, taken together with the carbon atom to which they are attached, form a C(O) group; and
each R^{b4} is independently selected from C₁₋₆ alkyl and C₁₋₆ haloalkyl.

25. The compound of claim 1, or a pharmaceutically acceptable salt thereof, wherein R⁴ and R^{4a} are each independently selected from H, halo, CN, CH₃ and CF₃.

26. The compound of claim 1, or a pharmaceutically acceptable salt thereof, wherein each R⁵ is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-10 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, NHOR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-10 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents.

27. The compound of claim 1, or a pharmaceutically acceptable salt thereof, wherein each R⁵ is independently selected from D, halo, CN, C₁₋₆ alkyl, C₂₋₆ alkenyl, C₂₋₆ alkynyl, C₁₋₆ haloalkyl, OR^{a5} and NR^{c5}R^{d5}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{5A} substituents.

28. The compound of claim 1, or a pharmaceutically acceptable salt thereof, wherein:
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, wherein said C₁₋₆ alkyl, C₂₋₆ alkenyl, C₂₋₆ alkynyl, C₁₋₆ haloalkyl, C₃₋₄ cycloalkyl, and 4-5 membered heterocycloalkyl are each optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents; and
each R^{5A} is independently selected from OH, CN, halo, C₁₋₃ alkyl, C₁₋₃ haloalkyl, amino, C₁₋₃ alkoxy, C₁₋₃ alkylamino, and di(C₁₋₃ alkyl)amino.

29. The compound of claim 1, or a pharmaceutically acceptable salt thereof, wherein each R⁵ is independently selected from halo, CN, C₁₋₃ alkyl, C₁₋₃ haloalkyl, OH, C₁₋₃ alkoxy, C₁₋₃ haloalkoxy, phenyl, pyridinyl, pyrimidinyl, anisolyl, and 2,3-dihydrobenzofuranyl.

30. The compound of claim 1, or a pharmaceutically acceptable salt thereof, wherein each R⁵ is independently selected from Cl, F, CN, CH₃, OH, and OCH₃.

31. The compound of claim 1, or a pharmaceutically acceptable salt thereof, wherein 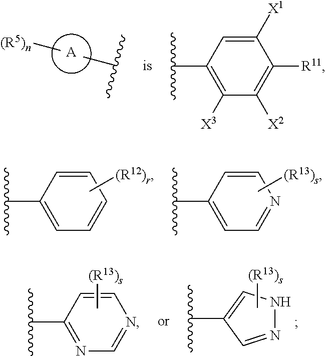
X¹ is H, Cl, F, or OCH₃;
X² is H, Cl, or F;
X³ is H, Cl, F, or OCH₃;
R¹¹ is H, OH, or CN;
r is 2;
s is 0, 1, or 2;
two R¹² substituents, together with the carbon atoms to which they are attached, form a tetrahydrofuranyl, pyrazole, pyrrole, or pyrimidine ring, which is optionally substituted by 1 or 2 independently selected R⁵ substituents selected from halo, CN, and C₁₋₃ alkyl; and
each R¹³ is independently selected from halo, CN, C₁₋₃ alkyl, and OCH₃.

32. The compound of claim 1, or a pharmaceutically acceptable salt thereof, wherein

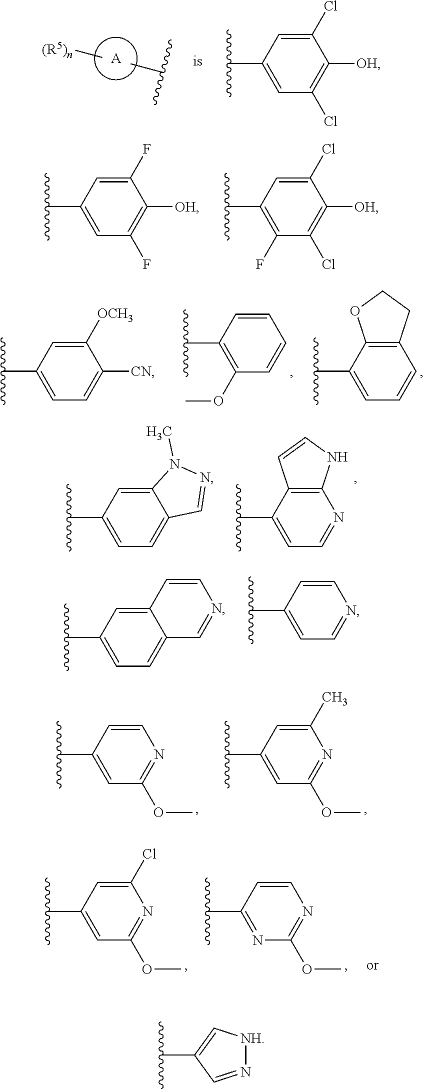

33. The compound of claim 1, or a pharmaceutically acceptable salt thereof, wherein each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents.

34. The compound of claim 1, or a pharmaceutically acceptable salt thereof, wherein:
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents; and
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino.

35. The compound of claim 1, or a pharmaceutically acceptable salt thereof, wherein:
n is 0, 1, 2, 3, or 4;
W is N;
X is CR⁶R^{6a}, C(O), or S(O)₂;
Z is Cy¹;
Cy¹ is phenyl, naphthyl, 5-6 membered heteroaryl, 9-10 membered heteroaryl, or 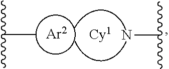
each of which is optionally substituted by 1, 2, 3, 4, 5, or 6 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl;
R¹ is H;
Ring moiety A is phenyl, 5-6 membered heteroaryl, or 9-10 membered heteroaryl;
R² and R^{2a} are each independently selected from H, D, halo, CN, C₁₋₆ alkyl, and C₁₋₆ haloalkyl, and OR^{b2};
or R² and R^{2a}, taken together with the carbon atom to which they are attached, form a C₃₋₇ cycloalkyl ring or a 3-7 membered heterocycloalkyl ring;
each R^{a2} is independently selected from C₁₋₆ alkyl and C₁₋₆ haloalkyl, which are each optionally substituted with 1, 2, 3, or 4 independently selected R^{G} substituents;
R³ and R^{3a} are each independently selected from H, D, halo, CN, C₁₋₆ alkyl, and C₁₋₆ haloalkyl, and OR^{b3};
or R³ and R^{3a}, taken together with the carbon atom to which they are attached, form a C₃₋₇ cycloalkyl ring or a 3-7 membered heterocycloalkyl ring;
R⁴ and R^{4a} are each independently selected from H, D, halo, CN, C₁₋₆ alkyl, and C₁₋₆ haloalkyl, and OR^{b4}, wherein said C₁₋₆ alkyl and C₁₋₆ haloalkyl are each optionally substituted with 1, 2, 3, or 4 independently selected R^{G} substituents;
or R⁴ and R^{4a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
each R^{b4} is independently selected from C₁₋₆ alkyl and C₁₋₆ haloalkyl, which are each optionally substituted with 1, 2, 3, or 4 independently selected R^{G} substituents;
R⁵ is independently selected from D, halo, CN, C₁₋₆ alkyl, C₂₋₆ alkenyl, C₂₋₆ alkynyl, C₁₋₆ haloalkyl, C₃₋₄ cycloalkyl, C₃₋₇ cycloalkyl, phenyl, 4-7 membered heterocycloalkyl, 5-6 membered heteroaryl, OR^{a5} and NR^{c5}R^{d5}, wherein said C₁₋₆ alkyl, C₂₋₆ alkenyl, C₂₋₆ alkynyl, C₁₋₆ haloalkyl, C₃₋₄ cycloalkyl C₃₋₇ cycloalkyl, phenyl, 4-7 membered heterocycloalkyl, and 5-6 membered heteroaryl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents;
each R^{5A} is independently selected from OH, OCH₃, CN, halo, C₁₋₃ alkyl, C₁₋₃ haloalkyl, amino, C₁₋₃ alkylamino, and di(C₁₋₃ alkyl)amino;
R⁶ and R^{6a} are each independently selected from H and C₁₋₃ alkyl;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl;
each R^{b71} is independently selected from C₁₋₆ alkyl and C₁₋₆ haloalkyl;
R^{W} is selected from: 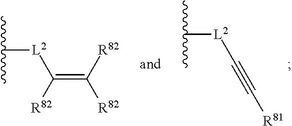
L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

36. The compound of claim 1, or a pharmaceutically acceptable salt thereof, wherein:
n is 0, 1, 2, 3, or 4;
W is N;
X is CH₂, C(O), or S(O)₂;
Z is Cy¹;
Cy¹ is phenyl, naphthyl, 5-6 membered heteroaryl, 9-10 membered heteroaryl, or 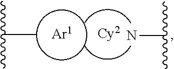
each of which is optionally substituted by 1, 2, or 3 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl, 5-6 membered heteroaryl, or 9-10 membered heteroaryl;
R² and R^{2a} are each independently selected from H, halo, CN, C₁₋₆ alkyl, and C₁₋₆ haloalkyl;
R³ and R^{3a} are each independently selected from H, halo, CN, C₁₋₆ alkyl, and C₁₋₆ haloalkyl;
R⁴ and R^{4a} are each independently selected H, halo, CN, C₁₋₆ alkyl, and C₁₋₆ haloalkyl;
each R⁵ is independently selected from halo, CN, C₁₋₃ alkyl, C₁₋₃ haloalkyl, OH, C₁₋₃ alkoxy, C₁₋₃ haloalkoxy, phenyl, and 5-6 membered heteroaryl, wherein said phenyl and 5-6 membered heteroaryl are each optionally substituted with 1 or 2 independently selected R^{5A} substituents;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂-3 alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from: 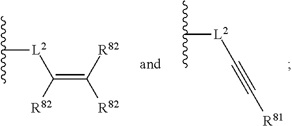
L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl; and
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

37. The compound of claim 1, or a pharmaceutically acceptable salt thereof, wherein:
n is 0, 1, 2, 3, or 4;
W is N;
X is CH₂, C(O), or S(O)₂;
Z is Cy¹;
Cy¹ is phenyl, naphthyl, 5-6 membered heteroaryl, 9-10 membered heteroaryl, or 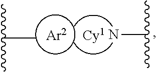
each of which is optionally substituted by 1, 2, or 3 independently selected R⁷ substituents;
Ar¹ is fused to Cy³ and Ar¹ is bonded to X;
Ar¹ is phenyl or 5-6 membered heteroaryl;
Cy³ is 4-8 membered heterocycloalkyl; and
R¹ is H;
Ring moiety A is phenyl, 5-6 membered heteroaryl, or 9-10 membered heteroaryl;
R² and R^{2a} are each independently selected from H, halo, CN, C₁₋₆ alkyl, and C₁₋₆ haloalkyl;
or R² and R^{2a}, taken together with the carbon atom to which they are attached, form a cyclopropyl ring;
R³ and R^{3a} are each independently selected from H, halo, CN, C₁₋₆ alkyl, and C₁₋₆ haloalkyl;
R⁴ and R^{4a} are each independently selected H, halo, CN, C₁₋₆ alkyl, and C₁₋₆ haloalkyl; each R⁵ is independently selected from halo, CN, C₁₋₃ alkyl, C₁₋₃ haloalkyl, OH, C₁₋₃ alkoxy, C₁₋₃ haloalkoxy, phenyl, and 5-6 membered heteroaryl, wherein said phenyl and 5-6 membered heteroaryl are each optionally substituted with 1 or 2 independently selected R^{5A} substituents;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂-3 alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from: 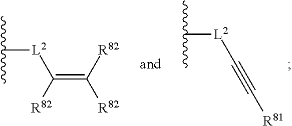
L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl; and
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

38. The compound of claim 1, wherein the compound is selected from:
N-(4-(3-(3,5-dichloro-4-hydroxyphenyl)-4,5,6,7-tetrahydropyrazolo[1,5-a]pyrazine-5-carbonyl)phenyl)acrylamide;
N-(4-(3-(3,5-dichloro-4-hydroxyphenyl)-4,5,6,7-tetrahydropyrazolo[1,5-a]pyrazine-5-carbonyl)-2-methoxyphenyl)acrylamide;
1-(5-(3-(3,5-dichloro-4-hydroxyphenyl)-4,5,6,7-tetrahydropyrazolo[1,5-a]pyrazine-5-carbonyl)isoindolin-2-yl)prop-2-en-1-one;
N-(5-(3-(3,5-dichloro-4-hydroxyphenyl)-4,5,6,7-tetrahydropyrazolo[1,5-a]pyrazine-5-carbonyl)-6-methoxypyridin-2-yl)acrylamide;
N-(6-(3-(3,5-dichloro-4-hydroxyphenyl)-4,5,6,7-tetrahydropyrazolo[1,5-a]pyrazine-5-carbonyl)pyridin-3-yl)acrylamide;
N-(6-(3-(3,5-dichloro-4-hydroxyphenyl)-4,5,6,7-tetrahydropyrazolo[1,5-a]pyrazine-5-carbonyl)-2-methoxypyridin-3-yl)acrylamide;
N-(3-(3-(3,5-dichloro-4-hydroxyphenyl)-4,5,6,7-tetrahydropyrazolo[1,5-a]pyrazine-5-carbonyl)-1-methyl-1H-pyrazol-5-yl)acrylamide;
N-(2-(3-(3,5-dichloro-4-hydroxyphenyl)-4,5,6,7-tetrahydropyrazolo[1,5-a]pyrazine-5-carbonyl)thiazol-5-yl)acrylamide;
N-(5-(3-(3,5-dichloro-4-hydroxyphenyl)-4,5,6,7-tetrahydropyrazolo[1,5-a]pyrazine-5-carbonyl)quinolin-8-yl)acrylamide;
1-(6-(3-(3,5-dichloro-4-hydroxyphenyl)-4,5,6,7-tetrahydropyrazolo[1,5-a]pyrazine-5-carbonyl)-1,3-dihydro-2H-pyrrolo[3,4-c]pyridin-2-yl)prop-2-en-1-one;
1-(5-(3-(3,5-dichloro-4-hydroxyphenyl)-4,5,6,7-tetrahydropyrazolo[1,5-a]pyrazine-5-carbonyl)indolin-1-yl)prop-2-en-1-one;
1-(6-(3-(3,5-dichloro-4-hydroxyphenyl)-4,5,6,7-tetrahydropyrazolo[1,5-a]pyrazine-5-carbonyl)-3,4-dihydroquinolin-1(2H)-yl)prop-2-en-1-one;
N-(4-(3-(3,5-dichloro-4-hydroxyphenyl)-4,5,6,7-tetrahydropyrazolo[1,5-a]pyrazine-5-carbonyl)naphthalen-1-yl)acrylamide;
N-(8-(3-(3,5-dichloro-4-hydroxyphenyl)-4,5,6,7-tetrahydropyrazolo[1,5-a]pyrazine-5-carbonyl)-2,3-dihydrobenzo[b][1,4]dioxin-5-yl)acrylamide;
N-(5-(3-(3,5-dichloro-4-hydroxyphenyl)-4,5,6,7-tetrahydropyrazolo[1,5-a]pyrazine-5-carbonyl)isoquinolin-8-yl)acrylamide;
N-(8-(3-(3,5-dichloro-4-hydroxyphenyl)-4,5,6,7-tetrahydropyrazolo[1,5-a]pyrazine-5-carbonyl)isoquinolin-5-yl)acrylamide;
N-(5-(3-(3,5-dichloro-4-hydroxyphenyl)-4,5,6,7-tetrahydropyrazolo[1,5-a]pyrazine-5-carbonyl)-4-methoxypyridin-2-yl)acrylamide;
N-(6-(3-(3,5-dichloro-4-hydroxyphenyl)-4,5,6,7-tetrahydropyrazolo[1,5-a]pyrazine-5-carbonyl)-2-(trifluoromethyl)pyridin-3-yl)acrylamide;
1-(5-(3-(3,5-dichloro-4-hydroxyphenyl)-4,5,6,7-tetrahydropyrazolo[1,5-a]pyrazine-5-carbonyl)-2,3-dihydro-1H-pyrrolo[2,3-b]pyridin-1-yl)prop-2-en-1-one;
1-(7-((3-(3,5-dichloro-4-hydroxyphenyl)-6,7-dihydropyrazolo[1,5-a]pyrazin-5(4H)-yl)sulfonyl)-2,3-dihydro-4H-benzo[b][1,4]oxazin-4-yl)prop-2-en-1-one;
N-(7-((3-(3,5-dichloro-4-hydroxyphenyl)-6,7-dihydropyrazolo[1,5-a]pyrazin-5(4H)-yl)sulfonyl)naphthalen-2-yl)acrylamide;
N-(4-((3-(3,5-dichloro-4-hydroxyphenyl)-6,7-dihydropyrazolo[1,5-a]pyrazin-5(4H)-yl)sulfonyl)phenyl)acrylamide;
N-(5-((3-(3,5-dichloro-4-hydroxyphenyl)-6,7-dihydropyrazolo[1,5-a]pyrazin-5(4H)-yl)methyl)pyridin-2-yl)acrylamide;
N-(4-((3-(3,5-dichloro-4-hydroxyphenyl)-6,7-dihydropyrazolo[1,5-a]pyrazin-5(4H)-yl)methyl)-2-methoxyphenyl)acrylamide;
1-(5-((3-(3,5-dichloro-4-hydroxyphenyl)-6,7-dihydropyrazolo[1,5-a]pyrazin-5(4H)-yl)methyl)isoindolin-2-yl)prop-2-en-1-one;
N-(2-chloro-4-((3-(3,5-dichloro-4-hydroxyphenyl)-6,7-dihydropyrazolo[1,5-a]pyrazin-5(4H)-yl)methyl)phenyl)acrylamide;
N-(4-(3-(3,5-dichloro-4-hydroxyphenyl)-6-methyl-4,5,6,7-tetrahydropyrazolo[1,5-a]pyrazine-5-carbonyl)phenyl)acrylamide;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(dimethylamino)but-2-enamide;
(E)-N-(5-(3-(3,5-dichloro-4-hydroxyphenyl)-4,5,6,7-tetrahydropyrazolo[1,5-a]pyrazine-5-carbonyl)quinolin-8-yl)-4-(dimethylamino)but-2-enamide;
(E)-N-(4-(3-(3,5-dichloro-4-hydroxyphenyl)-4,5,6,7-tetrahydropyrazolo[1,5-a]pyrazine-5-carbonyl)phenyl)-4-(dimethylamino)but-2-enamide;
(E)-N-(4-(3-(3,5-dichloro-4-hydroxyphenyl)-4,5,6,7-tetrahydropyrazolo[1,5-a]pyrazine-5-carbonyl)-2-fluorophenyl)-4-(dimethylamino)but-2-enamide;
(E)-1-(5-(3-(3,5-dichloro-4-hydroxyphenyl)-4,5,6,7-tetrahydropyrazolo[1,5-a]pyrazine-5-carbonyl)indolin-1-yl)-4-(dimethylamino)but-2-en-1-one;
(E)-N-(4-(3-(3,5-dichloro-4-hydroxyphenyl)-4,5,6,7-tetrahydropyrazolo[1,5-a]pyrazine-5-carbonyl)phenyl)-4-(piperidin-1-yl)but-2-enamide;
(E)-N-(4-(3-(3,5-dichloro-4-hydroxyphenyl)-4,5,6,7-tetrahydropyrazolo[1,5-a]pyrazine-5-carbonyl)phenyl)-4-morpholinobut-2-enamide;
(E)-N-(4-(3-(3,5-dichloro-4-hydroxyphenyl)-4,5,6,7-tetrahydropyrazolo[1,5-a]pyrazine-5-carbonyl)phenyl)-4-(diethylamino)but-2-enamide;
(E)-1-(5-(3-(3,5-dichloro-4-hydroxyphenyl)-4,5,6,7-tetrahydropyrazolo[1,5-a]pyrazine-5-carbonyl)isoindolin-2-yl)-4-(dimethylamino)but-2-en-1-one;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(piperidin-1-yl)but-2-enamide;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(pyrrolidin-1-yl)but-2-enamide;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-morpholinobut-2-enamide;
(E)-N-(4-(3-(3,5-dichloro-4-hydroxyphenyl)-4,5,6,7-tetrahydropyrazolo[1,5-a]pyrazine-5-carbonyl)-2-methoxyphenyl)-4-(diethylamino)but-2-enamide;
(E)-N-(4-(3-(3,5-dichloro-4-hydroxyphenyl)-4,5,6,7-tetrahydropyrazolo[1,5-a]pyrazine-5-carbonyl)-3-methoxyphenyl)-4-(dimethylamino)but-2-enamide;
N-(2-chloro-4-(3-(3,5-dichloro-4-hydroxyphenyl)-4,5,6,7-tetrahydropyrazolo[1,5-a]pyrazine-5-carbonyl)phenyl)acrylamide;
N-(4-(3-(3,5-dichloro-4-hydroxyphenyl)-4,5,6,7-tetrahydropyrazolo[1,5-a]pyrazine-5-carbonyl)-2-fluorophenyl)acrylamide;
N-(3-chloro-4-(3-(3,5-dichloro-4-hydroxyphenyl)-4,5,6,7-tetrahydropyrazolo[1,5-a]pyrazine-5-carbonyl)phenyl)acrylamide;
N-(4-(3-(3,5-dichloro-4-hydroxyphenyl)-4,5,6,7-tetrahydropyrazolo[1,5-a]pyrazine-5-carbonyl)-3-(trifluoromethyl)phenyl)acrylamide;
N-(5-(3-(3,5-dichloro-4-hydroxyphenyl)-4,5,6,7-tetrahydropyrazolo[1,5-a]pyrazine-5-carbonyl)pyridin-2-yl)acrylamide;
N-(4-(3-(3,5-dichloro-4-hydroxyphenyl)-4,5,6,7-tetrahydropyrazolo[1,5-a]pyrazine-5-carbonyl)-3-methoxyphenyl)acrylamide;
N-(4-(3-(3,5-dichloro-4-hydroxyphenyl)-4,5,6,7-tetrahydropyrazolo[1,5-a]pyrazine-5-carbonyl)-2-ethoxyphenyl)acrylamide;
N-(4-(3-(3,5-dichloro-4-hydroxyphenyl)-4,5,6,7-tetrahydropyrazolo[1,5-a]pyrazine-5-carbonyl)-2-isopropoxyphenyl)acrylamide;
N-(4-(3-(3,5-dichloro-4-hydroxyphenyl)-4,5,6,7-tetrahydropyrazolo[1,5-a]pyrazine-5-carbonyl)-2-(trifluoromethoxy)phenyl)acrylamide;
N-(4-(3-(3,5-dichloro-4-hydroxyphenyl)-4,5,6,7-tetrahydropyrazolo[1,5-a]pyrazine-5-carbonyl)-2-(methoxy-d₃)phenyl)acrylamide; and
N-(3-cyano-4-(3-(3,5-dichloro-4-hydroxyphenyl)-4,5,6,7-tetrahydropyrazolo[1,5-a]pyrazine-5-carbonyl)phenyl)acrylamide;
or a pharmaceutically acceptable salt of any of the aforementioned.

39. The compound of claim 1, wherein the compound is selected from:
(E)-1-(5-((3-(3,5-dichloro-4-hydroxyphenyl)-6,7-dihydropyrazolo[1,5-a]pyrazin-5(4H)-yl)methyl)-2,3-dihydro-1H-pyrrolo[3,2-b]pyridin-1-yl)-4-(piperidin-1-yl)but-2-en-1-one; and
(E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)indolin-1-yl)-4-morpholinobut-2-en-1-one;
or a pharmaceutically acceptable salt of any of the aforementioned.

40. The compound of claim 1, wherein the compound is selected from:
(E)-1-(5-(3′-(2-(2,3-dihydrobenzofuran-7-yl)pyridin-4-yl)-4′,5′-dihydro-7′H-spiro[cyclopropane-1,6′-pyrazolo[1,5-a]pyrazine]-5′-carbonyl)indolin-1-yl)-4-(dimethylamino)but-2-en-1-one;
(E)-4-(dimethylamino)-1-(5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one;
(E)-4-(3-azabicyclo[3.1.1]heptan-3-yl)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)but-2-en-1-one;
(E)-1-(5-(3′-(2-methoxypyridin-4-yl)-4′,5′-dihydro-7′H-spiro[cyclopropane-1,6′-pyrazolo[1,5-a]pyrazine]-5′-carbonyl)indolin-1-yl)-4-(piperidin-1-yl)but-2-en-1-one;
(E)-1-(5-(3′-(3,5-dichloro-4-hydroxyphenyl)-4′,5′-dihydro-7′H-spiro[cyclobutane-1,6′-pyrazolo[1,5-a]pyrazine]-5′-carbonyl)indolin-1-yl)-4-(dimethylamino)but-2-en-1-one;
(E)-4-(3-azabicyclo[3.1.1]heptan-3-yl)-1-(5-((3-(3,5-dichloro-4-hydroxyphenyl)-6,7-dihydropyrazolo[1,5-a]pyrazin-5(4H)-yl)methyl)-2,3-dihydro-1H-pyrrolo[3,2-b]pyridin-1-yl)but-2-en-1-one;
(E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)indolin-1-yl)-4-(dimethylamino)but-2-en-1-one;
(E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)-4-morpholinobut-2-en-1-one;
(E)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)-4-(piperidin-1-yl)but-2-en-1-one;
(E)-4-(dimethylamino)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one;
(E)-1-(5-(3′-(3,5-dichloro-4-hydroxyphenyl)-4′,5′-dihydro-7′H-spiro[cyclobutane-1,6′-pyrazolo[1,5-a]pyrazine]-5′-carbonyl)indolin-1-yl)-4-morpholinobut-2-en-1-one;
(R,E)-1-(5-(3-(3,5-dichloro-4-hydroxyphenyl)-6-methyl-4,5,6,7-tetrahydropyrazolo[1,5-a]pyrazine-5-carbonyl)-4-methoxyindolin-1-yl)-4-morpholinobut-2-en-1-one;
(E)-1-(5-(3′-(2-methoxypyrimidin-4-yl)-4′,5′-dihydro-7′H-spiro[cyclopropane-1,6′-pyrazolo[1,5-a]pyrazine]-5′-carbonyl)indolin-1-yl)-4-morpholinobut-2-en-1-one;
(R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-(2,2-dimethylpyrrolidin-1-yl)but-2-en-1-one;
N-(4-(3′-(3,5-dichloro-4-hydroxyphenyl)-4′,5′-dihydro-7′H-spiro[cyclopropane-1,6′-pyrazolo[1,5-a]pyrazine]-5′-carbonyl)-2-methoxyphenyl)acrylamide;
(R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-(piperidin-1-yl)but-2-en-1-one;
(E)-4-(3-fluoroazetidin-1-yl)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one;
(R)-1-(5-(3-(3,5-dichloro-4-hydroxyphenyl)-6-methyl-4,5,6,7-tetrahydropyrazolo[1,5-a]pyrazine-5-carbonyl)isoindolin-2-yl)prop-2-en-1-one;
(R,E)-N-(4-(3-(3,5-dichloro-4-hydroxyphenyl)-6-methyl-4,5,6,7-tetrahydropyrazolo[1,5-a]pyrazine-5-carbonyl)-2-fluorophenyl)-4-morpholinobut-2-enamide;
(R,E)-1-(5-(3-(3,5-dichloro-4-hydroxyphenyl)-6-methyl-4,5,6,7-tetrahydropyrazolo[1,5-a]pyrazine-5-carbonyl)indolin-1-yl)-4-morpholinobut-2-en-1-one;
(R,E)-N-(4-(3-(3,5-dichloro-4-hydroxyphenyl)-6-isopropyl-4,5,6,7-tetrahydropyrazolo[1,5-a]pyrazine-5-carbonyl)-2-methoxyphenyl)-4-(dimethylamino)but-2-enamide; and
(E)-4-(3-fluoroazetidin-1-yl)-1-(5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one;
or a pharmaceutically acceptable salt of any of the aforementioned.

41. The compound of claim 1, wherein the compound is (E)-4-(3-azabicyclo[3.1.1]heptan-3-yl)-1-(5-(3′-(3,5-dichloro-4-hydroxyphenyl)-4′,5′-dihydro-7′H-spiro[cyclopropane-1,6′-pyrazolo[1,5-a]pyrazine]-5′-carbonyl)-4-methoxyindolin-1-yl)but-2-en-1-one, or a pharmaceutically acceptable salt thereof.

42. The compound of claim 1, wherein the compound is (E)-4-(dimethylamino)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one, or a pharmaceutically acceptable salt thereof.

43. The compound of claim 1, wherein the compound is (E)-4-(3-fluoroazetidin-1-yl)-1-(4-methoxy-5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one, or a pharmaceutically acceptable salt thereof.

44. The compound of claim 1, wherein the compound is (E)-4-(3-fluoroazetidin-1-yl)-1-(5-(3′-(2-(2-methoxyphenyl)pyridin-4-yl)-4′,5′-dihydro-7′H-spiro[cyclopropane-1,6′-pyrazolo[1,5-a]pyrazine]-5′-carbonyl)indolin-1-yl)but-2-en-1-one, or a pharmaceutically acceptable salt thereof.

45. The compound of claim 1, or a pharmaceutically acceptable salt thereof, wherein the compound is deuterated.

46. A pharmaceutical composition comprising the compound of claim 1, or a pharmaceutically acceptable salt thereof, and a pharmaceutically acceptable carrier.

47. A method of inhibiting VRK1, comprising contacting the VRK1 with the compound of claim 1, or a pharmaceutically acceptable salt thereof.

48. A method of inhibiting VRK1 in a patient, comprising administering to the patient a compound of claim 1, or a pharmaceutically acceptable salt thereof.

49. A method of selectively inhibiting VRK1 over VRK2, comprising contacting the VRK1 and VRK2 with a VRK1 inhibitor, or a pharmaceutically acceptable salt thereof.

50. A method of covalently binding a cysteine in VRK1 with a VRK1 inhibitor, or a pharmaceutically acceptable salt thereof.

51. The method of claim 50, further comprising binding with a water in the binding pocket of VRK1.

52. The method of claim 50, further comprising binding with Phe134 of VRK1.

53. The method of claim 50, further comprising binding with Lys71 of VRK1.

54. The method of claim 50, further comprising binding with Asp132 of VRK1.

55. The method of claim 50, further comprising binding with the phosphate-binding loop of VRK1.

56. The method of claim 49, wherein the VRK1 inhibitor is a compound of Formula (I): 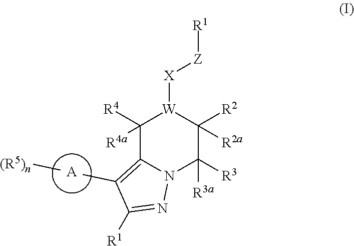
or a pharmaceutically acceptable salt thereof, wherein:
n is 0, 1, 2, 3, 4, 5, or 6;
W is CH or N;
when W is N, then X is CR⁶R^{6a}, C(O), S(O), or S(O)₂;
when W is CH, then X is NR^{6b}, CR⁶R^{6a} C(O), S(O), or S(O)₂;
Z is -Cy¹- or -Cy¹-Cy²-, wherein Cy¹ is bound to X, provided that when Z is -Cy¹-Cy²-, then Cy² is bound to R^{W};
Cy¹ is C₃₋₁₂ cycloalkyl, 6-10 membered aryl, 4-12 membered heterocycloalkyl, and 5-10 membered heteroaryl, each of which is optionally substituted by 1, 2, 3, 4, 5, or 6 independently selected R⁷ substituents;
Cy² is C₃₋₁₂ cycloalkyl, 6-10 membered aryl, 4-12 membered heterocycloalkyl, and 5-10 membered heteroaryl, each of which is optionally substituted by 1, 2, 3, 4, 5, or 6 independently selected R⁷ substituents;
Ring moiety A is C₃₋₁₀ cycloalkyl, 6-10 membered aryl, 4-10 membered heterocycloalkyl, or 5-10 membered heteroaryl;
R¹ is H, D, halo, CN, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, or C₁₋₃ alkoxy-C₁₋₄ alkyl;
R² and R^{2a} are each independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or R² and R^{2a}, taken together with the carbon atom to which they are attached, form a C₃₋₇ cycloalkyl ring or a 3-7 membered heterocycloalkyl ring, which is optionally substituted with 1, 2, 3, or 4 independently selected R^{G} substituents;
or R² and R^{2a}, taken together with the carbon atom to which they are attached, form a C(O) group;
each R^{a2}, R^{c2}, and R^{d2} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c2} and R^{d2} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
R³ and R^{3a} are each independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)NR^{c3}R^{d3}, NR^{c3}S(O)R^{d3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c3} and R^{d3} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents;
each R^{b3} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
R⁴ and R^{4a} are each independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c4} and R^{d4} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{G} substituents;
each R^{b4} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R⁵ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
each R^{a}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
or, any R^{c5} and R^{d5} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)NR^{c51}R^{d51}, NR^{c51}S(O)R^{b51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, S(O)(═NR^{e51})R^{b51}, SF₅, P(O)R^{f51}R^{g51}, OP(O)(OR^{h51})(ORⁱ⁵¹), P(O)(OR^{h51})(ORⁱ⁵¹), and BR^{j51}R^{k51}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5B} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5B} substituents;
or, any R^{c51} and R^{d51} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{5B} substituents;
each R^{b51} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{5B} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f51} and R^{g51} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h51} and Rⁱ⁵¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a52}, SR^{a52}, NHOR^{a52}, C(O)R^{b52}, C(O)NR^{c52}R^{d52}, C(O)NR^{c52}(OR^{a52}), C(O)OR^{a52}, OC(O)R^{b52}, OC(O)NR^{c52}R^{d52}, NR^{c52}R^{d52}, NR^{c52}NR^{c52}R^{d52}, NR^{c52}C(O)R^{b52}, NR^{c52}C(O)OR^{a52}, NR^{c52}C(O)NR^{c52}R^{d52}, C(═NR^{e52})R^{b52}, C(═NR^{e52})NR^{c52}R^{d52}, NR^{c52}C(═NR^{e52})NR^{c52}R^{d52}, NR^{c52}C(═NR^{e52})R^{b52}, NR^{c52}S(O)NR^{c52}R^{d52}, NR^{c52}S(O)R^{b52}, NR^{c52}S(O)₂R^{b52}, NR^{c52}S(O)(═NR^{e52})R^{b52}, NR^{c52}S(O)₂NR^{c52}R^{d52}, S(O)R^{b52}, S(O)NR^{c52}R^{d52}, S(O)₂R^{b52}, S(O)₂NR^{c52}R^{d52}, OS(O)(═NR^{e52})R^{b52}, OS(O)₂R^{b52}, S(O)(═NR^{e52})R^{b52}, SF₅, P(O)R^{f52}R^{g52}, OP(O)(OR^{h52})(ORⁱ⁵²), P(O)(OR^{h52})(ORⁱ⁵²), and BR^{j52}R^{k52}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a52}, R^{c52}, and R^{d52} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c52} and R^{d52} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b52} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e52} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f52} and R^{g52} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h52} and Rⁱ⁵² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j52} and R^{k52} is independently selected from OH, C₁₋₆ alkoxy, and C₁₋₆ haloalkoxy;
or any R^{j52} and R^{k52} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁶ and R^{6a} are each independently selected from H, D, halo, CN, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
R^{6b} is selected from H, C₁₋₆ alkyl, C₂₋₆ alkenyl, C₂₋₆ alkynyl, C₁₋₆ haloalkyl, and C₃₋₄ cycloalkyl, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R⁷ is independently selected from D, halo, CN, NO₂, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═R^{e7})NR^{c7}R^{d7}, NR^{c7}C(═NR^{e7})R^{b7}, NR^{c7}S(O)NR^{c7}R^{d7}, NR^{c7}S(O)R^{b7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, OS(O)(═NR^{e7})R^{b7}, OS(O)₂R^{b7}, S(O)(═NR^{e7})R^{b7}, SF₅, P(O)R^{f7}R^{g7}, OP(O)(OR^{h7})(ORⁱ⁷), P(O)(OR^{h7})(ORⁱ⁷), and BR^{j7}R^{k7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
or, any R^{c7} and R^{d7} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h7} and Rⁱ⁷ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j7} and R^{k7} is independently selected from OH, C₁₋₆ alkoxy, and C₁₋₆ haloalkoxy;
or any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, NHOR^{a71}, C(O)R^{b71}, C(O)NR^{c71}R^{d71}, C(O)NR^{c71}(OR^{a71}), C(O)OR^{a71}, OC(O)R^{b71}, OC(O)NR^{c71}R^{d71}, NR^{c71}R^{d71}, NR^{c71}NR^{c71}R^{d71}, NR^{c71}C(O)R^{b71}, NR^{c71}C(O)OR^{a71}, NR^{c71}C(O)NR^{c71}R^{d71}, C(═NR^{e71})R^{b71}, C(═NR^{e71})NR^{c71}R^{d71}, NR^{c71}C(═NR^{e71})NR^{c71}R^{d71}, NR^{c71}C(═NR^{e71})R^{b71}, NR^{c71}S(O)NR^{c71}R^{d71}, NR^{c71}S(O)R^{b71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, OS(O)₂R^{b71}, S(O)(═NR^{e71})R^{b71}, SF₅, P(O)R^{f71}R^{g71}, OP(O)(OR^{h71})(ORⁱ⁷¹), P(O)(OR^{h71})(ORⁱ⁷¹), and BR^{j71}R^{k71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c7}l, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f71} and R^{g71} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h71} and Rⁱ⁷¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j71} and R^{k71} is independently selected from OH, C₁₋₆ alkoxy, and C₁₋₆ haloalkoxy;
or any R^{j71} and R^{k71} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a72}, SR^{a72}, NHOR^{a72}, C(O)R^{b72}, C(O)NR^{c72}R^{d72}, C(O)NR^{c72}(OR^{a72}), C(O)OR^{a72}, OC(O)R^{b72}, OC(O)NR^{c72}R^{d72}, NR^{c72}R^{d72}, NR^{c72}NR^{c72}R^{d72}, NR^{c72}C(O)R^{b72}, NR^{c72}C(O)OR^{a72}, NR^{c72}C(O)NR^{c72}R^{d72}, C(═NR^{e72})R^{b72}, C(═NR^{e72})NR^{c72}R^{d72}, NR^{c72}C(═NR^{e72})NR^{c72}R^{d72}, NR^{c72}C(═NR^{e72})R^{b72}, NR^{c72}S(O)NR^{c72}R^{d72}, NR^{c72}S(O)R^{b72}, NR^{c72}S(O)₂R^{b72}, NR^{c72}S(O)(═NR^{e72})R^{b72}, NR^{c72}S(O)₂NR^{c72}R^{d72}, S(O)R^{b72}, S(O)NR^{c72}R^{d72}, S(O)₂R^{b72}, S(O)₂NR^{c72}R^{d72}, OS(O)(═NR^{e72})R^{b72}, OS(O)₂R^{b72}, S(O)(═NR^{e72})R^{b72}, SF₅, P(O)R^{f72}R^{g72}, OP(O)(OR^{h72})(ORⁱ⁷²), P(O)(OR^{h72})(ORⁱ⁷²), and BR^{j72}R^{k72}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a72}, R^{c72}, and R^{d72} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c72} and R^{d72} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b72} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e72} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f72} and R^{g72} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h72} and Rⁱ⁷² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j72} and R^{k72} is independently selected from OH, C₁₋₆ alkoxy, and C₁₋₆ haloalkoxy;
or any R^{j72} and R^{k72} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{W} is a covalent warhead moiety having a reactive functional group capable of covalently binding to one or more cysteine residues in a protein; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

57. A method of treating a disease or disorder associated with VRK1 in a patient, comprising administering to the patient a therapeutically effective amount of the compound of claim 1, or pharmaceutically acceptable salt thereof.

58. The method of claim 57, wherein the disease or disorder is cancer.

59. The method of claim 58, wherein the cancer is sarcoma, colorectal adenocarcinoma, acute myeloid leukemia, glioblastoma, and neuroblastoma.

60. The method of claim 59, wherein the glioblastoma or neuroblastoma have overexpression of VRK1.

61. The method of claim 59, wherein the glioblastoma or neuroblastoma have underexpression of VRK2.

62. The method of claim 58, wherein the cancer is bone cancer, cancer of the neck and head, rectal cancer, esophageal cancer, sarcoma of soft tissue, neoplasm of the central nervous system (CNS), brain stem, glioma, colon cancer, Ewing's sarcoma, brain cancer, astrocytoma, neuroblastoma, intestinal cancer, nervous system cancers, osteosarcoma, cancer of the small bowel, adenocarcinoma of the small bowel, lymphoma of the small bowel, carcinoid tumors of the small bowel, Kaposi's sarcoma of the small bowel, leiomyoma of the small bowel, hemangioma of the small bowel, lipoma of the small bowel, neurofibroma of the small bowel, fibroma of the small bowel, cancer of the large bowel, adenocarcinoma of the large bowel, tubular adenoma of the large bowel, villous adenoma of the large bowel, hamartoma of the large bowel, or leiomyoma of the large bowel.
